(19) United States
(12) Patent Application Publication
(10) Pub. No.: US 2024/0417706 A1
(43) Pub. Date: Dec. 19, 2024

(54) SYSTEMS AND METHODS FOR THE TREATMENT OF HEMOGLOBINOPATHIES

(71) Applicant: EDITAS MEDICINE, INC., Cambridge, MA (US)

(72) Inventors: Jennifer Leah GORI, Jamaica Plain, MA (US); Luis A. BARRERA, Somerville, MA (US); Edouard AUPEPIN DE LAMOTHE-DREUZY, Boston, MA (US); Jack HEATH, Boston, MA (US)

(73) Assignee: EDITAS MEDICINE, INC., Cambridge, MA (US)

(21) Appl. No.: 18/394,385

(22) Filed: Dec. 22, 2023

Related U.S. Application Data
(63) Continuation of application No. 16/569,336, filed on Sep. 12, 2019, now Pat. No. 11,851,690.
Application No. 16/569,336 is a continuation of application No. PCT/US2018/022516, filed on Mar. 14, 2018.
(60) Provisional application No. 62/471,342, filed on Mar. 14, 2017.

(51) Int. Cl. C12N 9/22; A61K 31/7105; A61K 38/46; C12N 15/11

(52) U.S. Cl. CPC C12N 9/22; A61K 31/7105; A61K 38/465; C12N 15/111; C12N 2310/20; C12N 2320/30

(57) ABSTRACT

Genome editing systems, guide RNAs, and CRISPR-mediated methods are provided for altering portions of the HBG1 and HBG2 loci, portions of the erythroid specific enhancer of the BCL11A gene, or a combination thereof, in cells and increasing expression of fetal hemoglobin.

### REFERENCE TO RELATED APPLICATIONS

The present application is a continuation of U.S. patent application Ser. No. 16/569,336, filed Sep. 12, 2019, which is a continuation of International Application No. PCT/US2018/022516, filed Mar. 14, 2018, which claims the benefit of U.S. Provisional Application No. 62/471,342, filed Mar. 14, 2017, the contents of which are hereby incorporated by reference in their entirety.

### SEQUENCE LISTING

This application contains a ST.26 compliant Sequence Listing, which was submitted in XML format via Patent Center, and is hereby incorporated by reference in its entirety. The XML copy, created on Apr. 5, 2024, is named SubstituteSequenceListing.xml and is 1,350,000 bytes in size.

### FIELD

This disclosure relates to genome editing systems and methods for altering a target nucleic acid sequence, or modulating expression of a target nucleic acid sequence, and applications thereof in connection with the alteration of genes encoding hemoglobin subunits and/or treatment of hemoglobinopathies.

### BACKGROUND

Hemoglobin (Hb) carries oxygen in erythrocytes or red blood cells (RBCs) from the lungs to tissues. During prenatal development and until shortly after birth, hemoglobin is present in the form of fetal hemoglobin (HbF), a tetrameric protein composed of two alpha (α)-globin chains and two gamma (γ)-globin chains. HbF is largely replaced by adult hemoglobin (HbA), a tetrameric protein in which the γ-globin chains of HbF are replaced with beta (β)-globin chains, through a process known as globin switching. The average adult makes less than 1% HbF out of total hemoglobin (Thein 2009). The α-hemoglobin gene is located on chromosome 16, while the β-hemoglobin gene (HBB), A gamma (Aγ)-globin chain (HBG1, also known as gamma globin A), and G gamma (Gγ)-globin chain (HBG2, also known as gamma globin G) are located on chromosome 11 within the globin gene cluster (also referred to as the globin locus).

Mutations in HBB can cause hemoglobin disorders (i.e., hemoglobinopathies) including sickle cell disease (SCD) and beta-thalassemia (β-Thal). Approximately 93,000 people in the United States are diagnosed with a hemoglobinopathy. Worldwide, 300,000 children are born with hemoglobinopathies every year (Angastiniotis 1998). Because these conditions are associated with HBB mutations, their symptoms typically do not manifest until after globin switching from HbF to HbA.

SCD is the most common inherited hematologic disease in the United States, affecting approximately 80,000 people (Brousseau 2010). SCD is most common in people of African ancestry, for whom the prevalence of SCD is 1 in 500. In Africa, the prevalence of SCD is 15 million (Aliyu 2008). SCD is also more common in people of Indian, Saudi Arabian and Mediterranean descent. In those of Hispanic-American descent, the prevalence of sickle cell disease is 1 in 1,000 (Lewis 2014).

SCD is caused by a single homozygous mutation in the HBB gene, c. 17A>T (HbS mutation). The sickle mutation is a point mutation (GAG>GTG) on HBB that results in substitution of valine for glutamic acid at amino acid position 6 in exon 1. The valine at position 6 of the β-hemoglobin chain is hydrophobic and causes a change in conformation of the β-globin protein when it is not bound to oxygen. This change of conformation causes HbS proteins to polymerize in the absence of oxygen, leading to deformation (i.e., sickling) of RBCs. SCD is inherited in an autosomal recessive manner, so that only patients with two HbS alleles have the disease. Heterozygous subjects have sickle cell trait, and may suffer from anemia and/or painful crises if they are severely dehydrated or oxygen deprived.

Sickle shaped RBCs cause multiple symptoms, including anemia, sickle cell crises, vaso-occlusive crises, aplastic crises, and acute chest syndrome. Sickle shaped RBCs are less clastic than wild-type RBCs and therefore cannot pass as easily through capillary beds and cause occlusion and ischemia (i.e., vaso-occlusion). Vaso-occlusive crisis occurs when sickle cells obstruct blood flow in the capillary bed of an organ leading to pain, ischemia, and necrosis. These episodes typically last 5-7 days. The spleen plays a role in clearing dysfunctional RBCs, and is therefore typically enlarged during early childhood and subject to frequent vaso-occlusive crises. By the end of childhood, the spleen in SCD patients is often infarcted, which leads to autosplenectomy. Hemolysis is a constant feature of SCD and causes anemia. Sickle cells survive for 10-20 days in circulation, while healthy RBCs survive for 90-120 days. SCD subjects are transfused as necessary to maintain adequate hemoglobin levels. Frequent transfusions place subjects at risk for infection with HIV, Hepatitis B, and Hepatitis C. Subjects may also suffer from acute chest crises and infarcts of extremities, end organs, and the central nervous system.

Subjects with SCD have decreased life expectancies. The prognosis for patients with SCD is steadily improving with careful, life-long management of crises and anemia. As of 2001, the average life expectancy of subjects with sickle cell disease was the mid-to-late 50's. Current treatments for SCD involve hydration and pain management during crises, and transfusions as needed to correct anemia.

Thalassemias (e.g., β-Thal, δ-Thal, and β/δ-Thal) cause chronic anemia. β-Thal is estimated to affect approximately 1 in 100,000 people worldwide. Its prevalence is higher in certain populations, including those of European descent, where its prevalence is approximately 1 in 10,000. β-Thal major, the more severe form of the disease, is life-threatening unless treated with lifelong blood transfusions and chelation therapy. In the United States, there are approximately 3,000 subjects with β-Thal major. β-Thal intermedia does not require blood transfusions, but it may cause growth delay and significant systemic abnormalities, and it frequently requires lifelong chelation therapy. Although HbA makes up the majority of hemoglobin in adult RBCs, approximately 3% of adult hemoglobin is in the form of HbA2, an HbA variant in which the two γ-globin chains are replaced with two delta (Δ)-globin chains. δ-Thal is associated with mutations in the A hemoglobin gene (HBD) that cause a loss of HBD expression. Co-inheritance of the HBD mutation can mask a diagnosis of β-Thal (i.e., β/δ-Thal) by decreasing the level of HbA2 to the normal range (Bouva 2006). β/δ-Thal is usually caused by deletion of the HBB and HBD sequences in both alleles. In homozygous (δo/δo βo/βo) patients, HBG is expressed, leading to production of HbF alone.

Like SCD, β-Thal is caused by mutations in the HBB gene. The most common HBB mutations leading to β-Thal are: c.-136C>G, c.92+1G>A, c.92+6T>C, c.93-21G>A, c.118C>T, c.316-106C>G, c.25_26delAA, c.27_28insG, c.92+5G>C, c.118C>T. c.135delC, c.315+1G>A, c.-78A>G, c.52A>T, c.59A>G, c.92+5G>C, c. 124_127delTTCT, c.316-197C>T, c.-78A>G, c.52A>T, c. 124_127delTTCT, c.316-197C>T, c.-138C>T, c.-79A>G, c.92+5G>C, c.75T>A, c.316-2A>G, and c.316-2A>C. These and other mutations associated with β-Thal cause mutated or absent β-globin chains, which causes a disruption of the normal Hb α-hemoglobin to β-hemoglobin ratio. Excess α-globin chains precipitate in erythroid precursors in the bone marrow.

In β-Thal major, both alleles of HBB contain nonsense, frameshift, or splicing mutations that leads to complete absence of β-globin production (denoted β⁰/β⁰). β-Thal major results in severe reduction in β-globin chains, leading to significant precipitation of α-globin chains in RBCs and more severe anemia.

β-Thal intermedia results from mutations in the 5′ or 3′ untranslated region of HBB, mutations in the promoter region or polyadenylation signal of HBB, or splicing mutations within the HBB gene. Patient genotypes are denoted βo/β+ or β+/β+. βo represents absent expression of a β-globin chain; β+ represents a dysfunctional but present β-globin chain. Phenotypic expression varies among patients. Since there is some production of β-globin, β-Thal intermedia results in less precipitation of α-globin chains in the erythroid precursors and less severe anemia than β-Thal major. However, there are more significant consequences of erythroid lineage expansion secondary to chronic anemia.

Subjects with β-Thal major present between the ages of 6 months and 2 years, and suffer from failure to thrive, fevers, hepatosplenomegaly, and diarrhea. Adequate treatment includes regular transfusions. Therapy for β-Thal major also includes splenectomy and treatment with hydroxyurea. If patients are regularly transfused, they will develop normally until the beginning of the second decade. At that time, they require chelation therapy (in addition to continued transfusions) to prevent complications of iron overload. Iron overload may manifest as growth delay or delay of sexual maturation. In adulthood, inadequate chelation therapy may lead to cardiomyopathy, cardiac arrhythmias, hepatic fibrosis and/or cirrhosis, diabetes, thyroid and parathyroid abnormalities, thrombosis, and osteoporosis. Frequent transfusions also put subjects at risk for infection with HIV, hepatitis B and hepatitis C.

β-Thal intermedia subjects generally present between the ages of 2-6 years. They do not generally require blood transfusions. However, bone abnormalities occur due to chronic hypertrophy of the erythroid lineage to compensate for chronic anemia. Subjects may have fractures of the long bones due to osteoporosis. Extramedullary erythropoiesis is common and leads to enlargement of the spleen, liver, and lymph nodes. It may also cause spinal cord compression and neurologic problems. Subjects also suffer from lower extremity ulcers and are at increased risk for thrombotic events, including stroke, pulmonary embolism, and deep vein thrombosis. Treatment of β-Thal intermedia includes splenectomy, folic acid supplementation, hydroxyurea therapy, and radiotherapy for extramedullary masses. Chelation therapy is used in subjects who develop iron overload.

Life expectancy is often diminished in β-Thal patients. Subjects with β-Thal major who do not receive transfusion therapy generally die in their second or third decade. Subjects with β-Thal major who receive regular transfusions and adequate chelation therapy can live into their fifth decade and beyond. Cardiac failure secondary to iron toxicity is the leading cause of death in β-Thal major subjects due to iron toxicity.

A variety of new treatments are currently in development for SCD and β-Thal. Delivery of an anti-sickling HBB gene via gene therapy is currently being investigated in clinical trials. However, the long-term efficacy and safety of this approach is unknown. Transplantation with hematopoietic stem cells (HSCs) from an HLA-matched allogeneic stem cell donor has been demonstrated to cure SCD and β-Thal, but this procedure involves risks including those associated with ablation therapy, which is required to prepare the subject for transplant, increases risk of life-threatening opportunistic infections, and risk of graft vs. host disease after transplantation. In addition, matched allogeneic donors often cannot be identified. Thus, there is a need for improved methods of managing these and other hemoglobinopathies.

### SUMMARY

Provided herein are genome editing systems, guide RNAs, and CRISPR-mediated methods for altering one or more γ-globin genes (e.g., HBG1, HBG2, or HBG1 and HBG2), the erythroid specific enhancer of the BCL11A gene (BCL11Ae), or a combination thereof, and increasing expression of fetal hemoglobin (HbF). In certain embodiments, genome editing systems, guide RNAs, and CRISPR-mediated methods may alter a 13 nucleotide (nt) target region that is 5′ of the transcription site of the HBG1, HBG2, or HBG1 and HBG2 gene (“13 nt target region”). In certain embodiments, one or more gRNAs comprising a targeting domain set forth in SEQ ID NOs:251-901 or 940-942 may be used to introduce alterations in the 13 nt target region. In certain embodiments, one or more gRNAs comprising a sequence set forth in SEQ ID NOs:970-979 may be used to introduce alterations in the 13 nt target region. In certain embodiments, genome editing systems, guide RNAs, and CRISPR-mediated methods may alter a GATA1 binding motif in BCL11Ae that is in the +58 DNase I hypersensitive site (DHS) region of intron 2 of the BCL11A gene (“GATA1 binding motif in BCL11Ae”). In certain embodiments, one or more gRNAs comprising a targeting domain set forth in SEQ ID NOs:952-955 may be used to introduce alterations in the GATA1 binding motif in BCL11Ae. In certain embodiments, one or more gRNAs may be used to introduce alterations in the GATA1 binding motif in BCL11Ae and one or more gRNAs may be used to introduce alterations in the 13 nt target region of HBG1 and/or HBG2.

Provided herein in certain embodiments are gRNAs for use in CRISPR/Cas-mediated methods of increasing expression (i.e., transcriptional activity) of one or more γ-globin genes (e.g., HBG1, HBG2, or HBG1 and HBG2). In certain embodiments, the gRNAs may be generated via in vitro transcription or chemical synthesis. In certain embodiments, the gRNA may comprise one or more modifications. In certain embodiments, the one or more modifications may be selected from the group consisting of phosphorothioate and 2′-O-methyl groups. In certain embodiments, the one or more modifications may be at or near the 5′ end, 3′ end, or both of the gRNA. In certain embodiments, the gRNA may comprise a truncated form of a full-length gRNA (e.g., SEQ ID NO:970). In certain embodiments, the gRNA may comprise, consist essentially of, or consist of a sequence selected from the group consisting of SEQ ID NOs:339 and 970-979. In certain embodiments, the gRNA may differ by no more than 3 nucleotides from a sequence selected from the group consisting of SEQ ID NOs:339 and 970-979.

Provided herein in certain embodiments are the use of optional genome editing system components such as oligonucleotide donor templates. In certain embodiments, donor templates for use in targeting the 13 nt target region may include, without limitation, donor templates encoding alterations (e.g., deletions) of HBG1 c.-114 to -102, HBG1 c.-225 to -222, and/or HBG2 c.-114 to -102. In certain embodiments, 5′ and 3′ homology arms and exemplary full-length donor templates encoding deletions such as c.-114 to -102 are also presented below (e.g., SEQ ID NOS: 904-909, 990-995). In certain embodiments, the template nucleic acid may be a single stranded oligodeoxynucleotide (ssODN). In certain embodiments, the template nucleic acid may be a positive strand or a negative strand. In certain embodiments, the ssODN may comprise a 5′ homology arm, a replacement sequence, and a 3′ homology arm. In certain embodiments, the 5′ homology arm may be about 25 to about 200 nucleotides or more in length, e.g., at least about 25, 50, 75, 100, 125, 150, 175, or 200 nucleotides in length; the replacement sequence may comprise 0 nucleotides in length; and the 3′ homology arm may be about 25 to about 200 nucleotides or more in length, e.g., at least about 25, 50, 75, 100, 125, 150, 175, or 200 nucleotides in length. In certain embodiments, the 5′ homology arm may comprise about 50 to 100 bp, e.g., 55 to 95, 60 to 90, 70 to 90, or 80 to 90 bp, homology 5′ to the 13 nt target region and the 3′ homology arm may comprise about 50 to 100 bp, e.g., 55 to 95, 60 to 90, 70 to 90, or 80 to 90 bp, homology 3′ to the 13 nt target region. In certain embodiments, the 13 nt target region may be HBG1 c.-114 to -102 (e.g., nucleotides 2824-2836 of SEQ ID NO:902 (HBG1)), HBG2 c.-114 to -102 (e.g., nucleotides 2748-2760 of SEQ ID NO:903 (HBG2)), or a combination thereof. In certain embodiments, the ssODN may comprise one or more phosphorothioates.

### BRIEF DESCRIPTION OF THE DRAWINGS

The accompanying drawings are intended to provide illustrative, and schematic rather than comprehensive, examples of certain aspects and embodiments of the present disclosure. The drawings are not intended to be limiting or binding to any particular theory or model, and are not necessarily to scale. Without limiting the foregoing, nucleic acids and polypeptides may be depicted as linear sequences, or as schematic two-or three-dimensional structures; these depictions are intended to be illustrative rather than limiting or binding to any particular model or theory regarding their structure.

FIG. 1 depicts, in schematic form, HBG1 and HBG2 gene(s) in the context of the β-globin gene cluster on human chromosome 11. FIG. 1. Each gene in the β-globin gene cluster is transcriptionally regulated by a proximal promoter. While not wishing to be bound by any particular theory, it is generally thought that A_{γ} and/or G_{γ} expression is activated by engagement between the proximal promoter with the distal strong erythroid-specific enhancer, the locus control region (LCR). Long-range transactivation by the LCR is thought to be mediated by alteration of chromatin configuration/confirmation. The LCR is marked by 4 erythroid specific Dnase I hypersensitive sites (HS1-4) and 2 distal enhancer elements (5′ HS and 3′ HS1). β-like gene globin gene expression is regulated in a developmental stage-specific manner, and expression of globin genes changes coincide with changes in the main site of blood production.

FIGS. 2A-2B depict HBG1 and HBG2 genes, coding sequences (CDS) and small deletions and point mutations in and upstream of the HBG1 and HBG2 proximal promoters that have been identified in patients and associated with elevation of fetal hemoglobin (HbF). Core elements within the proximal promoters (CAAT box, 13 nt sequence) that have been deleted in some patients with hereditary persistence of fetal hemoglobin (HPFH). The ‘target sequence’ region of each locus, which has been screened for gRNA binding target sites, is also identified.

FIGS. 3A-C show data from gRNA screening for incorporation of the 13 nt deletion in human K562 erythroleukemia cells. FIG. 3A Gene editing as determined by T7E1 endonuclease assay analysis (referred to interchangeably as a “T7E1 analysis”) of HBG1 and HBG2 locus-specific PCR products amplified from genomic DNA extracted from K562 cells after electroporation with DNA encoding S. pyogenes-specific gRNAs and plasmid DNA encoding S. pyogenes Cas9. FIG. 3B Gene editing as determined by DNA sequence analysis of PCR products amplified from the HBG1 locus in genomic DNA extracted from K562 cells after electroporation with DNA encoding the indicated gRNA and Cas9 plasmid. FIG. 3C Gene editing as determined by DNA sequence analysis of PCR products amplified from the HBG2 locus in genomic DNA extracted from K562 cells after electroporation with DNA encoding the indicated gRNA and Cas9 plasmid. For FIG. 3B-C, the types of editing events (insertions, deletions) and subtypes of deletions (13 nt target partially [12 nt HPFH] or fully [13-26 nt HPFH] deleted, other sequences deleted [other deletions]) are indicated by the differently shaded/patterned bars.

FIGS. 4A-C depict results of gene editing in human cord blood (CB) and human adult CD34⁺ cells after electroporation with RNPs complexed to in vitro transcribed S. pyogenes gRNAs that target a specific 13 nt sequence for deletion (HBG sgRNAs Sp35 and Sp37). FIG. 4A depicts the percentage of indels detected by T7E1 analysis of HBG1 and HBG2 specific PCR products amplified from gDNA extracted from CB CD34⁺ cells treated with the indicated RNPs or donor matched untreated control cells (n=3 CB CD34⁺ cells, 3 separate experiments). Data shown represent the mean and error bars correspond to standard deviation across the 3 separate donors/experiments. FIG. 4B depicts the percentage of indels detected by T7E1 analysis of HBG2 specific PCR product amplified from gDNA extracted from CB CD34⁺ cells or adult CD34⁺ cells treated with the indicated RNPs or donor matched untreated control cells (n=3 CB CD34⁺ cells, n=3 mobilized peripheral blood (mPB) CD34⁺ cells, 3 separate experiments). Data shown represent the mean and error bars correspond to standard deviation across the 3 separate donors/experiments. FIG. 4C (Top panel) depicts indels as detected by T7E1 analysis of HBG2 PCR products amplified from gDNA extracted from human CB CD34⁺ cells electroporated with HBG Sp35 RNP or HBG Sp37 RNP+/−ssODN (unmodified or with PhTx modified 5′ and 3′ ends). The lower left panel shows the level of gene editing as determined by Sanger DNA sequence analysis of gDNA from cells edited with HBG Sp37 RNP and ssODN. The lower right panel shows the specific types of deletions detected within total deletions.

FIGS. 5A-B depict gene editing of HBG in adult human mobilized peripheral blood (mPB) CD34⁺ cells and induction of fetal hemoglobin in erythroid progeny of RNP treated cells after electroporation of mPB CD34⁺ cells with HBG Sp37 RNP+/−ssODN encoding the 13 nt deletion. FIG. 5A depicts the percentage of indels detected by T7E1 analysis of HBG2 PCR product amplified from gDNA extracted from mPB CD34⁺ cells treated with the RNP or donor matched untreated control cells. FIG. 5B depicts the fold change in HBG mRNA expression in day 7 erythroblasts that were differentiated from RNP treated and untreated donor matched control mPB CD34⁺ cells. mRNA levels are normalized to GAPDH and calibrated to the levels detected in untreated controls on the corresponding days of differentiation.

FIGS. 6A-B depict the ex vivo differentiation potential of RNP treated and untreated mPB CD34⁺ cells from the same donor. FIG. 6A shows hematopoietic myeloid/erythroid colony forming cell (CFC) potential, where the number and subtype of colonies are indicated (GEMM: granulocyte-erythroid-monocyte-macrophage colony, E: erythroid colony, GM: granulocyte-macrophage colony, M: macrophage colony, G: granulocyte colony). FIG. 6B depicts the percentage of Glycophorin A expressed over the time course of erythroid differentiation as determined by flow cytometry analysis at the indicated time points and for the indicated samples.

FIG. 7A depicts indels detected by T7E1 analysis of HBG PCR product amplified from gDNA extracted from human mPB CD34⁺ cells treated with HBG RNPs (D10A paired nickases). For a subset of samples, cells also received ssODN encoding the 13 nt deletion plus silent SNPs to monitor for HDR (ssODN).

FIG. 7B depicts DNA sequencing analysis for select subset of samples shown in FIG. 7A. The indels were subdivided according to the type of indel (insertion, 13 nt deletion, or other deletion).

FIG. 8A depicts the indels at the HBG target site after electroporation of mPB CD34⁺ cells with the indicated pairs of gRNAs complexed in D10A nickase and WT RNP pairs.

FIG. 8B depicts the large deletion events (e.g. deletion of HBG2) after electroporation of mPB CD34⁺ cells with the indicated pairs of gRNAs complexed in D10A nickase and WT RNPs.

FIG. 8C depicts DNA sequencing analysis and the subtypes of events (insertions, deletions) detected in gDNA from mPB CD34⁺ cells treated with paired D10A nickase pairs.

FIG. 8D depicts DNA sequencing analysis and the subtypes of events (insertions, deletions) detected in gDNA from mPB CD34⁺ cells treated with paired WT RNP pairs.

FIG. 9 depicts the summary of HbF protein and mRNA expression in the progeny of mPB CD34⁺ cells treated with paired RNPs targeting HBG, for the experiments shown in FIGS. 7 and 8. HbF protein (by HPLC analysis) and HbF mRNA expression (ddPCR analysis) were evaluated in erythroid progeny of RNP treated human mPB CD34⁺ cells (background levels of HbF detected in donor matched untreated controls were subtracted from the levels detected in progeny of RNP treated CD34⁺ cells).

FIGS. 10A-H depict the indel frequencies and ex vivo and in vivo short-term hematopoietic potential of CD34⁺ cells after treatment with different concentrations (0, 2.5, 3.7 μM) of paired D10A nickase RNPs (SpA+Sp85). Indels were evaluated by T7E1 analysis (FIG. 10A) and by Illumina sequencing analysis (insertions and deletions, FIG. 10B). FIG. 10C depicts the % of HbF protein detected by HPLC analysis (% HbF =100%×HbF/(HbF+HbA). FIG. 10D depicts the hematopoietic activity of the RNP treated and donor matched untreated control CD34⁺ cells in colony forming cell (CFC) assays. CFCs shown are per thousand CD34⁺ cells plated. FIG. 10E depicts human blood CD45⁺ cell reconstitution of the peripheral blood in immunodeficient mice (NSG) 1 month after transplantation with donor matched human mPB CD34⁺ that were either untreated (0 μM), or treated with one of two doses (2.5 and 3.75 μM) of D10A RNP and paired gRNAs. FIG. 10F depicts human blood CD45⁺ cell reconstitution of the peripheral blood in immunodeficient mice (NSG) 2 months after transplantation. FIGS. 10G and 10H depict the lineage distributions following human CD45⁺ blood cell reconstitution of NSG mice at 1 month (FIG. 10G) and 2 months (FIG. 10H).

FIG. 11A correlates HbF levels as assayed by HPLC and indel frequency as assessed by T7E1 analysis for two D10A nickase RNP pairs (SP37+SPB and SP37+SPA) delivered at the indicated concentrations to mPB CD34⁺ cells. HbF levels were analyzed in erythroid progeny (day 18) of edited CD34⁺ cells. HbF protein detected in donor-matched untreated controls were subtracted from edited samples.

FIG. 11B depicts indel rates overlaid on hematopoietic colony forming cell (CFC) activity associated with CD34⁺ cells treated with the indicated D10A nickase pairs or untreated controls.

FIG. 11C depicts human CD45⁺ blood cell reconstitution of immunodeficient NSG mice one

month after transplantation of mPB CD34⁺ cells treated with indicated D10 RNP nickase pairs at the concentrations given or donor matched untreated controls.

FIG. 11D depicts the human blood lineage distribution detected in the human CD45⁺ fraction in mouse peripheral blood one month post-transplant.

FIG. 12 depicts a target site for derepression of HbF, the GATA1 motif of the +58 DNase I hypersensitive site (DHS) erythroid specific enhancer of BCL11A (BCL11Ae) (genomic coordinates: chr2:60,495,265 to 60,495,270).

FIG. 13A depicts the percentage of indels detected by T7E1 endonuclease analysis of BCL11A PCR products amplified from gDNA extracted from CB CD34⁺ cells treated with the indicated RNP+/−ssODN or donor matched untreated control cells. Data shown represent the mean of three 3 separate donors/experiments.

FIG. 13B depicts indels detected by T7E1 endonuclease analysis of BCL11A PCR products amplified from gDNA extracted from CB CD34⁺ cells treated with the indicated WT RNP (single gRNA targeting the BCL11A erythroid enhancer complexed to WT S. pyogenes Cas9 having both RuvC and HNH activity) or paired nickase RNP (paired gRNAs targeting the BCL11A erythroid enhancer complexed to S. pyogenes Cas9 nickases sharing the same HNH single stranded cutting activity (e.g. D10A), as well as the hematopoietic activity of cells in each condition.

FIG. 14A depicts the editing frequency of BCL11Ae (using single gRNA approach targeting the GATA1 motif) in adult human BM CD34⁺ cells.

FIG. 14B depicts the monoallelic and bialleleic editing detected in hematopoietic colonies (GEMMs, clonal progeny of BCL11Ae RNP treated CD34⁺ cells) as determined by DNA sequencing analysis.

FIG. 14C depicts the kinetics of erythroblast maturation (enucleation as determined by DRAQ5⁻ cells detected by flow cytometry analysis).

FIG. 14D depicts the acquisition of erythroid phenotype (Glycophorin A⁺ cells) in differentiated control and RNP-treated BM CD34⁺ cells.

FIG. 14E shows the fold increase in HbF⁺ cells as determined by flow cytometry analysis relative to HbF+ cells in untreated donor matched control samples.

FIGS. 15A-C depict gene editing of BCL11Ae in adult human mPB CD34⁺ cells and induction of fetal hemoglobin in erythroid progeny of RNP and ssODN treated cells after electroporation of mPB CD34⁺ cells with BCL11Ae RNP+nonspecific ssODN. FIG. 15A depicts the percentage of indels detected by T7E1 analysis of HBG2 PCR product amplified from gDNA extracted from mPB CD34⁺ cells treated with the BCL11Ae RNP and nonspecific ssODN or donor matched untreated control cells. FIG. 15B depicts the fold change in HBG mRNA expression in day 10 erythroblasts that were differentiated from BCL11Ae RNP treated and untreated donor matched control mPB CD34⁺ cells (mRNA levels are normalized to GAPDH and calibrated to the levels detected in untreated controls on the corresponding days of differentiation). FIG. 15C depicts the percentage of Glycophorin A expressed over the time course of erythroid differentiation of mPB CD34⁺ cells+/−treatment with BCL11Ae RNP and nonspecific ssODN, as determined by flow cytometry analysis at the indicated time points and for the indicated samples.

FIGS. 16A-B depict gene editing of the HBG promoter in and viability of adult human CD34⁺ cells from mPB electroporated with Cas9 complexed with Sp37 gRNA (targeting domain set forth in SEQ ID NO:933), which was in vitro transcribed (Sp37 IVT) or chemically synthesized (Sp37 Synthetic). FIG. 16A depicts the percentage of indels detected by sequencing the HBG PCR product amplified from adult human CD34⁺ cells from mPB electroporated with Cas9 complexed with Sp37 IVT or Sp37 Synthetic gRNAs. Next generation sequencing (NGS) (co-amplification and sequencing of HBG1 and HBG2 PCR product) was used to detect the indels. FIG. 16B depicts the percentage viability of the adult human mPB CD34⁺ cells 48 hours after electroporation with Cas9 complexed with Sp37 IVT or Sp37 Synthetic gRNAs.

FIG. 17 depicts gene editing of the HBG promoter in adult human CD34⁺ cells from mPB electroporated with Cas9 complexed with Sp35 gRNA (SEQ ID NO:970) and modified Sp35 gRNAs as disclosed in Table 10. The percentage of indels at the HBG promoter was determined by sequencing the HBG PCR product amplified from gDNA extracted post-electroporation with RNP.

FIGS. 18A-C depict gene editing of the HBG promoter in and viability of adult human CD34⁺ cells from mPB electroporated with ssODN “13 nt-strand” (i.e., OLI16412 (ssODN encoding the 13 nt deletion, negative (−) strand) or “13 nt +strand” (i.e., OLI16414 (ssODN encoding the 13 nt deletion, positive (+) strand) (Table 11) and RNP targeting HBG containing Sp35 gRNA (SEQ ID NO:970) (OLI7066-sp35-RNP). FIG. 18A depicts the percentage of indels detected by sequencing the HBG PCR product from genomic DNA extracted at 72 hours post-electroporation. The grey portion of the bar represents the 13 nt indel deletion, whereas the white portion of the bar represents other indels. FIG. 18B depicts the percent viability of cells 48 hours post-electroporation. FIG. 18C depicts the percentage of relative expression levels of gamma-globin chains over total beta-like globin chains (gamma chains/[gamma chains+beta chain]) detected by ultra-performance liquid chromatography (UPLC).

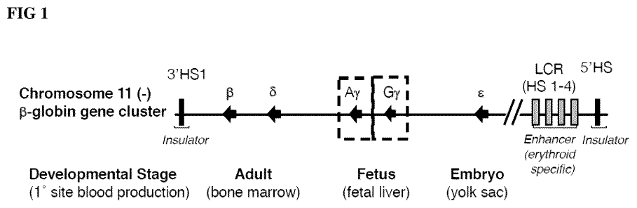

### DETAILED DESCRIPTION

### Definitions and Abbreviations

Unless otherwise specified, each of the following terms has the meaning associated with it in this section.

The indefinite articles “a” and “an” refer to at least one of the associated noun, and are used interchangeably with the terms “at least one” and “one or more.” For example, “a module” means at least one module, or one or more modules.

The conjunctions “or” and “and/or” are used interchangeably as non-exclusive disjunctions.

“Domain” is used to describe a segment of a protein or nucleic acid. Unless otherwise indicated, a domain is not required to have any specific functional property.

An “indel” is an insertion and/or deletion in a nucleic acid sequence. An indel may be the product of the repair of a DNA double strand break, such as a double strand break formed by a genome editing system of the present disclosure. An indel is most commonly formed when a break is repaired by an “error prone” repair pathway such as the NHEJ pathway described below.

“Gene conversion” refers to the alteration of a DNA sequence by incorporation of an endogenous homologous sequence (e.g. a homologous sequence within a gene array). “Gene correction” refers to the alteration of a DNA sequence by incorporation of an exogenous homologous sequence, such as an exogenous single- or double stranded donor template DNA. Gene conversion and gene correction are products of the repair of DNA double-strand breaks by HDR pathways such as those described below.

Indels, gene conversion, gene correction, and other genome editing outcomes are typically assessed by sequencing (most commonly by “next-gen” or “sequencing-by-synthesis” methods, though Sanger sequencing may still be used) and are quantified by the relative frequency of numerical changes (e.g., ±1, ±2 or more bases) at a site of interest among all sequencing reads. DNA samples for sequencing may be prepared by a variety of methods known in the art, and may involve the amplification of sites of interest by polymerase chain reaction (PCR), the capture of DNA ends generated by double strand breaks, as in the GUIDEseq process described in Tsai 2016 (incorporated by reference herein) or by other means well known in the art. Genome editing outcomes may also be assessed by in situ hybridization methods such as the FiberComb™ system commercialized by Genomic Vision (Bagneux, France), and by any other suitable methods known in the art.

“Alt-HDR,” “alternative homology-directed repair,” or “alternative HDR” are used interchangeably to refer to the process of repairing DNA damage using a homologous nucleic acid (e.g., an endogenous homologous sequence, e.g., a sister chromatid, or an exogenous nucleic acid, e.g., a template nucleic acid). Alt-HDR is distinct from canonical HDR in that the process utilizes different pathways from canonical HDR, and can be inhibited by the canonical HDR mediators, RAD51 and BRCA2. Alt-HDR is also distinguished by the involvement of a single-stranded or nicked homologous nucleic acid template, whereas canonical HDR generally involves a double-stranded homologous template.

“Canonical HDR,” “canonical homology-directed repair” or “cHDR” refer to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single stranded portion of DNA. In a normal cell, cHDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2, and the homologous nucleic acid is typically double-stranded.

Unless indicated otherwise, the term “HDR” as used herein encompasses both canonical HDR and alt-HDR.

“Non-homologous end joining” or “NHEJ” refers to ligation mediated repair and/or non-template mediated repair including canonical NHEJ (cNHEJ) and alternative NHEJ (altNHEJ), which in turn includes microhomology-mediated end joining (MMEJ), single-strand annealing (SSA), and synthesis-dependent microhomology-mediated end joining (SD-MMEJ).

“Replacement” or “replaced,” when used with reference to a modification of a molecule (e.g. a nucleic acid or protein), does not require a process limitation but merely indicates that the replacement entity is present.

“Subject” means a human, mouse, or non-human primate. A human subject can be any age (e.g., an infant, child, young adult, or adult), and may suffer from a disease, or may be in need of alteration of a gene.

“Treat,” “treating,” and “treatment” mean the treatment of a disease in a subject (e.g., a human subject), including one or more of inhibiting the disease, i.e., arresting or preventing its development or progression; relieving the disease, i.e., causing regression of the disease state; relieving one or more symptoms of the disease; and curing the disease.

“Prevent,” “preventing,” and “prevention” refer to the prevention of a disease in a subject, including (a) avoiding or precluding the disease; (b) affecting the predisposition toward the disease; or (c) preventing or delaying the onset of at least one symptom of the disease.

A “kit” refers to any collection of two or more components that together constitute a functional unit that can be employed for a specific purpose. By way of illustration (and not limitation), one kit according to this disclosure can include a guide RNA complexed or able to complex with an RNA-guided nuclease, and accompanied by (e.g. suspended in, or suspendable in) a pharmaceutically acceptable carrier. The kit can be used to introduce the complex into, for example, a cell or a subject, for the purpose of causing a desired genomic alteration in such cell or subject. The components of a kit can be packaged together, or they may be separately packaged. Kits according to this disclosure also optionally include directions for use (DFU) that describe the use of the kit e.g., according to a method of this disclosure. The DFU can be physically packaged with the kit, or it can be made available to a user of the kit, for instance by electronic means.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides, nucleotide sequences, nucleic acids etc. can be chimeric mixtures or derivatives or modified versions thereof, single-stranded or double-stranded. They can be modified at the base moiety, sugar moiety, or phosphate backbone, for example, to improve stability of the molecule, its hybridization parameters, etc. A nucleotide sequence typically carries genetic information, including, but not limited to, the information used by cellular machinery to make proteins and enzymes. These terms include double- or single-stranded genomic DNA, RNA, any synthetic and genetically manipulated polynucleotide, and both sense and antisense polynucleotides. These terms also include nucleic acids containing modified bases.

Conventional IUPAC notation is used in nucleotide sequences presented herein, as shown in Table 1, below (see also Cornish-Bowden A, Nucleic Acids Res. 1985 May 10; 13 (9):3021-30,incorporated by reference herein). It should be noted, however, that “T” denotes “Thymine or Uracil” in those instances where a sequence may be encoded by either DNA or RNA, for example in gRNA targeting domains.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “protein,” “peptide” and “polypeptide” are used interchangeably to refer to a sequential chain of amino acids linked together via peptide bonds. The terms include individual proteins, groups or complexes of proteins that associate together, as well as fragments or portions, variants, derivatives and analogs of such proteins. Peptide sequences are presented herein using conventional notation, beginning with the amino or N-terminus on the left, and proceeding to the carboxyl or C-terminus on the right. Standard one-letter or three-letter abbreviations can be used.

The notations “c.-114 to -102 region,” “13 nt target region” and the like refer to a sequence that is 5′ of the transcription start site (TSS) of the HBG1 and/or HBG2 gene. The HBG1 c.-114 to -102 region is exemplified in SEQ ID NO:902 (HBG1) at positions 2824-2836 and the HBG2 c.-114 to -102 region is exemplified in SEQ ID NO:903 (HBG2) at positions 2748-2760. The term “13 nt deletion” and the like refer to deletions of the 13 nt target region.

The term “proximal HBG1/2 promoter target sequence” denotes the region within 50, 100, 200, 300, 400, or 500 bp of a proximal HBG1/2 promoter sequence including the 13 nt target region. Alterations by genome editing systems according to this disclosure facilitate (e.g. cause, promote or tend to increase the likelihood of) upregulation of HbF production in erythroid progeny.

The term “GATA1 binding motif in BCL11Ae” refers to the sequence that is the GATA1 binding motif in the erythroid specific enhancer of BCL11A (BCL11Ae) that is in the +58 DNase I hypersensitive site (DHS) region of intron 2 of the BCL11A gene. The genomic coordinates for the GATA1 binding motif in BCL11Ae are chr2: 60,495,265 to 60,495,270. The +58 DHS site comprises a 115 base pair (bp) sequence as set forth in SEQ ID NO:968. The +58 DHS site sequence, including ˜500 bp upstream and ˜200 bp downstream is set forth in SEQ ID NO:969.

### Overview

The various embodiments of this disclosure generally relate to genome editing systems configured to introduce alterations (e.g., a deletion or insertion, or other mutation) into chromosomal DNA that enhance transcription of the HBG1 and/or HBG2 genes, which encode the Aγ and Gγ subunits of hemoglobin, respectively. Exemplary mutations are made in or around the 13 nt target region of HBG1 and/or HBG2.

Fetal hemoglobin (HbF) expression can also be induced through targeted disruption of the erythroid cell specific expression of a transcriptional repressor, BCL11A, which encodes a repressor that silences HBG1 and HBG2 (Canvers 2015). Another gene editing strategy disclosed herein is to increase HbF expression by targeting disruption the of the erythroid specific enhancer of BCL11A (BCL11Ae) (also discussed in commonly-assigned International Patent Publication No. WO 2015/148860 by Friedland et al. (“Friedland,” incorporated by reference herein in its entirety). In certain embodiments, the region of BCL11Ae targeted for disruption may be the GATA1 binding motif in BCL11Ae. In certain embodiments, genome editing systems disclosed herein may be used to introduce alterations into the GATA1 binding motif in BCL11Ae and the 13 nt target region of HBG1 and/or HBG2.

The genome editing systems of this disclosure can include an RNA-guided nuclease such as Cas9 or Cpf1 and one or more gRNAs having a targeting domain that is complementary to a sequence in or near the target region, and optionally one or more of a DNA donor template that encodes a specific mutation (such as a deletion or insertion) in or near the target region, and/or an agent that enhances the efficiency with which such mutations are generated including, without limitation, a random oligonucleotide, a small molecule agonist or antagonist of a gene product involved in DNA repair or a DNA damage response, or a peptide agent.

A variety of approaches to the introduction of mutations into the 13 nt target region, proximal HBG1/2 promoter target sequence, and/or the GATA1 binding motif in BCL11Ae may be employed in the embodiments of the present disclosure. In one approach, a single alteration, such as a double-strand break, is made within the 13 nt target region, proximal HBG1/2 promoter target sequence, and/or the GATA1 binding motif in BCL11Ae, and is repaired in a way that disrupts the function of the region, for example by the formation of an indel or by the incorporation of a donor template sequence that encodes the deletion of the region. In a second approach, two or more alterations are made on either side of the region, resulting in the deletion of the intervening sequence, including the 13 nt target region and/or the GATA1 binding motif in BCL11Ae.

The treatment of hemogolobinopathies by gene therapy and/or genome editing is complicated by the fact that the cells that are phenotypically affected by the disease, erythrocytes or RBCs, are enucleated, and do not contain genetic material encoding either the aberrant hemoglobin protein (Hb) subunits nor the Aγ or Gγ subunits targeted in the exemplary genome editing approaches described above. This complication is addressed, in certain embodiments of this disclosure, by the alteration of cells that are competent to differentiate into, or otherwise give rise to, erythrocytes. Cells within the erythroid lineage that are altered according to various embodiments of this disclosure include, without limitation, hematopoietic stem and progenitor cells (HSCs), erythroblasts (including basophilic, polychromatic and/or orthochromatic erythroblasts), proerythroblasts, polychromatic erythrocytes or reticulocytes, embryonic stem(ES) cells, and/or induced pluripotent stem (iPSC) cells. These cells may be altered in situ (e.g. within a tissue of a subject) or ex vivo. Implementations of genome editing systems for in situ and ex vivo alteration of cells is described under the heading “Implementation of genome editing systems: delivery, formulations, and routes of administration” below.

In certain embodiments, alterations that result in induction of Aγ and/or Gγ expression are obtained through the use of a genome editing system comprising an RNA-guided nuclease and at least one gRNA having a targeting domain complementary to a sequence within the 13 nt target region of HBG1 and/or HBG2 or proximate thereto (e.g., within 10, 20, 30, 40, or 50, 100, 200, 300, 400 or 500 bases of the 13 nt target region). As is discussed in greater detail below, the RNA-guided nuclease and gRNA form a complex that is capable of associating with and altering the 13 nt target region or a region proximate thereto. Examples of suitable targeting domains directed to the 13 nt target region of HBG1 and/or HBG2 or proximate thereto for use in the embodiments disclosed herein include, without limitation, those set forth in SEQ ID NOs:251-901, 940-942. In certain embodiments, gRNAs targeting the 13 nt target region of HBG1 and/or HBG2 or proximate thereto may include modifications including, without limitation, addition of one or more PhTx or 2′-O-methyl groups. The modifications may be at or near the 5′ end or 3′ end of the gRNA or at or near the 5′ and 3′ ends of the gRNA. In certain embodiments, the gRNAs may be in vitro transcribed gRNAs or chemically synthesized gRNAs. Examples of suitable gRNAs directed to the 13 nt target region of HBG1 and/or HBG2 for use in the embodiments disclosed herein include, without limitation, those set forth in SEQ ID NOs:970-979.

In certain embodiments, alterations that result in induction of HbF expression are obtained through the use of a genome editing system comprising an RNA-guided nuclease and at least one gRNA having a targeting domain complementary to a sequence within the GATA1 binding motif in BCL11Ae or proximate thereto (e.g., within 10, 20, 30, 40, or 50, 100, 200, 300, 400 or 500 bases of the GATA1 binding motif in BCL11Ae). In certain embodiments, the RNA-guided nuclease and gRNA form a complex that is capable of associating with and altering the GATA1 binding motif in BCL11Ae. Examples of suitable targeting domains directed to the GATA1 binding motif in BCL11Ae for use in the embodiments disclosed herein include, without limitation, those set forth in SEQ ID NOs: 952-955.

The genome editing system can be implemented in a variety of ways, as is discussed below in detail. As an example, a genome editing system of this disclosure can be implemented as a ribonucleoprotein complex or a plurality of complexes in which multiple gRNAs are used. This ribonucleoprotein complex can be introduced into a target cell using art-known methods, including electroporation, as described in commonly-assigned International Patent Publication No. WO 2016/182959 by Jennifer Gori (“Gori,” incorporated by reference herein in its entirety).

The ribonucleoprotein complexes within these compositions are introduced into target cells by art-known methods, including without limitation electroporation (e.g. using the Nucleofection™ technology commercialized by Lonza, Basel, Switzerland or similar technologies commercialized by, for example, Maxcyte Inc. Gaithersburg, Maryland) and lipofection (e.g. using Lipofectamine™ reagent commercialized by Thermo Fisher Scientific, Waltham Massachusetts). Alternatively, or additionally, ribonucleoprotein complexes are formed within the target cells themselves following introduction of nucleic acids encoding the RNA-guided nuclease and/or gRNA. These and other delivery modalities are described in general terms below and in Gori.

Cells that have been altered ex vivo according to this disclosure can be manipulated (e.g. expanded, passaged, frozen, differentiated, de-differentiated, transduced with a transgene, etc.) prior to their delivery to a subject. The cells are, variously, delivered to a subject from which they are obtained (in an “autologous” transplant), or to a recipient who is immunologically distinct from a donor of the cells (in an “allogeneic” transplant).

In some cases, an autologous transplant includes the steps of obtaining, from the subject, a plurality of cells, either circulating in peripheral blood, or within the marrow or other tissue (e.g. spleen, skin, etc.), and manipulating those cells to enrich for cells in the erythroid lineage (e.g. by induction to generate iPSCs, purification of cells expressing certain cell surface markers such as CD34, CD90, CD49f and/or not expressing surface markers characteristic of non-erythroid lineages such as CD10, CD14, CD38, etc.). The cells are, optionally or additionally, expanded, transduced with a transgene, exposed to a cytokine or other peptide or small molecule agent, and/or frozen/thawed prior to transduction with a genome editing system targeting the 13 nt target region, proximal HBG1/2 promoter target sequence, and/or the GATA1 binding motif in BCL11Ae. The genome editing system can be implemented or delivered to the cells in any suitable format, including as a ribonucleoprotein complex, as separated protein and nucleic acid components, and/or as nucleic acids encoding the components of the genome editing system.

However it is implemented, a genome editing system may include, or may be co-delivered with, one or more factors that improve the viability of the cells during and after editing, including without limitation an aryl hydrocarbon receptor antagonist such as StemRegenin-1 (SR1), UM171, LGC0006, alpha-napthoflavone, and CH-223191, and/or an innate immune response antagonist such as cyclosporin A, dexamethasone, reservatrol, a MyD88 inhibitory peptide, an RNAi agent targeting Myd88, a B18R recombinant protein, a glucocorticoid, OxPAPC, a TLR antagonist, rapamycin, BX795, and a RLR shRNA. These and other factors that improve the viability of the cells during and after editing are described in Gori, under the heading “I. Optimization of Stem Cells” from page 36 through page 61, which is incorporated by reference herein.

The cells, following delivery of the genome editing system, are optionally manipulated e.g. to enrich for HSCs and/or cells in the erythroid lineage and/or for edited cells, to expand them, freeze/thaw, or otherwise prepare the cells for return to the subject. The edited cells are then returned to the subject. for instance in the circulatory system by means of intravenous delivery or delivery or into a solid tissue such as bone marrow.

Functionally, alteration of the 13 nt target region, proximal HBG1/2 promoter target sequence, and/or the GATA1 binding motif in BCL11Ae using the compositions, methods and genome editing systems of this disclosure results in significant induction, among hemoglobin-expressing cells, of Aγ and/or Gγ subunits (referred to interchangeably as HbF expression), e.g. at least 5%, 10%, 15%, 20%, 25%, 30%, 35%, 40%, 45%, 50% or greater induction of Aγ and/or Gγ subunit expression relative to unmodified controls. This induction of protein expression is generally the result of alteration of the 13 nt target region, proximal HBG1/2 promoter target sequence, and/or the GATA1 binding motif in BCL11Ae (expressed, e.g. in terms of the percentage of total genomes comprising indel mutations within the plurality of cells) in some or all of the plurality of cells that are treated, e.g. at least 5%, 10%, 15%, 20%, 25%, 30%, 35%, 40%, 45%, 50% of the plurality of cells comprise at least one allele comprising a sequence alteration, including, without limitation, an indel, insertion, or deletion in or near the 13 nt target region, proximal HBG1/2 promoter target sequence, and/or the GATA1 binding motif in BCL11Ae.

The functional effects of alterations caused or facilitated by the genome editing systems and methods of the present disclosure can be assessed in any number of suitable ways. For example, the effects of alterations on expression of fetal hemoglobin can be assessed at the protein or mRNA level. Expression of HBG1 and HBG2 mRNA can be assessed by digital droplet PCR (ddPCR), which is performed on cDNA samples obtained by reverse transcription of mRNA harvested from treated or untreated samples. Primers for HBG1, HBG2, HBB, and/or HBA may be used individually or multiplexed using methods known in the art. For example, ddPCR analysis of samples may be conducted using the QX200™ ddPCR system commercialized by Bio Rad (Hercules, CA), and associated protocols published by BioRad. Fetal hemoglobin protein may be assessed by high pressure liquid chromatography (HPLC), for example, according to the methods discussed on pp. 143-44 in Chang 2017, incorporated by reference herein, or fast protein liquid chromatography (FPLC), using ion-exchange and/or reverse phase columns to resolve HbF, HbB and HbA and/or Aγ and Gγ globin chains as is known in the art.

It should be noted that the rate at which the 13 nt target region, proximal HBG1/2 promoter target sequence, and/or the GATA1 binding motif in BCL11Ae is altered in the target cells can be modified by the use of optional genome editing system components such as oligonucleotide donor templates. Donor template design is described in general terms below under the heading “Donor template design.” Donor templates for use in targeting the 13 nt target region may include, without limitation, donor templates encoding alterations (e.g., deletions) of HBG1 c.-114 to -102 (corresponding to nucleotides 2824-2836 of SEQ ID NO: 902), HBG1 c.-225 to -222 (corresponding to nucleotides 2716-2719 of SEQ ID NO:902)), and/or HBG2 c.-114 to -102 (corresponding to nucleotides 2748-2760 of SEQ ID NO:903). Exemplary 5′ and 3′ homology arms, and exemplary full-length donor templates encoding deletions such as c.-114 to -102 are also presented below (SEQ ID NOS: 904-909, 990-995). Donor templates used herein may be non-specific templates that are non-homologous to regions of DNA within or near the target sequence. In certain embodiments, donor templates for use in targeting the 13 nt target region may include, without limitation, non-target specific templates that are nonhomologous to regions of DNA within or near the 13 nt target region. For example, a non-specific donor template for use in targeting the 13 nt target region may be non-homologous to the regions of DNA within or near the 13 nt target region and may comprise a donor template encoding the deletion of HBG1 c.-225 to -222 (corresponding to nucleotides 2716-2719 of SEQ ID NO:902). In certain embodiments, donor templates for use in targeting the GATA1 binding motif in BCL11Ae may include, without limitation, non-target specific templates that are nonhomologous to regions of DNA within or near GATA1 binding motif in BCL11Ae target sequence. Other donor templates for use in targeting BCL11Ae may include, without limitation, donor templates including alterations (e.g., deletions) of BCL11Ae, including, without limitation, the GATA1 motif in BCL11Ae.

The embodiments described herein may be used in all classes of vertebrate including, but not limited to, primates, mice, rats, rabbits, pigs, dogs, and cats.

This overview has focused on a handful of exemplary embodiments that illustrate the principles of genome editing systems and CRISPR-mediated methods of altering cells. For clarity, however, this disclosure encompasses modifications and variations that have not been expressly addressed above, but will be evident to those of skill in the art. With that in mind, the following disclosure is intended to illustrate the operating principles of genome editing systems more generally. What follows should not be understood as limiting, but rather illustrative of certain principles of genome editing systems and CRISPR-mediated methods utilizing these systems, which, in combination with the instant disclosure, will inform those of skill in the art about additional implementations and modifications that are within its scope.

### Genome Editing Systems

The term “genome editing system” refers to any system having RNA-guided DNA editing activity. Genome editing systems of the present disclosure include at least two components adapted from naturally occurring CRISPR systems: a guide RNA (gRNA) and an RNA-guided nuclease. These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation.

Naturally occurring CRISPR systems are organized evolutionarily into two classes and five types (Makarova 2011, incorporated by reference herein), and while genome editing systems of the present disclosure may adapt components of any type or class of naturally occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cpf1) and one or more guide RNAs (e.g., a crRNA and, optionally, a tracrRNA) that form ribonucleoprotein (RNP) complexes that associate with (i.e. target) and cleave specific loci complementary to a targeting (or spacer) sequence of the crRNA. Genome editing systems according to the present disclosure similarly target and edit cellular DNA sequences, but differ significantly from CRISPR systems occurring in nature. For example, the unimolecular guide RNAs described herein do not occur in nature, and both guide RNAs and RNA-guided nucleases according to this disclosure may incorporate any number of non-naturally occurring modifications.

Genome editing systems can be implemented (e.g. administered or delivered to a cell or a subject) in a variety of ways, and different implementations may be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as, without limitation, a lipid or polymer micro-or nano-particle, micelle, or liposome. In certain embodiments, a genome editing system is implemented as one or more nucleic acids encoding the RNA-guided nuclease and guide RNA components described above (optionally with one or more additional components); in certain embodiments, the genome editing system is implemented as one or more vectors comprising such nucleic acids, for instance a viral vector such as an adeno-associated virus (see section below under the heading “Implementation of genome editing systems: delivery, formulations, and routes of administration”); and in certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence, or may be targeted to—and capable of editing in parallel—two or more specific nucleotide sequences through the use of two or more guide RNAs. The use of multiple gRNAs is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (“Maeder,” incorporated by reference herein), describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i.e. flanking) the point mutation, and forms DSBs that flank the mutation. This, in turn, promotes deletion of the intervening sequence, including the mutation, thereby eliminating the cryptic splice site and restoring normal gene function.

As another example, International Patent Publication No. WO 2016/073990 by Cotta-Ramusino et al. (“Cotta-Ramusino,” incorporated by reference herein), describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, International Patent Publication No. WO 2015/070083 by Palestrant et al. (incorporated by reference herein) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

As disclosed herein, in certain embodiments, genome editing systems may comprise multiple gRNAs that may be used to introduce mutations into the GATA1 binding motif in BCL11Ae or the 13 nt target region of HBG1 and/or HBG2. In certain embodiments, genome editing systems disclosed herein may comprise multiple gRNAs used to introduce mutations into the GATA1 binding motif in BCL11Ae and the 13 nt target region of HBG1 and/or HBG2.

Genome editing systems can, in some instances, form double strand breaks that are repaired by cellular DNA double-strand break mechanisms such as NHEJ or HDR. These mechanisms are described throughout the literature (see, e.g., Davis & Maizels 2014 (describing Alt-HDR); Frit 2014 (describing Alt-NHEJ); Iyama & Wilson 2013 (describing canonical HDR and NHEJ pathways generally)).

Where genome editing systems operate by forming DSBs, such systems optionally include one or more components that promote or facilitate a particular mode of double-strand break repair or a particular repair outcome. For instance, Cotta-Ramusino also describes genome editing systems in which a single stranded oligonucleotide “donor template” is added; the donor template is incorporated into a target region of cellular DNA that is cleaved by the genome editing system, and can result in a change in the target sequence.

In certain embodiments, genome editing systems modify a target sequence, or modify expression of a gene in or near the target sequence, without causing single-or double-strand breaks. For example, a genome editing system may include an RNA-guided nuclease fused to a functional domain that acts on DNA, thereby modifying the target sequence or its expression. As one example, an RNA-guided nuclease can be connected to (e.g. fused to) a cytidine deaminase functional domain, and may operate by generating targeted C-to-A substitutions. Exemplary nuclease/deaminase fusions are described in Komor 2016, which is incorporated by reference. Alternatively, a genome editing system may utilize a cleavage-inactivated (i.e. a “dead”) nuclease, such as a dead Cas9 (dCas9), and may operate by forming stable complexes on one or more targeted regions of cellular DNA, thereby interfering with functions involving the targeted region(s) including, without limitation, mRNA transcription, chromatin remodeling, etc.

### Guide RNA (gRNA) Molecules

The terms “guide RNA” and “gRNA” refer to any nucleic acid that promotes the specific association (or “targeting”) of an RNA-guided nuclease such as a Cas9 or a Cpf1 to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner 2014 (incorporated herein by reference) and Cotta-Ramusino. Examples of modular and unimolecular gRNAs that may be used according to the embodiments herein include, without limitation, the sequences set forth in SEQ ID NOs: 29-31 and 38-51. Examples of gRNA proximal and tail domains that may be used according to the embodiments herein include, without limitation, the sequences set forth in SEQ ID NOs: 32-37.

In bacteria and archea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g. GAAA) “tetraloop” or “linker” sequence bridging complementary regions of the crRNA (at its 3′ end) and the tracrRNA (at its 5′ end) (Mali 2013; Jiang 2013; Jinek 2012; all incorporated by reference herein).

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu 2013, incorporated by reference herein), “complementarity regions” (Cotta-Ramusino), “spacers” (Briner 2014) and generically as “crRNAs” (Jiang 2013). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that may influence the formation or activity of gRNA/Cas9 complexes. For instance, as mentioned above, the duplexed structure formed by first and secondary complementarity domains of a gRNA (also referred to as a repeat: anti-repeat duplex) interacts with the recognition (REC) lobe of Cas9 and can mediate the formation of Cas9/gRNA complexes (Nishimasu 2014; Nishimasu 2015; both incorporated by reference herein). It should be noted that the first and/or second complementarity domains may contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner 2014, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop one near the 3′ portion of the second complementarity domain is referred to variously as the “proximal domain.” (Cotta-Ramusino) “stem loop 1” (Nishimasu 2014 and 2015) and the “nexus” (Briner 2014). One or more additional stem loop structures are generally present near the 3′ end of the gRNA, with the number varying by species: S. pyogenes gRNAs typically include two 3′ stem loops (for a total of four stem loop structures including the repeat: anti-repeat duplex), while S. aureus and other species have only one (for a total of three stem loop structures). A description of conserved stem loop structures (and gRNA structures more generally) organized by species is provided in Briner 2014.

While the foregoing description has focused on gRNAs for use with Cas9, it should be appreciated that other RNA-guided nucleases exist which utilize gRNAs that differ in some ways from those described to this point. For instance, Cpf1 (“CRISPR from Prevotella and Franciscella 1”) is a recently discovered RNA-guided nuclease that does not require a tracrRNA to function. (Zetsche 2015a, incorporated by reference herein). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Those of skill in the art will appreciate, however, that although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

### gRNA Design

Methods for selection and validation of target sequences as well as off-target analyses have been described previously (see, e.g., Mali 2013; Hsu 2013; Fu 2014;, Heigwer 2014; Bae 2014; Xiao 2014; all incorporated by reference herein). As a non-limiting example, gRNA design may involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in Maeder and Cotta-Ramusino.

With respect to selection of gRNA targeting domain sequences directed to HBG1/2 target sites (e.g. the 13 nt target region), an in-silico gRNA target domain identification tool was utilized, and the hits were stratified into four tiers. For S. pyogenes, tier 1 targeting domains were selected based on (1) distance upstream or downstream from either end of the target site (i.e., HBG1/2 13 nt target region), specifically within 400 bp of either end of the target site, (2) a high level of orthogonality, and (3) the presence of 5′ G. Tier 2 targeting domains were selected based on (1) distance upstream or downstream from either end of the target site (i.e., HBG1/2 13 nt target region), specifically within 400 bp of either end of the target site, and (2) a high level of orthogonality. Tier 3 targeting domains were selected based on (1) distance upstream or downstream from either end of the target site (i.e., HBG1/2 13 nt target region), specifically within 400 bp of either end of the target site and (2) the presence of 5′ G. Tier 4 targeting domains were selected based on distance upstream or downstream from either end of the target site (i.e., HBG1/2 13 nt target region), specifically within 400 bp of either end of the target site.

For S. aureus, tier 1 targeting domains were selected based on (1) distance upstream or downstream from either end of the target site (i.e., HBG1/2 13 nt target region), specifically within 400 bp of either end of the target site, (2) a high level of orthogonality, (3) the presence of 5′ G, and (4) PAM having the sequence NNGRRT (SEQ ID NO:204). Tier 2 targeting domains were selected based on (1) distance upstream or downstream from either end of the target site (i.e., HBG1/2 13 nt target), specifically within 400 bp of either end of the target site, (2) a high level of orthogonality, and (3) PAM having the sequence NNGRRT (SEQ ID NO:204). Tier 3 targeting domains were selected based on (1) distance upstream or downstream from either end of the target site (i.e., HBG1/2 13 nt target region), specifically within 400 bp of either end of the target site, and (2) PAM having the sequence NNGRRT (SEQ ID NO:204). Tier 4 targeting domains were selected based on (1) distance upstream or downstream from either end of the target site (i.e., HBG1/2 13 nt target), specifically within 400 bp of either end of the target site, and (2) PAM having the sequence NNGRRV (SEQ ID NO:205).

Table 2, below, presents targeting domains for S. pyogenes and S. aureus gRNAs, broken out by (a) tier (1, 2, 3 or 4) and (b) HBG1 or HBG2.

TABLE 2
gRNA targeting domain sequences for HBG1/2 target sites

| | |
|---|---|
| HBG1 | HBG2 |

S. pyogenes

| | | |
|---|---|---|
| Tier 1 | 251-256 | 760-764 |
| Tier 2 | 257-274 | 765-781 |
| Tier 3 | 275-300 | 275-281, 283-300 |
| Tier 4 | 301-366 | 301-311, 313-342, 344-348, |
| | | 350-366, 782, 783 |

S. aureus

| | | |
|---|---|---|
| Tier 1 | 367-376 | 784-791 |
| Tier 2 | 343, 377-393 | 778, 792-803 |
| Tier 3 | 357, 365, 394-461 | 357, 365, 394-461 |
| Tier 4 | 252-254, 256, 268, | 292, 295. 347, 348, 353, |
| | 272-274, 292, 295, | 360-362, 366, 462-468 476-481, |
| | 347, 348, 353, 360- | 489-587, 601-607, 614-620, |
| | 362, 366, 598-759 | 640-666, 674-679, 687-693, |
| | | 708-714, 733-753, 762-764, |
| | | 775, 779-781, 804-901 |

In certain embodiments, the gRNAs described herein may be chemically synthesized. As shown in Example 8 below, chemically synthesized Sp37 gRNA (targeting domain set forth in SEQ ID NO:933), which targets the 13 nt deletion associated with HPFH, produced an increased amount of indels compared to in vitro transcribed Sp37 gRNA. Additional exemplary gRNAs that may be chemically synthesized are set forth in SEQ ID NO:970-979 (Table 10).

gRNAs may be designed to target the erythroid specific enhancer of BCL11A (BCL11Ae) to disrupt expression of a transcriptional repressor, BCL11A (described in Friedland). gRNAs were designed to target the GATA1 binding motif that is in the erythroid specific enhancer of BCL11A that is in the +58 DHS region of intron 2 (i.e., the GATA1 binding motif in BCL11Ae), where the +58 DHS enhancer region comprises the sequence set forth in SEQ ID NO:968. Targeting domain sequences of gRNAs that were designed to target disruption of the GATA1 binding motif in BCL11Ae, include, but are not limited to, the sequences set forth in SEQ ID NOs:952-955. Targeting domain sequences plus PAM (NGG) of gRNAs that were designed to target disruption of the GATA1 binding motif in BCL11Ae, include, but are not limited to, the sequences set forth in SEQ ID NOs:960-963.

### gRNA Modifications

The activity, stability, or other characteristics of gRNAs can be altered through the incorporation of certain modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not wishing to be bound by theory it is also believed that certain modified gRNAs described herein can exhibit a reduced innate immune response when introduced into cells. Those of skill in the art will be aware of certain cellular responses commonly observed in cells, e.g., mammalian cells, in response to exogenous nucleic acids, particularly those of viral or bacterial origin. Such responses, which can include induction of cytokine expression and release and cell death, may be reduced or eliminated altogether by the modifications presented herein.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

As one example, the 5′ end of a gRNA can include a eukaryotic mRNA cap structure or cap analog (e.g., a G(5′)ppp(5′)G cap analog, a m7G(5′)ppp(5′)G cap analog, or a 3′-O-Me-m7G(5′)ppp(5′)G anti reverse cap analog (ARCA)), as shown below:

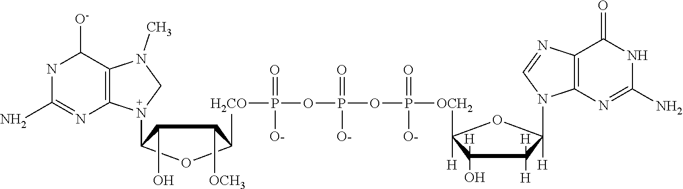

The cap or cap analog can be included during either chemical synthesis or in vitro transcription of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a polyA tract. The polyA tract can be added to a gRNA during chemical synthesis, following in vitro transcription using a polyadenosine polymerase (e.g., E. coli Poly(A)Polymerase), or in vivo by means of a polyadenylation sequence, as described in Maeder.

It should be noted that the modifications described herein can be combined in any suitable manner, e.g. a gRNA, whether transcribed in vivo from a DNA vector, or in vitro transcribed gRNA, can include either or both of a 5′ cap structure or cap analog and a 3′ polyA tract.

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

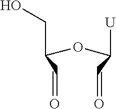

wherein “U” can be an unmodified or modified uridine.

The 3′ terminal U ribose can be modified with a 2′3′ cyclic phosphate as shown below:

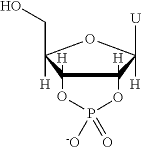

wherein “U” can be an unmodified or modified uridine.

Guide RNAs can contain 3′ nucleotides which can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphorothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, include without limitation methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O (CH₂) n-amino (wherein amino can be, e.g., NH₂; alkylamino, dialkylamino, heterocyclyl, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, O-and N-alkylated nucleotides, e.g., N6-methyl adenosine, can be incorporated into the gRNA. In certain embodiments, one or more or all of the nucleotides in a gRNA are deoxynucleotides.

Examples of gRNAs that include modifications and that may be used according to the embodiments herein include, without limitation, gRNAs that target the 13 nt target region. In certain embodiments, gRNAs may contain one or more modifications. In certain embodiments, the modifications may be at or near the 5′end. 3′ end or both the 5′ and 3′ ends of the gRNA. In certain embodiments, modifications may include, without limitation, addition of one or more PhTx groups or one or more 2′-O-methyl groups. In certain embodiments, the Sp35 gRNA (SEQ ID NO:970) may contain modifications at or near the 5′end and/or 3′ ends. Exemplary gRNAs that include modifications of Sp35 gRNA, such as additions of one or more of PhTx groups and/or 2′-O-methyl groups are set forth in Table 10.

In certain embodiments, gRNAs used according to the embodiments herein may include truncated forms of a full length gRNA. In certain embodiments, truncated gRNAs may have one or more nucleotides removed from the 5′ or 3′ end of the gRNA compared with a full-length gRNA. In certain embodiments, truncated gRNAs have one or more nucleotides removed from the protospacer region of the gRNA. Examples of gRNAs that are truncated that may be used according to the embodiments herein include, without limitation, the truncated forms of the gRNA Sp35 (SEQ ID NO:970), such as the sequences set forth in SEQ ID NOs:976-978 (Table 10).

### RNA-Guided Nucleases

RNA-guided nucleases according to the present disclosure include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g. complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g. Cas9 vs. Cpf1), species (e.g. S. pyogenes vs. S. aureus) or variation (e.g. full-length vs. truncated or split; naturally-occurring PAM specificity vs. engineered PAM specificity, etc.) of RNA-guided nuclease.

The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. In general, Cas9s recognize PAM sequences that are 3′ of the protospacer. Cpf1, on the other hand, generally recognizes PAM sequences that are 5′ of the protospacer.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov 2015. It should also be noted that engineered RNA-guided nucleases can have PAM specificities that differ from the PAM specificities of reference molecules (for instance, in the case of an engineered RNA-guided nuclease, the reference molecule may be the naturally occurring variant from which the RNA-guided nuclease is derived, or the naturally occurring variant having the greatest amino acid sequence homology to the engineered RNA-guided nuclease). Examples of PAMs that may be used according to the embodiments herein include, without limitation, the sequences set forth in SEQ ID NOs:199-205.

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above and in Ran & Hsu 2013, incorporated by reference herein), or that do not cut at all.

### Cas9

Crystal structures have been determined for S. pyogenes Cas9 (Jinek 2014), and for S. aureus Cas9 in complex with a unimolecular guide RNA and a target DNA (Nishimasu 2014; Anders 2014; and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g. a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, while the REC domain is thought to interact with the repeat: anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e. bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e. top) strand of the target nucleic acid. The PI domain, as its name suggests, contributes to PAM specificity. Examples of polypeptide sequences encoding Cas9 RuvC-like and Cas9 HNH-like domains that may be used according to the embodiments herein are set forth in SEQ ID NOs:15-23, 52-123 (RuvC-like domains) and SEQ ID NOs:24-28, 124-198 (HNH-like domains).

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat: antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains). Examples of polypeptide sequences encoding Cas9 molecules that may be used according to the embodiments herein are set forth in SEQ ID NOs: 1-2. 4-6, 12, 14.

### Cpf1

The crystal structure of Acidaminococcus sp. Cpf1 in complex with crRNA and a double-stranded (ds) DNA target including a TTTN PAM sequence has been solved by Yamano 2016 (incorporated by reference herein). Cpf1, like Cas9, has two lobes: a REC (recognition) lobe, and a NUC (nuclease) lobe. The REC lobe includes RECI and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, -II and -III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat: antirepeat duplex in Cas9 gRNAs.

### Modifications of RNA-Guided Nucleases

The RNA-guided nucleases described above have activities and properties that can be useful in a variety of applications, but the skilled artisan will appreciate that RNA-guided nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran & Hsu 2013 and Yamano 2016, as well as in Cotta-Ramusino. In general, mutations that reduce or eliminate activity in one of the two nuclease domains result in RNA-guided nucleases with nickase activity, but it should be noted that the type of nickase activity varies depending on which domain is inactivated. As one example, inactivation of a RuvC domain of a Cas9 will result in a nickase that cleaves the complementary or top strand. On the other hand, inactivation of a Cas9 HNH domain results in a nickase that cleaves the bottom or non-complementary strand.

Modifications of PAM specificity relative to naturally occurring Cas9 reference molecules has been described by Kleinstiver et al. for both S. pyogenes (Kleinstiver 2015a) and S. aureus (Kleinstiver 2015b. Kleinstiver et al. have also described modifications that improve the targeting fidelity of Cas9 (Kleinstiver 2016). Each of these references is incorporated by reference herein.

RNA-guided nucleases have been split into two or more parts, as described by Zetsche 2015b and by Fine 2015, both incorporated by reference herein.

RNA-guided nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger 2014, which is incorporated by reference for all purposes herein.

RNA-guided nucleases also optionally include a tag, such as, but not limited to, a nuclear localization signal to facilitate movement of RNA-guided nuclease protein into the nucleus. In certain embodiments, the RNA-guided nuclease can incorporate C- and/or N-terminal nuclear localization signals. Nuclear localization sequences are known in the art and are described in Maeder and elsewhere.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular RNA-guided nucleases, but it should be understood that the RNA-guided nucleases used may be modified in ways that do not alter their operating principles. Such modifications are within the scope of the present disclosure.

### Nucleic Acids Encoding RNA-Guided Nucleases

Nucleic acids encoding RNA-guided nucleases, e.g., Cas9, Cpf1 or functional fragments thereof, are provided herein. Examples of nucleic acid sequences encoding Cas9 molecules that may be used according to the embodiments herein are set forth in SEQ ID NOs:3, 7-11, 13. Exemplary nucleic acids encoding RNA-guided nucleases have been described previously (see, e.g., Cong 2013; Wang 2013; Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in Cotta-Ramusino.

In addition, or alternatively, a nucleic acid encoding an RNA-guided nuclease may comprise a nuclear localization sequence (NLS). Nuclear localization sequences are known in the art.

### Functional Analysis of Candidate Molecules

Candidate RNA-guided nucleases, gRNAs, and complexes thereof, can be evaluated by standard methods known in the art. See, e.g. Cotta-Ramusino. The stability of RNP complexes may be evaluated by differential scanning fluorimetry, as described below.

### Differential Scanning Fluorimetry (DSF)

The thermostability of ribonucleoprotein (RNP) complexes comprising gRNAs and RNA-guided nucleases can be measured via DSF. The DSF technique measures the thermostability of a protein, which can increase under favorable conditions such as the addition of a binding RNA molecule, e.g., a gRNA.

A DSF assay can be performed according to any suitable protocol, and can be employed in any suitable setting, including without limitation (a) testing different conditions (e.g. different stoichiometric ratios of gRNA: RNA-guided nuclease protein, different buffer solutions, etc.) to identify optimal conditions for RNP formation; and (b) testing modifications (e.g. chemical modifications, alterations of sequence, etc.) of an RNA-guided nuclease and/or a gRNA to identify those modifications that improve RNP formation or stability. One readout of a DSF assay is a shift in melting temperature of the RNP complex; a relatively high shift suggests that the RNP complex is more stable (and may thus have greater activity or more favorable kinetics of formation, kinetics of degradation, or another functional characteristic) relative to a reference RNP complex characterized by a lower shift. When the DSF assay is deployed as a screening tool, a threshold melting temperature shift may be specified, so that the output is one or more RNPs having a melting temperature shift at or above the threshold. For instance, the threshold can be 5-10° C. (e.g. 5°, 6°, 7°, 8°, 9°, 10°) or more, and the output may be one or more RNPs characterized by a melting temperature shift greater than or equal to the threshold.

Two non-limiting examples of DSF assay conditions are set forth below:

To determine the best solution to form RNP complexes, a fixed concentration (e.g. 2 μM) of Cas9 in water+10×SYPRO Orange® (Life Technologies cat #S-6650) is dispensed into a 384 well plate. An equimolar amount of gRNA diluted in solutions with varied pH and salt is then added. After incubating at room temperature for 10′and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

The second assay consists of mixing various concentrations of gRNA with fixed concentration (e.g. 2 μM) Cas9 in optimal buffer from assay 1 above and incubating (e.g. at RT for 10′) in a 384 well plate. An equal volume of optimal buffer+10×SYPRO Orange® (Life Technologies cat #S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

### Genome Editing Strategies

The genome editing systems described above are used, in various embodiments of the present disclosure, to generate edits in (i.e. to alter) targeted regions of DNA within or obtained from a cell. Various strategies are described herein to generate particular edits, and these strategies are generally described in terms of the desired repair outcome, the number and positioning of individual edits (e.g. SSBs or DSBs), and the target sites of such edits.

Genome editing strategies that involve the formation of SSBs or DSBs are characterized by repair outcomes including: (a) deletion of all or part of a targeted region; (b) insertion into or replacement of all or part of a targeted region; or (c) interruption of all or part of a targeted region. This grouping is not intended to be limiting, or to be binding to any particular theory or model, and is offered solely for economy of presentation. Skilled artisans will appreciate that the listed outcomes are not mutually exclusive and that some repairs may result in other outcomes. The description of a particular editing strategy or method should not be understood to require a particular repair outcome unless otherwise specified.

Replacement of a targeted region generally involves the replacement of all or part of the existing sequence within the targeted region with a homologous sequence, for instance through gene correction or gene conversion, two repair outcomes that are mediated by HDR pathways. HDR is promoted by the use of a donor template, which can be single-stranded or double stranded, as described in greater detail below. Single or double stranded templates can be exogenous, in which case they will promote gene correction, or they can be endogenous (e.g. a homologous sequence within the cellular genome), to promote gene conversion. Exogenous templates can have asymmetric overhangs (i.e. the portion of the template that is complementary to the site of the DSB may be offset in a 3′ or 5′ direction, rather than being centered within the donor template), for instance as described by Richardson 2016 (incorporated by reference herein). In instances where the template is single stranded, it can correspond to either the complementary (top) or non-complementary (bottom) strand of the targeted region.

Gene conversion and gene correction are facilitated, in some cases, by the formation of one or more nicks in or around the targeted region, as described in Ran and Cotta-Ramusino. In some cases, a dual-nickase strategy is used to form two offset SSBs that, in turn, form a single DSB having an overhang (e.g. a 5′ overhang).

Interruption and/or deletion of all or part of a targeted sequence can be achieved by a variety of repair outcomes. As one example, a sequence can be deleted by simultaneously generating two or more DSBs that flank a targeted region, which is then excised when the DSBs are repaired, as is described in Maeder for the LCA10 mutation. As another example, a sequence can be interrupted by a deletion generated by formation of a double strand break with single-stranded overhangs, followed by exonucleolytic processing of the overhangs prior to repair.

One specific subset of target sequence interruptions is mediated by the formation of an indel within the targeted sequence, where the repair outcome is typically mediated by NHEJ pathways (including Alt-NHEJ). NHEJ is referred to as an “error prone” repair pathway because of its association with indel mutations. In some cases, however, a DSB is repaired by NHEJ without alteration of the sequence around it (a so-called “perfect” or “scarless” repair); this generally requires the two ends of the DSB to be perfectly ligated. Indels, meanwhile, are thought to arise from enzymatic processing of free DNA ends before they are ligated that adds and/or removes nucleotides from either or both strands of either or both free ends.

Because the enzymatic processing of free DSB ends may be stochastic in nature, indel mutations tend to be variable, occurring along a distribution, and can be influenced by a variety of factors, including the specific target site, the cell type used, the genome editing strategy used, etc. Even so, it is possible to draw limited generalizations about indel formation: deletions formed by repair of a single DSB are most commonly in the 1-50 bp range, but can reach greater than 100-200 bp. Insertions formed by repair of a single DSB tend to be shorter and often include short duplications of the sequence immediately surrounding the break site. However, it is possible to obtain large insertions, and in these cases, the inserted sequence has often been traced to other regions of the genome or to plasmid DNA present in the cells.

Indel mutations—and genome editing systems configured to produce indels—are useful for interrupting target sequences, for example, when the generation of a specific final sequence is not required and/or where a frameshift mutation would be tolerated. They can also be useful in settings where particular sequences are preferred, insofar as the certain sequences desired tend to occur preferentially from the repair of an SSB or DSB at a given site. Indel mutations are also a useful tool for evaluating or screening the activity of particular genome editing systems and their components. In these and other settings, indels can be characterized by (a) their relative and absolute frequencies in the genomes of cells contacted with genome editing systems and (b) the distribution of numerical differences relative to the unedited sequence, e.g. ±1, ±2, ±3, etc. As one example, in a lead-finding setting, multiple gRNAs can be screened to identify those gRNAs that most efficiently drive cutting at a target site based on an indel readout under controlled conditions. Guides that produce indels at or above a threshold frequency, or that produce a particular distribution of indels, can be selected for further study and development. Indel frequency and distribution can also be useful as a readout for evaluating different genome editing system implementations or formulations and delivery methods, for instance by keeping the gRNA constant and varying certain other reaction conditions or delivery methods.

### Multiplex Strategies

Genome editing systems according to this disclosure may also be employed for multiplex gene editing to generate two or more DSBs, either in the same locus or in different loci. Any of the RNA-guided nucleases and gRNAs disclosed herein may be used in genome editing systems for multiplex gene editing. Strategies for editing that involve the formation of multiple DSBs, or SSBs, are described in, for instance, Cotta-Ramusino.

As disclosed herein, multiple gRNAs may be used in genome editing systems to introduce alterations (e.g., deletions, insertions) into the 13 nt target region of HBG1 and/or HBG2. In certain embodiments, one or more gRNAs comprising a targeting domain set forth in SEQ ID NOs:251-901, 940-942 may be used to introduce alterations in the 13 nt target region of HBG1 and/or HBG2. In certain embodiments, one or more gRNAs comprising a sequence set forth in SEQ ID NOs:970-979 may be used to introduce alterations in the 13 nt target region of HBG1 and/or HBG2. In other embodiments, multiple gRNAs may be used in genome editing systems to introduce alterations into the GATA1 binding motif in BCL11Ae. In certain embodiments, one or more gRNAs comprising a targeting domain set forth in SEQ ID NOs:952-955 may be used to introduce alterations in the GATA1 binding motif in BCL11Ae. Multiple gRNAs may also be used in genome editing systems to introduce alterations into the GATA1 binding motif in BCL11Ae and the 13 nt target region of HBG1 and/or HBG2. In certain embodiments, one or more gRNAs comprising a targeting domain set forth in SEQ ID NOs: 952-955 may be used to introduce alterations in the GATA1 binding motif in BCL11Ae and one or more gRNAs comprising a targeting domain set forth in SEQ ID NOs:251-901, 940-942 and/or one or more gRNAs comprising a sequence set forth in SEQ ID NOs:970-979 may be used to introduce alterations in the 13 nt target region of HBG1 and/or HBG2.

### Donor Template Design

Donor template design is described in detail in the literature, for instance in Cotta-Ramusino. DNA oligomer donor templates (oligodeoxynucleotides, ODNs, or template nucleic acids), which can be single stranded (ssODNs) or double-stranded (dsODNs), can be used to facilitate HDR-based repair of DSBs or to boost overall editing rate, and are particularly useful for introducing alterations into a target DNA sequence, inserting a new sequence into the target sequence, or replacing the target sequence altogether.

Whether single-stranded or double stranded, donor templates generally include regions that are homologous to regions of DNA within or near (e.g. flanking or adjoining) a target sequence to be cleaved. These homologous regions are referred to here as “homology arms,” and are illustrated schematically below:
[5′ homology arm]-[replacement sequence]--[3′ homology arm].

The homology arms can have any suitable length (including 0 nucleotides if only one homology arm is used), and 3′ and 5′ homology arms can have the same length, or can differ in length. The selection of appropriate homology arm lengths can be influenced by a variety of factors, such as the desire to avoid homologies or microhomologies with certain sequences such as Alu repeats or other very common elements. For example, a 5′ homology arm can be shortened to avoid a sequence repeat element. In other embodiments, a 3′ homology arm can be shortened to avoid a sequence repeat element. In some embodiments, both the 5′ and the 3′ homology arms can be shortened to avoid including certain sequence repeat elements. In addition, some homology arm designs can improve the efficiency of editing or increase the frequency of a desired repair outcome. For example, Richardson 2016, which is incorporated by reference, found that the relative asymmetry of 3′ and 5′ homology arms of single stranded donor templates influenced repair rates and/or outcomes.

Replacement sequences in donor templates have been described elsewhere, including in Cotta-Ramusino et al. A replacement sequence can be any suitable length (including zero nucleotides, where the desired repair outcome is a deletion), and typically includes one, two, three or more sequence modifications relative to the naturally-occurring sequence within a cell in which editing is desired. One common sequence modification involves the alteration of the naturally-occurring sequence to repair a mutation that is related to a disease or condition of which treatment is desired. Another common sequence modification involves the alteration of one or more sequences that are complementary to, or then, the PAM sequence of the RNA-guided nuclease or the targeting domain of the gRNA(s) being used to generate an SSB or DSB, to reduce or eliminate repeated cleavage of the target site after the replacement sequence has been incorporated into the target site.

Where a linear ssODN is used, it can be configured to (i) anneal to the nicked strand of the target nucleic acid, (ii) anneal to the intact strand of the target nucleic acid, (iii) anneal to the plus strand of the target nucleic acid, and/or (iv) anneal to the minus strand of the target nucleic acid. An ssODN may have any suitable length, e.g., about, at least, or no more than 150-200 nucleotides (e.g., 150, 160, 170, 180, 190, or 200 nucleotides).

It should be noted that a template nucleic acid can also be a nucleic acid vector, such as a viral genome or circular double stranded DNA, e.g., a plasmid. Nucleic acid vectors comprising donor templates can include other coding or non-coding elements. For example, a template nucleic acid can be delivered as part of a viral genome (e.g. in an AAV or lentiviral genome) that includes certain genomic backbone elements (e.g. inverted terminal repeats, in the case of an AAV genome) and optionally includes additional sequences coding for a gRNA and/or an RNA-guided nuclease. In certain embodiments, the donor template can be adjacent to, or flanked by, target sites recognized by one or more gRNAs, to facilitate the formation of free DSBs on one or both ends of the donor template that can participate in repair of corresponding SSBs or DSBs formed in cellular DNA using the same gRNAs. Exemplary nucleic acid vectors suitable for use as donor templates are described in Cotta-Ramusino, which is incorporated by reference.

Whatever format is used, a template nucleic acid can be designed to avoid undesirable sequences. In certain embodiments, one or both homology arms can be shortened to avoid overlap with certain sequence repeat elements, e.g., Alu repeats, LINE elements, etc.

In certain embodiments, silent, non-pathogenic SNPs may be included in the ssODN donor template to allow for identification of a gene editing event.

In certain embodiments, a donor template may be a non-specific template that is non-homologous to regions of DNA within or near a target sequence to be cleaved. In certain embodiments, donor templates for use in targeting the GATA1 binding motif in BCL11Ae may include, without limitation, non-target specific templates that are nonhomologous to regions of DNA within or near the GATA1 binding motif in BCL11Ae. In certain embodiments, donor templates for use in targeting the 13 nt target region may include, without limitation, non-target specific templates that are nonhomologous to regions of DNA within or near the 13 nt target region.

A donor template or template nucleic acid, as that term is used herein, refers to a nucleic acid sequence which can be used in conjunction with an RNA nuclease molecule and one or more gRNA molecules to alter (e.g., delete, disrupt, or modify) a target DNA sequence. In certain embodiments, the template nucleic acid results in a 13 nt deletion at the 13 nt target region of HBG1 and/or HBG2. In certain embodiments, the alteration (e.g., deletion) may be selected from HBG1 13 del c.-114 to -102, HBG2 13 del c.-114 to -102, or a combination thereof. In certain embodiments, the 13 nt target region may be selected from HBG1 c.-114 to -102 (e.g., nucleotides 2824-2836 of SEQ ID NO:902 (HBG1)), and HBG2 c.-114 to -102 (e.g., nucleotides 2748-2760 of SEQ ID NO:903 (HBG2)), or a combination thereof. In certain embodiments, the template nucleic acid may be a positive strand or a negative strand.

For example, a template nucleic acid for introducing the 13 nt deletion at the 13 nt target region (HBG1 c.-114 to -102 (e.g., nucleotides 2824-2836 of SEQ ID NO:902 (HBG1)), HBG2 c.-114 to -102 (e.g., nucleotides 2748-2760 of SEQ ID NO:903 (HBG2)), or a combination thereof may comprise a 5′ homology arm, a replacement sequence, and a 3′ homology arm, where the replacement sequence is 0 nucleotides or 0 bp. In certain embodiments, the 5′ homology arm comprises about 200 nucleotides in length, e.g., at least 25, 50, 75, 100, 125, 150, 175, or 200 nucleotides in length. In certain embodiments, the 5′ homology arm comprises about 50 to 100 bp, e.g., 55 to 95, 60 to 90, 70 to 90, or 80 to 90 bp, homology 5′ of the 13 nt target region (e.g., HBG1 c.-114 to -102 (e.g., nucleotides 2824-2836 of SEQ ID NO:902 (HBG1)) or HBG2 c.-114 to -102 (e.g., nucleotides 2748-2760 of SEQ ID NO:903 (HBG2)). In certain embodiments, the 5′ homology arm comprises, consists essentially of, or consists of SEQ ID NO:904 (ssODN1 5′ homology arm), SEQ ID NO:907 (PhTx ssODN1 5′homology arm), SEQ ID NO:991 (OLI16414 5′ homology arm), or SEQ ID NO:994 (OLI16412 5′ homology arm). In certain embodiments, the 3′ homology arm comprises about 200 nucleotides in length, e.g., at least 25, 50, 75, 100, 125, 150, 175, or 200 nucleotides in length. In certain embodiments, the 3′ homology arm comprises about 50 to 100 bp, e.g., 55 to 95, 60 to 90, 70 to 90, or 80 to 90 bp, homology 3′ of the 13 nt target region (e.g., HBG1 c.-114 to -102 (e.g., nucleotides 2824-2836 of SEQ ID NO:902 (HBG1)) or HBG2 c.-114 to -102 (e.g., nucleotides 2748-2760 of SEQ ID NO:903 (HBG2)). In certain embodiments, the 3′ homology arm comprises, consists essentially of, or consists of SEQ ID NO:905 (SSODN1 3′ homology arm), SEQ ID NO:908 (PhTx ssODN1 3′homology arm), SEQ ID NO:992 (OLI16414 3′ homology arm), or SEQ ID NO:995 (OLI16412 3′ homology arm). In certain embodiments, the template nucleic acid comprises, consists essentially of, or consists of SEQ ID NO:906 (sSODN1), SEQ ID NO:909 (PhTx ssODN1), SEQ ID NO:990 (OLI16414), or SEQ ID NO:995 (OLI16412).

In certain embodiments, the template nucleic acid comprises one or more phosphorothioate (PhTx) modifications. In certain embodiments, the 5′ homology arm comprises one or more PhTx modifications. In certain embodiments, the 3′ homology arm comprises one or more PhTx modifications. In certain embodiments, the template nucleic acid comprises one or more PhTx modifications at or near the 5′ end and/or 3′ end.

In certain embodiments, the ssODNs for introducing the 13 nt deletion at the 13 nt target region described herein may be used in conjunction with an RNA nuclease and one or more gRNAs that target the 13 nt target region, for example, the gRNAs disclosed in Table 10. In certain embodiments, the gRNAs may be chemically synthesized gRNAs.

### Target Cells

Genome editing systems according to this disclosure can be used to manipulate or alter a cell, e.g., to edit or alter a target nucleic acid. The manipulating can occur, in various embodiments, in vivo or ex vivo.

A variety of cell types can be manipulated or altered according to the embodiments of this disclosure, and in some cases, such as in vivo applications, a plurality of cell types are altered or manipulated, for example by delivering genome editing systems according to this disclosure to a plurality of cell types. In other cases, however, it may be desirable to limit manipulation or alteration to a particular cell type or types. For instance, it can be desirable in some instances to edit a cell with limited differentiation potential or a terminally differentiated cell, such as a photoreceptor cell in the case of Maeder, in which modification of a genotype is expected to result in a change in cell phenotype. In other cases, however, it may be desirable to edit a less differentiated, multipotent or pluripotent, stem or progenitor cell. By way of example, the cell may be an embryonic stem cell, induced pluripotent stem cell (iPSC), hematopoietic stem/progenitor cell (HSPC), or other stem or progenitor cell type that differentiates into a cell type of relevance to a given application or indication.

As a corollary, the cell being altered or manipulated is, variously, a dividing cell or a non-dividing cell, depending on the cell type(s) being targeted and/or the desired editing outcome.

When cells are manipulated or altered ex vivo, the cells can be used (e.g. administered to a subject) immediately, or they can be maintained or stored for later use. Those of skill in the art will appreciate that cells can be maintained in culture or stored (e.g. frozen in liquid nitrogen) using any suitable method known in the art.

### Implementation of Genome Editing Systems: Delivery, Formulations, and Routes of Administration

As discussed above, the genome editing systems of this disclosure can be implemented in any suitable manner, meaning that the components of such systems, including without limitation the RNA-guided nuclease, gRNA, and optional donor template nucleic acid, can be delivered, formulated, or administered in any suitable form or combination of forms that results in the transduction, expression or introduction of a genome editing system and/or causes a desired repair outcome in a cell, tissue or subject. Tables 3 and 4 set forth several, non-limiting examples of genome editing system implementations. Those of skill in the art will appreciate, however, that these listings are not comprehensive, and that other implementations are possible. With reference to Table 3 in particular, the table lists several exemplary implementations of a genome editing system comprising a single gRNA and an optional donor template. However, genome editing systems according to this disclosure can incorporate multiple gRNAs, multiple RNA-guided nucleases, and other components such as proteins, and a variety of implementations will be evident to the skilled artisan based on the principles illustrated in the table. In the table, [N/A] indicates that the genome editing system does not include the indicated component.

TABLE 3
Genome Editing System Components

| RNA-guided | | Donor | |
|---|---|---|---|
| Nuclease | gRNA | Template | Comments |
| Protein | RNA | [N/A] | An RNA-guided nuclease protein |
| | | | complexed with a gRNA molecule |
| | | | (an RNP complex) |
| Protein | RNA | DNA | An RNP complex as described |
| | | | above plus a single-stranded |
| | | | or double stranded donor template. |
| Protein | DNA | [N/A] | An RNA-guided nuclease protein |
| | | | plus gRNA transcribed from DNA. |
| Protein | DNA | DNA | An RNA-guided nuclease protein |
| | | | plus gRNA-encoding DNA and a |
| | | | separate DNA donor template. |
| Protein | DNA | | An RNA-guided nuclease protein |
| | | | and a single DNA encoding both |
| | | | a gRNA and a donor template. |
| DNA | | | A DNA or DNA vector encoding an |
| | | | RNA-guided nuclease, a gRNA and |
| | | | a donor template. |
| DNA | DNA | [N/A] | Two separate DNAs, or two |
| | | | separate DNA vectors, encoding |
| | | | the RNA-guided nuclease and the |
| | | | gRNA, respectively. |
| DNA | DNA | DNA | Three separate DNAs, or three |
| | | | separate DNA vectors, encoding |
| | | | the RNA-guided nuclease, the |
| | | | gRNA and the donor template, |
| | | | respectively. |
| DNA | | [N/A] | A DNA or DNA vector encoding |
| | | | an RNA-guided nuclease and a |
| | | | gRNA |
| DNA | | DNA | A first DNA or DNA vector |
| | | | encoding an RNA-guided |
| | | | nuclease and a gRNA, and a |
| | | | second DNA or DNA vector |
| | | | encoding a donor template. |
| DNA | DNA | | A first DNA or DNA vector |
| | | | encoding an RNA-guided |
| | | | nuclease and second DNA or |
| | | | DNA vector encoding a gRNA |
| | | | and a donor template. |
| DNA | | | A first DNA or DNA vector |
| | DNA | | encoding an RNA-guided |
| | | | nuclease and a donor template, |
| | | | and a second DNA or DNA vector |
| | | | encoding a gRNA |
| DNA | | | A DNA or DNA vector encoding |
| | RNA | | an RNA-guided nuclease and a |
| | | | donor template, and a gRNA |
| RNA | | [N/A] | An RNA or RNA vector encoding |
| | | | an RNA-guided nuclease and |
| | | | comprising a gRNA |
| RNA | | DNA | An RNA or RNA vector encoding |
| | | | an RNA-guided nuclease and |
| | | | comprising a gRNA, and a DNA or |
| | | | DNA vector encoding a donor |
| | | | template. |

Table 4 summarizes various delivery methods for the components of genome editing systems, as described herein. Again, the listing is intended to be exemplary rather than limiting.

TABLE 4

| | | Delivery | | | |
|---|---|---|---|---|---|
| | | into Non- | | | Type of |
| | | Dividing | Duration of | Genome | Molecule |
| Delivery Vector/Mode | | Cells | Expression | Integration | Delivered |
| Physical (e.g., electroporation, | | YES | Transient | NO | Nucleic Acids |
| particle gun, Calcium Phosphate | | | | | and Proteins |
| transfection, cell compression or | | | | | |
| squeezing) | | | | | |
| Viral | Retrovirus | NO | Stable | YES | RNA |
| | Lentivirus | YES | Stable | YES/NO with | RNA |
| | | | | modifications | |
| | Adenovirus | YES | Transient | NO | DNA |
| | Adeno- | YES | Stable | NO | DNA |
| | Associated Virus | | | | |
| | (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

### Nucleic Acid-Based Delivery of Genome Editing Systems

Nucleic acids encoding the various elements of a genome editing system according to the present disclosure can be administered to subjects or delivered into cells by art-known methods or as described herein. For example, RNA-guided nuclease-encoding and/or gRNA-encoding DNA, as well as donor template nucleic acids can be delivered by, e.g., vectors (e.g., viral or non-viral vectors), non-vector based methods (e.g., using naked DNA or DNA complexes), or a combination thereof.

Nucleic acids encoding genome editing systems or components thereof can be delivered directly to cells as naked DNA or RNA, for instance by means of transfection or electroporation, or can be conjugated to molecules (e.g., N-acetylgalactosamine) promoting uptake by the target cells (e.g., erythrocytes, HSCs). Nucleic acid vectors, such as the vectors summarized in Table 4, can also be used.

Nucleic acid vectors can comprise one or more sequences encoding genome editing system components, such as an RNA-guided nuclease, a gRNA and/or a donor template. A vector can also comprise a sequence encoding a signal peptide (e.g., for nuclear localization, nucleolar localization, or mitochondrial localization), associated with (e.g., inserted into or fused to) a sequence coding for a protein. As one example, a nucleic acid vectors can include a Cas9 coding sequence that includes one or more nuclear localization sequences (e.g., a nuclear localization sequence from SV40).

The nucleic acid vector can also include any suitable number of regulatory/control elements, e.g., promoters, enhancers, introns, polyadenylation signals, Kozak consensus sequences, or internal ribosome entry sites (IRES). These elements are well known in the art, and are described in Cotta-Ramusino.

Nucleic acid vectors according to this disclosure include recombinant viral vectors. Exemplary viral vectors are set forth in Table 4, and additional suitable viral vectors and their use and production are described in Cotta-Ramusino. Other viral vectors known in the art can also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One important category of non-viral nucleic acid vectors are nanoparticles, which can be organic or inorganic. Nanoparticles are well known in the art, and are summarized in Cotta-Ramusino. Any suitable nanoparticle design can be used to deliver genome editing system components or nucleic acids encoding such components. For instance, organic (e.g. lipid and/or polymer) nonparticles can be suitable for use as delivery vehicles in certain embodiments of this disclosure. Exemplary lipids for use in nanoparticle formulations, and/or gene transfer are shown in Table 5, and Table 6 lists exemplary polymers for use in gene transfer and/or nanoparticle formulations.

TABLE 5
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3- | DOPE | Helper |
| phosphatidylethanolamine | | |
| Cholesterol | | Helper |
| N-[1-(2,3-Dioleyloxy)propyl]N,N,N- | DOTMA | Cationic |
| trimethylammonium chloride | | |
| 1,2-Dioleoyloxy-3-trimethylammonium- | DOTAP | Cationic |
| propane | | |
| Dioctadecylamidoglycylspermine | DOGS | Cationic |
| N-(3-Aminopropyl)-N,N-dimethyl-2,3- | GAP-DLRIE | Cationic |
| bis(dodecyloxy)-1-propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6- | 20c | Cationic |
| trimethylpyridinium | | |
| 2,3-Diolcyloxy-N-[2(sperminecarboxamido- | DOSPA | Cationic |
| ethyl]-N,N-dimethyl-1-propanaminium | | |
| trifluoroacetate | | |
| 1,2-Dioleyl-3-trimethylammonium-propane | DOPA | Cationic |
| N-(2-Hydroxyethyl)-N,N-dimethyl-2,3- | MDRIE | Cationic |
| bis(tetradecyloxy)-1-propanaminium bromide | | |
| Dimyristooxypropyl dimethyl hydroxyethyl | DMRI | Cationic |
| ammonium bromide | | |
| 3β-[N-(N′,N′-Dimethylaminoethane)- | DC-Chol | Cationic |
| carbamoyl]cholesterol | | |
| Bis-guanidium-tren-cholesterol | BGTC | Cationic |
| 1,3-Diodeoxy-2-(6-carboxy-spermyl)- | DOSPER | Cationic |
| propylamide | | |
| Dimethyloctadecylammonium bromide | DDAB | Cationic |
| Dioctadecylamidoglicylspermidin | DSL | Cationic |
| rac-[(2,3-Dioctadecyloxypropyl)(2- | CLIP-1 | Cationic |
| hydroxyethyl)]-dimethylammonium chloride | | |
| rac-[2(2,3-Dihexadecyloxypropyl- | CLIP-6 | Cationic |
| oxymethyloxy)ethyl]trimethylammonium | | |
| bromide | | |
| Ethyldimyristoylphosphatidylcholine | EDMPC | Cationic |
| 1,2-Distcaryloxy-N,N-dimethyl-3- | DSDMA | Cationic |
| aminopropane | | |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3- | DSEPC | Cationic |
| ethylphosphocholine | | |
| N-Palmitoyl D-erythro-sphingosyl carbamoyl- | CCS | Cationic |
| spermine | | |
| N-t-Butyl-N0-tetradecyl-3- | diC14- | Cationic |
| tetradecylaminopropionamidine | amidine | |
| Octadecenolyoxy[ethyl-2-heptadecenyl-3 | DOTIM | Cationic |
| hydroxyethyl] imidazolinium chloride | | |
| N1-Cholesteryloxycarbonyl-3,7-diazanonane- | CDAN | Cationic |
| 1,9-diamine | | |
| 2-(3-[Bis(3-amino-propyl)- | RPR209120 | Cationic |
| amino]propylamino)-N- | | |
| ditetradecylcarbamoylme-ethyl-acetamide | | |
| 1,2-dilinoleyloxy-3- dimethylaminopropane | DLinDMA | Cationic |
| 2,2-dilinoleyl-4-dimethylaminoethyl-[1,3]- | DLin-KC2- | Cationic |
| dioxolane | DMA | |
| dilinoleyl-methyl-4-dimethylaminobutyrate | DLin-MC3- | Cationic |
| | DMA | |

TABLE 6
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethylene imine) biscarbamate | PEIC |
| Poly(L-lysine) | PLL |
| Histidine modified PLL | |
| Poly(N-vinylpyrrolidonc) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly(2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Non-viral vectors optionally include targeting modifications to improve uptake and/or selectively target certain cell types. These targeting modifications can include e.g., cell specific antigens, monoclonal antibodies, single chain antibodies, aptamers, polymers, sugars (e.g., N-acetylgalactosamine (GalNAc)), and cell penetrating peptides. Such vectors also optionally use fusogenic and endosome-destabilizing peptides/polymers, undergo acid-triggered conformational changes (e.g., to accelerate endosomal escape of the cargo), and/or incorporate a stimuli-cleavable polymer, e.g., for release in a cellular compartment. For example, disulfide-based cationic polymers that are cleaved in the reducing cellular environment can be used.

In certain embodiments, one or more nucleic acid molecules (e.g., DNA molecules) other than the components of a genome editing system, e.g., the RNA-guided nuclease component and/or the gRNA component described herein, are delivered. In certain embodiments, the nucleic acid molecule is delivered at the same time as one or more of the components of the Genome editing system. In certain embodiments, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Genome editing system are delivered. In certain embodiments, the nucleic acid molecule is delivered by a different means than one or more of the components of the genome editing system, e.g., the RNA-guided nuclease component and/or the gRNA component, are delivered. The nucleic acid molecule can be delivered by any of the delivery methods described herein. For example, the nucleic acid molecule can be delivered by a viral vector, e.g., an integration-deficient lentivirus, and the RNA-guided nuclease molecule component and/or the gRNA component can be delivered by electroporation, e.g., such that the toxicity caused by nucleic acids (e.g., DNAs) can be reduced. In certain embodiments, the nucleic acid molecule encodes a therapeutic protein, e.g., a protein described herein. In certain embodiments, the nucleic acid molecule encodes an RNA molecule, e.g., an RNA molecule described herein.

### Delivery of RNPs and/or RNA Encoding Genome Editing System Components

RNPs (complexes of gRNAs and RNA-guided nucleases) and/or RNAs encoding RNA-guided nucleases and/or gRNAs, can be delivered into cells or administered to subjects by art-known methods, some of which are described in Cotta-Ramusino. In vitro, RNA-guided nuclease-encoding and/or gRNA-encoding RNA can be delivered, e.g., by microinjection, electroporation, transient cell compression or squeezing (see, e.g., Lee 2012). Lipid-mediated transfection, peptide-mediated delivery, GalNAc- or other conjugate-mediated delivery, and combinations thereof, can also be used for delivery in vitro and in vivo. A protective, interactive, non-condensing (PINC) system may be used for delivery.

In vitro delivery via electroporation comprises mixing the cells with the RNA encoding RNA-guided nucleases and/or gRNAs, with or without donor template nucleic acid molecules, in a cartridge, chamber or cuvette and applying one or more electrical impulses of defined duration and amplitude. Systems and protocols for electroporation are known in the art, and any suitable electroporation tool and/or protocol can be used in connection with the various embodiments of this disclosure.

### Route of Administration

Genome editing systems, or cells altered or manipulated using such systems, can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically can be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In certain embodiments, significantly smaller amounts of the components (compared with systemic approaches) can exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration can be provided as a periodic bolus (for example, intravenously) or as continuous infusion from an internal reservoir or from an external reservoir (for example, from an intravenous bag or implantable pump). Components can be administered locally, for example, by continuous release from a sustained release drug delivery device.

In addition, components can be formulated to permit release over a prolonged period of time. A release system can include a matrix of a biodegradable material or a material which releases the incorporated components by diffusion. The components can be homogeneously or heterogeneously distributed within the release system. A variety of release systems can be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically the microspheres are composed of a polymer of lactic acid and glycolic acid, which are structured to form hollow spheres. The spheres can be approximately 15-30 microns in diameter and can be loaded with components described herein. In some embodiments, genome editing systems, system components and/or nucleic acids encoding system components, are delivered with a block copolymer such as a poloxamer or a poloxamine.

### Multi-Modal or Differential Delivery of Components

Skilled artisans will appreciate, in view of the instant disclosure, that different components of genome editing systems disclosed herein can be delivered together or separately and simultaneously or nonsimultaneously. Separate and/or asynchronous delivery of genome editing system components can be particularly desirable to provide temporal or spatial control over the function of genome editing systems and to limit certain effects caused by their activity.

Different or differential modes as used herein refer to modes of delivery that confer different pharmacodynamic or pharmacokinetic properties on the subject component molecule, e.g., a RNA-guided nuclease molecule, gRNA, template nucleic acid, or payload. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV or lentivirus, delivery.

By way of example, the components of a genome editing system, e.g., a RNA-guided nuclease and a gRNA, can be delivered by modes that differ in terms of resulting half-life or persistent of the delivered component the body, or in a particular compartment, tissue or organ. In certain embodiments, a gRNA can be delivered by such modes. The RNA-guided nuclease molecule component can be delivered by a mode which results in less persistence or less exposure to the body or a particular compartment or tissue or organ.

More generally, in certain embodiments, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In certain embodiments, the first pharmacodynamic or pharmacokinetic property, e.g., distribution, persistence or exposure, is more limited than the second pharmacodynamic or pharmacokinetic property.

In certain embodiments, the first mode of delivery is selected to optimize, e.g., minimize, a pharmacodynamic or pharmacokinetic property, e.g., distribution, persistence or exposure.

In certain embodiments, the second mode of delivery is selected to optimize, e.g., maximize, a pharmacodynamic or pharmacokinetic property, e.g., distribution, persistence or exposure.

In certain embodiments, the first mode of delivery comprises the use of a relatively persistent element, e.g., a nucleic acid, e.g., a plasmid or viral vector, e.g., an AAV or lentivirus. As such vectors are relatively persistent product transcribed from them would be relatively persistent.

In certain embodiments, the second mode of delivery comprises a relatively transient element, e.g., an RNA or protein.

In certain embodiments, the first component comprises gRNA, and the delivery mode is relatively persistent, e.g., the gRNA is transcribed from a plasmid or viral vector, e.g., an AAV or lentivirus. Transcription of these genes would be of little physiological consequence because the genes do not encode for a protein product, and the gRNAs are incapable of acting in isolation. The second component, a RNA-guided nuclease molecule, is delivered in a transient manner, for example as mRNA or as protein, ensuring that the full RNA-guided nuclease molecule/gRNA complex is only present and active for a short period of time.

Furthermore, the components can be delivered in different molecular form or with different delivery vectors that complement one another to enhance safety and tissue specificity.

Use of differential delivery modes can enhance performance, safety, and/or efficacy, e.g., the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by MHC molecules. A two-part delivery system can alleviate these drawbacks.

Differential delivery modes can be used to deliver components to different, but overlapping target regions. The formation active complex is minimized outside the overlap of the target regions. Thus, in certain embodiments, a first component, e.g., a gRNA is delivered by a first delivery mode that results in a first spatial, e.g., tissue, distribution. A second component, e.g., a RNA-guided nuclease molecule is delivered by a second delivery mode that results in a second spatial, e.g., tissue, distribution. In certain embodiments, the first mode comprises a first element selected from a liposome, nanoparticle, e.g., polymeric nanoparticle, and a nucleic acid, e.g., viral vector. The second mode comprises a second element selected from the group. In certain embodiments, the first mode of delivery comprises a first targeting element, e.g., a cell specific receptor or an antibody, and the second mode of delivery does not include that element. In certain embodiments, the second mode of delivery comprises a second targeting element, e.g., a second cell specific receptor or second antibody.

When the RNA-guided nuclease molecule is delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA and the RNA-guided nuclease molecule are packaged in separated delivery vehicles with distinct but overlapping tissue tropism, the fully functional complex is only be formed in the tissue that is targeted by both vectors.

### EXAMPLES

The principles and embodiments described above are further illustrated by the non-limiting examples that follow:

### Example 1: Screening of S. pyogenes gRNAs Delivered to K562 Cells as Ribonucleoprotein Complexes for Use in Causing 13 nt Deletions in HBG1 and HBG2 Regulatory Regions

gRNAs targeting a 26 nt fragment spanning and including the 13 nucleotides at the 13 nt target region of HBG1 and HBG2 were designed by standard methods. After gRNAs were designed in silico and tiered, a subset of the gRNAs were selected and screened for activity and specificity in human K562 cells. The gRNAs selected for screening are set forth in Table 7. Briefly, gRNAs were in vitro transcribed and then complexed with S. pyogenes wildtype (Wt) Cas9 protein to form ribonucleoprotein complexes (RNPs). The gRNAs complexed to S. pyogenes Cas9 protein were modified sgRNAs ((e.g., 5′ ARCA capped and 3′ polyA (20A) tail; Table 7) and target the HBG1 and HBG2 regulatory regions. To allow for direct comparison of the activity of these RNPs in K562 cells and human CD34⁺ cells, RNPs were first delivered to K562 cells by electroporation (Amaxa Nucleofector).

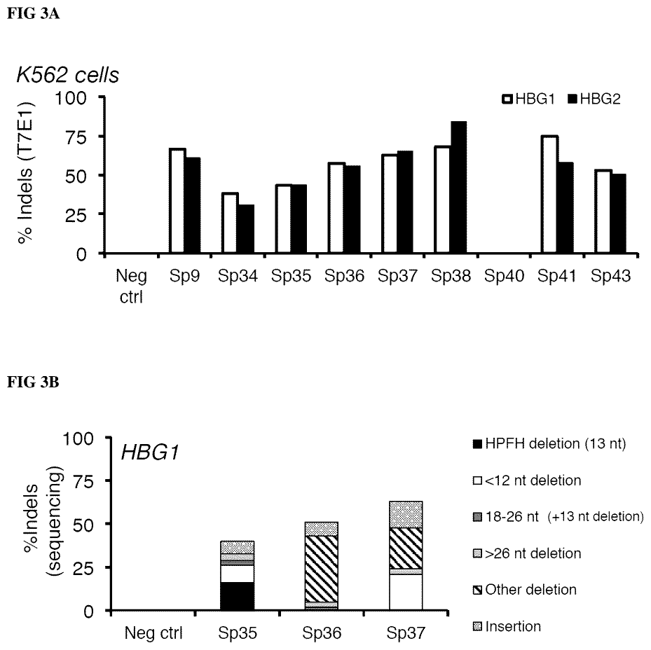

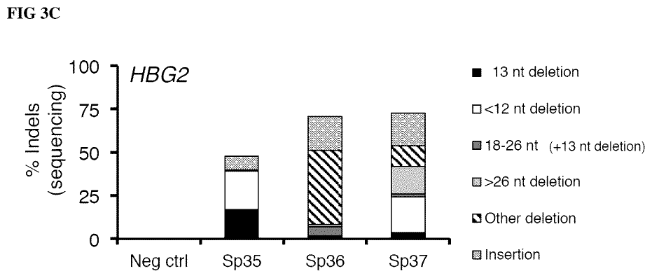

Three days after RNP electroporation, gDNA was extracted from K562 cells and then the HBG1and HBG2 loci were PCR amplified from the gDNA. Gene editing was evaluated in the PCR products by T7E1 endonuclease assay analysis. Eight out of nine RNPs supported a high percentage of NHEJ. Sp37 RNP, the only gRNA shown to be active in human CD34⁺ cells (<10% editing in CD34⁺ cells) was highly active in K562 cells, with >60% indels detected at both HBG1 and HBG2 and eight cut in both the HBG1 and HBG2 targeted regions in the promoter sequences (FIG. 3A).

TABLE 7
Selected gRNAs for screening in K562
cells or CD34⁺ cells

| | | | | Targeting | |
|---|---|---|---|---|---|
| | | | Targeting | domain | |
| | Targeting | Targeting | domain | sequence | |
| | domain | domain | sequence | plus | |
| gRNA | sequence | sequence | plus PAM | PAM (NGG) | |
| ID | (RNA) | (DNA) | (NGG)(RNA) | (DNA) | Sense |
| Sp9 | GGCUAUUG | GGCTATTG | GGCUAUUGG | GGCTATT | Anti- |
| | GUCAAGGC | GTCAAGGC | UCAAGGCAA | GGTCAAGG | sense |
| | A | A | GG(SEQ ID | CAAGG | |
| | (SEQ ID | (SEQ ID | NO: 920) | (SEQ ID | |
| | NO: 277) | NO: 910) | | NO: 930) | |
| Sp36 | CAAGGCUA | CAAGGCTAT | CAAGGCUAU | CAAGGCTA | Anti- |
| | UUGGUCAA | TGGTCAAGG | UGGUCAAGG | TTGGTCAA | sense |
| | GGCA | CA | CAAGG | GGCAAGG | |
| | (SEQ ID | (SEQ ID | (SEQ ID | (SEQ ID | |
| | NO: 338) | NO: 911) | NO: 921) | NO: 931) | |
| Sp40 | UGCCUUGU | TGCCTTGTC | UGCCUUGUC | TGCCTTGT | Anti- |
| | CAAGGCUA | AAGGCTAT | AAGGCUAUU | CAAGGCTA | sense |
| | U | (SEQ ID | GG | TTGG | |
| | (SEQ ID | NO: 912) | (SEQ ID | (SEQ ID | |
| | NO: 327) | | NO: 922) | NO: 932) | |
| Sp42 | GUUUGCCU | GTTTGCCTT | GUUUGCCUU | GTTTGCCT | Anti- |
| | UGUCAAGG | GTCAAGGCT | GUCAAGGCU | TGTCAAGG | sense |
| | CUAU | AT | AUUGG | CTATTGG | |
| | (SEQ ID | (SEQ ID | (SEQ ID | (SEQ ID | |
| | NO: 299) | NO: 913) | NO: 923) | NO: 933) | |
| Sp38 | GACCAAUA | GACCAATAG | GACCAAUAG | GACCAATA | Sense |
| | GCCUUGAC | CCTTGACA | CCUUGACAA | GCCTTGAC | |
| | A | (SEQ ID | GG | AAGG | |
| | (SEQ ID | NO: 914) | (SEQ ID | (SEQ ID | |
| | NO: 276) | | NO: 924) | NO: 934) | |
| Sp37 | CUUGACCA | CTTGACCAA | CUUGACCAA | CTTGACCA | Sense |
| | AUAGCCUU | TAGCCTTGA | UAGCCUUGA | ATAGCCTT | |
| | GACA | CA | CAAGG | GACAAGG | |
| | (SEQ ID | (SEQ ID | (SEQ ID | (SEQ ID | |
| | NO: 333) | NO: 915) | NO: 925) | NO: 935) | |
| Sp43 | GUCAAGGC | GTCAAGGCT | GUCAAGGCU | GTCAAGGC | Anti- |
| | UAUUGGUC | ATTGGTCA | AUUGGUCAA | TATTGGTC | sense |
| | A | (SEQ ID | GG | AAGG | |
| | (SEQ ID | NO: 916) | (SEQ ID | (SEQ ID | |
| | NO: 278) | | NO: 926) | NO: 936) | |
| Sp35 | CUUGUCAA | CTTGTCAAG | CUUGUCAAG | CTTGTCAA | Anti- |
| | GGCUAUUG | GCTATTGGT | GCUAUUGGU | GGCTATTG | sense |
| | GUCA | CA | CAAGG | GTCAAGG | |
| | (SEQ ID | (SEQ ID | (SEQ ID | (SEQ ID | |
| | NO: 339) | NO: 917) | NO: 927) | NO: 937) | |
| Sp41 | UCAAGUUU | TCAAGTTTG | UCAAGUUUG | TCAAGTTT | Anti- |
| | GCCUUGUC | CCTTGTCA | CCUUGUCAA | GCCTTGTC | sense |
| | A | (SEQ ID | GG | AAGG | |
| | (SEQ ID | NO: 918) | (SEQ ID | (SEQ ID | |
| | NO: 310) | | NO: 928) | NO: 938) | |
| Sp34 | UGGUCAAG | TGGTCAAGT | UGGUCAAGU | TGGTCAAG | Anti- |
| | UUUGCCUU | TTGCCTTGT | UUGCCUUGU | TTTGCCTT | sense |
| | GUCA | CA | CAAGG | GTCAAGG | |
| | (SEQ ID | (SEQ ID | (SEQ ID | (SEQ ID | |
| | NO: 340) | NO: 919) | NO: 929) | NO: 939) | |
| Sp85 | AGUAUCCA | AGTATCCAG | AGUAUCCAG | AGTATCCA | Anti- |
| | GUGAGGCC | TGAGGCCA | UGAGGCCAG | GTGAGGCC | sense |
| | A | (SEQ ID | GG | AGGG | |
| | (SEQ ID | NO: 943) | (SEQ ID | (SEQ ID | |
| | NO: 940) | | NO: 946) | NO: 949) | |
| SpA | GGCAAGGC | GGCAAGGCT | GGCAAGGCU | GGCAAGGC | Sense |
| | UGGCCAAC | GGCCAACCC | GGCCAACCC | TGGCCAAC | |
| | CCAU | AT | AUGGG | CCATGGG | |
| | (SEQ ID | (SEQ ID | (SEQ ID | (SEQ ID | |
| | NO: 941) | NO: 944) | NO: 947) | NO: 950) | |
| SpB | UAUUUGCA | TATTTGCAT | UAUUUGCAU | TATTTGCA | Sense |
| | UUGAGAUA | TGAGATAGT | UGAGAUAGU | TTGAGATA | |
| | GUGU | GT | GUGGG | GTGTGGG | |
| | (SEQ ID | (SEQ ID | (SEQ ID | (SEQ ID | |
| | NO: 942) | NO: 945) | NO: 948) | NO: 951) | |

The HBG1 and HBG2 PCR products for the K562 cells that were targeted with the eight active sgRNAs were then analyzed by DNA sequencing analysis and scored for insertions and deletions detected. The deletions were subdivided into precise 13 nt deletions at the target site, 13 nt target site inclusive and proximal small deletions (18-26 nt), 12 nt deletions (i.e., partial deletion) of the 13 nt target site, >26 nt deletions that span a portion of the HPFH target site, and other deletions, e.g., deletions proximal to but outside the HPFH target site. Seven of the eight sgRNAs targeted deletion of the 13 nt (HPFH mutation induction) (FIG. 3B) for HBG1. At least five of the eight sgRNAs also supported targeted deletion of the 13 nt in HBG2 promoter region (FIG. 3C). Note that DNA sequence results for HBG2 in cells treated with HBG Sp34 sgRNA were not available. These data indicate that Cas9 and sgRNA support precise induction of the 13 nt deletions. FIGS. 3B-3C depict examples of the types of deletions observed in target sequences in HBG1.

### Example 2: Cas9 RNP Containing gRNA Targeting the 13 nt Deletion Mutation Supports Gene Editing in Human Hematopoietic Stem/Progenitor Cells

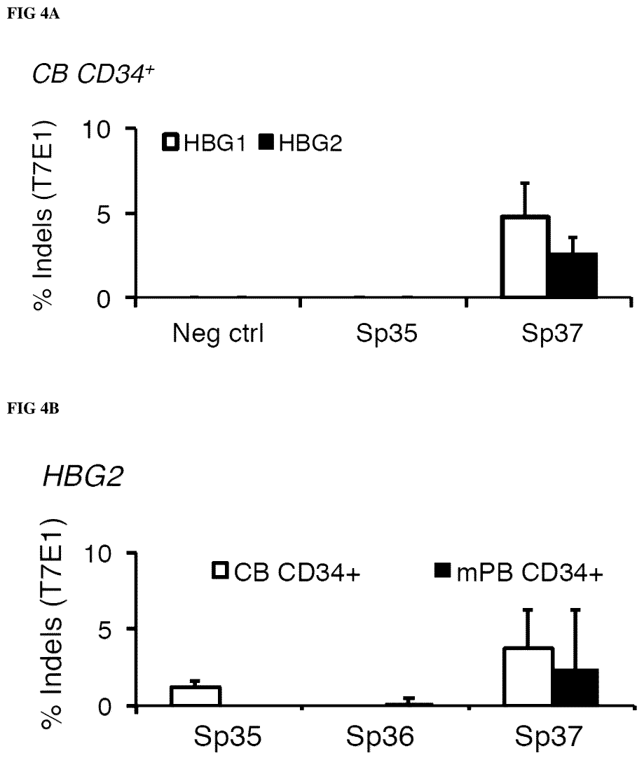

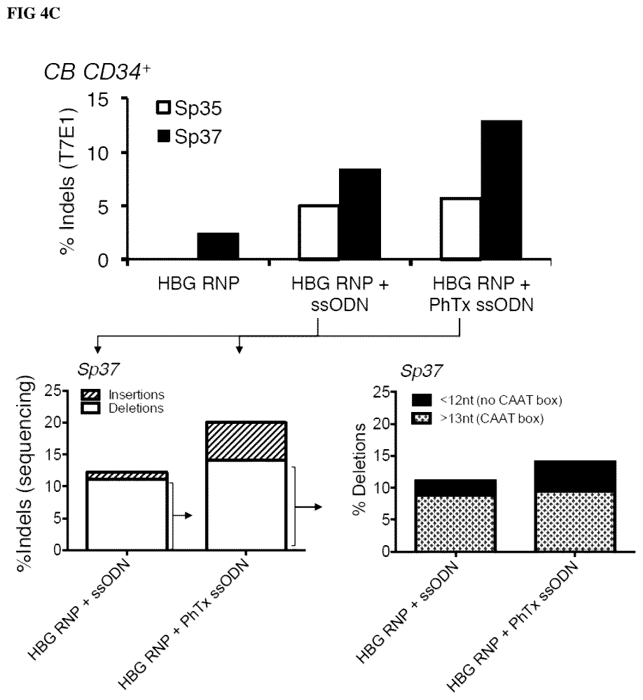

Of the RNPs containing different gRNAs tested in human cord blood (CB) CD34⁺ cells, only Sp37 resulted in detectable editing at the target site in the HBG1 and HBG2 promoters as determined by T7E1 analysis of indels in HBG1 and HBG2 specific PCR products amplified from gDNA extracted from electroporated CB CD34⁺ cells from a three cord blood donors (FIG. 4A). The average level of editing detected in cells electroporated with Cas9 protein complexed to Sp37 was 5±2% indels at HBG1 and 3±1% indels detected at HBG2 (3 separate experiments, and CB donors).

Next, three S. pyogenes gRNAs whose target sites are within the HBG promoter (Sp35, Sp36,Sp37) were complexed to wild-type S. pyogenes Cas9 protein to form ribonucleoprotein complexes. These HBG targeted RNPS were electroporated into CB CD34⁺ cells (n=3 donors) and adult mobilized peripheral blood (mPB) CD34⁺ cell donors (n=3 donors). Then the level of editing at the target site was analyzed by T7El endonuclease analysis of the HBG2 PCR products amplified from genomic DNA extracted from the samples approximately 3 days after Cas9 RNP delivery. Each of these RNPs supported only low level gene editing in both the CB and adult CD34⁺ cells across 3 donors and 3 separate experiments (FIG. 4B).

To increase gene editing and the occurrence of the 13 nt deletion at the target site, single strand deoxynucleotide donor repair templates (ssODNs) that encoded 87 nt and 89 nt of homology on each side of the targeted deletion site was generated. The ssODNs, either unmodified at the ends (i.e., ssODN1, SEQ ID NO:906, Table 8) or modified to contain phosphorothioates (PhTx) at the 5′ and 3′ ends (i.e., PhTx ssODN1, SEQ ID NO:909, Table 8). The ssODN was designed to ‘encode’ the 13 nt deletion with sequence homology arms engineered flanking this absent sequence to create a perfect deletion.

TABLE 8
Single strand deoxynucleotide donor repair
templates (ssODN)

| | SEQ | |
|---|---|---|
| | ID | |
| ssODN ID | NO | Sequence |
| ssODN1 | 904 | GGGTGCTTCCTTTTATTCTTCATCCCTAGCCAGCCGC |
| 5′ | | CGGCCCCTGGCCTCACTGGATACTCTAAGACTATTGG |
| homology | | TCAAGTTTGCCTT |
| arm | | |
| ssODN1 | 905 | GTCAAGGCAAGGCTGGCCAACCCATGGGTGGAGTTTA |
| 3′ | | GCCAGGGACCGTTTCAGACAGATATTTGCATTGAGAT |
| homology | | AGTGTGGGGAAGGGG |
| arm | | |
| ssODN1 | 906 | GGGTGCTTCCTTTTATTCTTCATCCCTAGCCAGCCGC |
| | | CGGCCCCTGGCCTCACTGGATACTCTAAGACTATTGG |
| | | TCAAGTTTGCCTTGTCAAGGCAAGGCTGGCCAACCCA |
| | | TGGGTGGAGTTTAGCCAGGGACCGTTTCAGACAGATA |
| | | TTTGCATTGAGATAGTGTGGGGAAGGGG |
| PhTx | 907 | G*GGTGCTTCCTTTTATTCTTCATCCCTAGCCAGCCG |
| ssODN1 5′ | | CCGGCCCCTGGCCTCACTGGATACTCTAAGACTATTG |
| homology | | GTCAAGTTTGCCTT |
| arm | | |
| PhTx | 908 | GTCAAGGCAAGGCTGGCCAACCCATGGGTGGAGTTTA |
| ssODN1 3′ | | GCCAGGGACCGTTTCAGACAGATATTTGCATTGAGAT |
| homology | | AGTGTGGGGAAGGG*G |
| arm | | |
| PhTx | 909 | G*GGTGCTTCCTTTTATTCTTCATCCCTAGCCAGCCG |
| ssODN1 | | CCGGCCCCTGGCCTCACTGGATACTCTAAGACTATTG |
| | | GTCAAGTTTGCCTTGTCAAGGCAAGGCTGGCCAACCC |
| | | ATGGGTGGAGTTTAGCCAGGGACCGTTTCAGACAGAT |
| | | ATTTGCATTGAGATAGTGTGGGGAAGGG*G |

The homology arms flanking the deletion are indicated by bold [5′ homology arm] and underline [3′ homology arm]).
Note
the absence of the 13 bp sequence in ssODN1 and PhTx ssODN1.
*Represents modification by phosphorothioate.

ssODN1 and PhTx ssODN1 were co-delivered with RNP targeting HBG containing the Sp37 gRNA (HBG Sp37 RNP) or HBG Sp35 (HBG Sp35 RNP) to CB CD34⁺ cells. Co-delivery of the ssODN donor encoding the 13 nt deletion with HBG Sp35 RNP or HBG Sp37 RNP led to a 6-fold and 5-fold increase in gene editing of the target site, respectively, as determined by T7E1 analysis of the HBG2 PCR product (FIG. 4C). DNA sequencing analysis (Sanger sequencing) of the HBG2 PCR product indicated that 20% gene editing in cells that were treated with HBG Sp37 RNP and the PhTx modified ssODN1,with 15% deletions and 5% insertions (FIG. 4C, lower left panel). Further analysis of the specific type and size of deletions at the target site revealed that 75% of the total deletions detected contained the 13 nt deletion (which included deletion at c.-110 of the CAAT box in the proximal promoter), the absence of which is associated with elevation of HbF expression (FIG. 4C, lower right panel). The remaining ¼ of deletions were partial deletions that did not span the full 13 nt deletion. These data indicate that co-delivery of a homologous ssODN that is engineered to have a deletion supported precise gene editing (deletion) at HBG in human CD34⁺ cells.

### Example 3: Cas9 RNP Targeting the 13 nt Deletion Mutation Supports Gene Editing in Human Adult Mobilized Peripheral Blood Hematopoietic Stem/Progenitor Cells With Increased HBG Expression in Erythroblast Progeny

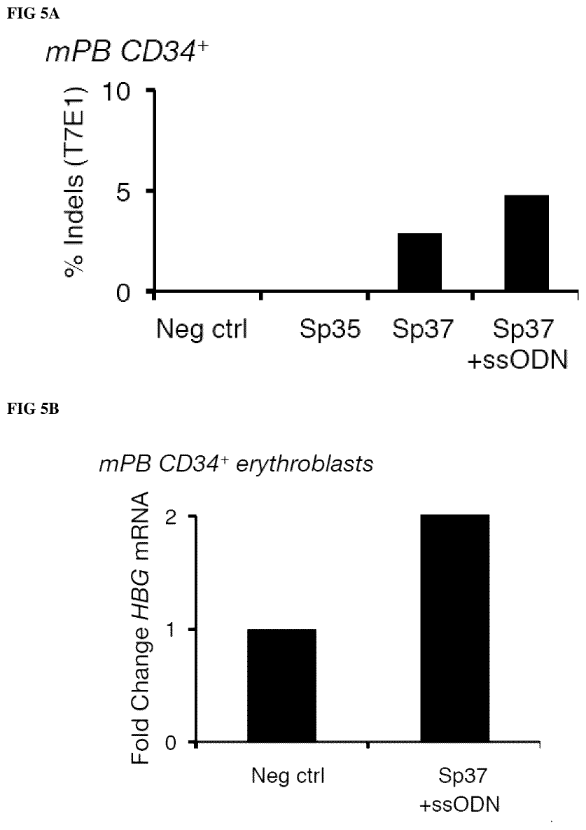

To determine whether editing HBG with Cas9 RNP complexed to Sp37 gRNA or Sp35 gRNA (i.e., the gRNAs that target the 13 nt deletion that is associated with HPFH) in the HBG promoter supports an increase in HBG expression in erythroid progeny of edited CD34⁺ cells, human adult CD34⁺ cells from mobilized peripheral blood (mPB) were electroporated with the RNPs. Briefly, mPB CD34⁺ cells were prestimulated for 2 days with human cytokines and PGE2 in StemSpan SFEM and then electroporated with Cas9 protein precomplexed to Sp35 and Sp37, respectively. T7E1 analysis of HBG PCR product indicated ˜3% indels detected for mPB CD34⁺ cells treated with RNP complexed to Sp37while no editing was detected for cells that were treated with RNP complexed to Sp35 (FIG. 5A).

In order to increase gene editing at the target site and to increase the occurrence of the 13 nt deletion at the target site, PhTx ssODN1 (SEQ ID NO:909) was co-delivered with the precomplexed RNP targeting HBG containing the Sp37 gRNA. Co-delivery of the ssODN donor encoding the 13 nt deletion led to a nearly 2-fold increase in gene editing of the target site (FIG. 5A). To determine whether editing HBG increases production of fetal hemoglobin in erythroid progeny of edited adult CD34⁺ cells, the cells were differentiated into erythroblasts by culture for up to 18 days in the presence of human cytokines (erythropoietin, SCF, IL3), human plasma (Octoplas), and other supplements (hydrocortisone, heparin, transferrin). Over the time course of differentiation, mRNA was collected to evaluate HBG gene expression in the erythroid progeny of RNP treated mPB CD34⁺ cells and donor matched negative (untreated) controls. By day 7 of differentiation, erythroblast progeny of human CD34⁺ cells that were treated with HBG Sp37 RNP and 13 nt deletion encoding ssODN (˜5% indels detected in gDNA from the bulk cell population by T7E1 analysis) exhibited a 2-fold increase in HBG mRNA production (FIG. 5B). Importantly, CD34⁺ cells that were electroporated with HBG RNP maintained their ex vivo hematopoietic activity (i.e., no difference in the quantity or diversity of erythroid and myeloid colonies compared to untreated donor matched CD34⁺ cell negative control), as determined in hematopoietic colony forming cell (CFC) assays (FIG. 6A). Furthermore, the erythroblasts differentiated from RNP treated CD34⁺ cells maintained the kinetics of differentiation observed for donor matched untreated control cells as determined by flow analysis for acquisition of erythroid phenotype (% Glycophorin A+cells) (FIG. 6B). These data indicate that targeted disruption of HBG1/HBG2 proximal promoter region supported an increase in HBG expression in erythroid progeny of RNP treated adult hematopoietic stem/progenitor cells without altering differentiation potential.

### Example 4: Cas9 RNP Targeting the HPFH Mutation Supports Gene Editing in Human Adult Mobilized Peripheral Blood Hematopoietic Stem/Progenitor Cells With Increased HBG Expression in Erythroblast Progeny

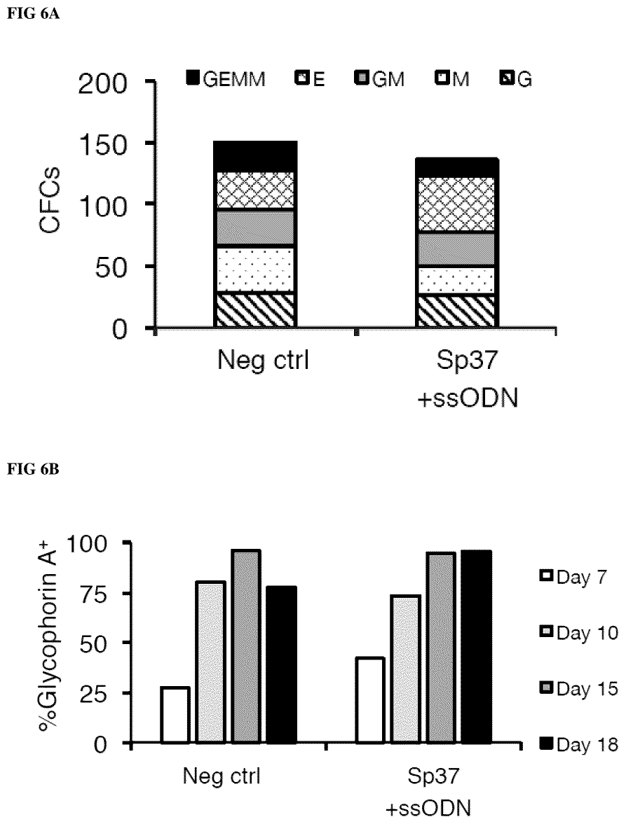

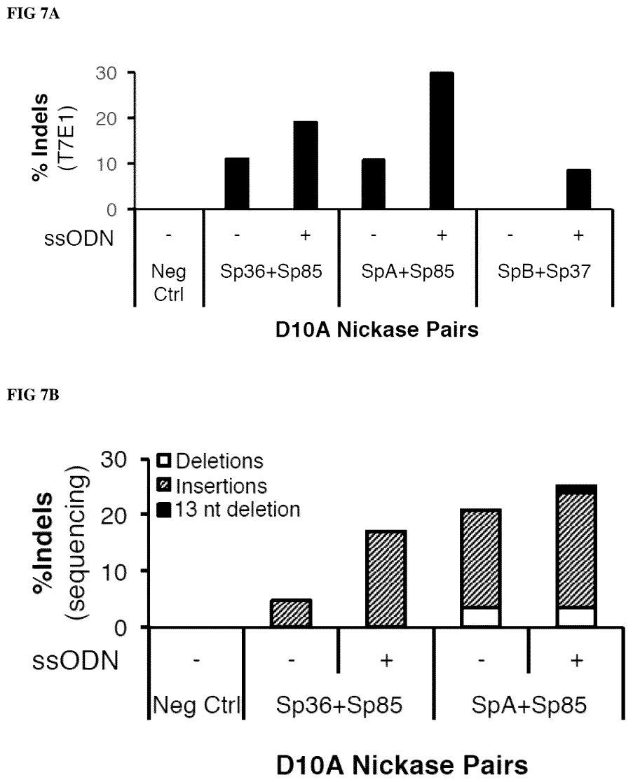

To determine whether co-delivery of paired nickase RNPs targeting HBG would increase targeted disruption of the proximal HBG promoter, mPB CD34⁺ cells were cultured for 2 days with human cytokines and PGE2 in StemSpan SFEM and then electroporated with S. pyogenes D10A Cas9 protein precomplexed to two gRNAs that target sites flanking the site of the 13 nt deletion. The targeting domain sequences for gRNAs used in nickase pairs in this example (including, without limitation, SpA. Sp85 and SpB) are presented in Table 7. D10A nickase pairs were selected such that the PAMs for the targets were oriented outward and the distance between the cut sites were <100 nt. gRNAs were complexed with D10A Cas9 protein to form RNP complexes and then human CD34⁺ cells and paired nickase were subject to electroporation. To determine whether co-delivery of an ssODN that encoded the 13 nt deletion would increase editing and introduction of the mutation into the cells, in some experiments, ssODN1 was added to the cell RNP mixture prior to electroporation. Approximately 3 days after electroporation, gDNA was extracted from the RNP treated cells and analyzed by T7E1 endonuclease assay and/or Sanger DNA sequencing of HBG2 PCR products amplified from the extracted gDNA. Of the three D10A nickase pairs tested, indels detected by T7E1 endonuclease analysis were increased for one nickase pair (gRNAs SpA+Sp85) samples for which ssODN1 was included (FIG. 7A). DNA sequencing analysis was performed on limited samples shown in FIG. 7A. DNA sequencing analysis showed up to ˜27% indels at the target site, with insertions as the dominant indel detected, followed by deletions of the targeted region (area between the cut sites of the paired nickases), and the 13 nt deletion mutation was also detected at a frequency of 2-3% when ssODN1 encoding the deletion was co-delivered (FIG. 7B). Silent, non-pathogenic SNPs were included in the ssODN1 donor template, and were detected in the sequences that contained the 13 nt deletion, indicating that creation of the HFPH mutation occurred through an HDR event.

### Example 5: D10A Paired RNPs Electroporated Into Adult CD34⁺ Cells Supports Induction of HbF Protein in Erythroid Progeny

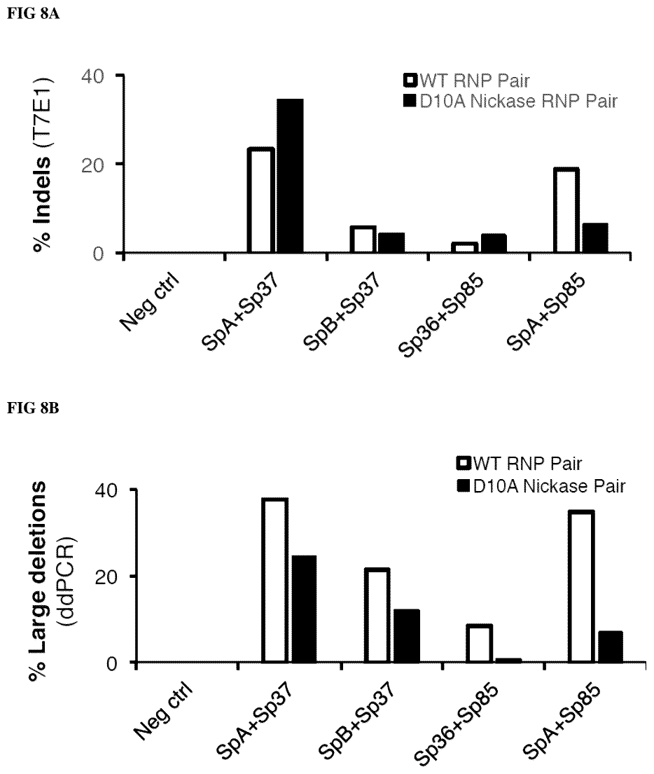

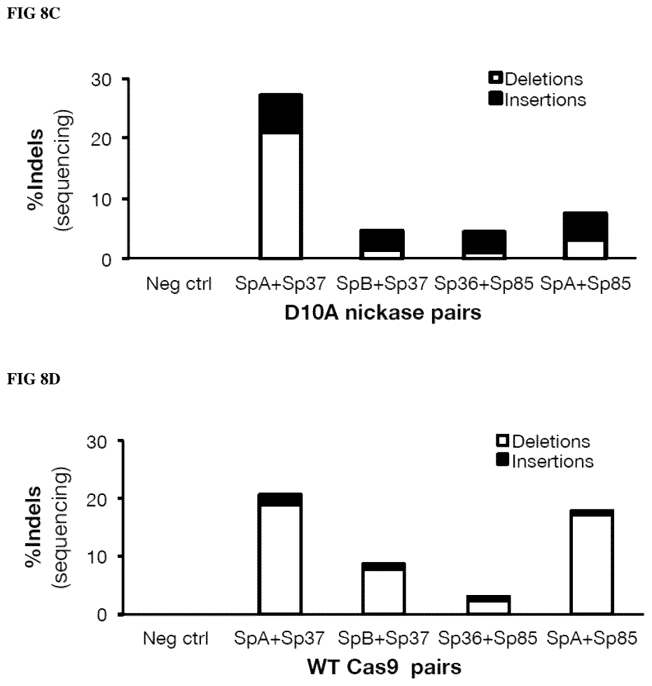

To further optimize editing conditions in mPB CD34⁺ cells at the target site and to evaluate editing in additional human cell donors, human mPB CD34⁺ cells were electroporated with D10A Cas9 and WT Cas9 paired RNPs targeting HBG. The most efficient guide pair for both D10A Cas9 and WT Cas9 RNPs was Sp37+SpA, which supported >30% indels as determined by T7E1 endonuclease analysis of HBG2 PCR products (FIG. 8A). Given that editing at both HBG1 and HBG2 could result in large deletions of HBG2 and the intergenic region between HBG2 and HBG1, indels were further characterized in order to capture local indels by T7E1 endonuclease assay and sequencing and large deletion by ddPCR analysis. Large deletions were detected in all samples at variable frequencies for both D10A Cas9 and WT Cas9 RNP nickase pairs (FIG. 8B). Illumina sequencing analysis of indels correlated with indels determined by T7E1 analysis (FIG. 8C-8D).

To determine whether CD34⁺ cells edited with dual nickases at the HBG promoter gave rise to erythroid progeny with elevated HbF expression, donor matched RNP treated and untreated controls were induced toward erythroid differentiation and then evaluated for maintenance of indels during differentiation and for expression of HbF mRNA and protein. The level of editing (as determined by T7E1 endonuclease assay) was evaluated over the first 2 weeks of erythroid differentiation in the progeny of RNP treated cells prior to enucleation. Indels were detected in the erythroid progeny at every time point assayed suggesting that the editing that occurred in the CD34⁺ cells was maintained during erythroid differentiation and that edited CD34⁺ cells maintain erythroid differentiation potential.

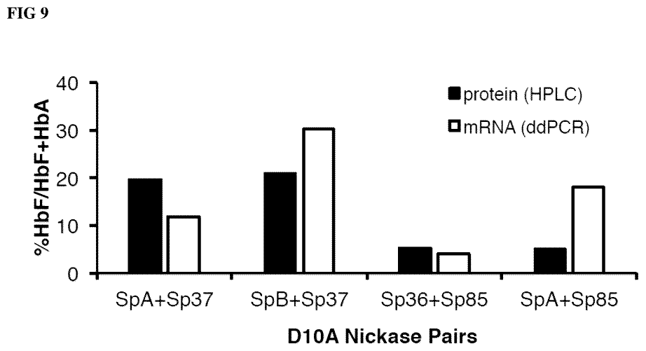

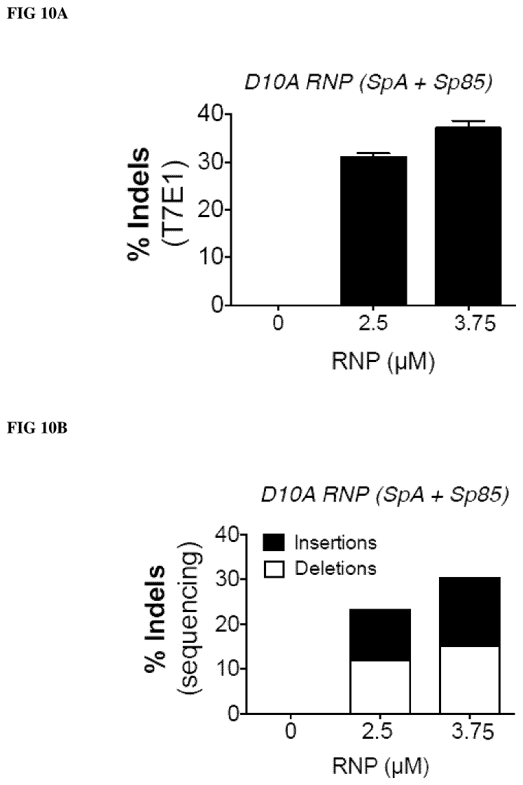

The levels of HBG mRNA (day 10 of differentiation) and HbF protein (day 20-23 of differentiation) were quantified by ddPCR and HPLC analysis (according to the HPLC method described in Chang 2017 at pp. 143-44, incorporated by reference herein), respectively (FIG. 9). A ˜2-fold increase (+40% in in HBG transcripts vs. unedited donor matched control) was observed for HBG: HBA ratio (data not shown) and the ratio of HbF/HbF+HbA (i.e. HBG mRNA/HGB+HBB mRNA) increased to 30% above the level detected in donor matched untreated control samples.

For the D10A Cas9 nickase pairs, upregulation of HbF mRNA and protein was detected in erythroid progeny (FIG. 9). With respect to HbF protein analysis, two pairs supported 20% HbF induction for two D10A nickase pairs. No HbF upregulation was detected in erythroid progeny of WT Cas9 RNP treated CD34⁺ cells (data not shown).

### Example 6: Increasing the Dose of RNP Increases Total Editing Efficiency in Human Adult CD34⁺ Cells at the HBG Locus

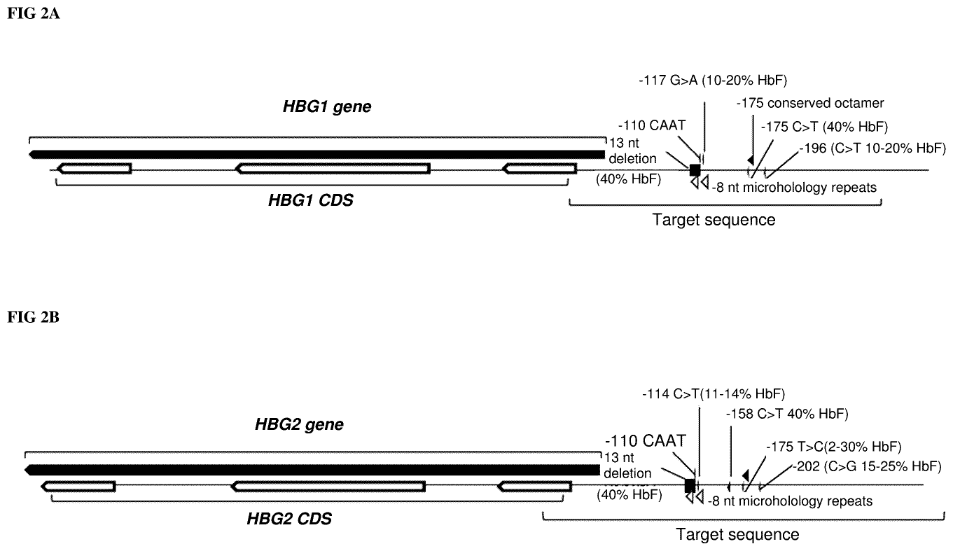

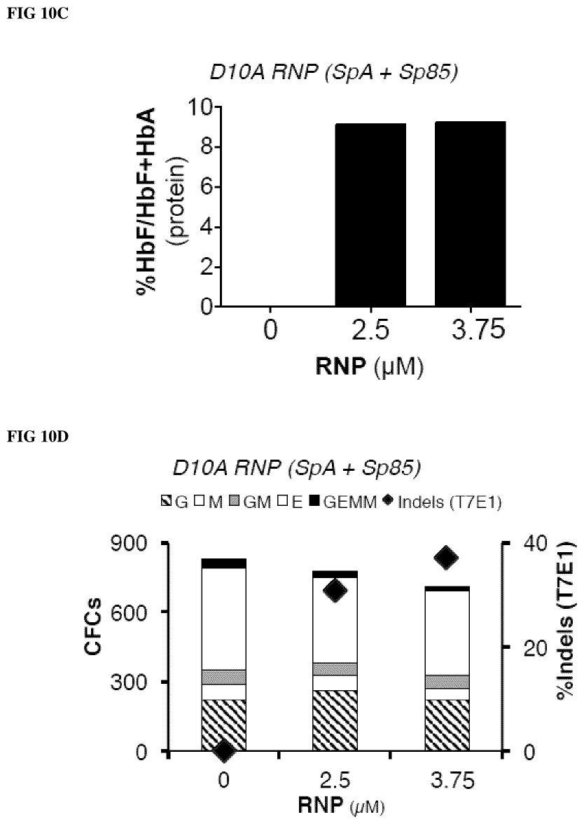

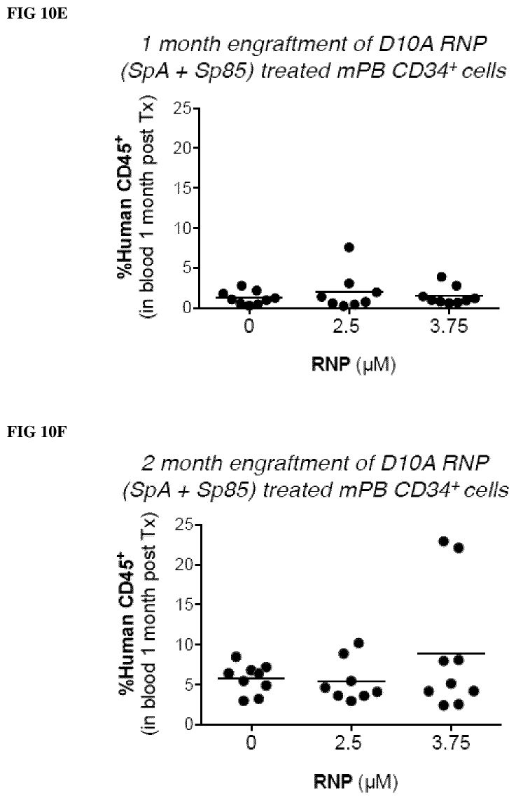

The concentration of D10A Cas9 RNP for the nickase pair SpA+Sp85 was increased (2.5 μM standard concentration and 3.7 μM) and delivered to mPB CD34⁺ cells by electroporation. The increased RNP concentration supported an increase in indels at the HBG target site to >30% (FIG. 10A) as determined by T7E1 endonuclease analysis of the HBG PCR product amplified for gDNA extracted 3 days after electroporation of CD34⁺ cells. Sequencing analysis indicated that increasing the RNP concentration increased insertions (FIG. 10B). Erythroid progeny of RNP treated CD34⁺ cells also had an increase in HbF protein production (FIG. 10C). Importantly, the hematopoietic colony forming potential was maintained after editing (FIG. 10D). These cells were then transplanted into immunodeficient mice and their engraftment 1 month (FIG. 10E) and 2 months (FIG. 10F) after transplantation was evaluated by sampling the peripheral blood and measuring the percentage of human CD45⁺ cells. Early engraftment data showed no difference in engraftment between recipient cohorts of donor matched untreated controls (0 μM RNP) and mice transplanted with RNP treated cells. Furthermore, there was no difference in human blood lineage distribution (myeloid, B cell, T cell) within the human CD45⁺ fraction among cohorts at indicated time points (FIG. 10G-H).

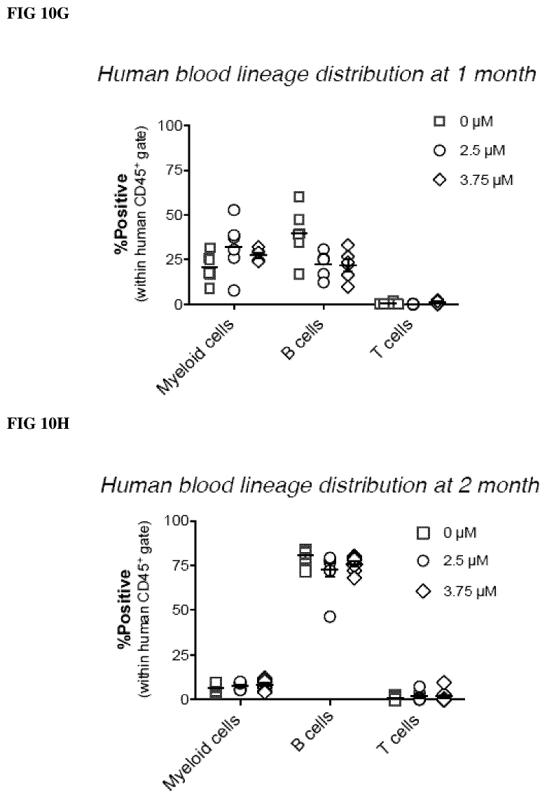

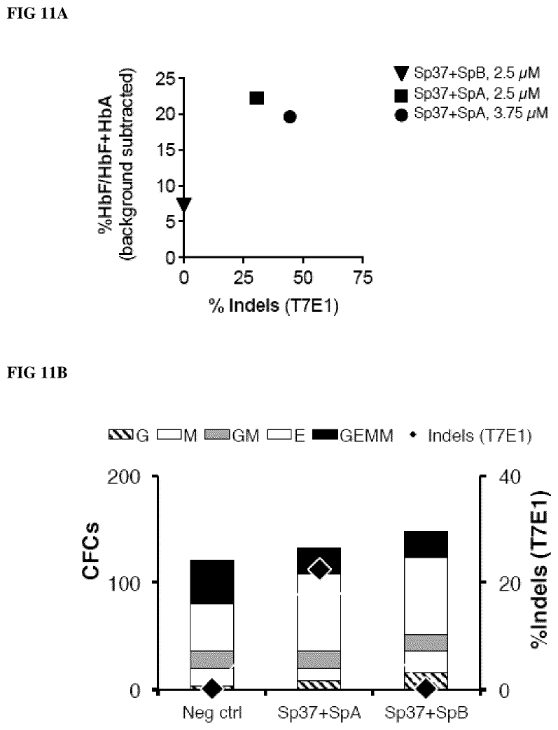

Two additional D10A nickase pairs were also tested in RNP dose response studies in adult mPB CD34⁺ cells (Sp37+SpA, Sp37+SpB). Here, mPB CD34⁺ cells were electroporated with D10A paired nickases delivered at 0, 2.5, and 3.75 μM of total RNP. RNP treated cells were differentiated into erythroid progeny and the HbF protein levels (% HbF/HbF+HbA) were analyzed by HPLC analysis. The indel frequency detected in CD34⁺ cells was plotted with the HbF levels detected in erythroid progeny in order to correlate editing and HbF induction (FIG. 11A). RNP treated and untreated control mPB CD34⁺ cells were also differentiated into colonies to evaluate ex vivo hematopoietic activity. Colony forming cell (CFC) activity was maintained for the progeny of RNP treated and donor matched untreated control CD34⁺ cells (FIG. 11B). There was no difference in the percentage of human CD45⁺ cells in the mouse peripheral blood 1 month after transplantation and no difference in blood lineage distribution (FIG. 11C-D) for cells exposed to different D10A RNP pairs at different doses compared to untreated donor matched control CD34⁺ cells.

### Example 7: Co-Delivery of RNP Targeting the Erythroid Specific Enhancer of BCL11A and a Non-Specific (N) Single Strand Deoxynucleotide Sequence or Paired RNPs Increases Gene Editing in Human CD34⁺ Cells and Supports Induction of Fetal Hemoglobin Expression in Erythroid Progeny

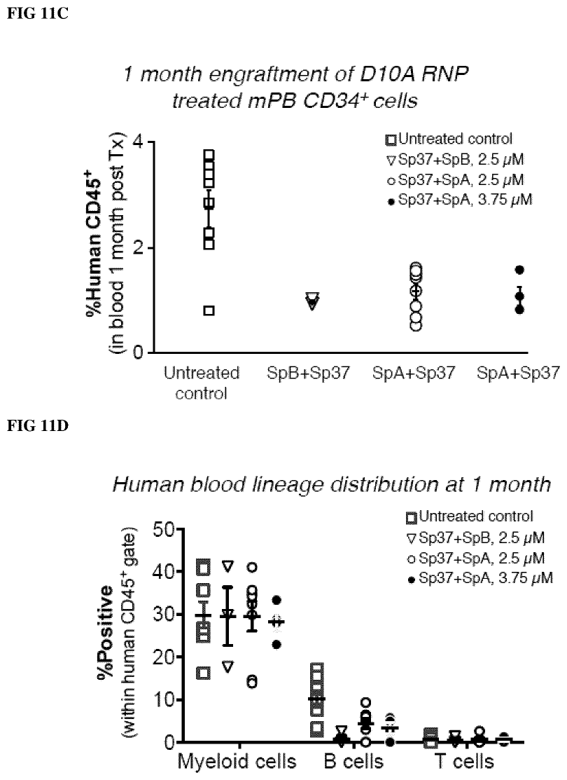

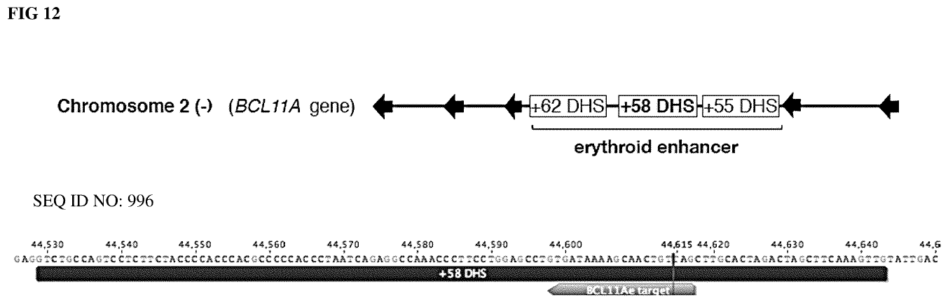

Fetal hemoglobin expression can be induced through targeted disruption of the erythroid cell specific expression of a transcriptional repressor, BCL11A (Canvers 2015). One potential strategy to increase HbF expression through a gene editing strategy is to multiplex gene editing for introduction of 13 nt deletion associated in the HBG proximal promoter and also for targeted disruption of the GATA1binding motif in the erythroid specific enhancer of BCL11A that is in the +58 DHS region of intron 2 of the BCL11A gene (FIG. 12). In order to accomplish this multiplex strategy to increase HbF expression through multiplex gene editing, the effect of disruption of BCL11A erythroid enhancer (BCL11Ae) must first be determined as a single editing event.

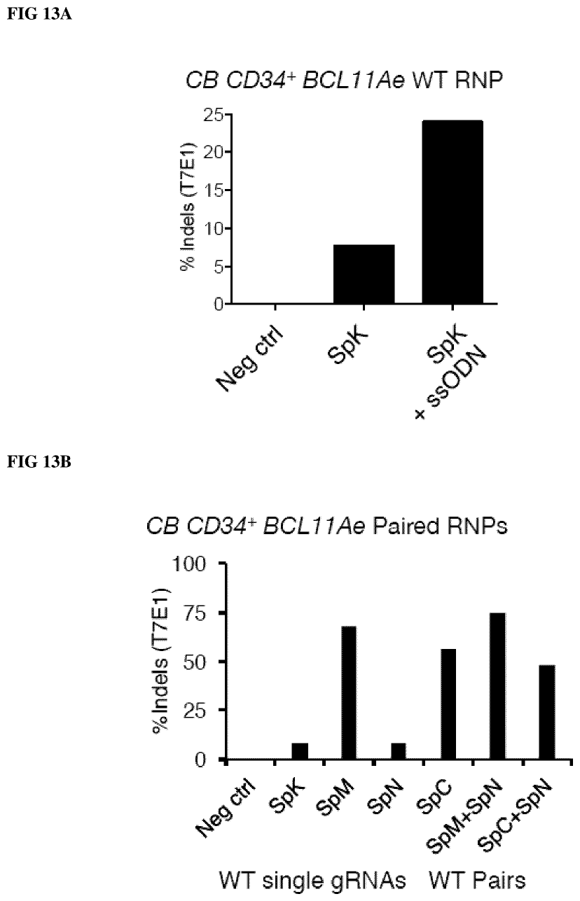

In this experiment, CB CD34⁺ cells were electroporated with S. pyogenes WT Cas9 complexed to in vitro transcribed sgRNA targeting the GATA1 motif in the +58 DHS region of intron 2 of BCL11A gene (gRNA SpK. Table 9) (FIG. 13A). To determine whether co-delivery of a non-target specific ssODN would increase editing of the target sequence, BCL11Ae RNP was co-delivered with ssODN (which is nonhomologous to the BCL11Ae target sequence) in CB CD34⁺ cells. T7E1 analysis of BCL11A erythroid enhancer PCR product from gDNA extracted from CB CD34⁺ cells treated with BCL11Ae RNP indicated that ˜5% indels was achieved (FIG. 13A). Co-delivery of BCL11Ae RNP with a non-target specific ssODN increase in indels by 5-fold to 20% as detected by T7E1 endonuclease analysis. Illumina sequencing analysis indicated that >90% of edits had disruption of the GATA1 motif in the +DHS 58 region enhancer in intron 2 of the BCL11A gene (data not shown). To increase editing, human CB CD34⁺ cells were electroporated with WT Cas9 RNP (single gRNAs complexed to WT Cas9) or with WT Cas9 paired RNPs (paired gRNAs complexed to WT Cas9), so that the cut sites in each pair flank the target site for excision of the GATA1 motif (gRNAs SpC, SpK, SpM, SpN) (Table 9). Two of the single gRNAs and two pairs had >50% indels as determined by T7E1 endonuclease analysis (FIG. 13B).

TABLE 9
Select gRNA sequences targeting BCL11A erythroid
enhancer for screening in CD34⁺ cells

| | | | Targeting | Targeting | |
|---|---|---|---|---|---|
| | Targeting | Targeting | domain | domain | |
| | domain | domain | sequence | sequence | |
| gRNA | sequence | sequence | plus PAM | plus PAM | |
| ID | (RNA) | (DNA) | (NGG)(RNA) | (NGG)(DNA) | Sense |
| SpK | CUAACA | CTAACAG | CUAACAG | CTAACAG | Anti- |
| | GUUGCU | TTGCTTT | UUGCUUU | TTGCTTT | sense |
| | UUUAUC | TATCAC | UAUCACA | TATCACA | |
| | AC | (SEQ ID | GG | GG | |
| | (SEQ ID | NO: 956) | (SEQ ID | (SEQ ID | |
| | NO: 952) | | NO: 960) | NO: 964) | |
| SpM | GGGCGU | GGGCGTG | GGGCGUG | GGGCGTG | Anti- |
| | GGGUGG | GGTGGGG | GGUGGGG | GGTGGGG | sense |
| | GGUAGA | TAGAAG | UAGAAGA | TAGAAGA | |
| | AG | (SEQ ID | GG | GG | |
| | (SEQ ID | NO: 957) | (SEQ ID | (SEQ ID | |
| | NO: 953) | | NO: 961) | NO: 965) | |
| SpN | CUCUUA | CTCTTAG | CUCUUAG | CTCTTAG | Anti- |
| | GACAUAA | ACATAAC | ACAUAAC | ACATAAC | sense |
| | CACACCA | ACACCA | ACACCAG | ACACCAG | |
| | (SEQ ID | (SEQ ID | GG | GG | |
| | NO: 954) | NO: 958) | (SEQ ID | (SEQ ID | |
| | | | NO: 962) | NO: 966) | |
| SpC | AUCAGAG | ATCAGAG | AUCAGAG | ATCAGAG | Sense |
| | GCCAAAC | GCCAAAC | GCCAAAC | GCCAAAC | |
| | CCUUCC | CCTTCC | CCUUCCU | CCTTCCT | |
| | (SEQ ID | (SEQ ID | GG | GG | |
| | NO: 955) | NO: 959) | (SEQ ID | (SEQ ID | |
| | | | NO: 963) | NO: 967) | |

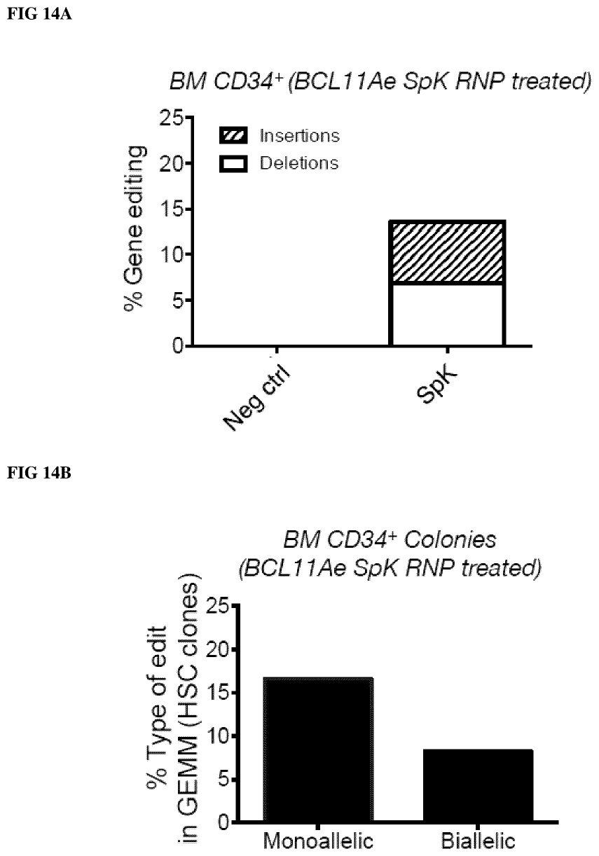

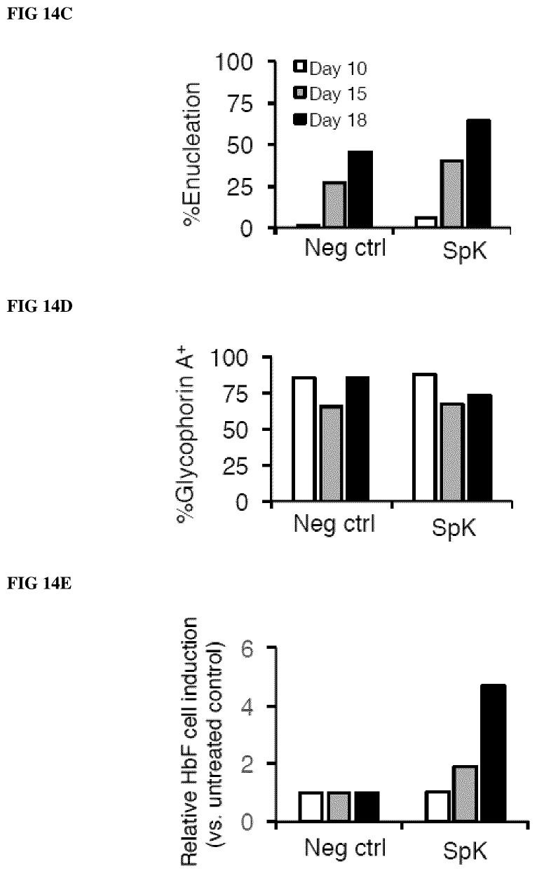

Next, human adult bone marrow CD34⁺ cells were electroporated with the BCL11Ae RNP. DNA sequencing analysis of the BCL11A PCR product amplified from gDNA extracted from marrow CD34⁺ cells indicated 15% gene editing comprised of insertions and deletions (FIG. 14A). Importantly, all deletions resulted in deletion of the GATA1 motif and all insertions disrupted GATA1 motif through addition of a small number of bp in the motif. CD34⁺ cells were plated into colony forming assays and the mixed hematopoietic colonies (GEMMs), which correspond to CD34⁺ cell clones, were picked. gDNA was isolated and analyzed by Illumina sequencing to quantify monoallelic and biallelic disruption of the target site. Most GEMMs differentiated from the CD34⁺ cell clones had monoallelic disruption and biallelic disruption was also detected, with the overall indel rate ˜2/3 higher compared to what was detected in the bulk CD34⁺ cell population (FIG. 14B). This was likely a reflection of the percentage of common myeloid progenitors (CMPs) that give rise to GEMMs that make up a larger fraction of the heterogenous CD34⁺ cells versus the other lineages present, but not captured/differentiated in the short-term CFC assays. The RNP treated marrow CD34⁺ cells also maintained similar kinetics of erythroid maturation (enucleation, FIG. 14C) and differentiation (phenotype acquisition, FIG. 14D) compared to donor matched untreated control cells. Erythroid progeny of edited marrow CD34⁺ cells exhibited ˜5-fold increase in HbF induction as determined by flow cytometry analysis (FIG. 14E).

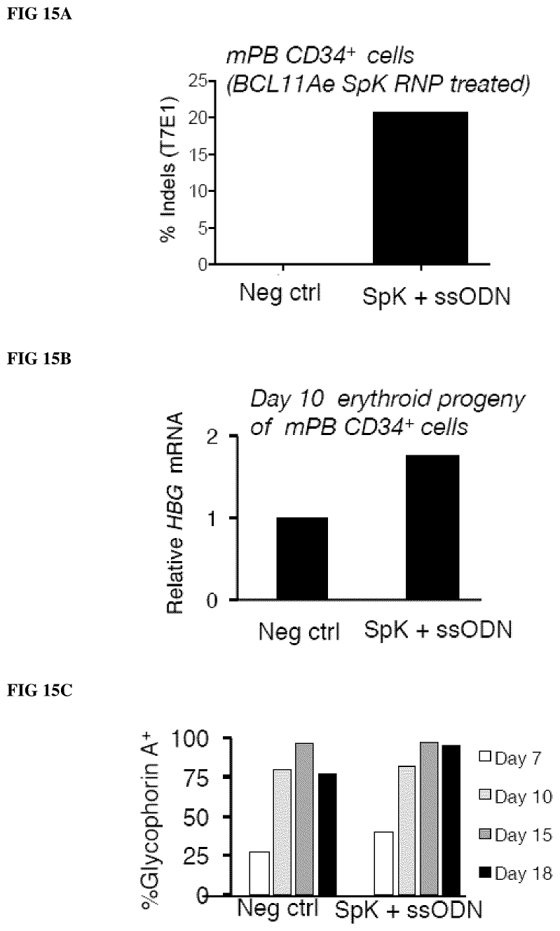

Gene editing and induction of fetal hemoglobin was also evaluated in human adult mPB CD34⁺ cells. Co-delivery of BCL11Ae RNP and nonspecific ssODN supported ˜20% indels at the target site (FIG. 15A). To evaluate early induction of fetal hemoglobin in erythroid progeny of edited cells, mPB CD34+ cells were differentiated into erythroblasts and induction of fetal hemoglobin transcription (HBG mRNA) was evaluated by qRT-PCR analysis. The erythroid progeny of BCL11Ae RNP treated CD34⁺ cells exhibited a 2-fold induction of HBG mRNA compared to untreated controls, suggesting induction of fetal hemoglobin expression (FIG. 15B). The RNP treated marrow CD34⁺ cells also maintained similar kinetics of differentiation (phenotype acquisition, FIG. 15C) compared to donor matched untreated control cells.

### Example 8: RNP Targeting the CAAT Box Have Higher Activity When Complexed With Chemically Synthesized Guide RNA Than In Vitro Transcribed Synthesized gRNA

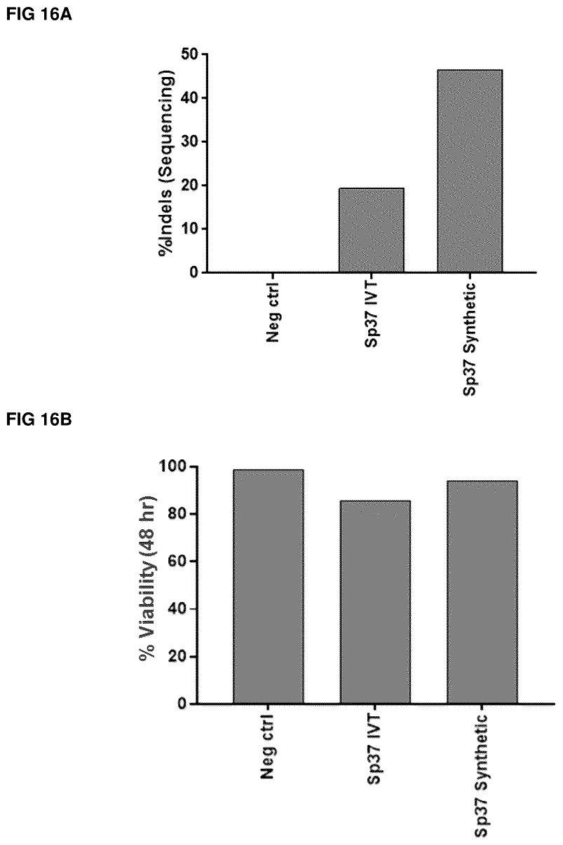

To determine whether complexing Cas9 RNP with a chemically synthesized gRNA enhances editing in the promoter region of HBG, Sp37 gRNA (i.e., a gRNA that targets the 13 nt deletion associated with HPFH; targeting domain set forth in SEQ ID NO:933) was in vitro transcribed (IVT) or chemically synthesized. Human adult CD34+ cells from mobilized peripheral blood (mPB) were electroporated with the RNPs. Briefly, mPB CD34+ cells were prestimulated for 3 days with human cytokines and PGE2 in StemSpan SFEM and then electroporated with Cas9 protein precomplexed to Sp37 (IVT) or Sp37 (synthetic). The use of synthetic guide RNA supported an increase in indels at the HBG target of more than 2 fold (FIG. 16A) as determined by sequencing analysis of the HBG PCR product amplified from gDNA extracted 4 days after electroporation of CD34⁺ cells. The viability of the cells at 48 hours post electroporation was not affected by the use of a chemically synthesized gRNA (FIG. 16A).

Example 9: Sequence Mismatches and End Modifications of Synthetic Guide RNAs Increase the Activity of RNP Targeting the CAAT Box

To evaluate if end modifications of chemically synthesized gRNA enhance the editing of RNP in the promoter region of HBG, the Sp35 gRNA (i.e., a gRNA that targets the 13 nt deletion associated with HPFH; full length sequence set forth in SEQ ID NO:970, targeting domain sequence set forth in SEQ ID NO:339) was chemically synthesized. One or a combination of the following modifications were made at one or both of the ends of the Sp35 gRNA sequence: 3 nucleotides with a phosphorothioate (PhTx) group and 3 nucleotides with a 2′-O-methyl group (Table 10).

To evaluate if truncations of the protospacer or nucleotide mismatches enhance the editing of RNP in the HBG promoter, 5′ end truncations of the Sp35 gRNA were chemically synthesized (Sp35 97mer (SEQ ID NO:976). Sp35 98mer (SEQ ID NO:977), Sp35 99mer (SEQ ID NO:978). Table 10). An Sp35 guide RNA with a C-to-A sequence substitution at the 5′ end was also synthesized (Sp35-5′ C->A (SEQ ID NO:979). Table 10).

TABLE 10
Sequences of chemically synthesized gRNAs
targeting the CAAT box, with end modification,
sequence substitution or truncations

| | Oli- | gRNA sequence | gRNA sequence |
|---|---|---|---|
| Name | ID | (RNA) | (DNA) |
| Sp35 | OLI7066 | CUUGUCAAGGCUAUU | CTTGTCAAGGCTATTG |
| unmodified | | GGUCAGUUUUAGAGC | GTCAGTTTTAGAGCTA |
| | | UAGAAAUAGCAAGUU | GAAATAGCAAGTTAAA |
| | | AAAAUAAGGCUAGUC | ATAAGGCTAGTCCGTT |
| | | CGUUAUCAACUUGAA | ATCAACTTGAAAAAGT |
| | | AAAGUGGCACCGAGU | GGCACCGAGTCGGTGC |
| | | CGGUGCUUUU | TTTT |
| | | (SEQ ID NO: 970) | (SEQ ID NO: 980) |
| Sp35 5′ | OLI8568 | C*U*U*GUCAAGGCU | C*T*T*GTCAAGGCTA |
| PS | | AUUGGUCAGUUUUAG | TTGGTCAGTTTTAGAG |
| | | AGCUAGAAAUAGCAA | CTAGAAATAGCAAGTT |
| | | GUUAAAAUAAGGCUA | AAATAAGGCTAGTCCG |
| | | GUCCGUUAUCAACUU | TTATCAACTTGAAAAA |
| | | GAAAAAGUGGCACCG | GTGGCACCGAGTCGGT |
| | | AGUCGGUGCUUUU | GCTTTT |
| | | (SEQ ID NO: 971) | (SEQ ID NO: 981) |
| Sp35 5′ | OLI8569 | mCmUmUGUCAAGGCU | mCmTmTGTCAAGGCTA |
| OMe | | AUUGGUCAGUUUUAG | TTGGTCAGTTTTAGAG |
| | | AGCUAGAAAUAGCAA | CTAGAAATAGCAAGTT |
| | | GUUAAAAUAAGGCUA | AAAATAAGGCTAGTCC |
| | | GUCCGUUAUCAACUU | GTTATCAACTTGAAAA |
| | | GAAAAAGUGGCACCG | AGTGGCACCGAGTCGG |
| | | AGUCGGUGCUUUU | TGCTTTT |
| | | (SEQ ID NO: 972) | (SEQ ID NO: 982) |
| Sp35 3′ | OLI8396 | CUUGUCAAGGCUAUU | CTTGTCAAGGCTATTG |
| POSMe | | GGUCAGUUUUAGAGC | GTCAGTTTTAGAGCTA |
| | | UAGAAAUAGCAAGUU | GAAATAGCAAGTTAAA |
| | | AAAAUAAGGCUAGUC | ATAAGGCTAGTCCGTT |
| | | CGUUAUCAACUUGAA | ATCAACTTGAAAAAGT |
| | | AAAGUGGCACCGAGU | GGCACCGAGTCGGTGC |
| | | CGGUGCmU*mU* | mT*mT*mT*T |
| | | Mu*U | (SEQ ID NO: 983) |
| | | (SEQ ID NO: 973) | |
| Sp35 5′ | OLI8395 | mC*mU*mU*GUCAAG | mC*mT*mT*GTCAAGG |
| PSOMe | | GCUAUUGGUCAGUUU | CTATTGGTCAGTTTTA |
| | | UAGAGCUAGAAAUAG | GAGCTAGAAATAGCAA |
| | | CAAGUUAAAAUAAGG | GTTAAAATAAGGCTAG |
| | | CUAGUCCGUUAUCAA | TCCGTTATCAACTTGA |
| | | CUUGAAAAGUGGCAC | AAAAGTGGCACCGAGT |
| | | CGAGUCGGUGCUUUU | CGGTGCTTTT |
| | | (SEQ ID NO: 974) | (SEQ ID NO: 984) |
| Sp35 5′-3′ | OLI8394 | mC*mU*mU*GUCAAG | mC*mT*mT*GTCAAGG |
| PSOMe | | GCUAUUGGUCAGUUU | CTATTGGTCAGTTTTA |
| | | UAGAGCUAGAAAUAG | GAGCTAGAAATAGCAA |
| | | CAAGUUAAAAUAAGG | GTTAAAATAAGGCTAG |
| | | CUAGUCCGUUAUCAA | TCCGTTATCAACTTGA |
| | | CUUGAAAAAGUGGCA | AAAAGTGGCACCGAGT |
| | | CCGAGUCGGUGCmU* | CGGTGCmT*mT*mT*T |
| | | mU*mU*U | (SEQ ID NO: 985) |
| | | (SEQ ID NO: 975) | |
| Sp35 | OLI8018 | GUCAAGGCUAUUGGU | GTCAAGGCTATTGGTC |
| 97mer | | CAGUUUUAGAGCUAG | AGTTTTAGAGCTAGAA |
| | | AAAUAGCAAGUUAAA | ATAGCAAGTTAAAATA |
| | | AUAAGGCUAGUCCGU | AGGCTAGTCCGTTATC |
| | | UAUCAACUUGAAAAA | AACTTGAAAAAGTGGC |
| | | GUGGCACCGAGUCGG | ACCGAGTCGGTGCTTT |
| | | UGCUUUU | T |
| | | (SEQ ID NO: 976) | (SEQ ID NO: 986) |
| Sp35 | OLI8019 | UGUCAAGGCUAUUGG | TGTCAAGGCTATTGGT |
| 98mer | | UCAGUUUUUAGAGCU | CAGTTTTAGAGCTAGA |
| | | AGAAAUAGCAAGUUA | AATAGCAAGTTAAAAT |
| | | AAAUAAGGCUAGUCC | AAGGCTAGTCCGTTAT |
| | | GUUAUCAACUUGAAA | CAACTTGAAAAAGTGG |
| | | AAGUGGCACCGAGUC | CACCGAGTCGGTGCTT |
| | | GGUGCUUUU | TT |
| | | (SEQ ID NO: 977) | (SEQ ID NO: 987) |
| Sp35 | OLI8020 | UUGUCAAGGCUAUUG | TTGTCAAGGCTATTGG |
| 99mer | | GUCAGUUUUAGAGCU | TCAGTTTTAGAGCTAG |
| | | AGAAAUAGCAAGUUA | AAATAGCAAGTTAAAA |
| | | AAAUAAGGCAGUCCG | TAAGGCTAGTCCGTTA |
| | | UUAUCAACUUGAAAA | TCAACTTGAAAAAGTG |
| | | AGUGGCACCGAGUCG | GCACCGAGTCGGTGCT |
| | | GUGCUUUU | TTT |
| | | (SEQ ID NO: 978) | (SEQ ID NO: 988) |
| Sp35-5′ | OLI8397 | AUUGUCAAGGCUAUU | ATTGTCAAGGCTATTG |
| C → A | | GGUCAGUUUUAGAGC | GTCAGTTTTAGAGCTA |
| | | UAGAAAUAGCAAGUU | GAAATAGCAAGTTAAA |
| | | AAAAUAAGGCUAGUC | ATAAGGCTAGTCCGTT |
| | | CGUUAUCAACUUGAA | ATCAACTTGAAAAAGT |
| | | AAAGUGGCACCGAGU | GGCACCGAGTCGGTGC |
| | | CGGUGCUUUU | TTTT |
| | | (SEQ ID NO: 979) | (SEQ ID NO: 989) |

*Represents phosphorothioate modification.
m:Represents, 2-o-methyl modification.

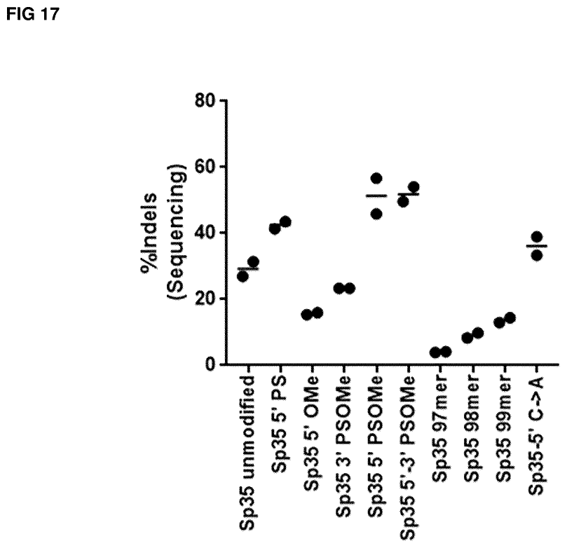

Next, Cas9 RNP were complexed with Sp35 gRNA or modified Sp35 guide RNAs (Table 10) and 7.27 μM RNP were electroporated to human adult CD34⁺ cells from mobilized peripheral blood (mPB). Sequence analysis of the HBG PCR product amplified from gDNA extracted post-electroporation was performed. Increased editing was observed with Sp35 guide RNAs containing 5′ and 5′+3′ modifications, as well as with the C-to-A substitution when compared to the unmodified Sp35 guide RNA (FIG. 17).

### Example 10: Co-Delivery of Cas9 RNP Containing Chemically Synthesized Guide RNA Targeting the 13 nt Deletion Mutation With ssODN Donors Supports Precise Gene Editing in Human Hematopoietic Stem/Progenitor Cells and Increased Gamma-Globin Expression in the Erythroid Progeny

To increase gene editing and the occurrence of the 13 nt deletion at the target site, single strand deoxynucleotide donor repair templates (ssODN) that encoded 90 nucleotides of homology on each side of the targeted deletion site were generated. The ssODNs were modified to contain phosphorothioates (PhTx) at the 5′ and 3′ ends (i.e., OLI16414 (SEQ ID NO:990), OLI16412 (SEQ ID NO:993), respectively, Table 11). The ssODNs were designed to “encode” the 13 nt deletion with sequence homology arms flanking this absent sequence to create a perfect deletion.

TABLE 11
Single strand deoxynucleotide donor repair
templates used in combination
with OLI7066-sp35-RNP

| | | SEQ | |
|---|---|---|---|
| | | ID | |
| Name | Oli-ID | NO | Sequence |
| PhTx | OLI16414 | 990 | A*AGGGTGCTTCCTTTTAT |
| ssODN | | | TCTTCATCCCTAGCCAGCC |
| 13nt- | | | GCCGGCCCCTGGCCTCACT |
| Positive | | | GGATACTCTAAGACTATTG |
| strand | | | GTCAAGTTTGCCTTGTCAA |
| | | | GGCAAGGCTGGCCAACCCA |
| | | | TGGGTGGAGTTTAGCCAGG |
| | | | GACCGTTTCAGACAGATAT |
| | | | TTGCATTGAGATAGTGTGG |
| | | | GGAAGGGGC*C |
| 5′ | 5′ | 991 | A*AGGGTGCTTCCTTTTAT |
| homology | homology | | TCTTCATCCCTAGCCAGCC |
| arm: PhTx | arm | | GCCGGCCCCTGGCCTCACT |
| ssODN 13nt- | OLI16414 | | GGATACTCTAAGACTATTG |
| Positive | | | GTCAAGTTTGCCTTG |
| strand | | | |
| 3′ | 3′ | 992 | TCAAGGCAAGGCTGGCCAA |
| homology | homology | | CCCATGGGTGGAGTTTAGC |
| arm: PhTx | arm | | CAGGGACCGTTTCAGACAG |
| ssODN 13nt- | OLI16414 | | ATATTTGCATTGAGATAGT |
| Positive | | | GTGGGGAAGGGGC*C |
| strand | | | |
| PhTx | OLI16412 | 993 | G*GCCCCTTCCCCACACTA |
| ssODN | | | TCTCAATGCAAATATCTGT |
| 13nt- | | | CTGAAACGGTCCCTGGCTA |
| Negative | | | AACTCCACCCATGGGTTGG |
| strand | | | CCAGCCTTGCCTTGACAAG |
| | | | GCAAACTTGACCAATAGTC |
| | | | TTAGAGTATCCAGTGAGGC |
| | | | CAGGGGCCGGCGGCTGGCT |
| | | | AGGGATGAAGAATAAAAGG |
| | | | AAGCACCCT*T |
| 5′ | 5′ | 994 | G*GCCCCTTCCCCACACTA |
| homology | homology | | TCTCAATGCAAATATCTGT |
| arm: PhTx | arm | | CTGAAACGGTCCCTGGCTA |
| ssODN 13nt- | OLI16412 | | AACTCCACCCATGGGTTGG |
| Negative | | | CCAGCCTTGCCTTGA |
| strand | | | |
| 3′ | 3′ | 995 | CAAGGCAAACTTGACCAAT |
| homology | homology | | AGTCTTAGAGTATCCAGTG |
| arm: PhTx | arm | | AGGCCAGGGGCCGGCGGCT |
| ssODN 13nt- | OLI16412 | | GGCTAGGGATGAAGAATAA |
| Negative | | | AAGGAAGCACCCT*T |
| strand | | | |

The homology arms flanking the deletion are indicated by bold [5′ homology arm] and underline [3′ homology arm]).
Note
the absence of the 13 bp sequence in OLI16414 and OLI16412.
*Represents phosphorothioate modification.

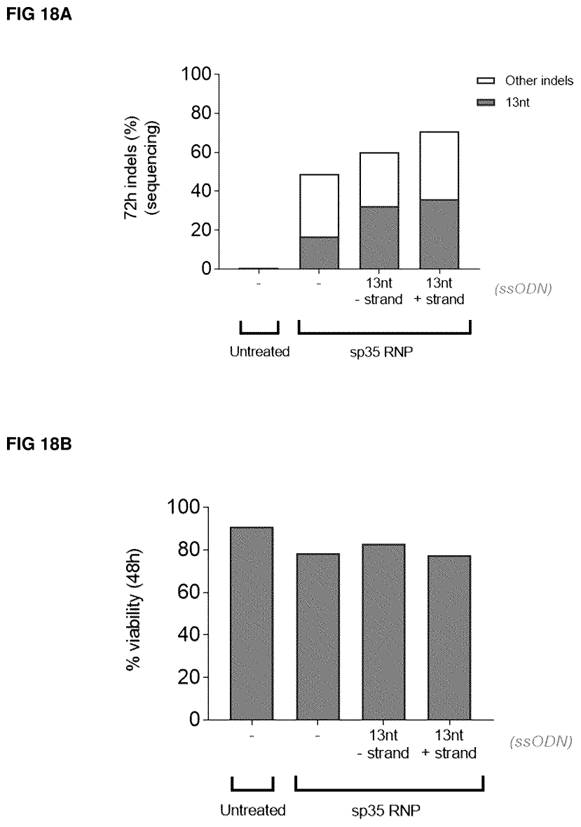

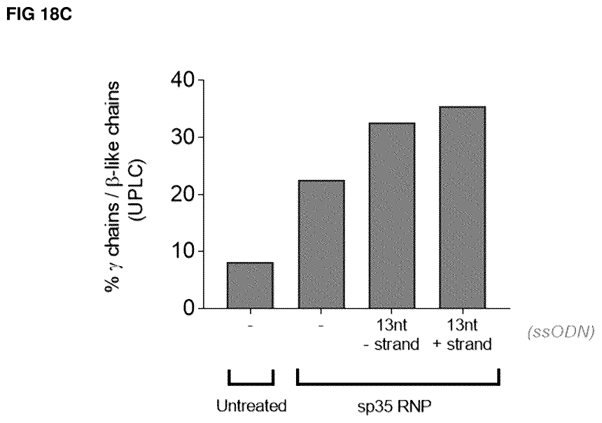

OLI16414 (SEQ ID NO:990) and OLI16412 (SEQ ID NO:993) were co-delivered with RNP targeting HBG containing the chemically synthesized Sp35 gRNA (SEQ ID NO:970) (“OLI7066-sp35-RNP”) to human adult CD34+ cells from mobilized peripheral blood (mPB). Briefly, mPB CD34+ cells were prestimulated for 2 days with human cytokines in X-Vivo-10 and then electroporated with a mixture composed of an ssODN donor and Cas9 protein precomplexed to Sp35 (OLI7066-sp35-RNP). Co-delivery of 2.5 μM of the ssODN donor encoding the 13 nt deletion as a positive strand donor (OLI16414 (SEQ ID NO:990)) or negative strand donor (OLI16412 (SEQ ID NO:993)) with 2 μM of OLI7066-sp35-RNP enhanced the editing frequency from 48.7% (without ssODN donor) to 60.11% and 70.7% respectively, as determined by sequencing analysis of the HBG PCR product from genomic DNA extracted at 72 hours post-electroporation (FIG. 18A).

Further analysis of the specific type and size of deletions at the target site revealed that 32.6% and 36.0% of the alleles carried the precise 13 nt deletion when OLI7066-sp35-RNP was co-delivered with the 13 nt positive strand donor (OLI16414 (SEQ ID NO:990)) and the 13 nt negative strand donor (OLI16412 (SEQ ID NO:993)), respectively, instead of 16.9% when OLI7066-sp35-RNP was delivered alone (FIG. 18A). No reduction in viability of CD34 cells at 48 hours post electroporation was observed when OLI7066-sp35-RNP was co-delivered with the ssODNs (FIG. 18B).

To determine whether co-delivering OLI7066-sp35-RNP with ssODNs “encoding” the 13 nt deletion increased the production of fetal hemoglobin in the erythroid progeny of edited adult CD34⁺ cells, the cells were differentiated into erythroblasts by culture for 18 days in the presence of human cytokines (erythropoietin, SCF, IL3), human plasma (Octoplas), and other supplements (hydrocortisone, heparin, transferrin, insulin). At day 18, the relative expression levels of gamma-globin chains over total beta-like globin chains (gamma chains/[gamma chains +beta chain]) was increased from 8.1% in the untreated sample to 22.6%, 32.6% and 35.4% when the CD34 cells were initially electroporated with OLI7066-sp35-RNP alone, OLI7066-sp35-RNP in combination with the negative strand ssODN donor (OLI16412 (SEQ ID NO:993)), and OLI7066-sp35-RNP in combination with the positive strand ssODN donor (OLI16414 (SEQ ID NO:990)), respectively, as determined by ultra-performance liquid chromatography (UPLC) analysis of the cell lysates (FIG. 18C). These results demonstrate that co-delivery of a chemically synthesized guide RNA targeting the HBG distal CAAT-box with a homologous ssODN that is engineered to have a deletion supported precise gene editing (deletion) at HBG in human mPB+ CD34 cells, resulting in increased level of HBG expression.

### SEQUENCES

Genome editing system components according to the present disclosure (including without limitation, RNA-guided nucleases, guide RNAs, donor template nucleic acids, nucleic acids encoding nucleases or guide RNAs, and portions or fragments of any of the foregoing), are exemplified by the nucleotide and amino acid sequences presented in the Sequence Listing. The sequences presented in the Sequence Listing are not intended to be limiting, but rather illustrative of certain principles of genome editing systems and their component parts, which, in combination with the instant disclosure, will inform those of skill in the art about additional implementations and modifications that are within the scope of this disclosure. A list of the sequences presented is provided in the following Table 12.

TABLE 12
Sequences presented in the Sequence Listing:

| SEQ ID NOS: | DESCRIPTION |
|---|---|
| 1-2, 4-6, 12, 14 | Cas9 polypeptides |
| 3, 7-11, 13 | Cas9 coding sequences |
| 15-23, 52-123 | Cas9 RuvC-like domains |
| 24-28, 124-198 | Cas9 HNH-like domains |
| 29-31, 38-51 | Full-length modular and unimolecular |
| | gRNAs |
| 32-37 | gRNA proximal and tail domains |
| 199-205 | PAM sequences |
| 251-901, 940-942, | gRNA targeting domains (RNA)—see |
| 952-955 | Tables 2, 7, 9 |
| 910-919, 943-945, | gRNA targeting domains (DNA)—see |
| 956-959 | Tables 7, 9 |
| 920-929, 946-948, | gRNA targeting domains plus PAM |
| 960-963 | (NGG) (RNA)—see Tables 7, 9 |
| 930-939, 949-951, | gRNA targeting domains plus PAM |
| 964-967 | (NGG) (DNA)—see Tables 7, 9 |
| 970-979 | Sp35 gRNA sequences (RNA)—see |
| | Table 10 |
| 980-989 | Sp35 gRNA sequences (DNA)—see |
| | Table 10 |
| 902, 903 | Human HBG1 and HBG2 promoter |
| | sequences including HPFH deletion site |
| 904-909, 990-995 | Oligonucleotide donor sequences and |
| | homology arms—see Tables 8, 11 |
| 968-969 | BCL11Ae sequences |

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments described herein. Such equivalents are intended to be encompassed by the following claims.

### REFERENCES

Ahern et al., Br J Haematol 25 (4): 437-444 (1973)
Akinbami Hemoglobin 40:64-65 (2016)
Aliyu et al. Am J Hematol 83:63-70 (2008)
Anders et al. Nature 513 (7519): 569-573 (2014)
Angastiniotis & Modell Ann N Y Acad Sci 850:251-269 (1998)
Bae et al. Bioinformatics 30 (10): 1473-1475 (2014)
Barbosa et al. Braz J Med Bio Res 43 (8): 705-711 (2010)
Bouva Hematologica 91 (1): 129-132 (2006)
Briner et al. Mol Cell 56 (2): 333-339 (2014)
Brousseau Am J Hematol 85 (1): 77-78 (2010)
Caldecott Nat Rev Genet 9 (8): 619-631 (2008)
Canvers et al. Nature 527 (12): 192-197 (2015)
Chang et al. Mol Ther Methods Clin Dev 4:137-148 (2017)
Chassanidis Ann Hematol 88 (6): 549-555 (2009)
Chylinski et al. RNA Biol 10 (5): 726-737 (2013)
Cong et al. Science 399 (6121): 819-823 (2013)
Costa et al., Cad Saude Publica 18 (5): 1469-1471 (2002)
Davis & Maizels 2 Proc Natl Acad Sci USA 111 (10): E924-932 (2014)
Fine et al. Sci Rep. 5:10777 (2015)
Frit et al. DNA Repair (Amst) 17:81-97 (2014)
Fu et al. Nat Biotechnol 32 (3): 279-284 (2014)
Guilinger et al. Nat Biotechnol 32:577-582 (2014)
Heigwer et al. Nat Methods 11 (2): 122-123 (2014)
Hsu et al. Nat Biotechnol 31 (9): 827-832 (2013)
Iyama & Wilson DNA Repair (Amst) 12 (8): 620-636 (2013)
Jiang et al. Nat Biotechnol 31 (3): 233-239 (2013)
Jinek et al. Science 337 (6096): 816-821 (2012)
Jinek et al. Science 343 (6176): 1247997 (2014)
Kleinstiver et al. Nature 523 (7561): 481-485 (2015a)
Kleinstiver et al. Nat Biotechnol 33 (12): 1293-1298 (2015b)
Kleinstiver et al. Nature 529 (7587): 490-495 (2016)
Komor et al. Nature 533 (7603): 420-424 (2016)
Lee et al. Nano Lett 12 (12): 6322-6327 (2012)
Lewis “Medical-Surgical Nursing: Assessment and Management of Clinical Problems” (2014)
Li Cell Res 18 (1): 85-98 (2008)
Maeder et al. Int'l Patent Publication No. WO 2015/138510 (2015)
Makarova et al. Nat Rev Microbiol 9 (6): 467-477 (2011)
Mali et al. Science 339 (6121): 823-826 (2013)
Mantovani et al. Nucleic Acids Res 16 (16): 7783-7797 (1988)
Marteijn et al. Nat Rev Mol Cell Biol 15 (7): 465-481 (2014)
Nishimasu et al. Cell 156 (5): 935-949 (2014)
Nishimasu et al. Cell 162 (5): 1113-1126 (2015)
Ran & Hsu et al. Cell 154 (6): 1380-1389 (2013)
Richardson et al. Nat Biotechnol 34:339-344 (2016)
Shmakov et al. Molecular Cell 60 (3): 385-397 (2015)
Sternberg et al. Nature 507 (7490): 62-67 (2014)
Superti-Furga et al. EMBO J 7 (10): 3099-3107 (1988)
Thein Hum Mol Genet 18 (R2): R216-223 (2009)
Tsai et al. Nat Biotechnol 34 (5): 483 (2016)
Waber et al. Blood 67 (2): 551-554 (1986)
Wang et al. Cell 153 (4): 910-918 (2013)
Xiao et al. Bioinformatics 30 (8): 1180-1182 (2014)
Xu et al. Genes Dev 24 (8): 783-798 (2010)
Yamano et al. Cell 165 (4): 949-962 (2016)
Zetsche et al. Cell 163 (3): 759-771 (2015a)
Zetsche et al. Nat Biotechnol 33 (2): 139-42 (2015b)

1. A genome editing system, comprising:
an RNA-guided nuclease;
a first guide RNA; and
a second guide RNA, wherein the first and second guide RNAs comprise first and second targeting domains complementary to first and second sequences on opposite sides of positions of a 13 nucleotide (nt) target region of a human HBG1 or HBG2 gene,
wherein one or both of the first and second sequences optionally overlaps the 13 nt target region of the human HBG1 or HBG2 gene.

2. (canceled)

3. The genome editing system of claim 1, wherein the RNA-guided nuclease is an S. pyogenes Cas9.

4. The genome editing system of claim 1, wherein the first and second targeting domains are complementary to sequences immediately adjacent to a protospacer adjacent motif recognized by S. pyogenes Cas9.

5. The genome editing system of claim 4, wherein the RNA-guided nuclease is a nickase, and optionally lacks RuvC activity.

6. (canceled)

7. The genome editing system of claim 1, wherein the first targeting domain is complementary to a sequence within positions c.-214 to -114 of a human HBG1 or HBG2 gene.

8. The genome editing system of claim 1, wherein one of the first and second targeting domains is complementary to a sequence within positions c.-102to -52 of a human HBG1 or HBG2 gene.

9. The genome editing system of claim 1, wherein the second targeting domain is complementary to a sequence within positions c.-102 to -2 of a human HBG1 or HBG2 gene.

10. The genome editing system of claim 1, wherein at least one of the first and second targeting domains differ by no more than 3 nucleotides from a targeting domain listed in Table 7.

11. The genome editing system of claim 1, comprising first and second RNA-guided nucleases.

12. The genome editing system of claim 11, wherein the first and second RNA-guided nucleases are complexed with the first and second guide RNAs, respectively, forming first and second ribonucleoprotein complexes.

13-24. (canceled)

25. A method of altering a cell, comprising contacting a cell with the genome editing system of claim 9.

26. The method of claim 25, wherein the step of contacting the cell with the genome editing system comprises contacting the cell with a solution comprising first and second ribonucleoprotein complexes.

27. The method of claim 26, wherein the step of contacting the cell with the solution further comprises electroporating the cells, thereby introducing the first and second ribonucleoprotein complexes into the cell.

28-29. (canceled)

30. The method of claim 25, wherein the cell is capable of differentiating into an erythroblast or a precursor of an erythroblast.

31. The method of claim 25, wherein the cell is capable of differentiating into an erythrocyte or a precursor of an erythrocyte.

32. The method of claim 25, wherein the cell is a CD34⁺ cell.

33. A CRISPR-mediated method of altering a cell, comprising:
introducing a first DNA single strand break (SSB) or double strand break (DSB) within a genome of the cell between positions c.-614 to -102 of a human HBG1 or HBG2 gene; and
introducing a second SSB or DSB within the genome of the cell between positions c.-114 to -1 of the human HBG1 or HBG2 gene,
wherein the first and second SSBs or DSBs are repaired by the cell in a manner that alters a 13 nt target region of the human HBG1 or HBG2 gene.

34. The CRISPR-mediated method of claim 33, wherein the first and second SSBs or DSBs are repaired by the cell in a manner that results in the deletion of all or part of a 13 nt target region of the human HBG1 or HBG2 gene.

35. The CRISPR-mediated method of claim 33, wherein the first and second SSBs or DSBs are repaired by the cell in a manner that results in the formation of at least one of an indel, a deletion, or an insertion in the 13 nt target region of the human HBG1 or HBG2 gene.

36. The CRISPR-mediated method of claim 33, wherein the first and second SSBs or DSBs are repaired by the cell in an error prone manner.

37-81. (canceled)


SEQUENCE LISTING (996 sequences)

SEQ ID NO: 1 - protein, 1345 aa, Streptococcus mutans
  REGION 10..21: note = misc_feature - N-terminal RuvC-like domain
  REGION 759..766: note = misc_feature - RuvC-like domain
  REGION 837..863: note = misc_feature - HNH-like domain
  REGION 982..989: note = misc_feature - RuvC-like domain
MKKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 2 - protein, 1368 aa, Streptococcus pyogenes [sequence omitted]

SEQ ID NO: 3 - DNA, 4107 bp, Streptococcus pyogenes
  source 1..4107: mol_type = other DNA
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 4 - protein, 1388 aa, Streptococcus thermophilus
  REGION 10..21: note = misc_feature - N-terminal RuvC-like domain
  REGION 760..767: note = misc_feature - RuvC-like domain
  REGION 844..870: note = misc_feature - HNH-like domain
  REGION 989..996: note = misc_feature - RuvC-like domain
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 5 - protein, 1334 aa, Listeria innocua
  REGION 10..21: note = misc_feature - N-terminal RuvC-like domain
  REGION 762..769: note = misc_feature - RuvC-like domain
  REGION 840..866: note = misc_feature - HNH-like domain
  REGION 985..992: note = misc_feature - RuvC-like domain
MKKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 6 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 7 - DNA, 3159 bp, Staphylococcus aureus
  source 1..3159: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 8 - DNA, 3159 bp, Staphylococcus aureus
  source 1..3159: mol_type = other DNA
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 9 - DNA, 3159 bp, Staphylococcus aureus
  source 1..3159: mol_type = other DNA
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 10 - DNA, 3159 bp, Staphylococcus aureus
  source 1..3159: mol_type = other DNA
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 11 - DNA, 3159 bp, Staphylococcus aureus
  source 1..3159: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 12 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 13 - DNA, 3249 bp, Neisseria meningitidis
  misc_feature 1..3249: note = Exemplary codon optimized Cas9
  source 1..3249: mol_type = other DNA
atggccgccttcaagcccaaccccatcaactacatcctgggcctggacatcggcatcgccagcgtgggctgggccatggtggagatcgacgaggacgagaaccccatctgcctgatcgacctgggtgtgcgcgtgttcgagcgcgctgaggtgcccaagactggtgacagtctggctatggctcgccggcttgctcgctctgttcggcgccttactcgccggcgcgctcaccgccttctgcgcgctcgccgcctgctgaagcgcgagggtgtgctgcaggctgccgacttcgacgagaacggcctgatcaagagcctgcccaacactccttggcagctgcgcgctgccgctctggaccgcaagctgactcctctggagtggagcgccgtgctgctgcacctgatcaagcaccgcggctacctgagccagcgcaagaacgagggcgagaccgccgacaaggagctgggtgctctgctgaagggcgtggccgacaacgcccacgccctgcagactggtgacttccgcactcctgctgagctggccctgaacaagttcgagaaggagagcggccacatccgcaaccagcgcggcgactacagccacaccttcagccgcaaggacctgcaggccgagctgatcctgctgttcgagaagcagaaggagttcggcaacccccacgtgagcggcggcctgaaggagggcatcgagaccctgctgatgacccagcgccccgccctgagcggcgacgccgtgcagaagatgctgggccactgcaccttcgagccagccgagcccaaggccgccaagaacacctacaccgccgagcgcttcatctggctgaccaagctgaacaacctgcgcatcctggagcagggcagcgagcgccccctgaccgacaccgagcgcgccaccctgatggacgagccctaccgcaagagcaagctgacctacgcccaggcccgcaagctgctgggtctggaggacaccgccttcttcaagggcctgcgctacggcaaggacaacgccgaggccagcaccctgatggagatgaaggcctaccacgccatcagccgcgccctggagaaggagggcctgaaggacaagaagagtcctctgaacctgagccccgagctgcaggacgagatcggcaccgccttcagcctgttcaagaccgacgaggacatcaccggccgcctgaaggaccgcatccagcccgagatcctggaggccctgctgaagcacatcagcttcgacaagttcgtgcagatcagcctgaaggccctgcgccgcatcgtgcccctgatggagcagggcaagcgctacgacgaggcctgcgccgagatctacggcgaccactacggcaagaagaacaccgaggagaagatctacctgcctcctatccccgccgacgagatccgcaaccccgtggtgctgcgcgccctgagccaggcccgcaaggtgatcaacggcgtggtgcgccgctacggcagccccgcccgcatccacatcgagaccgcccgcgaggtgggcaagagcttcaaggaccgcaaggagatcgagaagcgccaggaggagaaccgcaaggaccgcgagaaggccgccgccaagttccgcgagtacttccccaacttcgtgggcgagcccaagagcaaggacatcctgaagctgcgcctgtacgagcagcagcacggcaagtgcctgtacagcggcaaggagatcaacctgggccgcctgaacgagaagggctacgtggagatcgaccacgccctgcccttcagccgcacctgggacgacagcttcaacaacaaggtgctggtgctgggcagcgagaaccagaacaagggcaaccagaccccctacgagtacttcaacggcaaggacaacagccgcgagtggcaggagttcaaggcccgcgtggagaccagccgcttcccccgcagcaagaagcagcgcatcctgctgcagaagttcgacgaggacggcttcaaggagcgcaacctgaacgacacccgctacgtgaaccgcttcctgtgccagttcgtggccgaccgcatgcgcctgaccggcaagggcaagaagcgcgtgttcgccagcaacggccagatcaccaacctgctgcgcggcttctggggcctgcgcaaggtgcgcgccgagaacgaccgccaccacgccctggacgccgtggtggtggcctgcagcaccgtggccatgcagcagaagatcacccgcttcgtgcgctacaaggagatgaacgccttcgacggtaaaaccatcgacaaggagaccggcgaggtgctgcaccagaagacccacttcccccagccctgggagttcttcgcccaggaggtgatgatccgcgtgttcggcaagcccgacggcaagcccgagttcgaggaggccgacacccccgagaagctgcgcaccctgctggccgagaagctgagcagccgccctgaggccgtgcacgagtacgtgactcctctgttcgtgagccgcgcccccaaccgcaagatgagcggtcagggtcacatggagaccgtgaagagcgccaagcgcctggacgagggcgtgagcgtgctgcgcgtgcccctgacccagctgaagctgaaggacctggagaagatggtgaaccgcgagcgcgagcccaagctgtacgaggccctgaaggcccgcctggaggcccacaaggacgaccccgccaaggccttcgccgagcccttctacaagtacgacaaggccggcaaccgcacccagcaggtgaaggccgtgcgcgtggagcaggtgcagaagaccggcgtgtgggtgcgcaaccacaacggcatcgccgacaacgccaccatggtgcgcgtggacgtgttcgagaagggcgacaagtactacctggtgcccatctacagctggcaggtggccaagggcatcctgcccgaccgcgccgtggtgcagggcaaggacgaggaggactggcagctgatcgacgacagcttcaacttcaagttcagcctgcaccccaacgacctggtggaggtgatcaccaagaaggcccgcatgttcggctacttcgccagctgccaccgcggcaccggcaacatcaacatccgcatccacgacctggaccacaagatcggcaagaacggcatcctggagggcatcggcgtgaagaccgccctgagcttccagaagtaccagatcgacgagctgggcaaggagatccgcccctgccgcctgaagaagcgccctcctgtgcgctaa

SEQ ID NO: 14 - protein, 859 aa, synthetic construct
  REGION 1..859: note = Synthetic Cas9 consensus sequence derived from Sm, Sp, St, and Li Cas9 sequences
  SITE 4: note = misc_feature - Xaa can be any naturally occurring amino acid
  REGION 8..18: note = misc_feature - N-terminal RuvC-like domain
  SITE 21: note = misc_feature - Xaa can be any naturally occurring amino acid
  SITE 23: note = misc_feature - Xaa can be any naturally occurring amino acid
  SITE 24: note = misc_feature - Xaa can be any naturally occurring amino acid
  SITE 26: note = misc_feature - Xaa can be any naturally occurring amino acid
  REGION 29..31: note = misc_feature - Each Xaa can independently be any naturally occurring amino acid
  SITE 33: note = misc_feature - Xaa can be any naturally occurring amino acid
  SITE 36: note = misc_feature - Xaa can be any naturally occurring amino acid
  SITE 45: note = misc_feature - Xaa can be any naturally occurring amino acid
  SITE 54: note = misc_feature - Xaa can be any naturally occurring amino acid
  SITE 63: note = misc_feature - Xaa can be any naturally occurring amino acid
  SITE 71: note = misc_feature - Xaa can be any naturally occurring amino acid
  SITE 75: note = misc_feature - Xaa can be any naturally occurring amino acid
  SITE 76: note = misc_feature - Xaa can be any naturally occurring amino acid
  REGION 78..80: note = misc_feature - Each Xaa can independently be any naturally occurring amino acid
  SITE 82: note = misc_feature - Xaa can be any naturally occurring amino acid
  SITE 84: note = misc_feature - Xaa can be any naturally occurring amino acid
  SITE 85: note = misc_feature - Xaa can be any naturally occurring amino acid
  SITE 89: note = misc_feature - Xaa can be any naturally occurring amino acid
  SITE 90: note = misc_feature - Xaa can be any naturally occurring amino acid
  SITE 98: note = misc_feature - Xaa can be any naturally occurring amino acid
  SITE 100: note = misc_feature - Xaa can be any naturally occurring amino acid
  SITE 106: note = misc_feature - Xaa can be any naturally occurring amino acid
  SITE 113: note = misc_feature - Xaa can be any naturally occurring amino acid
  SITE 116: note = misc_feature - Xaa can be any naturally occurring amino acid
  SITE 125: note = misc_feature - Xaa can be any naturally occurring amino acid
  SITE 126: note = misc_feature - Xaa can be any naturally occurring amino acid
  REGION 128..133: note = misc_feature - Each Xaa can independently be any naturally occurring amino acid
  SITE 135: note = misc_feature - Xaa can be any naturally occurring amino acid
  SITE 137: note = misc_feature - Xaa can be any naturally occurring amino acid
  REGION 139..147: note = misc_feature - Each Xaa can independently be any naturally occurring amino acid
  REGION 153..155: note = misc_feature - Each Xaa can independently be any naturally occurring amino acid
  SITE 157: note = misc_feature - Xaa can be any naturally occurring amino acid
  SITE 159: note = misc_feature - Xaa can be any naturally occurring amino acid
  SITE 161: note = misc_feature - Xaa can be any naturally occurring amino acid
  SITE 163: note = misc_feature - Xaa can be any naturally occurring amino acid
  REGION 166..168: note = misc_feature - Each Xaa can independently be any naturally occurring amino acid
  SITE 170: note = misc_feature - Xaa can be any naturally occurring amino acid
  SITE 171: note = misc_feature - Xaa can be any naturally occurring amino acid
  REGION 173..175: note = misc_feature - Each Xaa can independently be any naturally occurring amino acid
  SITE 177: note = misc_feature - Xaa can be any naturally occurring amino acid
  SITE 183: note = misc_feature - Xaa can be any naturally occurring amino acid
  REGION 185..187: note = misc_feature - Each Xaa can independently be any naturally occurring amino acid
  SITE 189: note = misc_feature - Xaa can be any naturally occurring amino acid
  REGION 192..195: note = misc_feature - Each Xaa can independently be any naturally occurring amino acid
  SITE 198: note = misc_feature - Xaa can be any naturally occurring amino acid
  SITE 199: note = misc_feature - Xaa can be any naturally occurring amino acid
  SITE 202: note = misc_feature - Xaa can be any naturally occurring amino acid
  SITE 206: note = misc_feature - Xaa can be any naturally occurring amino acid
  SITE 207: note = misc_feature - Xaa can be any naturally occurring amino acid
  SITE 210: note = misc_feature - Xaa can be any naturally occurring amino acid
  SITE 212: note = misc_feature - Xaa can be any naturally occurring amino acid
  SITE 213: note = misc_feature - Xaa can be any naturally occurring amino acid
  SITE 219: note = misc_feature - Xaa can be any naturally occurring amino acid
  SITE 220: note = misc_feature - Xaa can be any naturally occurring amino acid
  SITE 222: note = misc_feature - Xaa can be any naturally occurring amino acid
  SITE 224: note = misc_feature - Xaa can be any naturally occurring amino acid
  SITE 226: note = misc_feature - Xaa can be any naturally occurring amino acid
  SITE 236: note = misc_feature - Xaa can be any naturally occurring amino acid
  SITE 240: note = misc_feature - Xaa can be any naturally occurring amino acid
  SITE 241: note = misc_feature - Xaa can be any naturally occurring amino acid
  REGION 244..246: note = misc_feature - Each Xaa can independently be any naturally occurring amino acid
  SITE 248: note = misc_feature - Xaa can be any naturally occurring amino acid
  SITE 249: note = misc_feature - Xaa can be any naturally occurring amino acid
  SITE 250: note = misc_feature - Xaa can be any naturally occurring amino acid
  REGION 252..254: note = misc_feature - Each Xaa can independently be any naturally occurring amino acid
  SITE 256: note = misc_feature - Xaa can be any naturally occurring amino acid
  SITE 257: note = misc_feature - Xaa can be any naturally occurring amino acid
  SITE 268: note = misc_feature - Xaa can be any naturally occurring amino acid
  SITE 271: note = misc_feature - Xaa can be any naturally occurring amino acid
  SITE 273: note = misc_feature - Xaa can be any naturally occurring amino acid
  SITE 277: note = misc_feature - Xaa can be any naturally occurring amino acid
  SITE 280: note = misc_feature - Xaa can be any naturally occurring amino acid
  SITE 281: note = misc_feature - Xaa can be any naturally occurring amino acid
  SITE 283: note = misc_feature - Xaa can be any naturally occurring amino acid
  SITE 289: note = misc_feature - Xaa can be any naturally occurring amino acid
  SITE 290: note = misc_feature - Xaa can be any naturally occurring amino acid
  REGION 292..294: note = misc_feature - Each Xaa can independently be any naturally occurring amino acid
  SITE 301: note = misc_feature - Xaa can be any naturally occurring amino acid
  SITE 308: note = misc_feature - Xaa can be any naturally occurring amino acid
  REGION 319..322: note = misc_feature - Each Xaa can independently be any naturally occurring amino acid
  SITE 328: note = misc_feature - Xaa can be any naturally occurring amino acid
  SITE 329: note = misc_feature - Xaa can be any naturally occurring amino acid
  REGION 335..337: note = misc_feature - Each Xaa can independently be any naturally occurring amino acid
  SITE 346: note = misc_feature - Xaa can be any naturally occurring amino acid
  SITE 347: note = misc_feature - Xaa can be any naturally occurring amino acid
  REGION 356..361: note = misc_feature - Each Xaa can independently be any naturally occurring amino acid
  SITE 363: note = misc_feature - Xaa can be any naturally occurring amino acid
  REGION 370..373: note = misc_feature - Each Xaa can independently be any naturally occurring amino acid
  SITE 375: note = misc_feature - Xaa can be any naturally occurring amino acid
  SITE 376: note = misc_feature - Xaa can be any naturally occurring amino acid
  SITE 379: note = misc_feature - Xaa can be any naturally occurring amino acid
  REGION 386..390: note = misc_feature - Each Xaa can independently be any naturally occurring amino acid
  SITE 393: note = misc_feature - Xaa can be any naturally occurring amino acid
  SITE 395: note = misc_feature - Xaa can be any naturally occurring amino acid
  SITE 396: note = misc_feature - Xaa can be any naturally occurring amino acid
  SITE 398: note = misc_feature - Xaa can be any naturally occurring amino acid
  SITE 400: note = misc_feature - Xaa can be any naturally occurring amino acid
  SITE 403: note = misc_feature - Xaa can be any naturally occurring amino acid
  SITE 407: note = misc_feature - Xaa can be any naturally occurring amino acid
  SITE 410: note = misc_feature - Xaa can be any naturally occurring amino acid
  SITE 411: note = misc_feature - Xaa can be any naturally occurring amino acid
  REGION 413..416: note = misc_feature - Each Xaa can independently be any naturally occurring amino acid
  SITE 418: note = misc_feature - Xaa can be any naturally occurring amino acid
  SITE 422: note = misc_feature - Xaa can be any naturally occurring amino acid
  SITE 428: note = misc_feature - Xaa can be any naturally occurring amino acid
  SITE 431: note = misc_feature - Xaa can be any naturally occurring amino acid
  SITE 433: note = misc_feature - Xaa can be any naturally occurring amino acid
  REGION 437..439: note = misc_feature - Each Xaa can independently be any naturally occurring amino acid
  SITE 445: note = misc_feature - Xaa can be any naturally occurring amino acid
  SITE 451: note = misc_feature - Xaa can be any naturally occurring amino acid
  SITE 456: note = misc_feature - Xaa can be any naturally occurring amino acid
  SITE 459: note = misc_feature - Xaa can be any naturally occurring amino acid
  REGION 465..469: note = misc_feature - Each Xaa can independently be any naturally occurring amino acid
  SITE 481: note = misc_feature - Xaa can be any naturally occurring amino acid
  SITE 482: note = misc_feature - Xaa can be any naturally occurring amino acid
  SITE 484: note = misc_feature - Xaa can be any naturally occurring amino acid
  SITE 490: note = misc_feature - Xaa can be any naturally occurring amino acid
  SITE 494: note = misc_feature - Xaa can be any naturally occurring amino acid
  REGION 495..502: note = misc_feature - RuvC-like domain
  SITE 497: note = misc_feature - Xaa can be any naturally occurring amino acid
  SITE 506: note = misc_feature - Xaa can be any naturally occurring amino acid
  SITE 510: note = misc_feature - Xaa can be any naturally occurring amino acid
  SITE 513: note = misc_feature - Xaa can be any naturally occurring amino acid
  SITE 514: note = misc_feature - Xaa can be any naturally occurring amino acid
  SITE 517: note = misc_feature - Xaa can be any naturally occurring amino acid
  SITE 520: note = misc_feature - Xaa can be any naturally occurring amino acid
  SITE 525: note = misc_feature - Xaa can be any naturally occurring amino acid
  SITE 526: note = misc_feature - Xaa can be any naturally occurring amino acid
  SITE 529: note = misc_feature - Xaa can be any naturally occurring amino acid
  SITE 531: note = misc_feature - Xaa can be any naturally occurring amino acid
  SITE 532: note = misc_feature - Xaa can be any naturally occurring amino acid
  SITE 534: note = misc_feature - Xaa can be any naturally occurring amino acid
  SITE 542: note = misc_feature - Xaa can be any naturally occurring amino acid
  SITE 546: note = misc_feature - Xaa can be any naturally occurring amino acid
  SITE 547: note = misc_feature - Xaa can be any naturally occurring amino acid
  SITE 553: note = misc_feature - Xaa can be any naturally occurring amino acid
  REGION 555..575: note = misc_feature - HNH-like domain
  SITE 556: note = misc_feature - Xaa can be any naturally occurring amino acid
  SITE 560: note = misc_feature - Xaa can be any naturally occurring amino acid
  SITE 563: note = misc_feature - Xaa can be any naturally occurring amino acid
  SITE 565: note = misc_feature - Xaa can be any naturally occurring amino acid
  SITE 567: note = misc_feature - Xaa can be any naturally occurring amino acid
  SITE 579: note = misc_feature - Xaa can be any naturally occurring amino acid
  SITE 582: note = misc_feature - Xaa can be any naturally occurring amino acid
  SITE 583: note = misc_feature - Xaa can be any naturally occurring amino acid
  SITE 585: note = misc_feature - Xaa can be any naturally occurring amino acid
  SITE 588: note = misc_feature - Xaa can be any naturally occurring amino acid
  SITE 590: note = misc_feature - Xaa can be any naturally occurring amino acid
  SITE 592: note = misc_feature - Xaa can be any naturally occurring amino acid
  REGION 594..596: note = misc_feature - Each Xaa can independently be any naturally occurring amino acid
  SITE 610: note = misc_feature - Xaa can be any naturally occurring amino acid
  SITE 616: note = misc_feature - Xaa can be any naturally occurring amino acid
  SITE 628: note = misc_feature - Xaa can be any naturally occurring amino acid
  SITE 631: note = misc_feature - Xaa can be any naturally occurring amino acid
  SITE 633: note = misc_feature - Xaa can be any naturally occurring amino acid
  SITE 634: note = misc_feature - Xaa can be any naturally occurring amino acid
  SITE 636: note = misc_feature - Xaa can be any naturally occurring amino acid
  REGION 638..641: note = misc_feature - Each Xaa can independently be any naturally occurring amino acid
  REGION 643..645: note = misc_feature - Each Xaa can independently be any naturally occurring amino acid
  SITE 653: note = misc_feature - Xaa can be any naturally occurring amino acid
  SITE 657: note = misc_feature - Xaa can be any naturally occurring amino acid
  SITE 659: note = misc_feature - Xaa can be any naturally occurring amino acid
  SITE 660: note = misc_feature - Xaa can be any naturally occurring amino acid
  SITE 665: note = misc_feature - Xaa can be any naturally occurring amino acid
  SITE 666: note = misc_feature - Xaa can be any naturally occurring amino acid
  SITE 668: note = misc_feature - Xaa can be any naturally occurring amino acid
  SITE 669: note = misc_feature - Xaa can be any naturally occurring amino acid
  REGION 670..677: note = misc_feature - RuvC-like domain
  SITE 680: note = misc_feature - Xaa can be any naturally occurring amino acid
  SITE 681: note = misc_feature - Xaa can be any naturally occurring amino acid
  SITE 683: note = misc_feature - Xaa can be any naturally occurring amino acid
  SITE 686: note = misc_feature - Xaa can be any naturally occurring amino acid
  SITE 696: note = misc_feature - Xaa can be any naturally occurring amino acid
  SITE 697: note = misc_feature - Xaa can be any naturally occurring amino acid
  SITE 704: note = misc_feature - Xaa can be any naturally occurring amino acid
  SITE 708: note = misc_feature - Xaa can be any naturally occurring amino acid
  SITE 710: note = misc_feature - Xaa can be any naturally occurring amino acid
  SITE 711: note = misc_feature - Xaa can be any naturally occurring amino acid
  SITE 714: note = misc_feature - Xaa can be any naturally occurring amino acid
  REGION 717..720: note = misc_feature - Each Xaa can independently be any naturally occurring amino acid
  SITE 722: note = misc_feature - Xaa can be any naturally occurring amino acid
  SITE 725: note = misc_feature - Xaa can be any naturally occurring amino acid
  SITE 727: note = misc_feature - Xaa can be any naturally occurring amino acid
  REGION 733..735: note = misc_feature - Each Xaa can independently be any naturally occurring amino acid
  SITE 740: note = misc_feature - Xaa can be any naturally occurring amino acid
  SITE 742: note = misc_feature - Xaa can be any naturally occurring amino acid
  REGION 749..754: note = misc_feature - Each Xaa can independently be any naturally occurring amino acid
  REGION 758..761: note = misc_feature - Each Xaa can independently be any naturally occurring amino acid
  REGION 763..768: note = misc_feature - Each Xaa can independently be any naturally occurring amino acid
  SITE 771: note = misc_feature - Xaa can be any naturally occurring amino acid
  REGION 774..777: note = misc_feature - Each Xaa can independently be any naturally occurring amino acid
  SITE 782: note = misc_feature - Xaa can be any naturally occurring amino acid
  REGION 784..786: note = misc_feature - Each Xaa can independently be any naturally occurring amino acid
  SITE 788: note = misc_feature - Xaa can be any naturally occurring amino acid
  SITE 790: note = misc_feature - Xaa can be any naturally occurring amino acid
  SITE 795: note = misc_feature - Xaa can be any naturally occurring amino acid
  SITE 799: note = misc_feature - Xaa can be any naturally occurring amino acid
  SITE 801: note = misc_feature - Xaa can be any naturally occurring amino acid
  SITE 802: note = misc_feature - Xaa can be any naturally occurring amino acid
  SITE 804: note = misc_feature - Xaa can be any naturally occurring amino acid
  REGION 806..813: note = misc_feature - Each Xaa can independently be any naturally occurring amino acid
  REGION 815..818: note = misc_feature - Each Xaa can independently be any naturally occurring amino acid
  SITE 820: note = misc_feature - Xaa can be any naturally occurring amino acid
  REGION 823..827: note = misc_feature - Each Xaa can independently be any naturally occurring amino acid
  SITE 829: note = misc_feature - Xaa can be any naturally occurring amino acid
  SITE 830: note = misc_feature - Xaa can be any naturally occurring amino acid
  SITE 832: note = misc_feature - Xaa can be any naturally occurring amino acid
  REGION 835..837: note = misc_feature - Each Xaa can independently be any naturally occurring amino acid
  REGION 842..844: note = misc_feature - Each Xaa can independently be any naturally occurring amino acid
  SITE 846: note = misc_feature - Xaa can be any naturally occurring amino acid
  SITE 848: note = misc_feature - Xaa can be any naturally occurring amino acid
  SITE 851: note = misc_feature - Xaa can be any naturally occurring amino acid
  SITE 857: note = misc_feature - Xaa can be any naturally occurring amino acid
  SITE 857: note = misc_feature - Xaa can be any naturally occurring amino acid
MKYXIGLDIGTNSVGWAVTDXYXXKXKGXXXIXKNXGLFDGTARXRTARRRRRXNRIYLQIFXEMDFFRLXSFVXXKXXXPXFXXEYHXXPTIYHLRXLXKDLRLXYLALAHXIKXRGNFLIEGXXNXXXXXXYXFXIXXXXXXXXXPEKGFXXXLXGXFXFXLEXXXKXXYXXXLXLLIGDXYXXXFXAKXXXXLSXXVTXALSXXMIXRXXHDLLKXXYXEXFXKGYAGYIDGXQFYXXKLXXXGXXXKXXXEXXLRKQRTFDNGXIPXQXHLEXAIXXQXYPFLNXXIXXXTFRIPYXVGPLAGXSFAWRKIPWNXXXXDSAFIXXMTDLPXXXVLPKHSLYXXVYNELTKVXXXXXXKXIFKRKVXXXXGXXFNXSTYHDLXXXXXLDXNXXEXIXLTXFEDXMIXXLXXXXKXLRRXYTGWGXLSXLXGIRXXXSTILDXLDNRNXMQLIXDLXFKIKQXXXXXGSPAIKKGILQXXKXVDELVXMGPXIVXEMARENQTXGNSXRKXXKEXGSXILKEXXNLXNXXLXLYYLQNGXDMYXXLDILSXYDXDHIXPQXFXDXSIDNVLSNRKDXVPXXVXKKXWXLXLXXXRKFDLTKAERGGLXDKAFIXRQLVETRQITKXVAXLXXNXDXXXXVXXXTLKSLVSXFRKXFXXLYKVXXNXXHHAHDAYLNVXXLXYPXLEEFVYGDYXXKATKFYXNIMXFXXGEXWKXXXXVXMQXNXVKKEQXXXPKNSXLXKDKYGGXXXXXXKGKXXXXIXXXXXXFLXGYXXXXLPKYXLXXXGXRXLASEXKGNXLXXLXAXXXXXXXXEXXXXFXANXXXXXLXXGXAFXXXIRRYXXXTXIXQSXTGLYEXRL

SEQ ID NO: 15 - protein, 8 aa, synthetic construct
  REGION 1..8: note = RuvC-like domain
  VARIANT 2: note = Xaa is Val or His
  VARIANT 3: note = Xaa is Ile, Leu, or Val
  VARIANT 5: note = Xaa is Met or Thr
IXXEXARE

SEQ ID NO: 16 - protein, 8 aa, synthetic construct
  REGION 1..8: note = RuvC-like domain
  VARIANT 3: note = Xaa is Ile, Leu, or Val
IVXEMARE

SEQ ID NO: 17 - protein, 8 aa, synthetic construct
  REGION 1..8: note = RuvC-like domain
  VARIANT 4: note = Xaa is His or Leu
  VARIANT 7: note = Xaa is Arg or Val
  VARIANT 8: note = Xaa is Glu or Val
HHAXDAXX

SEQ ID NO: 18 - protein, 8 aa, synthetic construct
  REGION 1..8: note = RuvC-like domain
HHAHDAYL

SEQ ID NO: 19 - protein, 30 aa, synthetic construct
  REGION 1..30: note = N-terminal RuvC-like domain
  VARIANT 2: note = Xaa is Lys or Pro
  VARIANT 4: note = Xaa is Val, Leu, Ile, or Phe
  VARIANT 5: note = Xaa is Gly, Ala, or Ser
  VARIANT 6: note = Xaa is Leu, Ile, Val, or Phe
  MOD_RES 7..26: note = N-terminal RuvC-like domain, each Xaa can be any amino acid or absent, region may encompass 5-20 residues
  VARIANT 29: note = Xaa is Asp, Glu, Asn, or Gln
KXYXXXXXXXXXXXXXXXXXXXXXXXTDXY

SEQ ID NO: 20 - skipped

SEQ ID NO: 21 - protein, 12 aa, synthetic construct
  REGION 1..12: note = N-terminal RuvC-like domain
  VARIANT 2: note = Xaa is Ile, Val, Met, Leu, or Thr
  VARIANT 4: note = Xaa is Thr, Ile, Val, Ser, Asn, Tyr, Glu, or Leu
  VARIANT 5: note = Xaa is Asn, Ser, Gly, Ala, Asp, Thr, Arg, Met, or Phe
  VARIANT 7: note = Xaa is Val, Ile, Leu, Cys, Thr, or Phe
  VARIANT 9: note = Xaa is Trp, Phe, Val, Tyr, Ser, or Leu
  VARIANT 10: note = Xaa is Ala, Ser, Cys, Val, or Gly
  VARIANT 11: note = Xaa is Val, Ile, Leu, Ala, Met, or His
  VARIANT 12: note = Any amino acid or absent
DXGXXSXGXXXX

SEQ ID NO: 22 - protein, 12 aa, synthetic construct
  REGION 1..12: note = N-terminal RuvC-like domain
  VARIANT 4: note = Xaa is Thr, Ile, Val, Ser, Asn, Tyr, Glu, or Leu
  VARIANT 5: note = Xaa is Asn, Ser, Gly, Ala, Asp, Thr, Arg, Met, or Phe
  VARIANT 11: note = Xaa is Val, Ile, Leu, Ala, Met, or His
  VARIANT 12: note = Any amino acid or absent
DIGXXSVGWAXX

SEQ ID NO: 23 - protein, 12 aa, synthetic construct
  REGION 1..12: note = N-terminal RuvC-like domain
  MOD_RES 12: note = Any non-polar alkyl amino acid or a hydroxyl amino acid
DIGTNSVGWAVX

SEQ ID NO: 24 - protein, 73 aa, synthetic construct
  REGION 1..73: note = HNH-like domain
  VARIANT 8: note = Xaa is Lys or Arg
  VARIANT 12: note = Xaa is Val or Thr
  VARIANT 13: note = Xaa is Gly or Asp
  VARIANT 14: note = Xaa is Glu, Gln, or Asp
  VARIANT 15: note = Xaa is Glu or Asp
  VARIANT 19: note = Xaa is Asp, Asn, or His
  VARIANT 20: note = Xaa is Tyr, Arg, or Asn
  VARIANT 23: note = Xaa is Gln, Asp, or Asn
  MOD_RES 25..64: note = HNH-like domain, each Xaa can be any amino acid or absent, region may encompass 15-40 residues
  VARIANT 67: note = Xaa is Gly or Glu
  VARIANT 69: note = Xaa is Ser or Gly
  VARIANT 71: note = Xaa is Asp or Asn
LYYLQNGXDMYXXXXLDIXXLSXYXXXXXXXXXXXXXXXXXXXXXXXXXXXXXXXXXXXXXXXXNRXKXDXVP

SEQ ID NO: 25 - protein, 27 aa, synthetic construct
  REGION 1..27: note = HNH-like domain
  VARIANT 1: note = Xaa is Asp, Glu, Gln, or Asn
  VARIANT 2: note = Xaa is Leu, Ile, Arg, Gln, Val, Met, or Lys
  VARIANT 3: note = Xaa is Asp or Glu
  VARIANT 5: note = Xaa is Ile, Val, Thr, Ala, or Leu
  VARIANT 6: note = Xaa is Val, Tyr, Ile, Leu, Phe, or Trp
  VARIANT 8: note = Xaa is Gln, His, Arg, Lys, Tyr, Ile, Leu, Phe, or Trp
  VARIANT 9: note = Xaa is Ser, Ala, Asp, Thr, or Lys
  VARIANT 10: note = Xaa is Phe, Leu, Val, Lys, Tyr, Met, Ile, Arg, Ala, Glu, Asp, or Gln
  VARIANT 11: note = Xaa is Leu, Arg, Thr, Ile, Val, Ser, Cys, Tyr, Lys, Phe, or Gly
  VARIANT 12: note = Xaa is Lys, Gln, Tyr, Thr, Phe, Leu, Trp, Met, Ala, Glu, Gly, or Ser
  VARIANT 13: note = Xaa is Asp, Ser, Asn, Arg, Leu, or Thr
  VARIANT 14: note = Xaa is Asp, Asn, or Ser
  VARIANT 15: note = Xaa is Ser, Ala, Thr, Gly, or Arg
  VARIANT 16: note = Xaa is Ile, Leu, Phe, Ser, Arg, Tyr, Gln, Trp, Asp, Lys, or His
  VARIANT 17: note = Xaa is Asp, Ser, Ile, Asn, Glu, Ala, His, Phe, Leu, Gln, Met, Gly, Tyr, or Val
  VARIANT 19: note = Xaa is Lys, Leu, Arg, Met, Thr, or Phe
  VARIANT 20: note = Xaa is Val, Leu, Ile, Ala, or Thr
  VARIANT 21: note = Xaa is Leu, Ile, Val, or Ala
  VARIANT 22: note = Xaa is Thr, Val, Cys, Glu, Ser, or Ala
  VARIANT 23: note = Xaa is Arg, Phe, Thr, Trp, Glu, Leu, Asn, Cys, Lys, Val, Ser, Gln, Ile, Tyr, His, or Ala
  VARIANT 24: note = Xaa is Ser, Pro, Arg, Lys, Asn, Ala, His, Gln, Gly, or Leu
  VARIANT 25: note = Xaa is Asp, Gly, Thr, Asn, Ser, Lys, Ala, Ile, Glu, Leu, Gln, Arg, or Tyr
  VARIANT 26: note = Xaa is Lys, Val, Ala, Glu, Tyr, Ile, Cys, Leu, Ser, Thr, Gly, Lys, Met, Asp, or Phe
XXXHXXPXXXXXXXXXXNXXXXXXXXN

SEQ ID NO: 26 - protein, 27 aa, synthetic construct
  REGION 1..27: note = HNH-like domain
  VARIANT 1: note = Xaa is Asp or Glu
  VARIANT 2: note = Xaa is Leu, Ile, Arg, Gln, Val, Met, or Lys
  VARIANT 3: note = Xaa is Asp or Glu
  VARIANT 5: note = Xaa is Ile, Val, Thr, Ala, or Leu
  VARIANT 6: note = Xaa is Val, Tyr, Ile, Leu, Phe, or Trp
  VARIANT 8: note = Xaa is Gln, His, Arg, Lys, Tyr, Ile, Leu, Phe, or Trp
  VARIANT 10: note = Xaa is Phe, Leu, Val, Lys, Tyr, Met, Ile, Arg, Ala, Glu, Asp, or Gln
  VARIANT 11: note = Xaa is Leu, Arg, Thr, Ile, Val, Ser, Cys, Tyr, Lys, Phe, or Gly
  VARIANT 12: note = Xaa is Lys, Gln, Tyr, Thr, Phe, Leu, Trp, Met, Ala, Glu, Gly, or Ser
  VARIANT 16: note = Xaa is Ile, Leu, Phe, Ser, Arg, Tyr, Gln, Trp, Asp, Lys, or His
  VARIANT 17: note = Xaa is Asp, Ser, Ile, Asn, Glu, Ala, His, Phe, Leu, Gln, Met, Gly, Tyr, or Val
  VARIANT 22: note = Xaa is Thr, Val, Cys, Glu, Ser, or Ala
  VARIANT 23: note = Xaa is Arg, Phe, Thr, Trp, Glu, Leu, Asn, Cys, Lys, Val, Ser, Gln, Ile, Tyr, His, or Ala
  VARIANT 24: note = Xaa is Ser, Pro, Arg, Lys, Asn, Ala, His, Gln, Gly, or Leu
  VARIANT 25: note = Xaa is Asp, Gly, Thr, Asn, Ser, Lys, Ala, Ile, Glu, Leu, Gln, Arg, or Tyr
  VARIANT 26: note = Xaa is Lys, Val, Ala, Glu, Tyr, Ile, Cys, Leu, Ser, Thr, Gly, Lys, Met, Asp, or Phe
XXXHXXPXSXXXDDSXXNKVLXXXXXN

SEQ ID NO: 27 - protein, 27 aa, synthetic construct
  REGION 1..27: note = HNH-like domain
  VARIANT 1: note = Xaa is Asp or Glu
  VARIANT 3: note = Xaa is Asp or Glu
  VARIANT 8: note = Xaa is Gln, His, Arg, Lys, Tyr, Ile, Leu, or Trp
  VARIANT 10: note = Xaa is Phe, Leu, Val, Lys, Tyr, Met, Ile, Arg, Ala, Glu, Asp, or Gln
  VARIANT 11: note = Xaa is Leu, Arg, Thr, Ile, Val, Ser, Cys, Tyr, Lys, Phe, or Gly
  VARIANT 12: note = Xaa is Lys, Gln, Tyr, Thr, Phe, Leu, Trp, Met, Ala, Glu, Gly, or Ser
  VARIANT 16: note = Xaa is Ile, Leu, Phe, Ser, Arg, Tyr, Gln, Trp, Asp, Lys, or His
  VARIANT 17: note = Xaa is Asp, Ser, Ile, Asn, Glu, Ala, His, Phe, Leu, Gln, Met, Gly, Tyr, or Val
  VARIANT 23: note = Xaa is Arg, Phe, Thr, Trp, Glu, Leu, Asn, Cys, Lys, Val, Ser, Gln, Ile, Tyr, His, or Ala
  VARIANT 24: note = Xaa is Ser, Pro, Arg, Lys, Asn, Ala, His, Gln, Gly, or Leu
  VARIANT 25: note = Xaa is Asp, Gly, Thr, Asn, Ser, Lys, Ala, Ile, Glu, Leu, Gln, Arg, or Tyr
  VARIANT 26: note = Xaa is Lys, Val, Ala, Glu, Tyr, Ile, Cys, Leu, Ser, Thr, Gly, Lys, Met, Asp, or Phe
XVXHIVPXSXXXDDSXXNKVLTXXXXN

SEQ ID NO: 28 - protein, 27 aa, synthetic construct
  REGION 1..27: note = HNH-like domain
  VARIANT 2: note = Xaa is Ile or Val
  VARIANT 6: note = Xaa is Ile or Val
  VARIANT 9: note = Xaa is Ala or Ser
  VARIANT 11: note = Xaa is Ile or Leu
  VARIANT 12: note = Xaa is Lys or Thr
  VARIANT 14: note = Xaa is Asp or Asn
  VARIANT 19: note = Xaa is Arg, Lys, or Leu
  VARIANT 22: note = Xaa is Thr or Val
  VARIANT 23: note = Xaa is Ser or Arg
  VARIANT 25: note = Xaa is Lys, Asp, or Ala
  VARIANT 26: note = Xaa is Glu, Lys, Gly, or Asn
DXDHIXPQXFXXDXSIDNXVLXXSXXN

SEQ ID NO: 29 - RNA, 116 nt, synthetic construct
  misc_feature 1..116: note = gRNA
  misc_feature 1..20: note = a, c, u, g, unknown or other
  misc_feature 1..20: note = targeting region
  misc_feature 21..42: note = first complementarity domain
  misc_feature 43..46: note = linking domain
  misc_feature 47..70: note = second complementarity domain
  source 1..116: mol_type = other RNA
nnnnnnnnnnnnnnnnnnnngttttagagctatgctgttttggaaacaaaacagcatagcaagttaaaataaggctagtccgttatcaacttgaaaaagtggcaccgagtcggtgc

SEQ ID NO: 30 - RNA, 116 nt, synthetic construct
  misc_feature 1..116: note = gRNA
  misc_feature 1..20: note = a, c, u, g, unknown or other
  misc_feature 1..20: note = targeting region
  misc_feature 21..42: note = first complementarity domain
  misc_feature 43..46: note = linking domain
  misc_feature 47..70: note = second complementarity domain
  source 1..116: mol_type = other RNA
nnnnnnnnnnnnnnnnnnnngtattagagctatgctgtattggaaacaatacagcatagcaagttaatataaggctagtccgttatcaacttgaaaaagtggcaccgagtcggtgc

SEQ ID NO: 31 - RNA, 96 nt, synthetic construct
  misc_feature 1..96: note = gRNA
  misc_feature 1..20: note = a, c, u, g, unknown or other
  misc_feature 1..20: note = targeting domain
  misc_feature 21..32: note = first complementarity domain
  misc_feature 33..36: note = linking domain
  misc_feature 37..50: note = second complementarity domain
  misc_feature 51..62: note = proximal domain
  source 1..96: mol_type = other RNA
nnnnnnnnnnnnnnnnnnnngtttaagagctagaaatagcaagtttaaataaggctagtccgttatcaacttgaaaaagtggcaccgagtcggtgc

SEQ ID NO: 32 - RNA, 47 nt, synthetic construct
  misc_feature 1..47: note = gRNA proximal and tail domains derived from S. pyogenes
  source 1..47: mol_type = other RNA
aaggctagtccgttatcaacttgaaaaagtggcaccgagtcggtgct

SEQ ID NO: 33 - RNA, 49 nt, synthetic construct
  misc_feature 1..49: note = gRNA proximal and tail domains
  source 1..49: mol_type = other RNA
aaggctagtccgttatcaacttgaaaaagtggcaccgagtcggtggtgc

SEQ ID NO: 34 - RNA, 51 nt, synthetic construct
  misc_feature 1..51: note = gRNA proximal and tail domains
  source 1..51: mol_type = other RNA
aaggctagtccgttatcaacttgaaaaagtggcaccgagtcggtgcggatc

SEQ ID NO: 35 - RNA, 31 nt, synthetic construct
  misc_feature 1..31: note = gRNA proximal and tail domains
  source 1..31: mol_type = other RNA
aaggctagtccgttatcaacttgaaaaagtg

SEQ ID NO: 36 - RNA, 18 nt, synthetic construct
  misc_feature 1..18: note = gRNA proximal and tail domains
  source 1..18: mol_type = other RNA
aaggctagtccgttatca

SEQ ID NO: 37 - RNA, 12 nt, synthetic construct
  misc_feature 1..12: note = gRNA proximal and tail domains
  source 1..12: mol_type = other RNA
aaggctagtccg

SEQ ID NO: 38 - RNA, 102 nt, synthetic construct
  misc_feature 1..102: note = Unimolecular gRNA derived from S. aureus
  misc_feature 1..20: note = n is a, c, g, or u
  misc_feature 1..20: note = Targeting domain
  source 1..102: mol_type = other RNA
nnnnnnnnnnnnnnnnnnnngttttagtactctggaaacagaatctactaaaacaaggcaaaatgccgtgtttatctcgtcaacttgttggcgagatttttt

SEQ ID NO: 39 - RNA, 42 nt, synthetic construct
  misc_feature 1..42: note = Modular gRNA derived from S. pyogenes
  misc_feature 1..20: note = a, c, u, g, unknown or other
  misc_feature 1..20: note = Targeting domain
  misc_feature 21..42: note = First complementarity domain
  source 1..42: mol_type = other RNA
nnnnnnnnnnnnnnnnnnnngttttagagctatgctgttttg

SEQ ID NO: 40 - RNA, 85 nt, synthetic construct
  misc_feature 1..85: note = Modular gRNA derived from S. pyogenes
  misc_feature 1..9: note = 5' extension domain
  misc_feature 10..33: note = Second complementarity domain
  misc_feature 34..45: note = Proximal domain
  misc_feature 46..85: note = Tail domain
  source 1..85: mol_type = other RNA
ggaaccattcaaaacagcatagcaagttaaaataaggctagtccgttatcaacttgaaaaagtggcaccgagtcggtgctttttt

SEQ ID NO: 41 - RNA, 62 nt, synthetic construct
  misc_feature 1..62: note = Unimolecular gRNA derived from S. pyogenes
  misc_feature 1..20: note = a, c, u, g, unknown or other
  misc_feature 1..20: note = Targeting domain
  misc_feature 21..32: note = First complementarity domain
  misc_feature 33..36: note = Linking domain
  misc_feature 37..50: note = Second complementarity domain
  misc_feature 51..62: note = Proximal domain
  source 1..62: mol_type = other RNA
nnnnnnnnnnnnnnnnnnnngttttagagctagaaatagcaagttaaaataaggctagtccg

SEQ ID NO: 42 - RNA, 102 nt, synthetic construct
  misc_feature 1..102: note = Unimolecular gRNA derived from S. pyogenes
  misc_feature 1..20: note = a, c, u, g, unknown or other
  misc_feature 1..20: note = Targeting domain
  misc_feature 21..32: note = First complementarity domain
  misc_feature 33..36: note = Linking domain
  misc_feature 37..50: note = Second complementarity domain
  misc_feature 51..62: note = Proximal domain
  misc_feature 63..102: note = Tail domain
  source 1..102: mol_type = other RNA
nnnnnnnnnnnnnnnnnnnngttttagagctagaaatagcaagttaaaataaggctagtccgttatcaacttgaaaaagtggcaccgagtcggtgctttttt

SEQ ID NO: 43 - RNA, 75 nt, synthetic construct
  misc_feature 1..75: note = Unimolecular gRNA derived from S. pyogenes
  misc_feature 1..20: note = Targeting domain
  misc_feature 1..20: note = a, c, u, g, unknown or other
  misc_feature 21..36: note = First complementarity domain
  misc_feature 37..40: note = Linking domain
  misc_feature 41..58: note = Second complementarity domain
  misc_feature 59..70: note = Proximal domain
  misc_feature 71..75: note = Tail domain
  source 1..75: mol_type = other RNA
nnnnnnnnnnnnnnnnnnnngttttagagctatgctgaaaagcatagcaagttaaaataaggctagtccgttatc

SEQ ID NO: 44 - RNA, 87 nt, synthetic construct
  misc_feature 1..87: note = Unimolecular gRNA derived from S. pyogenes
  misc_feature 1..20: note = a, c, u, g, unknown or other
  misc_feature 1..20: note = Targeting domain
  misc_feature 21..32: note = First complementarity domain
  misc_feature 43..46: note = Linking domain
  misc_feature 57..70: note = Second complementarity domain
  misc_feature 71..82: note = Proximal domain
  misc_feature 83..87: note = Tail domain
  source 1..87: mol_type = other RNA
nnnnnnnnnnnnnnnnnnnngttttagagctatgctgttttggaaacaaaacagcatagcaagttaaaataaggctagtccgttatc

SEQ ID NO: 45 - RNA, 42 nt, synthetic construct
  misc_feature 1..42: note = Modular gRNA derived from S. thermophilus
  misc_feature 1..20: note = a, c, u, g, unknown or other
  misc_feature 1..20: note = Targeting domain
  misc_feature 21..42: note = First complementarity domain
  source 1..42: mol_type = other RNA
nnnnnnnnnnnnnnnnnnnngttttagagctgtgttgtttcg

SEQ ID NO: 46 - RNA, 78 nt, synthetic construct
  misc_feature 1..78: note = Modular gRNA derived from S. thermophilus
  misc_feature 1..3: note = 5' extension domain
  misc_feature 4..27: note = Second complementarity domain
  misc_feature 28..40: note = Proximal domain
  misc_feature 41..78: note = Tail domain
  source 1..78: mol_type = other RNA
gggcgaaacaacacagcgagttaaaataaggcttagtccgtactcaacttgaaaaggtggcaccgattcggtgttttt

SEQ ID NO: 47 - RNA, 85 nt, synthetic construct
  misc_feature 1..85: note = Modular gRNA derived from S. pyogenes
  source 1..85: mol_type = other RNA
gaaccattcaaaacagcatagcaagttaaaataaggctagtccgttatcaacttgaaaaagtggcaccgagtcggtgcttttttt

SEQ ID NO: 48 - RNA, 96 nt, synthetic construct
  misc_feature 1..96: note = gRNA from S. pyogenes
  misc_feature 1..20: note = n is a, c, g, or u
  misc_feature 1..20: note = Targeting domain
  source 1..96: mol_type = other RNA
nnnnnnnnnnnnnnnnnnnngttttagagctagaaatagcaagttaaaataaggctagtccgttatcaacttgaaaaagtggcaccgagtcggtgc

SEQ ID NO: 49 - RNA, 96 nt, synthetic construct
  misc_feature 1..96: note = gRNA
  misc_feature 1..20: note = n is a, c, g, or u
  misc_feature 1..20: note = Targeting domain
  misc_feature 21..32: note = First complementarity domain
  misc_feature 33..36: note = Linking domain
  misc_feature 37..50: note = Second complementarity domain
  misc_feature 51..62: note = Proximal domain
  source 1..96: mol_type = other RNA
nnnnnnnnnnnnnnnnnnnngtattagagctagaaatagcaagttaatataaggctagtccgttatcaacttgaaaaagtggcaccgagtcggtgc

SEQ ID NO: 50 - RNA, 104 nt, synthetic construct
  misc_feature 1..104: note = gRNA
  misc_feature 1..20: note = n is a, c, g, or u
  misc_feature 1..20: note = Targeting domain
  misc_feature 21..36: note = First complementarity domain
  misc_feature 37..40: note = Linking domain
  misc_feature 41..58: note = Second complementarity domain
  source 1..104: mol_type = other RNA
nnnnnnnnnnnnnnnnnnnngttttagagctatgctgaaaagcatagcaagttaaaataaggctagtccgttatcaacttgaaaaagtggcaccgagtcggtgc

SEQ ID NO: 51 - RNA, 106 nt, synthetic construct
  misc_feature 1..106: note = gRNA
  misc_feature 1..20: note = n is a, c, g, or u
  misc_feature 1..20: note = Targeting domain
  misc_feature 21..37: note = First complementarity domain
  misc_feature 38..41: note = Linking domain
  misc_feature 42..60: note = Second complementarity domain
  source 1..106: mol_type = other RNA
nnnnnnnnnnnnnnnnnnnngttttagagctatgctggaaacagcatagcaagttaaaataaggctagtccgttatcaacttgaaaaagtggcaccgagtcggtgc

SEQ ID NO: 52 - protein, 12 aa, Peptoniphilus duerdenii
DIGTASVGWAVT

SEQ ID NO: 53 - protein, 12 aa, Treponema denticola
DVGTGSVGWAVT

SEQ ID NO: 54 - protein, 12 aa, Streptococcus mutans
DIGTNSVGWAVV

SEQ ID NO: 55 - protein, 12 aa, Streptococcus mutans
DIGTNSVGWAVI

SEQ ID NO: 56 - protein, 12 aa, Streptococcus mutans
DIGTNSVGWAVL

SEQ ID NO: 57 - protein, 12 aa, Streptococcus mutans
DLGTNSIGWAVV

SEQ ID NO: 58 - protein, 11 aa, Pedobacter glucosidilyticus
DLGTNSIGWAI

SEQ ID NO: 59 - protein, 12 aa, Streptococcus mutans
DLGTNSIGWALV

SEQ ID NO: 60 - protein, 12 aa, Fusobacterium nucleatum
DIGTNSVGWCVT

SEQ ID NO: 61 - protein, 12 aa, Streptococcus mutans
DIGTNSVGYAVT

SEQ ID NO: 62 - protein, 12 aa, Streptococcus mutans
DMGTGSLGWAVT

SEQ ID NO: 63 - protein, 12 aa, Streptococcus mutans
DIGTSSVGWAAI

SEQ ID NO: 64 - protein, 12 aa, Streptococcus mutans
DLGTGSVGWAVV

SEQ ID NO: 65 - protein, 12 aa, Streptococcus mutans
DLGVGSVGWAIV

SEQ ID NO: 66 - protein, 12 aa, Streptococcus mutans
DLGIASIGWAII

SEQ ID NO: 67 - protein, 12 aa, Streptococcus mutans
DLGIASVGWAIV

SEQ ID NO: 68 - protein, 12 aa, Streptococcus mutans
DLGVASVGWSIV

SEQ ID NO: 69 - protein, 12 aa, Eubacterium rectale
DIGIASVGWAIL

SEQ ID NO: 70 - protein, 12 aa, Streptococcus mutans
DLGISSVGWSVI

SEQ ID NO: 71 - protein, 12 aa, Streptococcus mutans
DIGIASVGWSVI

SEQ ID NO: 72 - protein, 12 aa, Streptococcus mutans
DVGIGSIGWAVI

SEQ ID NO: 73 - protein, 12 aa, Streptococcus mutans
DLGVGSIGFAIV

SEQ ID NO: 74 - protein, 12 aa, Akkermansia muciniphila
DIGYASIGWAVI

SEQ ID NO: 75 - protein, 12 aa, Prevotella ruminicola
DTGTNSLGWAIV

SEQ ID NO: 76 - protein, 12 aa, Cand. Puniceispirillum marinum
DLGTNSIGWCLL

SEQ ID NO: 77 - protein, 12 aa, Rhodospirillum rubrum
DIGTDSLGWAVF

SEQ ID NO: 78 - protein, 12 aa, Streptococcus mutans
DIGSNSIGFAVV

SEQ ID NO: 79 - protein, 12 aa, Streptococcus mutans
DLGVGSIGVAVA

SEQ ID NO: 80 - protein, 12 aa, Rhodopseudomonas palustris
DLGIASCGWGVV

SEQ ID NO: 81 - protein, 12 aa, Streptococcus mutans
DLGIASVGWCLT

SEQ ID NO: 82 - protein, 12 aa, Streptococcus mutans
DIGIGSVGVGIL

SEQ ID NO: 83 - protein, 12 aa, Streptococcus mutans
DIGITSVGYGLI

SEQ ID NO: 84 - protein, 12 aa, Streptococcus mutans
DIGITSVGFGII

SEQ ID NO: 85 - protein, 12 aa, Streptococcus mutans
DVGITSTGYAVL

SEQ ID NO: 86 - protein, 12 aa, Streptococcus mutans
DLGITSFGYAIL

SEQ ID NO: 87 - protein, 12 aa, Streptococcus mutans
DIGNASVGWSAF

SEQ ID NO: 88 - protein, 12 aa, Lactobacillus gasseri
DVGTNSCGWVAM

SEQ ID NO: 89 - protein, 12 aa, Streptococcus mutans
DVGERSIGLAAV

SEQ ID NO: 90 - protein, 12 aa, Streptococcus mutans
DVGLNSVGLAAV

SEQ ID NO: 91 - protein, 12 aa, Bifidobacterium dentium
DVGLMSVGLAAI

SEQ ID NO: 92 - protein, 12 aa, Corynebacterium diphtheriae
DVGTFSVGLAAI

SEQ ID NO: 93 - protein, 12 aa, Streptococcus mutans
DIGTGSVGYACM

SEQ ID NO: 94 - protein, 12 aa, Capnocytophaga ochracea
DLGTTSIGFAHI

SEQ ID NO: 95 - protein, 12 aa, Prevotella denticola
DLGTNSIGSSVR

SEQ ID NO: 96 - protein, 12 aa, Ralstonia solanacearum
DIGTNSIGWAVI

SEQ ID NO: 97 - protein, 12 aa, Streptococcus mutans
DLGIASVGWAVV

SEQ ID NO: 98 - protein, 12 aa, Comamonas granuli
DIGIASVGWAVL

SEQ ID NO: 99 - protein, 12 aa, Streptococcus mutans
DIGIASIGWAVI

SEQ ID NO: 100 - protein, 12 aa, Agathobacter rectalis
DIGIASVGWAII

SEQ ID NO: 101 - protein, 12 aa, Streptococcus mutans
DVGIASVGWAVI

SEQ ID NO: 102 - protein, 11 aa, Streptococcus mutans
DIGIASVGWAL

SEQ ID NO: 103 - protein, 12 aa, Neisseria meningitidis
DIGIASVGWAMV

SEQ ID NO: 104 - protein, 12 aa, Clostridium perfringens
DIGITSVGWAVI

SEQ ID NO: 105 - protein, 12 aa, Streptococcus mutans
DLGISSLGWAIV

SEQ ID NO: 106 - protein, 12 aa, Streptococcus mutans
DLGTNSIGWGLL

SEQ ID NO: 107 - protein, 12 aa, Verminephrobacter eiseniae
DLGSTSLGWAIF

SEQ ID NO: 108 - protein, 12 aa, Streptococcus mutans
DIGISSIGWAFS

SEQ ID NO: 109 - protein, 12 aa, Streptococcus mutans
DIGTTSIGFSVI

SEQ ID NO: 110 - protein, 12 aa, Streptococcus mutans
DIGTSSIGWWLY

SEQ ID NO: 111 - protein, 12 aa, Streptococcus mutans
DLGSNSLGWFVT

SEQ ID NO: 112 - protein, 12 aa, Streptococcus mutans
DLGANSLGWFVV

SEQ ID NO: 113 - protein, 15 aa, Bacillus cereus
DIGLRIGITSCGWSI

SEQ ID NO: 114 - protein, 12 aa, Sutterella wadsworthensis
DMGAKYTGVFYA

SEQ ID NO: 115 - protein, 12 aa, Streptococcus mutans
DLGGKNTGFFSF

SEQ ID NO: 116 - protein, 12 aa, Francisella tularensis
DLGVKNTGVFSA

SEQ ID NO: 117 - protein, 12 aa, Streptococcus mutans
DLGAKFTGVALY

SEQ ID NO: 118 - protein, 12 aa, Streptococcus mutans
DLGGKFTGVCLS

SEQ ID NO: 119 - protein, 12 aa, Parasutterella excrementihominis
DLGGTYTGTFIT

SEQ ID NO: 120 - protein, 12 aa, Streptococcus mutans
DIGTNSVGWAVT

SEQ ID NO: 121 - protein, 12 aa, Eubacterium yurii
DVGTNSVGWAVT

SEQ ID NO: 122 - protein, 12 aa, Butyrivibrio hungatei
DMGTNSVGWAVT

SEQ ID NO: 123 - protein, 12 aa, Streptococcus mutans
DVGTSSVGWAVT

SEQ ID NO: 124 - protein, 27 aa, Treponema denticola
DIDHIYPQSKIKDDSISNRVLVCSSCN

SEQ ID NO: 125 - protein, 27 aa, Streptococcus mutans
DIDHIYPQSKTMDDSLNNRVLVKKNYN

SEQ ID NO: 126 - protein, 27 aa, Peptoniphilus duerdenii
DQDHIYPKSKIYDDSLENRVLVKKNLN

SEQ ID NO: 127 - protein, 27 aa, Streptococcus mutans
QIDHIVPQSLVKDDSFDNRVLVVPSEN

SEQ ID NO: 128 - protein, 27 aa, Streptococcus mutans
DIDHIIPQAFIKDNSIDNRVLTSSKEN

SEQ ID NO: 129 - protein, 27 aa, Streptococcus mutans
DIDHIIPQAFLKDNSIDNKVLVSSASN

SEQ ID NO: 130 - protein, 27 aa, Streptococcus mutans
DIDHIIPQAYTKDNSLDNRVLVSNITN

SEQ ID NO: 131 - protein, 27 aa, Streptococcus mutans
DIDHIVPQSFITDNSIDNLVLTSSAGN

SEQ ID NO: 132 - protein, 27 aa, Streptococcus mutans
DVDHIVPQSFLKDDSIDNKVLTRSDKN

SEQ ID NO: 133 - protein, 27 aa, Streptococcus mutans
NIDHIYPQSMVKDDSLDNKVLVQSEIN

SEQ ID NO: 134 - protein, 27 aa, Streptococcus mutans
DIDHILPQSLIKDDSLDNRVLVNATIN

SEQ ID NO: 135 - protein, 27 aa, Lactobacillus gasseri
DIDHILPQSFIKDDSLENRVLVKKAVN

SEQ ID NO: 136 - protein, 27 aa, Streptococcus mutans
EVDHIFPRSFIKDDSIDNKVLVIKKMN

SEQ ID NO: 137 - protein, 27 aa, Olsenella uli
EVDHIIPRSYIKDDSFENKVLVYREEN

SEQ ID NO: 138 - protein, 27 aa, Streptococcus mutans
DIDHIIPQAVTQNDSIDNRVLVARAEN

SEQ ID NO: 139 - protein, 27 aa, Streptococcus mutans
EIDHIIPYSISFDDSSSNKLLVLAESN

SEQ ID NO: 140 - protein, 27 aa, Streptococcus mutans
EIDHIIPYSLCFDDSSANKVLVHKQSN

SEQ ID NO: 141 - protein, 27 aa, Streptococcus mutans
DIDHIIPYSRSMDDSYSNKVLVLSGEN

SEQ ID NO: 142 - protein, 27 aa, Streptococcus mutans
DIDHIIPYSKSMDDSFNNKVLCLAEEN

SEQ ID NO: 143 - protein, 27 aa, Campylobacter jejuni
EIDHIYPYSRSFDDSYMNKVLVFTKQN

SEQ ID NO: 144 - protein, 27 aa, Streptococcus mutans
QIDHIYPYSRSMDDSYMNKVLVLTDEN

SEQ ID NO: 145 - protein, 27 aa, Clostridium perfringens
EIDHIIPFSRSFDDSLSNKILVLGSEN

SEQ ID NO: 146 - protein, 27 aa, Streptococcus mutans
EIDHALPFSRTWDDSFNNKVLVLGSEN

SEQ ID NO: 147 - protein, 27 aa, Streptococcus mutans
EIDHALPFSRTWDDSFNNKVLVLASEN

SEQ ID NO: 148 - protein, 27 aa, Streptococcus mutans
EIDHIIPISISLDDSINNKVLVLSKAN

SEQ ID NO: 149 - protein, 27 aa, Streptococcus mutans
EVDHIIPISISLDDSITNKVLVTHREN

SEQ ID NO: 150 - protein, 27 aa, Acidovorax ebreus
QVDHALPYSRSYDDSKNNKVLVLTHEN

SEQ ID NO: 151 - protein, 27 aa, Streptococcus mutans
EVDHILPLSITFDDSLANKVLVYATAN

SEQ ID NO: 152 - protein, 27 aa, Eubacterium rectale
EIDHIIPRSISFDDARSNKVLVYRSEN

SEQ ID NO: 153 - protein, 27 aa, Streptococcus mutans
EVDHIIPRSVSFDNSYHNKVLVKQSEN

SEQ ID NO: 154 - protein, 27 aa, Roseburia intestinalis
DIDHILPYSITFDDSFRNKVLVTSQEN

SEQ ID NO: 155 - protein, 27 aa, Streptococcus mutans
EIDHILPRSRSADDSFANKVLCLARAN

SEQ ID NO: 156 - protein, 27 aa, Cand. Puniceispirillum marinum
EIEHLLPFSLTLDDSMANKTVCFRQAN

SEQ ID NO: 157 - protein, 27 aa, Streptococcus mutans
DIDHILPFSVSLDDSAANKVVCLREAN

SEQ ID NO: 158 - protein, 27 aa, Streptococcus mutans
DIDHLIPFSISWDDSAANKVVCMRYAN

SEQ ID NO: 159 - protein, 27 aa, Streptococcus mutans
DIDHILPVAMTLDDSPANKIICMRYAN

SEQ ID NO: 160 - protein, 27 aa, Dinoroseobacter shibae
DVDHILPYSRTLDDSFPNRTLCLREAN

SEQ ID NO: 161 - protein, 27 aa, Verminephrobacter eiseniae
EIEHILPFSRTLDDSLNNRTVAMRRAN

SEQ ID NO: 162 - protein, 27 aa, Streptococcus mutans
EVDHIIPYSISWDDSYTNKVLTSAKCN

SEQ ID NO: 163 - protein, 27 aa, Rhodopseudomonas palustris
QVDHILPWSRFGDDSYLNKTLCTARSN

SEQ ID NO: 164 - protein, 27 aa, Streptococcus mutans
QVDHILPFSKTLDDSFANKVLAQHDAN

SEQ ID NO: 165 - protein, 27 aa, Streptococcus mutans
QIDHAFPLSRSLDDSQSNKVLCLTSSN

SEQ ID NO: 166 - protein, 27 aa, Streptococcus mutans
DIDHIVPRSISFDDSFSNLVIVNKLDN

SEQ ID NO: 167 - protein, 27 aa, Streptococcus mutans
EIEHIIPYSMSYDNSQANKILTEKAEN

SEQ ID NO: 168 - protein, 27 aa, Streptococcus mutans
EIDHVIPYSKSADDSWFNKLLVKKSTN

SEQ ID NO: 169 - protein, 27 aa, Streptococcus mutans
EMDHILPYSRSLDNGWHNRVLVHGKDN

SEQ ID NO: 170 - protein, 27 aa, Streptococcus mutans
EVDHIVPYSLILDNTINNKALVYAEEN

SEQ ID NO: 171 - protein, 27 aa, Fibrobacter succinogenes
EIEHVIPQSLYFDDSFSNKVICEAEVN

SEQ ID NO: 172 - protein, 27 aa, Streptococcus mutans
DIEHIIPQARLFDDSFSNKTLEARSVN

SEQ ID NO: 173 - protein, 27 aa, Capnocytophaga sputigena
EIEHIVPKARVFDDSFSNKTLTFHRIN

SEQ ID NO: 174 - protein, 28 aa, Finegoldia magna
DKDHIIPQSMKKDDSIINNLVLVNKNAN

SEQ ID NO: 175 - protein, 27 aa, Streptococcus mutans
EVEHIWPRSRSFDNSPRNKTLCRKDVN

SEQ ID NO: 176 - protein, 27 aa, Bacillus cereus
IVNHIIPYNRSFDDTYHNRVLTLTETK

SEQ ID NO: 177 - protein, 27 aa, Prevotella micans
DMEHTIPKSISFDNSDQNLTLCESYYN

SEQ ID NO: 178 - protein, 27 aa, Prevotella ruminicola
DIEHTIPRSAGGDSTKMNLTLCSSRFN

SEQ ID NO: 179 - protein, 27 aa, Flavobacterium columnare
DIEHTIPRSISQDNSQMNKTLCSLKFN

SEQ ID NO: 180 - protein, 27 aa, Rhodospirillum rubrum
DIDHVIPLARGGRDSLDNMVLCQSDAN

SEQ ID NO: 181 - protein, 27 aa, Streptococcus mutans
DIEHLFPIAESEDNGRNNLVISHSACN

SEQ ID NO: 182 - protein, 27 aa, Streptococcus mutans
DVDHIFPRDDTADNSYGNKVVAHRQCN

SEQ ID NO: 183 - protein, 27 aa, Streptococcus mutans
DIEHIVPQSLGGLSTDYNTIVTLKSVN

SEQ ID NO: 184 - protein, 27 aa, Streptococcus mutans
ELDHIVPRTDGGSNRHENLAITCGACN

SEQ ID NO: 185 - protein, 28 aa, Streptococcus mutans
EMDHIVPRKGVGSTNTRTNFAAVCAECN

SEQ ID NO: 186 - protein, 28 aa, Bifidobacterium dentium
EMDHIVPRKGVGSTNTRVNLAAACAACN

SEQ ID NO: 187 - protein, 28 aa, Corynebacterium diphtheriae
EMDHIVPRAGQGSTNTRENLVAVCHRCN

SEQ ID NO: 188 - protein, 33 aa, Sutterella wadsworthensis
EIDHILPRSLIKDARGIVFNAEPNLIYASSRGN

SEQ ID NO: 189 - protein, 33 aa, Streptococcus mutans
EIDHIIPRSLTGRTKKTVFNSEANLIYCSSKGN

SEQ ID NO: 190 - protein, 33 aa, Parasutterella excrementihominis
EIDHIIPRSLTLKKSESIYNSEVNLIFVSAQGN

SEQ ID NO: 191 - protein, 33 aa, Streptococcus mutans
EIDHIYPRSLSKKHFGVIFNSEVNLIYCSSQGN

SEQ ID NO: 192 - protein, 33 aa, Streptococcus mutans
EIDHILPRSHTLKIYGTVFNPEGNLIYVHQKCN

SEQ ID NO: 193 - protein, 30 aa, Francisella tularensis
ELDHIIPRSHKKYGTLNDEANLICVTRGDN

SEQ ID NO: 194 - protein, 27 aa, Akkermansia muciniphila
ELEHIVPHSFRQSNALSSLVLTWPGVN

SEQ ID NO: 195 - protein, 27 aa, Streptococcus mutans
DIDHIYPRSKIKDDSITNRVLVEKDIN

SEQ ID NO: 196 - protein, 28 aa, Streptococcus mutans
YDIDHIYPRSLTKDDSFDNLVLCERTAN

SEQ ID NO: 197 - protein, 28 aa, Fusobacterium nucleatum
DIDHIYPRSKVIKDDSFDNLVLVLKNEN

SEQ ID NO: 198 - protein, 27 aa, Filifactor alocis
DRDHIYPQSKIKDDSIDNLVLVNKTYN

SEQ ID NO: 199-250 - skipped (52 entries)

SEQ ID NO: 251 - RNA, 17 nt, synthetic construct
  misc_feature 1..17: note = Targeting domain
  source 1..17: mol_type = other RNA
gaaggaaactagctaaa

SEQ ID NO: 252 - RNA, 20 nt, synthetic construct
  misc_feature 1..20: note = Targeting domain
  source 1..20: mol_type = other RNA
ggagaaggaaactagctaaa

SEQ ID NO: 253 - RNA, 20 nt, synthetic construct
  misc_feature 1..20: note = Targeting domain
  source 1..20: mol_type = other RNA
gggagaaggaaactagctaa

SEQ ID NO: 254 - RNA, 20 nt, synthetic construct
  misc_feature 1..20: note = Targeting domain
  source 1..20: mol_type = other RNA
gtatcctctatgatgggaga

SEQ ID NO: 255 - RNA, 20 nt, synthetic construct
  misc_feature 1..20: note = Targeting domain
  source 1..20: mol_type = other RNA
gtttccttctcccatcatag

SEQ ID NO: 256 - RNA, 20 nt, synthetic construct
  misc_feature 1..20: note = Targeting domain
  source 1..20: mol_type = other RNA
gtcctggtatcctctatgat

SEQ ID NO: 257 - RNA, 17 nt, synthetic construct
  misc_feature 1..17: note = Targeting domain
  source 1..17: mol_type = other RNA
agaaggaaactagctaa

SEQ ID NO: 258 - RNA, 17 nt, synthetic construct
  misc_feature 1..17: note = Targeting domain
  source 1..17: mol_type = other RNA
tcctctatgatgggaga

SEQ ID NO: 259 - RNA, 17 nt, synthetic construct
  misc_feature 1..17: note = Targeting domain
  source 1..17: mol_type = other RNA
cctggtatcctctatga

SEQ ID NO: 260 - RNA, 17 nt, synthetic construct
  misc_feature 1..17: note = Targeting domain
  source 1..17: mol_type = other RNA
ccatcatagaggatacc

SEQ ID NO: 261 - RNA, 17 nt, synthetic construct
  misc_feature 1..17: note = Targeting domain
  source 1..17: mol_type = other RNA
tccttctcccatcatag

SEQ ID NO: 262 - RNA, 17 nt, synthetic construct
  misc_feature 1..17: note = Targeting domain
  source 1..17: mol_type = other RNA
tagcagtatcctcttgg

SEQ ID NO: 263 - RNA, 17 nt, synthetic construct
  misc_feature 1..17: note = Targeting domain
  source 1..17: mol_type = other RNA
ttagcagtatcctcttg

SEQ ID NO: 264 - RNA, 17 nt, synthetic construct
  misc_feature 1..17: note = Targeting domain
  source 1..17: mol_type = other RNA
aactggaatgactgaat

SEQ ID NO: 265 - RNA, 17 nt, synthetic construct
  misc_feature 1..17: note = Targeting domain
  source 1..17: mol_type = other RNA
ctggtatcctctatgat

SEQ ID NO: 266 - RNA, 17 nt, synthetic construct
  misc_feature 1..17: note = Targeting domain
  source 1..17: mol_type = other RNA
aattagcagtatcctct

SEQ ID NO: 267 - RNA, 17 nt, synthetic construct
  misc_feature 1..17: note = Targeting domain
  source 1..17: mol_type = other RNA
attagcagtatcctctt

SEQ ID NO: 268 - RNA, 20 nt, synthetic construct
  misc_feature 1..20: note = Targeting domain
  source 1..20: mol_type = other RNA
agtcctggtatcctctatga

SEQ ID NO: 269 - RNA, 20 nt, synthetic construct
  misc_feature 1..20: note = Targeting domain
  source 1..20: mol_type = other RNA
ctcccatcatagaggatacc

SEQ ID NO: 270 - RNA, 20 nt, synthetic construct
  misc_feature 1..20: note = Targeting domain
  source 1..20: mol_type = other RNA
aattagcagtatcctcttgg

SEQ ID NO: 271 - RNA, 20 nt, synthetic construct
  misc_feature 1..20: note = Targeting domain
  source 1..20: mol_type = other RNA
aaattagcagtatcctcttg

SEQ ID NO: 272 - RNA, 20 nt, synthetic construct
  misc_feature 1..20: note = Targeting domain
  source 1..20: mol_type = other RNA
aaaaactggaatgactgaat

SEQ ID NO: 273 - RNA, 20 nt, synthetic construct
  misc_feature 1..20: note = Targeting domain
  source 1..20: mol_type = other RNA
aaaaattagcagtatcctct

SEQ ID NO: 274 - RNA, 20 nt, synthetic construct
  misc_feature 1..20: note = Targeting domain
  source 1..20: mol_type = other RNA
aaaattagcagtatcctctt

SEQ ID NO: 275 - RNA, 17 nt, synthetic construct
  misc_feature 1..17: note = Targeting domain
  source 1..17: mol_type = other RNA
gaatcggaacaaggcaa

SEQ ID NO: 276 - RNA, 17 nt, synthetic construct
  misc_feature 1..17: note = Targeting domain
  source 1..17: mol_type = other RNA
gaccaatagccttgaca

SEQ ID NO: 277 - RNA, 17 nt, synthetic construct
  misc_feature 1..17: note = Targeting domain
  source 1..17: mol_type = other RNA
ggctattggtcaaggca

SEQ ID NO: 278 - RNA, 17 nt, synthetic construct
  misc_feature 1..17: note = Targeting domain
  source 1..17: mol_type = other RNA
gtcaaggctattggtca

SEQ ID NO: 279 - RNA, 17 nt, synthetic construct
  misc_feature 1..17: note = Targeting domain
  source 1..17: mol_type = other RNA
gtgtgtggaactgctga

SEQ ID NO: 280 - RNA, 17 nt, synthetic construct
  misc_feature 1..17: note = Targeting domain
  source 1..17: mol_type = other RNA
gggccggcggctggcta

SEQ ID NO: 281 - RNA, 17 nt, synthetic construct
  misc_feature 1..17: note = Targeting domain
  source 1..17: mol_type = other RNA
gagtatccagtgaggcc

SEQ ID NO: 282 - RNA, 17 nt, synthetic construct
  misc_feature 1..17: note = Targeting domain
  source 1..17: mol_type = other RNA
gctgacaaaagaagtcc

SEQ ID NO: 283 - RNA, 17 nt, synthetic construct
  misc_feature 1..17: note = Targeting domain
  source 1..17: mol_type = other RNA
ggccaggggccggcggc

SEQ ID NO: 284 - RNA, 17 nt, synthetic construct
  misc_feature 1..17: note = Targeting domain
  source 1..17: mol_type = other RNA
gggaaggggcccccaag

SEQ ID NO: 285 - RNA, 17 nt, synthetic construct
  misc_feature 1..17: note = Targeting domain
  source 1..17: mol_type = other RNA
gagatagtgtggggaag

SEQ ID NO: 286 - RNA, 17 nt, synthetic construct
  misc_feature 1..17: note = Targeting domain
  source 1..17: mol_type = other RNA
gtatccagtgaggccag

SEQ ID NO: 287 - RNA, 17 nt, synthetic construct
  misc_feature 1..17: note = Targeting domain
  source 1..17: mol_type = other RNA
gtgaggccaggggccgg

SEQ ID NO: 288 - RNA, 17 nt, synthetic construct
  misc_feature 1..17: note = Targeting domain
  source 1..17: mol_type = other RNA
gctggccaacccatggg

SEQ ID NO: 289 - RNA, 17 nt, synthetic construct
  misc_feature 1..17: note = Targeting domain
  source 1..17: mol_type = other RNA
ggctaaactccacccat

SEQ ID NO: 290 - RNA, 17 nt, synthetic construct
  misc_feature 1..17: note = Targeting domain
  source 1..17: mol_type = other RNA
ggatactctaagactat

SEQ ID NO: 291 - RNA, 17 nt, synthetic construct
  misc_feature 1..17: note = Targeting domain
  source 1..17: mol_type = other RNA
ggggccggcggctggct

SEQ ID NO: 292 - RNA, 20 nt, synthetic construct
  misc_feature 1..20: note = Targeting domain
  source 1..20: mol_type = other RNA
ggctagggatgaagaataaa

SEQ ID NO: 293 - RNA, 20 nt, synthetic construct
  misc_feature 1..20: note = Targeting domain
  source 1..20: mol_type = other RNA
gagtgtgtggaactgctgaa

SEQ ID NO: 294 - RNA, 20 nt, synthetic construct
  misc_feature 1..20: note = Targeting domain
  source 1..20: mol_type = other RNA
ggaatgactgaatcggaaca

SEQ ID NO: 295 - RNA, 20 nt, synthetic construct
  misc_feature 1..20: note = Targeting domain
  source 1..20: mol_type = other RNA
gcattgagatagtgtgggga

SEQ ID NO: 296 - RNA, 20 nt, synthetic construct
  misc_feature 1..20: note = Targeting domain
  source 1..20: mol_type = other RNA
gctattggtcaaggcaaggc

SEQ ID NO: 297 - RNA, 20 nt, synthetic construct
  misc_feature 1..20: note = Targeting domain
  source 1..20: mol_type = other RNA
gtggggaaggggcccccaag

SEQ ID NO: 298 - RNA, 20 nt, synthetic construct
  misc_feature 1..20: note = Targeting domain
  source 1..20: mol_type = other RNA
ggcaaggctggccaacccat

SEQ ID NO: 299 - RNA, 20 nt, synthetic construct
  misc_feature 1..20: note = Targeting domain
  source 1..20: mol_type = other RNA
gtttgccttgtcaaggctat

SEQ ID NO: 300 - RNA, 20 nt, synthetic construct
  misc_feature 1..20: note = Targeting domain
  source 1..20: mol_type = other RNA
gctaaactccacccatgggt

SEQ ID NO: 301 - RNA, 17 nt, synthetic construct
  misc_feature 1..17: note = Targeting domain
  source 1..17: mol_type = other RNA
caaatatctgtctgaaa

SEQ ID NO: 302 - RNA, 17 nt, synthetic construct
  misc_feature 1..17: note = Targeting domain
  source 1..17: mol_type = other RNA
tagggatgaagaataaa

SEQ ID NO: 303 - RNA, 17 nt, synthetic construct
  misc_feature 1..17: note = Targeting domain
  source 1..17: mol_type = other RNA
tgagatagtgtggggaa

SEQ ID NO: 304 - RNA, 17 nt, synthetic construct
  misc_feature 1..17: note = Targeting domain
  source 1..17: mol_type = other RNA
tgtgtggaactgctgaa

SEQ ID NO: 305 - RNA, 17 nt, synthetic construct
  misc_feature 1..17: note = Targeting domain
  source 1..17: mol_type = other RNA
atgactgaatcggaaca

SEQ ID NO: 306 - RNA, 17 nt, synthetic construct
  misc_feature 1..17: note = Targeting domain
  source 1..17: mol_type = other RNA
caaggctggccaaccca

SEQ ID NO: 307 - RNA, 17 nt, synthetic construct
  misc_feature 1..17: note = Targeting domain
  source 1..17: mol_type = other RNA
tggctaaactccaccca

SEQ ID NO: 308 - RNA, 17 nt, synthetic construct
  misc_feature 1..17: note = Targeting domain
  source 1..17: mol_type = other RNA
tgggtggagtttagcca

SEQ ID NO: 309 - RNA, 17 nt, synthetic construct
  misc_feature 1..17: note = Targeting domain
  source 1..17: mol_type = other RNA
agtatccagtgaggcca

SEQ ID NO: 310 - RNA, 17 nt, synthetic construct
  misc_feature 1..17: note = Targeting domain
  source 1..17: mol_type = other RNA
tcaagtttgccttgtca

SEQ ID NO: 311 - RNA, 17 nt, synthetic construct
  misc_feature 1..17: note = Targeting domain
  source 1..17: mol_type = other RNA
ttgagatagtgtgggga

SEQ ID NO: 312 - RNA, 17 nt, synthetic construct
  misc_feature 1..17: note = Targeting domain
  source 1..17: mol_type = other RNA
ataaattagagaaaaac

SEQ ID NO: 313 - RNA, 17 nt, synthetic construct
  misc_feature 1..17: note = Targeting domain
  source 1..17: mol_type = other RNA
ccggcccctggcctcac

SEQ ID NO: 314 - RNA, 17 nt, synthetic construct
  misc_feature 1..17: note = Targeting domain
  source 1..17: mol_type = other RNA
agccagccgccggcccc

SEQ ID NO: 315 - RNA, 17 nt, synthetic construct
  misc_feature 1..17: note = Targeting domain
  source 1..17: mol_type = other RNA
ctgtctgaaacggtccc

SEQ ID NO: 316 - RNA, 17 nt, synthetic construct
  misc_feature 1..17: note = Targeting domain
  source 1..17: mol_type = other RNA
atgggtggagtttagcc

SEQ ID NO: 317 - RNA, 17 nt, synthetic construct
  misc_feature 1..17: note = Targeting domain
  source 1..17: mol_type = other RNA
catccctagccagccgc

SEQ ID NO: 318 - RNA, 17 nt, synthetic construct
  misc_feature 1..17: note = Targeting domain
  source 1..17: mol_type = other RNA
attggtcaaggcaaggc

SEQ ID NO: 319 - RNA, 17 nt, synthetic construct
  misc_feature 1..17: note = Targeting domain
  source 1..17: mol_type = other RNA
ccagtgaggccaggggc

SEQ ID NO: 320 - RNA, 17 nt, synthetic construct
  misc_feature 1..17: note = Targeting domain
  source 1..17: mol_type = other RNA
ttccacacactcgcttc

SEQ ID NO: 321 - RNA, 17 nt, synthetic construct
  misc_feature 1..17: note = Targeting domain
  source 1..17: mol_type = other RNA
cgcttctggaacgtctg

SEQ ID NO: 322 - RNA, 17 nt, synthetic construct
  misc_feature 1..17: note = Targeting domain
  source 1..17: mol_type = other RNA
tcttagagtatccagtg

SEQ ID NO: 323 - RNA, 17 nt, synthetic construct
  misc_feature 1..17: note = Targeting domain
  source 1..17: mol_type = other RNA
tttgcattgagatagtg

SEQ ID NO: 324 - RNA, 17 nt, synthetic construct
  misc_feature 1..17: note = Targeting domain
  source 1..17: mol_type = other RNA
ttccagaagcgagtgtg

SEQ ID NO: 325 - RNA, 17 nt, synthetic construct
  misc_feature 1..17: note = Targeting domain
  source 1..17: mol_type = other RNA
tgcattgagatagtgtg

SEQ ID NO: 326 - RNA, 17 nt, synthetic construct
  misc_feature 1..17: note = Targeting domain
  source 1..17: mol_type = other RNA
aaggctggccaacccat

SEQ ID NO: 327 - RNA, 17 nt, synthetic construct
  misc_feature 1..17: note = Targeting domain
  source 1..17: mol_type = other RNA
tgccttgtcaaggctat

SEQ ID NO: 328 - RNA, 17 nt, synthetic construct
  misc_feature 1..17: note = Targeting domain
  source 1..17: mol_type = other RNA
aaactccacccatgggt

SEQ ID NO: 329 - RNA, 17 nt, synthetic construct
  misc_feature 1..17: note = Targeting domain
  source 1..17: mol_type = other RNA
ttgcattgagatagtgt

SEQ ID NO: 330 - RNA, 20 nt, synthetic construct
  misc_feature 1..20: note = Targeting domain
  source 1..20: mol_type = other RNA
atgcaaatatctgtctgaaa

SEQ ID NO: 331 - RNA, 20 nt, synthetic construct
  misc_feature 1..20: note = Targeting domain
  source 1..20: mol_type = other RNA
actgaatcggaacaaggcaa

SEQ ID NO: 332 - RNA, 20 nt, synthetic construct
  misc_feature 1..20: note = Targeting domain
  source 1..20: mol_type = other RNA
cattgagatagtgtggggaa

SEQ ID NO: 333 - RNA, 20 nt, synthetic construct
  misc_feature 1..20: note = Targeting domain
  source 1..20: mol_type = other RNA
cttgaccaatagccttgaca

SEQ ID NO: 334 - RNA, 20 nt, synthetic construct
  misc_feature 1..20: note = Targeting domain
  source 1..20: mol_type = other RNA
aggcaaggctggccaaccca

SEQ ID NO: 335 - RNA, 20 nt, synthetic construct
  misc_feature 1..20: note = Targeting domain
  source 1..20: mol_type = other RNA
ccctggctaaactccaccca

SEQ ID NO: 336 - RNA, 20 nt, synthetic construct
  misc_feature 1..20: note = Targeting domain
  source 1..20: mol_type = other RNA
ccatgggtggagtttagcca

SEQ ID NO: 337 - RNA, 20 nt, synthetic construct
  misc_feature 1..20: note = Targeting domain
  source 1..20: mol_type = other RNA
tagagtatccagtgaggcca

SEQ ID NO: 338 - RNA, 20 nt, synthetic construct
  misc_feature 1..20: note = Targeting domain
  source 1..20: mol_type = other RNA
caaggctattggtcaaggca

SEQ ID NO: 339 - RNA, 20 nt, synthetic construct
  misc_feature 1..20: note = Targeting domain
  source 1..20: mol_type = other RNA
cttgtcaaggctattggtca

SEQ ID NO: 340 - RNA, 20 nt, synthetic construct
  misc_feature 1..20: note = Targeting domain
  source 1..20: mol_type = other RNA
tggtcaagtttgccttgtca

SEQ ID NO: 341 - RNA, 20 nt, synthetic construct
  misc_feature 1..20: note = Targeting domain
  source 1..20: mol_type = other RNA
cgagtgtgtggaactgctga

SEQ ID NO: 342 - RNA, 20 nt, synthetic construct
  misc_feature 1..20: note = Targeting domain
  source 1..20: mol_type = other RNA
caggggccggcggctggcta

SEQ ID NO: 343 - RNA, 20 nt, synthetic construct
  misc_feature 1..20: note = Targeting domain
  source 1..20: mol_type = other RNA
agaataaattagagaaaaac

SEQ ID NO: 344 - RNA, 20 nt, synthetic construct
  misc_feature 1..20: note = Targeting domain
  source 1..20: mol_type = other RNA
ccgccggcccctggcctcac

SEQ ID NO: 345 - RNA, 20 nt, synthetic construct
  misc_feature 1..20: note = Targeting domain
  source 1..20: mol_type = other RNA
cctagccagccgccggcccc

SEQ ID NO: 346 - RNA, 20 nt, synthetic construct
  misc_feature 1..20: note = Targeting domain
  source 1..20: mol_type = other RNA
tatctgtctgaaacggtccc

SEQ ID NO: 347 - RNA, 20 nt, synthetic construct
  misc_feature 1..20: note = Targeting domain
  source 1..20: mol_type = other RNA
cccatgggtggagtttagcc

SEQ ID NO: 348 - RNA, 20 nt, synthetic construct
  misc_feature 1..20: note = Targeting domain
  source 1..20: mol_type = other RNA
ttagagtatccagtgaggcc

SEQ ID NO: 349 - RNA, 20 nt, synthetic construct
  misc_feature 1..20: note = Targeting domain
  source 1..20: mol_type = other RNA
acggctgacaaaagaagtcc

SEQ ID NO: 350 - RNA, 20 nt, synthetic construct
  misc_feature 1..20: note = Targeting domain
  source 1..20: mol_type = other RNA
cttcatccctagccagccgc

SEQ ID NO: 351 - RNA, 20 nt, synthetic construct
  misc_feature 1..20: note = Targeting domain
  source 1..20: mol_type = other RNA
tgaggccaggggccggcggc

SEQ ID NO: 352 - RNA, 20 nt, synthetic construct
  misc_feature 1..20: note = Targeting domain
  source 1..20: mol_type = other RNA
tatccagtgaggccaggggc

SEQ ID NO: 353 - RNA, 20 nt, synthetic construct
  misc_feature 1..20: note = Targeting domain
  source 1..20: mol_type = other RNA
cagttccacacactcgcttc

SEQ ID NO: 354 - RNA, 20 nt, synthetic construct
  misc_feature 1..20: note = Targeting domain
  source 1..20: mol_type = other RNA
attgagatagtgtggggaag

SEQ ID NO: 355 - RNA, 20 nt, synthetic construct
  misc_feature 1..20: note = Targeting domain
  source 1..20: mol_type = other RNA
agagtatccagtgaggccag

SEQ ID NO: 356 - RNA, 20 nt, synthetic construct
  misc_feature 1..20: note = Targeting domain
  source 1..20: mol_type = other RNA
ccagtgaggccaggggccgg

SEQ ID NO: 357 - RNA, 20 nt, synthetic construct
  misc_feature 1..20: note = Targeting domain
  source 1..20: mol_type = other RNA
aaggctggccaacccatggg

SEQ ID NO: 358 - RNA, 20 nt, synthetic construct
  misc_feature 1..20: note = Targeting domain
  source 1..20: mol_type = other RNA
actcgcttctggaacgtctg

SEQ ID NO: 359 - RNA, 20 nt, synthetic construct
  misc_feature 1..20: note = Targeting domain
  source 1..20: mol_type = other RNA
tagtcttagagtatccagtg

SEQ ID NO: 360 - RNA, 20 nt, synthetic construct
  misc_feature 1..20: note = Targeting domain
  source 1..20: mol_type = other RNA
atatttgcattgagatagtg

SEQ ID NO: 361 - RNA, 20 nt, synthetic construct
  misc_feature 1..20: note = Targeting domain
  source 1..20: mol_type = other RNA
acgttccagaagcgagtgtg

SEQ ID NO: 362 - RNA, 20 nt, synthetic construct
  misc_feature 1..20: note = Targeting domain
  source 1..20: mol_type = other RNA
atttgcattgagatagtgtg

SEQ ID NO: 363 - RNA, 20 nt, synthetic construct
  misc_feature 1..20: note = Targeting domain
  source 1..20: mol_type = other RNA
cctggctaaactccacccat

SEQ ID NO: 364 - RNA, 20 nt, synthetic construct
  misc_feature 1..20: note = Targeting domain
  source 1..20: mol_type = other RNA
actggatactctaagactat

SEQ ID NO: 365 - RNA, 20 nt, synthetic construct
  misc_feature 1..20: note = Targeting domain
  source 1..20: mol_type = other RNA
ccaggggccggcggctggct

SEQ ID NO: 366 - RNA, 20 nt, synthetic construct
  misc_feature 1..20: note = Targeting domain
  source 1..20: mol_type = other RNA
tatttgcattgagatagtgt

SEQ ID NO: 367 - RNA, 19 nt, synthetic construct
  misc_feature 1..19: note = Targeting domain
  source 1..19: mol_type = other RNA
gtttccttctcccatcata

SEQ ID NO: 368 - RNA, 23 nt, synthetic construct
  misc_feature 1..23: note = Targeting domain
  source 1..23: mol_type = other RNA
gctagtttccttctcccatcata

SEQ ID NO: 369 - RNA, 19 nt, synthetic construct
  misc_feature 1..19: note = Targeting domain
  source 1..19: mol_type = other RNA
gaataaattagagaaaaac

SEQ ID NO: 370 - RNA, 22 nt, synthetic construct
  misc_feature 1..22: note = Targeting domain
  source 1..22: mol_type = other RNA
gaagaataaattagagaaaaac

SEQ ID NO: 371 - RNA, 23 nt, synthetic construct
  misc_feature 1..23: note = Targeting domain
  source 1..23: mol_type = other RNA
ggaagaataaattagagaaaaac

SEQ ID NO: 372 - RNA, 24 nt, synthetic construct
  misc_feature 1..24: note = Targeting domain
  source 1..24: mol_type = other RNA
gggaagaataaattagagaaaaac

SEQ ID NO: 373 - RNA, 20 nt, synthetic construct
  misc_feature 1..20: note = Targeting domain
  source 1..20: mol_type = other RNA
gaaggaaactagctaaaggg

SEQ ID NO: 374 - RNA, 22 nt, synthetic construct
  misc_feature 1..22: note = Targeting domain
  source 1..22: mol_type = other RNA
gagaaggaaactagctaaaggg

SEQ ID NO: 375 - RNA, 23 nt, synthetic construct
  misc_feature 1..23: note = Targeting domain
  source 1..23: mol_type = other RNA
ggagaaggaaactagctaaaggg

SEQ ID NO: 376 - RNA, 24 nt, synthetic construct
  misc_feature 1..24: note = Targeting domain
  source 1..24: mol_type = other RNA
gggagaaggaaactagctaaaggg

SEQ ID NO: 377 - RNA, 18 nt, synthetic construct
  misc_feature 1..18: note = Targeting domain
  source 1..18: mol_type = other RNA
tttccttctcccatcata

SEQ ID NO: 378 - RNA, 20 nt, synthetic construct
  misc_feature 1..20: note = Targeting domain
  source 1..20: mol_type = other RNA
agtttccttctcccatcata

SEQ ID NO: 379 - RNA, 21 nt, synthetic construct
  misc_feature 1..21: note = Targeting domain
  source 1..21: mol_type = other RNA
tagtttccttctcccatcata

SEQ ID NO: 380 - RNA, 22 nt, synthetic construct
  misc_feature 1..22: note = Targeting domain
  source 1..22: mol_type = other RNA
ctagtttccttctcccatcata

SEQ ID NO: 381 - RNA, 24 nt, synthetic construct
  misc_feature 1..24: note = Targeting domain
  source 1..24: mol_type = other RNA
agctagtttccttctcccatcata

SEQ ID NO: 382 - RNA, 18 nt, synthetic construct
  misc_feature 1..18: note = Targeting domain
  source 1..18: mol_type = other RNA
agagaaaaactggaatga

SEQ ID NO: 383 - RNA, 19 nt, synthetic construct
  misc_feature 1..19: note = Targeting domain
  source 1..19: mol_type = other RNA
tagagaaaaactggaatga

SEQ ID NO: 384 - RNA, 20 nt, synthetic construct
  misc_feature 1..20: note = Targeting domain
  source 1..20: mol_type = other RNA
ttagagaaaaactggaatga

SEQ ID NO: 385 - RNA, 21 nt, synthetic construct
  misc_feature 1..21: note = Targeting domain
  source 1..21: mol_type = other RNA
attagagaaaaactggaatga

SEQ ID NO: 386 - RNA, 22 nt, synthetic construct
  misc_feature 1..22: note = Targeting domain
  source 1..22: mol_type = other RNA
aattagagaaaaactggaatga

SEQ ID NO: 387 - RNA, 23 nt, synthetic construct
  misc_feature 1..23: note = Targeting domain
  source 1..23: mol_type = other RNA
aaattagagaaaaactggaatga

SEQ ID NO: 388 - RNA, 24 nt, synthetic construct
  misc_feature 1..24: note = Targeting domain
  source 1..24: mol_type = other RNA
taaattagagaaaaactggaatga

SEQ ID NO: 389 - RNA, 18 nt, synthetic construct
  misc_feature 1..18: note = Targeting domain
  source 1..18: mol_type = other RNA
aataaattagagaaaaac

SEQ ID NO: 390 - RNA, 21 nt, synthetic construct
  misc_feature 1..21: note = Targeting domain
  source 1..21: mol_type = other RNA
aagaataaattagagaaaaac

SEQ ID NO: 391 - RNA, 18 nt, synthetic construct
  misc_feature 1..18: note = Targeting domain
  source 1..18: mol_type = other RNA
aggaaactagctaaaggg

SEQ ID NO: 392 - RNA, 19 nt, synthetic construct
  misc_feature 1..19: note = Targeting domain
  source 1..19: mol_type = other RNA
aaggaaactagctaaaggg

SEQ ID NO: 393 - RNA, 21 nt, synthetic construct
  misc_feature 1..21: note = Targeting domain
  source 1..21: mol_type = other RNA
agaaggaaactagctaaaggg

SEQ ID NO: 394 - RNA, 18 nt, synthetic construct
  misc_feature 1..18: note = Targeting domain
  source 1..18: mol_type = other RNA
tggggaaggggcccccaa

SEQ ID NO: 395 - RNA, 19 nt, synthetic construct
  misc_feature 1..19: note = Targeting domain
  source 1..19: mol_type = other RNA
gtggggaaggggcccccaa

SEQ ID NO: 396 - RNA, 20 nt, synthetic construct
  misc_feature 1..20: note = Targeting domain
  source 1..20: mol_type = other RNA
tgtggggaaggggcccccaa

SEQ ID NO: 397 - RNA, 21 nt, synthetic construct
  misc_feature 1..21: note = Targeting domain
  source 1..21: mol_type = other RNA
gtgtggggaaggggcccccaa

SEQ ID NO: 398 - RNA, 22 nt, synthetic construct
  misc_feature 1..22: note = Targeting domain
  source 1..22: mol_type = other RNA
agtgtggggaaggggcccccaa

SEQ ID NO: 399 - RNA, 23 nt, synthetic construct
  misc_feature 1..23: note = Targeting domain
  source 1..23: mol_type = other RNA
tagtgtggggaaggggcccccaa

SEQ ID NO: 400 - RNA, 24 nt, synthetic construct
  misc_feature 1..24: note = Targeting domain
  source 1..24: mol_type = other RNA
atagtgtggggaaggggcccccaa

SEQ ID NO: 401 - RNA, 18 nt, synthetic construct
  misc_feature 1..18: note = Targeting domain
  source 1..18: mol_type = other RNA
acctcagacgttccagaa

SEQ ID NO: 402 - RNA, 19 nt, synthetic construct
  misc_feature 1..19: note = Targeting domain
  source 1..19: mol_type = other RNA
aacctcagacgttccagaa

SEQ ID NO: 403 - RNA, 20 nt, synthetic construct
  misc_feature 1..20: note = Targeting domain
  source 1..20: mol_type = other RNA
taacctcagacgttccagaa

SEQ ID NO: 404 - RNA, 21 nt, synthetic construct
  misc_feature 1..21: note = Targeting domain
  source 1..21: mol_type = other RNA
ataacctcagacgttccagaa

SEQ ID NO: 405 - RNA, 22 nt, synthetic construct
  misc_feature 1..22: note = Targeting domain
  source 1..22: mol_type = other RNA
gataacctcagacgttccagaa

SEQ ID NO: 406 - RNA, 23 nt, synthetic construct
  misc_feature 1..23: note = Targeting domain
  source 1..23: mol_type = other RNA
tgataacctcagacgttccagaa

SEQ ID NO: 407 - RNA, 24 nt, synthetic construct
  misc_feature 1..24: note = Targeting domain
  source 1..24: mol_type = other RNA
ttgataacctcagacgttccagaa

SEQ ID NO: 408 - RNA, 18 nt, synthetic construct
  misc_feature 1..18: note = Targeting domain
  source 1..18: mol_type = other RNA
cgccggcccctggcctca

SEQ ID NO: 409 - RNA, 19 nt, synthetic construct
  misc_feature 1..19: note = Targeting domain
  source 1..19: mol_type = other RNA
ccgccggcccctggcctca

SEQ ID NO: 410 - RNA, 20 nt, synthetic construct
  misc_feature 1..20: note = Targeting domain
  source 1..20: mol_type = other RNA
gccgccggcccctggcctca

SEQ ID NO: 411 - RNA, 21 nt, synthetic construct
  misc_feature 1..21: note = Targeting domain
  source 1..21: mol_type = other RNA
agccgccggcccctggcctca

SEQ ID NO: 412 - RNA, 22 nt, synthetic construct
  misc_feature 1..22: note = Targeting domain
  source 1..22: mol_type = other RNA
cagccgccggcccctggcctca

SEQ ID NO: 413 - RNA, 23 nt, synthetic construct
  misc_feature 1..23: note = Targeting domain
  source 1..23: mol_type = other RNA
ccagccgccggcccctggcctca

SEQ ID NO: 414 - RNA, 24 nt, synthetic construct
  misc_feature 1..24: note = Targeting domain
  source 1..24: mol_type = other RNA
gccagccgccggcccctggcctca

SEQ ID NO: 415 - RNA, 18 nt, synthetic construct
  misc_feature 1..18: note = Targeting domain
  source 1..18: mol_type = other RNA
ggcaaggctggccaaccc

SEQ ID NO: 416 - RNA, 19 nt, synthetic construct
  misc_feature 1..19: note = Targeting domain
  source 1..19: mol_type = other RNA
aggcaaggctggccaaccc

SEQ ID NO: 417 - RNA, 20 nt, synthetic construct
  misc_feature 1..20: note = Targeting domain
  source 1..20: mol_type = other RNA
aaggcaaggctggccaaccc

SEQ ID NO: 418 - RNA, 21 nt, synthetic construct
  misc_feature 1..21: note = Targeting domain
  source 1..21: mol_type = other RNA
caaggcaaggctggccaaccc

SEQ ID NO: 419 - RNA, 22 nt, synthetic construct
  misc_feature 1..22: note = Targeting domain
  source 1..22: mol_type = other RNA
tcaaggcaaggctggccaaccc

SEQ ID NO: 420 - RNA, 23 nt, synthetic construct
  misc_feature 1..23: note = Targeting domain
  source 1..23: mol_type = other RNA
gtcaaggcaaggctggccaaccc

SEQ ID NO: 421 - RNA, 24 nt, synthetic construct
  misc_feature 1..24: note = Targeting domain
  source 1..24: mol_type = other RNA
ggtcaaggcaaggctggccaaccc

SEQ ID NO: 422 - RNA, 18 nt, synthetic construct
  misc_feature 1..18: note = Targeting domain
  source 1..18: mol_type = other RNA
ggctggccaacccatggg

SEQ ID NO: 423 - RNA, 19 nt, synthetic construct
  misc_feature 1..19: note = Targeting domain
  source 1..19: mol_type = other RNA
aggctggccaacccatggg

SEQ ID NO: 424 - RNA, 21 nt, synthetic construct
  misc_feature 1..21: note = Targeting domain
  source 1..21: mol_type = other RNA
caaggctggccaacccatggg

SEQ ID NO: 425 - RNA, 22 nt, synthetic construct
  misc_feature 1..22: note = Targeting domain
  source 1..22: mol_type = other RNA
gcaaggctggccaacccatggg

SEQ ID NO: 426 - RNA, 23 nt, synthetic construct
  misc_feature 1..23: note = Targeting domain
  source 1..23: mol_type = other RNA
ggcaaggctggccaacccatggg

SEQ ID NO: 427 - RNA, 24 nt, synthetic construct
  misc_feature 1..24: note = Targeting domain
  source 1..24: mol_type = other RNA
aggcaaggctggccaacccatggg

SEQ ID NO: 428 - RNA, 18 nt, synthetic construct
  misc_feature 1..18: note = Targeting domain
  source 1..18: mol_type = other RNA
gagtgtgtggaactgctg

SEQ ID NO: 429 - RNA, 19 nt, synthetic construct
  misc_feature 1..19: note = Targeting domain
  source 1..19: mol_type = other RNA
cgagtgtgtggaactgctg

SEQ ID NO: 430 - RNA, 20 nt, synthetic construct
  misc_feature 1..20: note = Targeting domain
  source 1..20: mol_type = other RNA
gcgagtgtgtggaactgctg

SEQ ID NO: 431 - RNA, 21 nt, synthetic construct
  misc_feature 1..21: note = Targeting domain
  source 1..21: mol_type = other RNA
agcgagtgtgtggaactgctg

SEQ ID NO: 432 - RNA, 22 nt, synthetic construct
  misc_feature 1..22: note = Targeting domain
  source 1..22: mol_type = other RNA
aagcgagtgtgtggaactgctg

SEQ ID NO: 433 - RNA, 23 nt, synthetic construct
  misc_feature 1..23: note = Targeting domain
  source 1..23: mol_type = other RNA
gaagcgagtgtgtggaactgctg

SEQ ID NO: 434 - RNA, 24 nt, synthetic construct
  misc_feature 1..24: note = Targeting domain
  source 1..24: mol_type = other RNA
agaagcgagtgtgtggaactgctg

SEQ ID NO: 435 - RNA, 18 nt, synthetic construct
  misc_feature 1..18: note = Targeting domain
  source 1..18: mol_type = other RNA
cctggctaaactccaccc

SEQ ID NO: 436 - RNA, 19 nt, synthetic construct
  misc_feature 1..19: note = Targeting domain
  source 1..19: mol_type = other RNA
ccctggctaaactccaccc

SEQ ID NO: 437 - RNA, 20 nt, synthetic construct
  misc_feature 1..20: note = Targeting domain
  source 1..20: mol_type = other RNA
tccctggctaaactccaccc

SEQ ID NO: 438 - RNA, 21 nt, synthetic construct
  misc_feature 1..21: note = Targeting domain
  source 1..21: mol_type = other RNA
gtccctggctaaactccaccc

SEQ ID NO: 439 - RNA, 22 nt, synthetic construct
  misc_feature 1..22: note = Targeting domain
  source 1..22: mol_type = other RNA
ggtccctggctaaactccaccc

SEQ ID NO: 440 - RNA, 23 nt, synthetic construct
  misc_feature 1..23: note = Targeting domain
  source 1..23: mol_type = other RNA
cggtccctggctaaactccaccc

SEQ ID NO: 441 - RNA, 24 nt, synthetic construct
  misc_feature 1..24: note = Targeting domain
  source 1..24: mol_type = other RNA
acggtccctggctaaactccaccc

SEQ ID NO: 442 - RNA, 18 nt, synthetic construct
  misc_feature 1..18: note = Targeting domain
  source 1..18: mol_type = other RNA
ggcggctggctagggatg

SEQ ID NO: 443 - RNA, 19 nt, synthetic construct
  misc_feature 1..19: note = Targeting domain
  source 1..19: mol_type = other RNA
cggcggctggctagggatg

SEQ ID NO: 444 - RNA, 20 nt, synthetic construct
  misc_feature 1..20: note = Targeting domain
  source 1..20: mol_type = other RNA
ccggcggctggctagggatg

SEQ ID NO: 445 - RNA, 21 nt, synthetic construct
  misc_feature 1..21: note = Targeting domain
  source 1..21: mol_type = other RNA
gccggcggctggctagggatg

SEQ ID NO: 446 - RNA, 22 nt, synthetic construct
  misc_feature 1..22: note = Targeting domain
  source 1..22: mol_type = other RNA
ggccggcggctggctagggatg

SEQ ID NO: 447 - RNA, 23 nt, synthetic construct
  misc_feature 1..23: note = Targeting domain
  source 1..23: mol_type = other RNA
gggccggcggctggctagggatg

SEQ ID NO: 448 - RNA, 24 nt, synthetic construct
  misc_feature 1..24: note = Targeting domain
  source 1..24: mol_type = other RNA
ggggccggcggctggctagggatg

SEQ ID NO: 449 - RNA, 18 nt, synthetic construct
  misc_feature 1..18: note = Targeting domain
  source 1..18: mol_type = other RNA
aggggccggcggctggct

SEQ ID NO: 450 - RNA, 19 nt, synthetic construct
  misc_feature 1..19: note = Targeting domain
  source 1..19: mol_type = other RNA
caggggccggcggctggct

SEQ ID NO: 451 - RNA, 21 nt, synthetic construct
  misc_feature 1..21: note = Targeting domain
  source 1..21: mol_type = other RNA
gccaggggccggcggctggct

SEQ ID NO: 452 - RNA, 22 nt, synthetic construct
  misc_feature 1..22: note = Targeting domain
  source 1..22: mol_type = other RNA
ggccaggggccggcggctggct

SEQ ID NO: 453 - RNA, 23 nt, synthetic construct
  misc_feature 1..23: note = Targeting domain
  source 1..23: mol_type = other RNA
aggccaggggccggcggctggct

SEQ ID NO: 454 - RNA, 24 nt, synthetic construct
  misc_feature 1..24: note = Targeting domain
  source 1..24: mol_type = other RNA
gaggccaggggccggcggctggct

SEQ ID NO: 455 - RNA, 18 nt, synthetic construct
  misc_feature 1..18: note = Targeting domain
  source 1..18: mol_type = other RNA
aaacttgaccaatagtct

SEQ ID NO: 456 - RNA, 19 nt, synthetic construct
  misc_feature 1..19: note = Targeting domain
  source 1..19: mol_type = other RNA
caaacttgaccaatagtct

SEQ ID NO: 457 - RNA, 20 nt, synthetic construct
  misc_feature 1..20: note = Targeting domain
  source 1..20: mol_type = other RNA
gcaaacttgaccaatagtct

SEQ ID NO: 458 - RNA, 21 nt, synthetic construct
  misc_feature 1..21: note = Targeting domain
  source 1..21: mol_type = other RNA
ggcaaacttgaccaatagtct

SEQ ID NO: 459 - RNA, 22 nt, synthetic construct
  misc_feature 1..22: note = Targeting domain
  source 1..22: mol_type = other RNA
aggcaaacttgaccaatagtct

SEQ ID NO: 460 - RNA, 23 nt, synthetic construct
  misc_feature 1..23: note = Targeting domain
  source 1..23: mol_type = other RNA
aaggcaaacttgaccaatagtct

SEQ ID NO: 461 - RNA, 24 nt, synthetic construct
  misc_feature 1..24: note = Targeting domain
  source 1..24: mol_type = other RNA
caaggcaaacttgaccaatagtct

SEQ ID NO: 462 - RNA, 18 nt, synthetic construct
  misc_feature 1..18: note = Targeting domain
  source 1..18: mol_type = other RNA
ttcagacagatatttgca

SEQ ID NO: 463 - RNA, 19 nt, synthetic construct
  misc_feature 1..19: note = Targeting domain
  source 1..19: mol_type = other RNA
tttcagacagatatttgca

SEQ ID NO: 464 - RNA, 20 nt, synthetic construct
  misc_feature 1..20: note = Targeting domain
  source 1..20: mol_type = other RNA
gtttcagacagatatttgca

SEQ ID NO: 465 - RNA, 21 nt, synthetic construct
  misc_feature 1..21: note = Targeting domain
  source 1..21: mol_type = other RNA
cgtttcagacagatatttgca

SEQ ID NO: 466 - RNA, 22 nt, synthetic construct
  misc_feature 1..22: note = Targeting domain
  source 1..22: mol_type = other RNA
ccgtttcagacagatatttgca

SEQ ID NO: 467 - RNA, 23 nt, synthetic construct
  misc_feature 1..23: note = Targeting domain
  source 1..23: mol_type = other RNA
accgtttcagacagatatttgca

SEQ ID NO: 468 - RNA, 24 nt, synthetic construct
  misc_feature 1..24: note = Targeting domain
  source 1..24: mol_type = other RNA
gaccgtttcagacagatatttgca

SEQ ID NO: 469 - RNA, 18 nt, synthetic construct
  misc_feature 1..18: note = Targeting domain
  source 1..18: mol_type = other RNA
agtttccttctcccatca

SEQ ID NO: 470 - RNA, 19 nt, synthetic construct
  misc_feature 1..19: note = Targeting domain
  source 1..19: mol_type = other RNA
tagtttccttctcccatca

SEQ ID NO: 471 - RNA, 20 nt, synthetic construct
  misc_feature 1..20: note = Targeting domain
  source 1..20: mol_type = other RNA
ctagtttccttctcccatca

SEQ ID NO: 472 - RNA, 21 nt, synthetic construct
  misc_feature 1..21: note = Targeting domain
  source 1..21: mol_type = other RNA
gctagtttccttctcccatca

SEQ ID NO: 473 - RNA, 22 nt, synthetic construct
  misc_feature 1..22: note = Targeting domain
  source 1..22: mol_type = other RNA
agctagtttccttctcccatca

SEQ ID NO: 474 - RNA, 23 nt, synthetic construct
  misc_feature 1..23: note = Targeting domain
  source 1..23: mol_type = other RNA
tagctagtttccttctcccatca

SEQ ID NO: 475 - RNA, 24 nt, synthetic construct
  misc_feature 1..24: note = Targeting domain
  source 1..24: mol_type = other RNA
ttagctagtttccttctcccatca

SEQ ID NO: 476 - RNA, 18 nt, synthetic construct
  misc_feature 1..18: note = Targeting domain
  source 1..18: mol_type = other RNA
attgagatagtgtgggga

SEQ ID NO: 477 - RNA, 19 nt, synthetic construct
  misc_feature 1..19: note = Targeting domain
  source 1..19: mol_type = other RNA
cattgagatagtgtgggga

SEQ ID NO: 478 - RNA, 21 nt, synthetic construct
  misc_feature 1..21: note = Targeting domain
  source 1..21: mol_type = other RNA
tgcattgagatagtgtgggga

SEQ ID NO: 479 - RNA, 22 nt, synthetic construct
  misc_feature 1..22: note = Targeting domain
  source 1..22: mol_type = other RNA
ttgcattgagatagtgtgggga

SEQ ID NO: 480 - RNA, 23 nt, synthetic construct
  misc_feature 1..23: note = Targeting domain
  source 1..23: mol_type = other RNA
tttgcattgagatagtgtgggga

SEQ ID NO: 481 - RNA, 24 nt, synthetic construct
  misc_feature 1..24: note = Targeting domain
  source 1..24: mol_type = other RNA
atttgcattgagatagtgtgggga

SEQ ID NO: 482 - RNA, 18 nt, synthetic construct
  misc_feature 1..18: note = Targeting domain
  source 1..18: mol_type = other RNA
tcccatcatagaggatac

SEQ ID NO: 483 - RNA, 19 nt, synthetic construct
  misc_feature 1..19: note = Targeting domain
  source 1..19: mol_type = other RNA
ctcccatcatagaggatac

SEQ ID NO: 484 - RNA, 20 nt, synthetic construct
  misc_feature 1..20: note = Targeting domain
  source 1..20: mol_type = other RNA
tctcccatcatagaggatac

SEQ ID NO: 485 - RNA, 21 nt, synthetic construct
  misc_feature 1..21: note = Targeting domain
  source 1..21: mol_type = other RNA
ttctcccatcatagaggatac

SEQ ID NO: 486 - RNA, 22 nt, synthetic construct
  misc_feature 1..22: note = Targeting domain
  source 1..22: mol_type = other RNA
cttctcccatcatagaggatac

SEQ ID NO: 487 - RNA, 23 nt, synthetic construct
  misc_feature 1..23: note = Targeting domain
  source 1..23: mol_type = other RNA
ccttctcccatcatagaggatac

SEQ ID NO: 488 - RNA, 24 nt, synthetic construct
  misc_feature 1..24: note = Targeting domain
  source 1..24: mol_type = other RNA
tccttctcccatcatagaggatac

SEQ ID NO: 489 - RNA, 18 nt, synthetic construct
  misc_feature 1..18: note = Targeting domain
  source 1..18: mol_type = other RNA
tgtggggaaggggccccc

SEQ ID NO: 490 - RNA, 19 nt, synthetic construct
  misc_feature 1..19: note = Targeting domain
  source 1..19: mol_type = other RNA
gtgtggggaaggggccccc

SEQ ID NO: 491 - RNA, 20 nt, synthetic construct
  misc_feature 1..20: note = Targeting domain
  source 1..20: mol_type = other RNA
agtgtggggaaggggccccc

SEQ ID NO: 492 - RNA, 21 nt, synthetic construct
  misc_feature 1..21: note = Targeting domain
  source 1..21: mol_type = other RNA
tagtgtggggaaggggccccc

SEQ ID NO: 493 - RNA, 22 nt, synthetic construct
  misc_feature 1..22: note = Targeting domain
  source 1..22: mol_type = other RNA
atagtgtggggaaggggccccc

SEQ ID NO: 494 - RNA, 23 nt, synthetic construct
  misc_feature 1..23: note = Targeting domain
  source 1..23: mol_type = other RNA
gatagtgtggggaaggggccccc

SEQ ID NO: 495 - RNA, 24 nt, synthetic construct
  misc_feature 1..24: note = Targeting domain
  source 1..24: mol_type = other RNA
agatagtgtggggaaggggccccc

SEQ ID NO: 496 - RNA, 18 nt, synthetic construct
  misc_feature 1..18: note = Targeting domain
  source 1..18: mol_type = other RNA
catgggtggagtttagcc

SEQ ID NO: 497 - RNA, 19 nt, synthetic construct
  misc_feature 1..19: note = Targeting domain
  source 1..19: mol_type = other RNA
ccatgggtggagtttagcc

SEQ ID NO: 498 - RNA, 21 nt, synthetic construct
  misc_feature 1..21: note = Targeting domain
  source 1..21: mol_type = other RNA
acccatgggtggagtttagcc

SEQ ID NO: 499 - RNA, 22 nt, synthetic construct
  misc_feature 1..22: note = Targeting domain
  source 1..22: mol_type = other RNA
aacccatgggtggagtttagcc

SEQ ID NO: 500 - RNA, 23 nt, synthetic construct
  misc_feature 1..23: note = Targeting domain
  source 1..23: mol_type = other RNA
caacccatgggtggagtttagcc

SEQ ID NO: 501 - RNA, 24 nt, synthetic construct
  misc_feature 1..24: note = Targeting domain
  source 1..24: mol_type = other RNA
ccaacccatgggtggagtttagcc

SEQ ID NO: 502 - RNA, 18 nt, synthetic construct
  misc_feature 1..18: note = Targeting domain
  source 1..18: mol_type = other RNA
ccatgggtggagtttagc

SEQ ID NO: 503 - RNA, 19 nt, synthetic construct
  misc_feature 1..19: note = Targeting domain
  source 1..19: mol_type = other RNA
cccatgggtggagtttagc

SEQ ID NO: 504 - RNA, 20 nt, synthetic construct
  misc_feature 1..20: note = Targeting domain
  source 1..20: mol_type = other RNA
acccatgggtggagtttagc

SEQ ID NO: 505 - RNA, 21 nt, synthetic construct
  misc_feature 1..21: note = Targeting domain
  source 1..21: mol_type = other RNA
aacccatgggtggagtttagc

SEQ ID NO: 506 - RNA, 22 nt, synthetic construct
  misc_feature 1..22: note = Targeting domain
  source 1..22: mol_type = other RNA
caacccatgggtggagtttagc

SEQ ID NO: 507 - RNA, 23 nt, synthetic construct
  misc_feature 1..23: note = Targeting domain
  source 1..23: mol_type = other RNA
ccaacccatgggtggagtttagc

SEQ ID NO: 508 - RNA, 24 nt, synthetic construct
  misc_feature 1..24: note = Targeting domain
  source 1..24: mol_type = other RNA
gccaacccatgggtggagtttagc

SEQ ID NO: 509 - RNA, 18 nt, synthetic construct
  misc_feature 1..18: note = Targeting domain
  source 1..18: mol_type = other RNA
tgataacctcagacgttc

SEQ ID NO: 510 - RNA, 19 nt, synthetic construct
  misc_feature 1..19: note = Targeting domain
  source 1..19: mol_type = other RNA
ttgataacctcagacgttc

SEQ ID NO: 511 - RNA, 20 nt, synthetic construct
  misc_feature 1..20: note = Targeting domain
  source 1..20: mol_type = other RNA
attgataacctcagacgttc

SEQ ID NO: 512 - RNA, 21 nt, synthetic construct
  misc_feature 1..21: note = Targeting domain
  source 1..21: mol_type = other RNA
tattgataacctcagacgttc

SEQ ID NO: 513 - RNA, 22 nt, synthetic construct
  misc_feature 1..22: note = Targeting domain
  source 1..22: mol_type = other RNA
ttattgataacctcagacgttc

SEQ ID NO: 514 - RNA, 23 nt, synthetic construct
  misc_feature 1..23: note = Targeting domain
  source 1..23: mol_type = other RNA
cttattgataacctcagacgttc

SEQ ID NO: 515 - RNA, 24 nt, synthetic construct
  misc_feature 1..24: note = Targeting domain
  source 1..24: mol_type = other RNA
gcttattgataacctcagacgttc

SEQ ID NO: 516 - RNA, 18 nt, synthetic construct
  misc_feature 1..18: note = Targeting domain
  source 1..18: mol_type = other RNA
cattgagatagtgtgggg

SEQ ID NO: 517 - RNA, 19 nt, synthetic construct
  misc_feature 1..19: note = Targeting domain
  source 1..19: mol_type = other RNA
gcattgagatagtgtgggg

SEQ ID NO: 518 - RNA, 20 nt, synthetic construct
  misc_feature 1..20: note = Targeting domain
  source 1..20: mol_type = other RNA
tgcattgagatagtgtgggg

SEQ ID NO: 519 - RNA, 21 nt, synthetic construct
  misc_feature 1..21: note = Targeting domain
  source 1..21: mol_type = other RNA
ttgcattgagatagtgtgggg

SEQ ID NO: 520 - RNA, 22 nt, synthetic construct
  misc_feature 1..22: note = Targeting domain
  source 1..22: mol_type = other RNA
tttgcattgagatagtgtgggg

SEQ ID NO: 521 - RNA, 23 nt, synthetic construct
  misc_feature 1..23: note = Targeting domain
  source 1..23: mol_type = other RNA
atttgcattgagatagtgtgggg

SEQ ID NO: 522 - RNA, 24 nt, synthetic construct
  misc_feature 1..24: note = Targeting domain
  source 1..24: mol_type = other RNA
tatttgcattgagatagtgtgggg

SEQ ID NO: 523 - RNA, 18 nt, synthetic construct
  misc_feature 1..18: note = Targeting domain
  source 1..18: mol_type = other RNA
aggctggccaacccatgg

SEQ ID NO: 524 - RNA, 19 nt, synthetic construct
  misc_feature 1..19: note = Targeting domain
  source 1..19: mol_type = other RNA
aaggctggccaacccatgg

SEQ ID NO: 525 - RNA, 20 nt, synthetic construct
  misc_feature 1..20: note = Targeting domain
  source 1..20: mol_type = other RNA
caaggctggccaacccatgg

SEQ ID NO: 526 - RNA, 21 nt, synthetic construct
  misc_feature 1..21: note = Targeting domain
  source 1..21: mol_type = other RNA
gcaaggctggccaacccatgg

SEQ ID NO: 527 - RNA, 22 nt, synthetic construct
  misc_feature 1..22: note = Targeting domain
  source 1..22: mol_type = other RNA
ggcaaggctggccaacccatgg

SEQ ID NO: 528 - RNA, 23 nt, synthetic construct
  misc_feature 1..23: note = Targeting domain
  source 1..23: mol_type = other RNA
aggcaaggctggccaacccatgg

SEQ ID NO: 529 - RNA, 24 nt, synthetic construct
  misc_feature 1..24: note = Targeting domain
  source 1..24: mol_type = other RNA
aaggcaaggctggccaacccatgg

SEQ ID NO: 530 - RNA, 18 nt, synthetic construct
  misc_feature 1..18: note = Targeting domain
  source 1..18: mol_type = other RNA
agcgagtgtgtggaactg

SEQ ID NO: 531 - RNA, 19 nt, synthetic construct
  misc_feature 1..19: note = Targeting domain
  source 1..19: mol_type = other RNA
aagcgagtgtgtggaactg

SEQ ID NO: 532 - RNA, 20 nt, synthetic construct
  misc_feature 1..20: note = Targeting domain
  source 1..20: mol_type = other RNA
gaagcgagtgtgtggaactg

SEQ ID NO: 533 - RNA, 21 nt, synthetic construct
  misc_feature 1..21: note = Targeting domain
  source 1..21: mol_type = other RNA
agaagcgagtgtgtggaactg

SEQ ID NO: 534 - RNA, 22 nt, synthetic construct
  misc_feature 1..22: note = Targeting domain
  source 1..22: mol_type = other RNA
cagaagcgagtgtgtggaactg

SEQ ID NO: 535 - RNA, 23 nt, synthetic construct
  misc_feature 1..23: note = Targeting domain
  source 1..23: mol_type = other RNA
ccagaagcgagtgtgtggaactg

SEQ ID NO: 536 - RNA, 24 nt, synthetic construct
  misc_feature 1..24: note = Targeting domain
  source 1..24: mol_type = other RNA
tccagaagcgagtgtgtggaactg

SEQ ID NO: 537 - RNA, 18 nt, synthetic construct
  misc_feature 1..18: note = Targeting domain
  source 1..18: mol_type = other RNA
atttgcattgagatagtg

SEQ ID NO: 538 - RNA, 19 nt, synthetic construct
  misc_feature 1..19: note = Targeting domain
  source 1..19: mol_type = other RNA
tatttgcattgagatagtg

SEQ ID NO: 539 - RNA, 21 nt, synthetic construct
  misc_feature 1..21: note = Targeting domain
  source 1..21: mol_type = other RNA
gatatttgcattgagatagtg

SEQ ID NO: 540 - RNA, 22 nt, synthetic construct
  misc_feature 1..22: note = Targeting domain
  source 1..22: mol_type = other RNA
agatatttgcattgagatagtg

SEQ ID NO: 541 - RNA, 23 nt, synthetic construct
  misc_feature 1..23: note = Targeting domain
  source 1..23: mol_type = other RNA
cagatatttgcattgagatagtg

SEQ ID NO: 542 - RNA, 24 nt, synthetic construct
  misc_feature 1..24: note = Targeting domain
  source 1..24: mol_type = other RNA
acagatatttgcattgagatagtg

SEQ ID NO: 543 - RNA, 18 nt, synthetic construct
  misc_feature 1..18: note = Targeting domain
  source 1..18: mol_type = other RNA
gttccagaagcgagtgtg

SEQ ID NO: 544 - RNA, 19 nt, synthetic construct
  misc_feature 1..19: note = Targeting domain
  source 1..19: mol_type = other RNA
cgttccagaagcgagtgtg

SEQ ID NO: 545 - RNA, 21 nt, synthetic construct
  misc_feature 1..21: note = Targeting domain
  source 1..21: mol_type = other RNA
gacgttccagaagcgagtgtg

SEQ ID NO: 546 - RNA, 22 nt, synthetic construct
  misc_feature 1..22: note = Targeting domain
  source 1..22: mol_type = other RNA
agacgttccagaagcgagtgtg

SEQ ID NO: 547 - RNA, 23 nt, synthetic construct
  misc_feature 1..23: note = Targeting domain
  source 1..23: mol_type = other RNA
cagacgttccagaagcgagtgtg

SEQ ID NO: 548 - RNA, 24 nt, synthetic construct
  misc_feature 1..24: note = Targeting domain
  source 1..24: mol_type = other RNA
tcagacgttccagaagcgagtgtg

SEQ ID NO: 549 - RNA, 18 nt, synthetic construct
  misc_feature 1..18: note = Targeting domain
  source 1..18: mol_type = other RNA
ttgcattgagatagtgtg

SEQ ID NO: 550 - RNA, 19 nt, synthetic construct
  misc_feature 1..19: note = Targeting domain
  source 1..19: mol_type = other RNA
tttgcattgagatagtgtg

SEQ ID NO: 551 - RNA, 21 nt, synthetic construct
  misc_feature 1..21: note = Targeting domain
  source 1..21: mol_type = other RNA
tatttgcattgagatagtgtg

SEQ ID NO: 552 - RNA, 22 nt, synthetic construct
  misc_feature 1..22: note = Targeting domain
  source 1..22: mol_type = other RNA
atatttgcattgagatagtgtg

SEQ ID NO: 553 - RNA, 23 nt, synthetic construct
  misc_feature 1..23: note = Targeting domain
  source 1..23: mol_type = other RNA
gatatttgcattgagatagtgtg

SEQ ID NO: 554 - RNA, 24 nt, synthetic construct
  misc_feature 1..24: note = Targeting domain
  source 1..24: mol_type = other RNA
agatatttgcattgagatagtgtg

SEQ ID NO: 555 - RNA, 18 nt, synthetic construct
  misc_feature 1..18: note = Targeting domain
  source 1..18: mol_type = other RNA
tatttgcattgagatagt

SEQ ID NO: 556 - RNA, 19 nt, synthetic construct
  misc_feature 1..19: note = Targeting domain
  source 1..19: mol_type = other RNA
atatttgcattgagatagt

SEQ ID NO: 557 - RNA, 20 nt, synthetic construct
  misc_feature 1..20: note = Targeting domain
  source 1..20: mol_type = other RNA
gatatttgcattgagatagt

SEQ ID NO: 558 - RNA, 21 nt, synthetic construct
  misc_feature 1..21: note = Targeting domain
  source 1..21: mol_type = other RNA
agatatttgcattgagatagt

SEQ ID NO: 559 - RNA, 22 nt, synthetic construct
  misc_feature 1..22: note = Targeting domain
  source 1..22: mol_type = other RNA
cagatatttgcattgagatagt

SEQ ID NO: 560 - RNA, 23 nt, synthetic construct
  misc_feature 1..23: note = Targeting domain
  source 1..23: mol_type = other RNA
acagatatttgcattgagatagt

SEQ ID NO: 561 - RNA, 24 nt, synthetic construct
  misc_feature 1..24: note = Targeting domain
  source 1..24: mol_type = other RNA
gacagatatttgcattgagatagt

SEQ ID NO: 562 - RNA, 18 nt, synthetic construct
  misc_feature 1..18: note = Targeting domain
  source 1..18: mol_type = other RNA
cgttccagaagcgagtgt

SEQ ID NO: 563 - RNA, 19 nt, synthetic construct
  misc_feature 1..19: note = Targeting domain
  source 1..19: mol_type = other RNA
acgttccagaagcgagtgt

SEQ ID NO: 564 - RNA, 20 nt, synthetic construct
  misc_feature 1..20: note = Targeting domain
  source 1..20: mol_type = other RNA
gacgttccagaagcgagtgt

SEQ ID NO: 565 - RNA, 21 nt, synthetic construct
  misc_feature 1..21: note = Targeting domain
  source 1..21: mol_type = other RNA
agacgttccagaagcgagtgt

SEQ ID NO: 566 - RNA, 22 nt, synthetic construct
  misc_feature 1..22: note = Targeting domain
  source 1..22: mol_type = other RNA
cagacgttccagaagcgagtgt

SEQ ID NO: 567 - RNA, 23 nt, synthetic construct
  misc_feature 1..23: note = Targeting domain
  source 1..23: mol_type = other RNA
tcagacgttccagaagcgagtgt

SEQ ID NO: 568 - RNA, 24 nt, synthetic construct
  misc_feature 1..24: note = Targeting domain
  source 1..24: mol_type = other RNA
ctcagacgttccagaagcgagtgt

SEQ ID NO: 569 - RNA, 18 nt, synthetic construct
  misc_feature 1..18: note = Targeting domain
  source 1..18: mol_type = other RNA
tttgcattgagatagtgt

SEQ ID NO: 570 - RNA, 19 nt, synthetic construct
  misc_feature 1..19: note = Targeting domain
  source 1..19: mol_type = other RNA
atttgcattgagatagtgt

SEQ ID NO: 571 - RNA, 21 nt, synthetic construct
  misc_feature 1..21: note = Targeting domain
  source 1..21: mol_type = other RNA
atatttgcattgagatagtgt

SEQ ID NO: 572 - RNA, 22 nt, synthetic construct
  misc_feature 1..22: note = Targeting domain
  source 1..22: mol_type = other RNA
gatatttgcattgagatagtgt

SEQ ID NO: 573 - RNA, 23 nt, synthetic construct
  misc_feature 1..23: note = Targeting domain
  source 1..23: mol_type = other RNA
agatatttgcattgagatagtgt

SEQ ID NO: 574 - RNA, 24 nt, synthetic construct
  misc_feature 1..24: note = Targeting domain
  source 1..24: mol_type = other RNA
cagatatttgcattgagatagtgt

SEQ ID NO: 575 - RNA, 18 nt, synthetic construct
  misc_feature 1..18: note = Targeting domain
  source 1..18: mol_type = other RNA
gaataaattagagaaaaa

SEQ ID NO: 576 - RNA, 19 nt, synthetic construct
  misc_feature 1..19: note = Targeting domain
  source 1..19: mol_type = other RNA
agaataaattagagaaaaa

SEQ ID NO: 577 - RNA, 20 nt, synthetic construct
  misc_feature 1..20: note = Targeting domain
  source 1..20: mol_type = other RNA
aagaataaattagagaaaaa

SEQ ID NO: 578 - RNA, 21 nt, synthetic construct
  misc_feature 1..21: note = Targeting domain
  source 1..21: mol_type = other RNA
gaagaataaattagagaaaaa

SEQ ID NO: 579 - RNA, 22 nt, synthetic construct
  misc_feature 1..22: note = Targeting domain
  source 1..22: mol_type = other RNA
ggaagaataaattagagaaaaa

SEQ ID NO: 580 - RNA, 23 nt, synthetic construct
  misc_feature 1..23: note = Targeting domain
  source 1..23: mol_type = other RNA
gggaagaataaattagagaaaaa

SEQ ID NO: 581 - RNA, 24 nt, synthetic construct
  misc_feature 1..24: note = Targeting domain
  source 1..24: mol_type = other RNA
agggaagaataaattagagaaaaa

SEQ ID NO: 582 - RNA, 18 nt, synthetic construct
  misc_feature 1..18: note = Targeting domain
  source 1..18: mol_type = other RNA
ctagggatgaagaataaa

SEQ ID NO: 583 - RNA, 19 nt, synthetic construct
  misc_feature 1..19: note = Targeting domain
  source 1..19: mol_type = other RNA
gctagggatgaagaataaa

SEQ ID NO: 584 - RNA, 21 nt, synthetic construct
  misc_feature 1..21: note = Targeting domain
  source 1..21: mol_type = other RNA
tggctagggatgaagaataaa

SEQ ID NO: 585 - RNA, 22 nt, synthetic construct
  misc_feature 1..22: note = Targeting domain
  source 1..22: mol_type = other RNA
ctggctagggatgaagaataaa

SEQ ID NO: 586 - RNA, 23 nt, synthetic construct
  misc_feature 1..23: note = Targeting domain
  source 1..23: mol_type = other RNA
gctggctagggatgaagaataaa

SEQ ID NO: 587 - RNA, 24 nt, synthetic construct
  misc_feature 1..24: note = Targeting domain
  source 1..24: mol_type = other RNA
ggctggctagggatgaagaataaa

SEQ ID NO: 588 - RNA, 18 nt, synthetic construct
  misc_feature 1..18: note = Targeting domain
  source 1..18: mol_type = other RNA
agaaggaaactagctaaa

SEQ ID NO: 589 - RNA, 19 nt, synthetic construct
  misc_feature 1..19: note = Targeting domain
  source 1..19: mol_type = other RNA
gagaaggaaactagctaaa

SEQ ID NO: 590 - RNA, 21 nt, synthetic construct
  misc_feature 1..21: note = Targeting domain
  source 1..21: mol_type = other RNA
gggagaaggaaactagctaaa

SEQ ID NO: 591 - RNA, 22 nt, synthetic construct
  misc_feature 1..22: note = Targeting domain
  source 1..22: mol_type = other RNA
tgggagaaggaaactagctaaa

SEQ ID NO: 592 - RNA, 23 nt, synthetic construct
  misc_feature 1..23: note = Targeting domain
  source 1..23: mol_type = other RNA
atgggagaaggaaactagctaaa

SEQ ID NO: 593 - RNA, 24 nt, synthetic construct
  misc_feature 1..24: note = Targeting domain
  source 1..24: mol_type = other RNA
gatgggagaaggaaactagctaaa

SEQ ID NO: 594 - RNA, 18 nt, synthetic construct
  misc_feature 1..18: note = Targeting domain
  source 1..18: mol_type = other RNA
aaaactggaatgactgaa

SEQ ID NO: 595 - RNA, 19 nt, synthetic construct
  misc_feature 1..19: note = Targeting domain
  source 1..19: mol_type = other RNA
aaaaactggaatgactgaa

SEQ ID NO: 596 - RNA, 20 nt, synthetic construct
  misc_feature 1..20: note = Targeting domain
  source 1..20: mol_type = other RNA
gaaaaactggaatgactgaa

SEQ ID NO: 597 - RNA, 21 nt, synthetic construct
  misc_feature 1..21: note = Targeting domain
  source 1..21: mol_type = other RNA
agaaaaactggaatgactgaa

SEQ ID NO: 598 - RNA, 22 nt, synthetic construct
  misc_feature 1..22: note = Targeting domain
  source 1..22: mol_type = other RNA
gagaaaaactggaatgactgaa

SEQ ID NO: 599 - RNA, 23 nt, synthetic construct
  misc_feature 1..23: note = Targeting domain
  source 1..23: mol_type = other RNA
agagaaaaactggaatgactgaa

SEQ ID NO: 600 - RNA, 24 nt, synthetic construct
  misc_feature 1..24: note = Targeting domain
  source 1..24: mol_type = other RNA
tagagaaaaactggaatgactgaa

SEQ ID NO: 601 - RNA, 18 nt, synthetic construct
  misc_feature 1..18: note = Targeting domain
  source 1..18: mol_type = other RNA
gctagggatgaagaataa

SEQ ID NO: 602 - RNA, 19 nt, synthetic construct
  misc_feature 1..19: note = Targeting domain
  source 1..19: mol_type = other RNA
ggctagggatgaagaataa

SEQ ID NO: 603 - RNA, 20 nt, synthetic construct
  misc_feature 1..20: note = Targeting domain
  source 1..20: mol_type = other RNA
tggctagggatgaagaataa

SEQ ID NO: 604 - RNA, 21 nt, synthetic construct
  misc_feature 1..21: note = Targeting domain
  source 1..21: mol_type = other RNA
ctggctagggatgaagaataa

SEQ ID NO: 605 - RNA, 22 nt, synthetic construct
  misc_feature 1..22: note = Targeting domain
  source 1..22: mol_type = other RNA
gctggctagggatgaagaataa

SEQ ID NO: 606 - RNA, 23 nt, synthetic construct
  misc_feature 1..23: note = Targeting domain
  source 1..23: mol_type = other RNA
ggctggctagggatgaagaataa

SEQ ID NO: 607 - RNA, 24 nt, synthetic construct
  misc_feature 1..24: note = Targeting domain
  source 1..24: mol_type = other RNA
cggctggctagggatgaagaataa

SEQ ID NO: 608 - RNA, 18 nt, synthetic construct
  misc_feature 1..18: note = Targeting domain
  source 1..18: mol_type = other RNA
gagaaggaaactagctaa

SEQ ID NO: 609 - RNA, 19 nt, synthetic construct
  misc_feature 1..19: note = Targeting domain
  source 1..19: mol_type = other RNA
ggagaaggaaactagctaa

SEQ ID NO: 610 - RNA, 21 nt, synthetic construct
  misc_feature 1..21: note = Targeting domain
  source 1..21: mol_type = other RNA
tgggagaaggaaactagctaa

SEQ ID NO: 611 - RNA, 22 nt, synthetic construct
  misc_feature 1..22: note = Targeting domain
  source 1..22: mol_type = other RNA
atgggagaaggaaactagctaa

SEQ ID NO: 612 - RNA, 23 nt, synthetic construct
  misc_feature 1..23: note = Targeting domain
  source 1..23: mol_type = other RNA
gatgggagaaggaaactagctaa

SEQ ID NO: 613 - RNA, 24 nt, synthetic construct
  misc_feature 1..24: note = Targeting domain
  source 1..24: mol_type = other RNA
tgatgggagaaggaaactagctaa

SEQ ID NO: 614 - RNA, 18 nt, synthetic construct
  misc_feature 1..18: note = Targeting domain
  source 1..18: mol_type = other RNA
atagtcttagagtatcca

SEQ ID NO: 615 - RNA, 19 nt, synthetic construct
  misc_feature 1..19: note = Targeting domain
  source 1..19: mol_type = other RNA
aatagtcttagagtatcca

SEQ ID NO: 616 - RNA, 20 nt, synthetic construct
  misc_feature 1..20: note = Targeting domain
  source 1..20: mol_type = other RNA
caatagtcttagagtatcca

SEQ ID NO: 617 - RNA, 21 nt, synthetic construct
  misc_feature 1..21: note = Targeting domain
  source 1..21: mol_type = other RNA
ccaatagtcttagagtatcca

SEQ ID NO: 618 - RNA, 22 nt, synthetic construct
  misc_feature 1..22: note = Targeting domain
  source 1..22: mol_type = other RNA
accaatagtcttagagtatcca

SEQ ID NO: 619 - RNA, 23 nt, synthetic construct
  misc_feature 1..23: note = Targeting domain
  source 1..23: mol_type = other RNA
gaccaatagtcttagagtatcca

SEQ ID NO: 620 - RNA, 24 nt, synthetic construct
  misc_feature 1..24: note = Targeting domain
  source 1..24: mol_type = other RNA
tgaccaatagtcttagagtatcca

SEQ ID NO: 621 - RNA, 18 nt, synthetic construct
  misc_feature 1..18: note = Targeting domain
  source 1..18: mol_type = other RNA
atcctctatgatgggaga

SEQ ID NO: 622 - RNA, 19 nt, synthetic construct
  misc_feature 1..19: note = Targeting domain
  source 1..19: mol_type = other RNA
tatcctctatgatgggaga

SEQ ID NO: 623 - RNA, 21 nt, synthetic construct
  misc_feature 1..21: note = Targeting domain
  source 1..21: mol_type = other RNA
ggtatcctctatgatgggaga

SEQ ID NO: 624 - RNA, 22 nt, synthetic construct
  misc_feature 1..22: note = Targeting domain
  source 1..22: mol_type = other RNA
tggtatcctctatgatgggaga

SEQ ID NO: 625 - RNA, 23 nt, synthetic construct
  misc_feature 1..23: note = Targeting domain
  source 1..23: mol_type = other RNA
ctggtatcctctatgatgggaga

SEQ ID NO: 626 - RNA, 24 nt, synthetic construct
  misc_feature 1..24: note = Targeting domain
  source 1..24: mol_type = other RNA
cctggtatcctctatgatgggaga

SEQ ID NO: 627 - RNA, 18 nt, synthetic construct
  misc_feature 1..18: note = Targeting domain
  source 1..18: mol_type = other RNA
tcctggtatcctctatga

SEQ ID NO: 628 - RNA, 19 nt, synthetic construct
  misc_feature 1..19: note = Targeting domain
  source 1..19: mol_type = other RNA
gtcctggtatcctctatga

SEQ ID NO: 629 - RNA, 21 nt, synthetic construct
  misc_feature 1..21: note = Targeting domain
  source 1..21: mol_type = other RNA
aagtcctggtatcctctatga

SEQ ID NO: 630 - RNA, 22 nt, synthetic construct
  misc_feature 1..22: note = Targeting domain
  source 1..22: mol_type = other RNA
gaagtcctggtatcctctatga

SEQ ID NO: 631 - RNA, 23 nt, synthetic construct
  misc_feature 1..23: note = Targeting domain
  source 1..23: mol_type = other RNA
agaagtcctggtatcctctatga

SEQ ID NO: 632 - RNA, 24 nt, synthetic construct
  misc_feature 1..24: note = Targeting domain
  source 1..24: mol_type = other RNA
aagaagtcctggtatcctctatga

SEQ ID NO: 633 - RNA, 18 nt, synthetic construct
  misc_feature 1..18: note = Targeting domain
  source 1..18: mol_type = other RNA
ggagaaggaaactagcta

SEQ ID NO: 634 - RNA, 19 nt, synthetic construct
  misc_feature 1..19: note = Targeting domain
  source 1..19: mol_type = other RNA
gggagaaggaaactagcta

SEQ ID NO: 635 - RNA, 20 nt, synthetic construct
  misc_feature 1..20: note = Targeting domain
  source 1..20: mol_type = other RNA
tgggagaaggaaactagcta

SEQ ID NO: 636 - RNA, 21 nt, synthetic construct
  misc_feature 1..21: note = Targeting domain
  source 1..21: mol_type = other RNA
atgggagaaggaaactagcta

SEQ ID NO: 637 - RNA, 22 nt, synthetic construct
  misc_feature 1..22: note = Targeting domain
  source 1..22: mol_type = other RNA
gatgggagaaggaaactagcta

SEQ ID NO: 638 - RNA, 23 nt, synthetic construct
  misc_feature 1..23: note = Targeting domain
  source 1..23: mol_type = other RNA
tgatgggagaaggaaactagcta

SEQ ID NO: 639 - RNA, 24 nt, synthetic construct
  misc_feature 1..24: note = Targeting domain
  source 1..24: mol_type = other RNA
atgatgggagaaggaaactagcta

SEQ ID NO: 640 - RNA, 18 nt, synthetic construct
  misc_feature 1..18: note = Targeting domain
  source 1..18: mol_type = other RNA
aaagggaagaataaatta

SEQ ID NO: 641 - RNA, 19 nt, synthetic construct
  misc_feature 1..19: note = Targeting domain
  source 1..19: mol_type = other RNA
taaagggaagaataaatta

SEQ ID NO: 642 - RNA, 20 nt, synthetic construct
  misc_feature 1..20: note = Targeting domain
  source 1..20: mol_type = other RNA
ctaaagggaagaataaatta

SEQ ID NO: 643 - RNA, 21 nt, synthetic construct
  misc_feature 1..21: note = Targeting domain
  source 1..21: mol_type = other RNA
gctaaagggaagaataaatta

SEQ ID NO: 644 - RNA, 22 nt, synthetic construct
  misc_feature 1..22: note = Targeting domain
  source 1..22: mol_type = other RNA
agctaaagggaagaataaatta

SEQ ID NO: 645 - RNA, 23 nt, synthetic construct
  misc_feature 1..23: note = Targeting domain
  source 1..23: mol_type = other RNA
tagctaaagggaagaataaatta

SEQ ID NO: 646 - RNA, 24 nt, synthetic construct
  misc_feature 1..24: note = Targeting domain
  source 1..24: mol_type = other RNA
ctagctaaagggaagaataaatta

SEQ ID NO: 647 - RNA, 18 nt, synthetic construct
  misc_feature 1..18: note = Targeting domain
  source 1..18: mol_type = other RNA
agagtatccagtgaggcc

SEQ ID NO: 648 - RNA, 19 nt, synthetic construct
  misc_feature 1..19: note = Targeting domain
  source 1..19: mol_type = other RNA
tagagtatccagtgaggcc

SEQ ID NO: 649 - RNA, 21 nt, synthetic construct
  misc_feature 1..21: note = Targeting domain
  source 1..21: mol_type = other RNA
cttagagtatccagtgaggcc

SEQ ID NO: 650 - RNA, 22 nt, synthetic construct
  misc_feature 1..22: note = Targeting domain
  source 1..22: mol_type = other RNA
tcttagagtatccagtgaggcc

SEQ ID NO: 651 - RNA, 23 nt, synthetic construct
  misc_feature 1..23: note = Targeting domain
  source 1..23: mol_type = other RNA
gtcttagagtatccagtgaggcc

SEQ ID NO: 652 - RNA, 24 nt, synthetic construct
  misc_feature 1..24: note = Targeting domain
  source 1..24: mol_type = other RNA
agtcttagagtatccagtgaggcc

SEQ ID NO: 653 - RNA, 18 nt, synthetic construct
  misc_feature 1..18: note = Targeting domain
  source 1..18: mol_type = other RNA
tagagtatccagtgaggc

SEQ ID NO: 654 - RNA, 19 nt, synthetic construct
  misc_feature 1..19: note = Targeting domain
  source 1..19: mol_type = other RNA
ttagagtatccagtgaggc

SEQ ID NO: 655 - RNA, 20 nt, synthetic construct
  misc_feature 1..20: note = Targeting domain
  source 1..20: mol_type = other RNA
cttagagtatccagtgaggc

SEQ ID NO: 656 - RNA, 21 nt, synthetic construct
  misc_feature 1..21: note = Targeting domain
  source 1..21: mol_type = other RNA
tcttagagtatccagtgaggc

SEQ ID NO: 657 - RNA, 22 nt, synthetic construct
  misc_feature 1..22: note = Targeting domain
  source 1..22: mol_type = other RNA
gtcttagagtatccagtgaggc

SEQ ID NO: 658 - RNA, 23 nt, synthetic construct
  misc_feature 1..23: note = Targeting domain
  source 1..23: mol_type = other RNA
agtcttagagtatccagtgaggc

SEQ ID NO: 659 - RNA, 24 nt, synthetic construct
  misc_feature 1..24: note = Targeting domain
  source 1..24: mol_type = other RNA
tagtcttagagtatccagtgaggc

SEQ ID NO: 660 - RNA, 18 nt, synthetic construct
  misc_feature 1..18: note = Targeting domain
  source 1..18: mol_type = other RNA
caggggccggcggctggc

SEQ ID NO: 661 - RNA, 19 nt, synthetic construct
  misc_feature 1..19: note = Targeting domain
  source 1..19: mol_type = other RNA
ccaggggccggcggctggc

SEQ ID NO: 662 - RNA, 20 nt, synthetic construct
  misc_feature 1..20: note = Targeting domain
  source 1..20: mol_type = other RNA
gccaggggccggcggctggc

SEQ ID NO: 663 - RNA, 21 nt, synthetic construct
  misc_feature 1..21: note = Targeting domain
  source 1..21: mol_type = other RNA
ggccaggggccggcggctggc

SEQ ID NO: 664 - RNA, 22 nt, synthetic construct
  misc_feature 1..22: note = Targeting domain
  source 1..22: mol_type = other RNA
aggccaggggccggcggctggc

SEQ ID NO: 665 - RNA, 23 nt, synthetic construct
  misc_feature 1..23: note = Targeting domain
  source 1..23: mol_type = other RNA
gaggccaggggccggcggctggc

SEQ ID NO: 666 - RNA, 24 nt, synthetic construct
  misc_feature 1..24: note = Targeting domain
  source 1..24: mol_type = other RNA
tgaggccaggggccggcggctggc

SEQ ID NO: 667 - RNA, 18 nt, synthetic construct
  misc_feature 1..18: note = Targeting domain
  source 1..18: mol_type = other RNA
aaaattagcagtatcctc

SEQ ID NO: 668 - RNA, 19 nt, synthetic construct
  misc_feature 1..19: note = Targeting domain
  source 1..19: mol_type = other RNA
aaaaattagcagtatcctc

SEQ ID NO: 669 - RNA, 20 nt, synthetic construct
  misc_feature 1..20: note = Targeting domain
  source 1..20: mol_type = other RNA
aaaaaattagcagtatcctc

SEQ ID NO: 670 - RNA, 21 nt, synthetic construct
  misc_feature 1..21: note = Targeting domain
  source 1..21: mol_type = other RNA
aaaaaaattagcagtatcctc

SEQ ID NO: 671 - RNA, 22 nt, synthetic construct
  misc_feature 1..22: note = Targeting domain
  source 1..22: mol_type = other RNA
aaaaaaaattagcagtatcctc

SEQ ID NO: 672 - RNA, 23 nt, synthetic construct
  misc_feature 1..23: note = Targeting domain
  source 1..23: mol_type = other RNA
taaaaaaaattagcagtatcctc

SEQ ID NO: 673 - RNA, 24 nt, synthetic construct
  misc_feature 1..24: note = Targeting domain
  source 1..24: mol_type = other RNA
ataaaaaaaattagcagtatcctc

SEQ ID NO: 674 - RNA, 18 nt, synthetic construct
  misc_feature 1..18: note = Targeting domain
  source 1..18: mol_type = other RNA
gttccacacactcgcttc

SEQ ID NO: 675 - RNA, 19 nt, synthetic construct
  misc_feature 1..19: note = Targeting domain
  source 1..19: mol_type = other RNA
agttccacacactcgcttc

SEQ ID NO: 676 - RNA, 21 nt, synthetic construct
  misc_feature 1..21: note = Targeting domain
  source 1..21: mol_type = other RNA
gcagttccacacactcgcttc

SEQ ID NO: 677 - RNA, 22 nt, synthetic construct
  misc_feature 1..22: note = Targeting domain
  source 1..22: mol_type = other RNA
agcagttccacacactcgcttc

SEQ ID NO: 678 - RNA, 23 nt, synthetic construct
  misc_feature 1..23: note = Targeting domain
  source 1..23: mol_type = other RNA
cagcagttccacacactcgcttc

SEQ ID NO: 679 - RNA, 24 nt, synthetic construct
  misc_feature 1..24: note = Targeting domain
  source 1..24: mol_type = other RNA
tcagcagttccacacactcgcttc

SEQ ID NO: 680 - RNA, 18 nt, synthetic construct
  misc_feature 1..18: note = Targeting domain
  source 1..18: mol_type = other RNA
tatcctctatgatgggag

SEQ ID NO: 681 - RNA, 19 nt, synthetic construct
  misc_feature 1..19: note = Targeting domain
  source 1..19: mol_type = other RNA
gtatcctctatgatgggag

SEQ ID NO: 682 - RNA, 20 nt, synthetic construct
  misc_feature 1..20: note = Targeting domain
  source 1..20: mol_type = other RNA
ggtatcctctatgatgggag

SEQ ID NO: 683 - RNA, 21 nt, synthetic construct
  misc_feature 1..21: note = Targeting domain
  source 1..21: mol_type = other RNA
tggtatcctctatgatgggag

SEQ ID NO: 684 - RNA, 22 nt, synthetic construct
  misc_feature 1..22: note = Targeting domain
  source 1..22: mol_type = other RNA
ctggtatcctctatgatgggag

SEQ ID NO: 685 - RNA, 23 nt, synthetic construct
  misc_feature 1..23: note = Targeting domain
  source 1..23: mol_type = other RNA
cctggtatcctctatgatgggag

SEQ ID NO: 686 - RNA, 24 nt, synthetic construct
  misc_feature 1..24: note = Targeting domain
  source 1..24: mol_type = other RNA
tcctggtatcctctatgatgggag

SEQ ID NO: 687 - RNA, 18 nt, synthetic construct
  misc_feature 1..18: note = Targeting domain
  source 1..18: mol_type = other RNA
gccggcggctggctaggg

SEQ ID NO: 688 - RNA, 19 nt, synthetic construct
  misc_feature 1..19: note = Targeting domain
  source 1..19: mol_type = other RNA
ggccggcggctggctaggg

SEQ ID NO: 689 - RNA, 20 nt, synthetic construct
  misc_feature 1..20: note = Targeting domain
  source 1..20: mol_type = other RNA
gggccggcggctggctaggg

SEQ ID NO: 690 - RNA, 21 nt, synthetic construct
  misc_feature 1..21: note = Targeting domain
  source 1..21: mol_type = other RNA
ggggccggcggctggctaggg

SEQ ID NO: 691 - RNA, 22 nt, synthetic construct
  misc_feature 1..22: note = Targeting domain
  source 1..22: mol_type = other RNA
aggggccggcggctggctaggg

SEQ ID NO: 692 - RNA, 23 nt, synthetic construct
  misc_feature 1..23: note = Targeting domain
  source 1..23: mol_type = other RNA
caggggccggcggctggctaggg

SEQ ID NO: 693 - RNA, 24 nt, synthetic construct
  misc_feature 1..24: note = Targeting domain
  source 1..24: mol_type = other RNA
ccaggggccggcggctggctaggg

SEQ ID NO: 694 - RNA, 18 nt, synthetic construct
  misc_feature 1..18: note = Targeting domain
  source 1..18: mol_type = other RNA
tggtatcctctatgatgg

SEQ ID NO: 695 - RNA, 19 nt, synthetic construct
  misc_feature 1..19: note = Targeting domain
  source 1..19: mol_type = other RNA
ctggtatcctctatgatgg

SEQ ID NO: 696 - RNA, 20 nt, synthetic construct
  misc_feature 1..20: note = Targeting domain
  source 1..20: mol_type = other RNA
cctggtatcctctatgatgg

SEQ ID NO: 697 - RNA, 21 nt, synthetic construct
  misc_feature 1..21: note = Targeting domain
  source 1..21: mol_type = other RNA
tcctggtatcctctatgatgg

SEQ ID NO: 698 - RNA, 22 nt, synthetic construct
  misc_feature 1..22: note = Targeting domain
  source 1..22: mol_type = other RNA
gtcctggtatcctctatgatgg

SEQ ID NO: 699 - RNA, 23 nt, synthetic construct
  misc_feature 1..23: note = Targeting domain
  source 1..23: mol_type = other RNA
agtcctggtatcctctatgatgg

SEQ ID NO: 700 - RNA, 24 nt, synthetic construct
  misc_feature 1..24: note = Targeting domain
  source 1..24: mol_type = other RNA
aagtcctggtatcctctatgatgg

SEQ ID NO: 701 - RNA, 18 nt, synthetic construct
  misc_feature 1..18: note = Targeting domain
  source 1..18: mol_type = other RNA
gtcctggtatcctctatg

SEQ ID NO: 702 - RNA, 19 nt, synthetic construct
  misc_feature 1..19: note = Targeting domain
  source 1..19: mol_type = other RNA
agtcctggtatcctctatg

SEQ ID NO: 703 - RNA, 20 nt, synthetic construct
  misc_feature 1..20: note = Targeting domain
  source 1..20: mol_type = other RNA
aagtcctggtatcctctatg

SEQ ID NO: 704 - RNA, 21 nt, synthetic construct
  misc_feature 1..21: note = Targeting domain
  source 1..21: mol_type = other RNA
gaagtcctggtatcctctatg

SEQ ID NO: 705 - RNA, 22 nt, synthetic construct
  misc_feature 1..22: note = Targeting domain
  source 1..22: mol_type = other RNA
agaagtcctggtatcctctatg

SEQ ID NO: 706 - RNA, 23 nt, synthetic construct
  misc_feature 1..23: note = Targeting domain
  source 1..23: mol_type = other RNA
aagaagtcctggtatcctctatg

SEQ ID NO: 707 - RNA, 24 nt, synthetic construct
  misc_feature 1..24: note = Targeting domain
  source 1..24: mol_type = other RNA
aaagaagtcctggtatcctctatg

SEQ ID NO: 708 - RNA, 18 nt, synthetic construct
  misc_feature 1..18: note = Targeting domain
  source 1..18: mol_type = other RNA
ctaaagggaagaataaat

SEQ ID NO: 709 - RNA, 19 nt, synthetic construct
  misc_feature 1..19: note = Targeting domain
  source 1..19: mol_type = other RNA
gctaaagggaagaataaat

SEQ ID NO: 710 - RNA, 20 nt, synthetic construct
  misc_feature 1..20: note = Targeting domain
  source 1..20: mol_type = other RNA
agctaaagggaagaataaat

SEQ ID NO: 711 - RNA, 21 nt, synthetic construct
  misc_feature 1..21: note = Targeting domain
  source 1..21: mol_type = other RNA
tagctaaagggaagaataaat

SEQ ID NO: 712 - RNA, 22 nt, synthetic construct
  misc_feature 1..22: note = Targeting domain
  source 1..22: mol_type = other RNA
ctagctaaagggaagaataaat

SEQ ID NO: 713 - RNA, 23 nt, synthetic construct
  misc_feature 1..23: note = Targeting domain
  source 1..23: mol_type = other RNA
actagctaaagggaagaataaat

SEQ ID NO: 714 - RNA, 24 nt, synthetic construct
  misc_feature 1..24: note = Targeting domain
  source 1..24: mol_type = other RNA
aactagctaaagggaagaataaat

SEQ ID NO: 715 - RNA, 18 nt, synthetic construct
  misc_feature 1..18: note = Targeting domain
  source 1..18: mol_type = other RNA
aaactggaatgactgaat

SEQ ID NO: 716 - RNA, 19 nt, synthetic construct
  misc_feature 1..19: note = Targeting domain
  source 1..19: mol_type = other RNA
aaaactggaatgactgaat

SEQ ID NO: 717 - RNA, 21 nt, synthetic construct
  misc_feature 1..21: note = Targeting domain
  source 1..21: mol_type = other RNA
gaaaaactggaatgactgaat

SEQ ID NO: 718 - RNA, 22 nt, synthetic construct
  misc_feature 1..22: note = Targeting domain
  source 1..22: mol_type = other RNA
agaaaaactggaatgactgaat

SEQ ID NO: 719 - RNA, 23 nt, synthetic construct
  misc_feature 1..23: note = Targeting domain
  source 1..23: mol_type = other RNA
gagaaaaactggaatgactgaat

SEQ ID NO: 720 - RNA, 24 nt, synthetic construct
  misc_feature 1..24: note = Targeting domain
  source 1..24: mol_type = other RNA
agagaaaaactggaatgactgaat

SEQ ID NO: 721 - RNA, 18 nt, synthetic construct
  misc_feature 1..18: note = Targeting domain
  source 1..18: mol_type = other RNA
cctggtatcctctatgat

SEQ ID NO: 722 - RNA, 19 nt, synthetic construct
  misc_feature 1..19: note = Targeting domain
  source 1..19: mol_type = other RNA
tcctggtatcctctatgat

SEQ ID NO: 723 - RNA, 21 nt, synthetic construct
  misc_feature 1..21: note = Targeting domain
  source 1..21: mol_type = other RNA
agtcctggtatcctctatgat

SEQ ID NO: 724 - RNA, 22 nt, synthetic construct
  misc_feature 1..22: note = Targeting domain
  source 1..22: mol_type = other RNA
aagtcctggtatcctctatgat

SEQ ID NO: 725 - RNA, 23 nt, synthetic construct
  misc_feature 1..23: note = Targeting domain
  source 1..23: mol_type = other RNA
gaagtcctggtatcctctatgat

SEQ ID NO: 726 - RNA, 24 nt, synthetic construct
  misc_feature 1..24: note = Targeting domain
  source 1..24: mol_type = other RNA
agaagtcctggtatcctctatgat

SEQ ID NO: 727 - RNA, 18 nt, synthetic construct
  misc_feature 1..18: note = Targeting domain
  source 1..18: mol_type = other RNA
aaattagcagtatcctct

SEQ ID NO: 728 - RNA, 19 nt, synthetic construct
  misc_feature 1..19: note = Targeting domain
  source 1..19: mol_type = other RNA
aaaattagcagtatcctct

SEQ ID NO: 729 - RNA, 21 nt, synthetic construct
  misc_feature 1..21: note = Targeting domain
  source 1..21: mol_type = other RNA
aaaaaattagcagtatcctct

SEQ ID NO: 730 - RNA, 22 nt, synthetic construct
  misc_feature 1..22: note = Targeting domain
  source 1..22: mol_type = other RNA
aaaaaaattagcagtatcctct

SEQ ID NO: 731 - RNA, 23 nt, synthetic construct
  misc_feature 1..23: note = Targeting domain
  source 1..23: mol_type = other RNA
aaaaaaaattagcagtatcctct

SEQ ID NO: 732 - RNA, 24 nt, synthetic construct
  misc_feature 1..24: note = Targeting domain
  source 1..24: mol_type = other RNA
taaaaaaaattagcagtatcctct

SEQ ID NO: 733 - RNA, 18 nt, synthetic construct
  misc_feature 1..18: note = Targeting domain
  source 1..18: mol_type = other RNA
cactcgcttctggaacgt

SEQ ID NO: 734 - RNA, 19 nt, synthetic construct
  misc_feature 1..19: note = Targeting domain
  source 1..19: mol_type = other RNA
acactcgcttctggaacgt

SEQ ID NO: 735 - RNA, 20 nt, synthetic construct
  misc_feature 1..20: note = Targeting domain
  source 1..20: mol_type = other RNA
cacactcgcttctggaacgt

SEQ ID NO: 736 - RNA, 21 nt, synthetic construct
  misc_feature 1..21: note = Targeting domain
  source 1..21: mol_type = other RNA
acacactcgcttctggaacgt

SEQ ID NO: 737 - RNA, 22 nt, synthetic construct
  misc_feature 1..22: note = Targeting domain
  source 1..22: mol_type = other RNA
cacacactcgcttctggaacgt

SEQ ID NO: 738 - RNA, 23 nt, synthetic construct
  misc_feature 1..23: note = Targeting domain
  source 1..23: mol_type = other RNA
ccacacactcgcttctggaacgt

SEQ ID NO: 739 - RNA, 24 nt, synthetic construct
  misc_feature 1..24: note = Targeting domain
  source 1..24: mol_type = other RNA
tccacacactcgcttctggaacgt

SEQ ID NO: 740 - RNA, 18 nt, synthetic construct
  misc_feature 1..18: note = Targeting domain
  source 1..18: mol_type = other RNA
ctcaatgcaaatatctgt

SEQ ID NO: 741 - RNA, 19 nt, synthetic construct
  misc_feature 1..19: note = Targeting domain
  source 1..19: mol_type = other RNA
tctcaatgcaaatatctgt

SEQ ID NO: 742 - RNA, 20 nt, synthetic construct
  misc_feature 1..20: note = Targeting domain
  source 1..20: mol_type = other RNA
atctcaatgcaaatatctgt

SEQ ID NO: 743 - RNA, 21 nt, synthetic construct
  misc_feature 1..21: note = Targeting domain
  source 1..21: mol_type = other RNA
tatctcaatgcaaatatctgt

SEQ ID NO: 744 - RNA, 22 nt, synthetic construct
  misc_feature 1..22: note = Targeting domain
  source 1..22: mol_type = other RNA
ctatctcaatgcaaatatctgt

SEQ ID NO: 745 - RNA, 23 nt, synthetic construct
  misc_feature 1..23: note = Targeting domain
  source 1..23: mol_type = other RNA
actatctcaatgcaaatatctgt

SEQ ID NO: 746 - RNA, 24 nt, synthetic construct
  misc_feature 1..24: note = Targeting domain
  source 1..24: mol_type = other RNA
cactatctcaatgcaaatatctgt

SEQ ID NO: 747 - RNA, 18 nt, synthetic construct
  misc_feature 1..18: note = Targeting domain
  source 1..18: mol_type = other RNA
agttccacacactcgctt

SEQ ID NO: 748 - RNA, 19 nt, synthetic construct
  misc_feature 1..19: note = Targeting domain
  source 1..19: mol_type = other RNA
cagttccacacactcgctt

SEQ ID NO: 749 - RNA, 20 nt, synthetic construct
  misc_feature 1..20: note = Targeting domain
  source 1..20: mol_type = other RNA
gcagttccacacactcgctt

SEQ ID NO: 750 - RNA, 21 nt, synthetic construct
  misc_feature 1..21: note = Targeting domain
  source 1..21: mol_type = other RNA
agcagttccacacactcgctt

SEQ ID NO: 751 - RNA, 22 nt, synthetic construct
  misc_feature 1..22: note = Targeting domain
  source 1..22: mol_type = other RNA
cagcagttccacacactcgctt

SEQ ID NO: 752 - RNA, 23 nt, synthetic construct
  misc_feature 1..23: note = Targeting domain
  source 1..23: mol_type = other RNA
tcagcagttccacacactcgctt

SEQ ID NO: 753 - RNA, 24 nt, synthetic construct
  misc_feature 1..24: note = Targeting domain
  source 1..24: mol_type = other RNA
ttcagcagttccacacactcgctt

SEQ ID NO: 754 - RNA, 18 nt, synthetic construct
  misc_feature 1..18: note = Targeting domain
  source 1..18: mol_type = other RNA
aattagcagtatcctctt

SEQ ID NO: 755 - RNA, 19 nt, synthetic construct
  misc_feature 1..19: note = Targeting domain
  source 1..19: mol_type = other RNA
aaattagcagtatcctctt

SEQ ID NO: 756 - RNA, 21 nt, synthetic construct
  misc_feature 1..21: note = Targeting domain
  source 1..21: mol_type = other RNA
aaaaattagcagtatcctctt

SEQ ID NO: 757 - RNA, 22 nt, synthetic construct
  misc_feature 1..22: note = Targeting domain
  source 1..22: mol_type = other RNA
aaaaaattagcagtatcctctt

SEQ ID NO: 758 - RNA, 23 nt, synthetic construct
  misc_feature 1..23: note = Targeting domain
  source 1..23: mol_type = other RNA
aaaaaaattagcagtatcctctt

SEQ ID NO: 759 - RNA, 24 nt, synthetic construct
  misc_feature 1..24: note = Targeting domain
  source 1..24: mol_type = other RNA
aaaaaaaattagcagtatcctctt

SEQ ID NO: 760 - RNA, 17 nt, synthetic construct
  misc_feature 1..17: note = Targeting domain
  source 1..17: mol_type = other RNA
gaagaaaactagctaaa

SEQ ID NO: 761 - RNA, 17 nt, synthetic construct
  misc_feature 1..17: note = Targeting domain
  source 1..17: mol_type = other RNA
gcagcagtatcctcttg

SEQ ID NO: 762 - RNA, 20 nt, synthetic construct
  misc_feature 1..20: note = Targeting domain
  source 1..20: mol_type = other RNA
ggagaagaaaactagctaaa

SEQ ID NO: 763 - RNA, 20 nt, synthetic construct
  misc_feature 1..20: note = Targeting domain
  source 1..20: mol_type = other RNA
gggagaagaaaactagctaa

SEQ ID NO: 764 - RNA, 20 nt, synthetic construct
  misc_feature 1..20: note = Targeting domain
  source 1..20: mol_type = other RNA
gtcctggtatcttctatggt

SEQ ID NO: 765 - RNA, 17 nt, synthetic construct
  misc_feature 1..17: note = Targeting domain
  source 1..17: mol_type = other RNA
agaagaaaactagctaa

SEQ ID NO: 766 - RNA, 17 nt, synthetic construct
  misc_feature 1..17: note = Targeting domain
  source 1..17: mol_type = other RNA
agtcctggtatcttcta

SEQ ID NO: 767 - RNA, 17 nt, synthetic construct
  misc_feature 1..17: note = Targeting domain
  source 1..17: mol_type = other RNA
ccaccatagaagatacc

SEQ ID NO: 768 - RNA, 17 nt, synthetic construct
  misc_feature 1..17: note = Targeting domain
  source 1..17: mol_type = other RNA
cctggtatcttctatgg

SEQ ID NO: 769 - RNA, 17 nt, synthetic construct
  misc_feature 1..17: note = Targeting domain
  source 1..17: mol_type = other RNA
cagcagtatcctcttgg

SEQ ID NO: 770 - RNA, 17 nt, synthetic construct
  misc_feature 1..17: note = Targeting domain
  source 1..17: mol_type = other RNA
aattggaatgactgaat

SEQ ID NO: 771 - RNA, 17 nt, synthetic construct
  misc_feature 1..17: note = Targeting domain
  source 1..17: mol_type = other RNA
ctggtatcttctatggt

SEQ ID NO: 772 - RNA, 17 nt, synthetic construct
  misc_feature 1..17: note = Targeting domain
  source 1..17: mol_type = other RNA
agcagcagtatcctctt

SEQ ID NO: 773 - RNA, 20 nt, synthetic construct
  misc_feature 1..20: note = Targeting domain
  source 1..20: mol_type = other RNA
agaagtcctggtatcttcta

SEQ ID NO: 774 - RNA, 20 nt, synthetic construct
  misc_feature 1..20: note = Targeting domain
  source 1..20: mol_type = other RNA
ctcccaccatagaagatacc

SEQ ID NO: 775 - RNA, 20 nt, synthetic construct
  misc_feature 1..20: note = Targeting domain
  source 1..20: mol_type = other RNA
agtcctggtatcttctatgg

SEQ ID NO: 776 - RNA, 20 nt, synthetic construct
  misc_feature 1..20: note = Targeting domain
  source 1..20: mol_type = other RNA
aagcagcagtatcctcttgg

SEQ ID NO: 777 - RNA, 20 nt, synthetic construct
  misc_feature 1..20: note = Targeting domain
  source 1..20: mol_type = other RNA
taagcagcagtatcctcttg

SEQ ID NO: 778 - RNA, 20 nt, synthetic construct
  misc_feature 1..20: note = Targeting domain
  source 1..20: mol_type = other RNA
agaataaattagagaaaaat

SEQ ID NO: 779 - RNA, 20 nt, synthetic construct
  misc_feature 1..20: note = Targeting domain
  source 1..20: mol_type = other RNA
aaaaattggaatgactgaat

SEQ ID NO: 780 - RNA, 20 nt, synthetic construct
  misc_feature 1..20: note = Targeting domain
  source 1..20: mol_type = other RNA
attaagcagcagtatcctct

SEQ ID NO: 781 - RNA, 20 nt, synthetic construct
  misc_feature 1..20: note = Targeting domain
  source 1..20: mol_type = other RNA
ttaagcagcagtatcctctt

SEQ ID NO: 782 - RNA, 17 nt, synthetic construct
  misc_feature 1..17: note = Targeting domain
  source 1..17: mol_type = other RNA
ataaattagagaaaaat

SEQ ID NO: 783 - RNA, 17 nt, synthetic construct
  misc_feature 1..17: note = Targeting domain
  source 1..17: mol_type = other RNA
aagcagcagtatcctct

SEQ ID NO: 784 - RNA, 20 nt, synthetic construct
  misc_feature 1..20: note = Targeting domain
  source 1..20: mol_type = other RNA
gaagaaaactagctaaaggg

SEQ ID NO: 785 - RNA, 22 nt, synthetic construct
  misc_feature 1..22: note = Targeting domain
  source 1..22: mol_type = other RNA
gagaagaaaactagctaaaggg

SEQ ID NO: 786 - RNA, 23 nt, synthetic construct
  misc_feature 1..23: note = Targeting domain
  source 1..23: mol_type = other RNA
ggagaagaaaactagctaaaggg

SEQ ID NO: 787 - RNA, 24 nt, synthetic construct
  misc_feature 1..24: note = Targeting domain
  source 1..24: mol_type = other RNA
gggagaagaaaactagctaaaggg

SEQ ID NO: 788 - RNA, 19 nt, synthetic construct
  misc_feature 1..19: note = Targeting domain
  source 1..19: mol_type = other RNA
gaataaattagagaaaaat

SEQ ID NO: 789 - RNA, 22 nt, synthetic construct
  misc_feature 1..22: note = Targeting domain
  source 1..22: mol_type = other RNA
gaagaataaattagagaaaaat

SEQ ID NO: 790 - RNA, 23 nt, synthetic construct
  misc_feature 1..23: note = Targeting domain
  source 1..23: mol_type = other RNA
ggaagaataaattagagaaaaat

SEQ ID NO: 791 - RNA, 24 nt, synthetic construct
  misc_feature 1..24: note = Targeting domain
  source 1..24: mol_type = other RNA
gggaagaataaattagagaaaaat

SEQ ID NO: 792 - RNA, 18 nt, synthetic construct
  misc_feature 1..18: note = Targeting domain
  source 1..18: mol_type = other RNA
agagaaaaattggaatga

SEQ ID NO: 793 - RNA, 19 nt, synthetic construct
  misc_feature 1..19: note = Targeting domain
  source 1..19: mol_type = other RNA
tagagaaaaattggaatga

SEQ ID NO: 794 - RNA, 20 nt, synthetic construct
  misc_feature 1..20: note = Targeting domain
  source 1..20: mol_type = other RNA
ttagagaaaaattggaatga

SEQ ID NO: 795 - RNA, 21 nt, synthetic construct
  misc_feature 1..21: note = Targeting domain
  source 1..21: mol_type = other RNA
attagagaaaaattggaatga

SEQ ID NO: 796 - RNA, 22 nt, synthetic construct
  misc_feature 1..22: note = Targeting domain
  source 1..22: mol_type = other RNA
aattagagaaaaattggaatga

SEQ ID NO: 797 - RNA, 23 nt, synthetic construct
  misc_feature 1..23: note = Targeting domain
  source 1..23: mol_type = other RNA
aaattagagaaaaattggaatga

SEQ ID NO: 798 - RNA, 24 nt, synthetic construct
  misc_feature 1..24: note = Targeting domain
  source 1..24: mol_type = other RNA
taaattagagaaaaattggaatga

SEQ ID NO: 799 - RNA, 18 nt, synthetic construct
  misc_feature 1..18: note = Targeting domain
  source 1..18: mol_type = other RNA
agaaaactagctaaaggg

SEQ ID NO: 800 - RNA, 19 nt, synthetic construct
  misc_feature 1..19: note = Targeting domain
  source 1..19: mol_type = other RNA
aagaaaactagctaaaggg

SEQ ID NO: 801 - RNA, 21 nt, synthetic construct
  misc_feature 1..21: note = Targeting domain
  source 1..21: mol_type = other RNA
agaagaaaactagctaaaggg

SEQ ID NO: 802 - RNA, 18 nt, synthetic construct
  misc_feature 1..18: note = Targeting domain
  source 1..18: mol_type = other RNA
aataaattagagaaaaat

SEQ ID NO: 803 - RNA, 21 nt, synthetic construct
  misc_feature 1..21: note = Targeting domain
  source 1..21: mol_type = other RNA
aagaataaattagagaaaaat

SEQ ID NO: 804 - RNA, 18 nt, synthetic construct
  misc_feature 1..18: note = Targeting domain
  source 1..18: mol_type = other RNA
agttttcttctcccacca

SEQ ID NO: 805 - RNA, 19 nt, synthetic construct
  misc_feature 1..19: note = Targeting domain
  source 1..19: mol_type = other RNA
tagttttcttctcccacca

SEQ ID NO: 806 - RNA, 20 nt, synthetic construct
  misc_feature 1..20: note = Targeting domain
  source 1..20: mol_type = other RNA
ctagttttcttctcccacca

SEQ ID NO: 807 - RNA, 21 nt, synthetic construct
  misc_feature 1..21: note = Targeting domain
  source 1..21: mol_type = other RNA
gctagttttcttctcccacca

SEQ ID NO: 808 - RNA, 22 nt, synthetic construct
  misc_feature 1..22: note = Targeting domain
  source 1..22: mol_type = other RNA
agctagttttcttctcccacca

SEQ ID NO: 809 - RNA, 23 nt, synthetic construct
  misc_feature 1..23: note = Targeting domain
  source 1..23: mol_type = other RNA
tagctagttttcttctcccacca

SEQ ID NO: 810 - RNA, 24 nt, synthetic construct
  misc_feature 1..24: note = Targeting domain
  source 1..24: mol_type = other RNA
ttagctagttttcttctcccacca

SEQ ID NO: 811 - RNA, 18 nt, synthetic construct
  misc_feature 1..18: note = Targeting domain
  source 1..18: mol_type = other RNA
tcccaccatagaagatac

SEQ ID NO: 812 - RNA, 19 nt, synthetic construct
  misc_feature 1..19: note = Targeting domain
  source 1..19: mol_type = other RNA
ctcccaccatagaagatac

SEQ ID NO: 813 - RNA, 20 nt, synthetic construct
  misc_feature 1..20: note = Targeting domain
  source 1..20: mol_type = other RNA
tctcccaccatagaagatac

SEQ ID NO: 814 - RNA, 21 nt, synthetic construct
  misc_feature 1..21: note = Targeting domain
  source 1..21: mol_type = other RNA
ttctcccaccatagaagatac

SEQ ID NO: 815 - RNA, 22 nt, synthetic construct
  misc_feature 1..22: note = Targeting domain
  source 1..22: mol_type = other RNA
cttctcccaccatagaagatac

SEQ ID NO: 816 - RNA, 23 nt, synthetic construct
  misc_feature 1..23: note = Targeting domain
  source 1..23: mol_type = other RNA
tcttctcccaccatagaagatac

SEQ ID NO: 817 - RNA, 24 nt, synthetic construct
  misc_feature 1..24: note = Targeting domain
  source 1..24: mol_type = other RNA
ttcttctcccaccatagaagatac

SEQ ID NO: 818 - RNA, 18 nt, synthetic construct
  misc_feature 1..18: note = Targeting domain
  source 1..18: mol_type = other RNA
agaagaaaactagctaaa

SEQ ID NO: 819 - RNA, 19 nt, synthetic construct
  misc_feature 1..19: note = Targeting domain
  source 1..19: mol_type = other RNA
gagaagaaaactagctaaa

SEQ ID NO: 820 - RNA, 21 nt, synthetic construct
  misc_feature 1..21: note = Targeting domain
  source 1..21: mol_type = other RNA
gggagaagaaaactagctaaa

SEQ ID NO: 821 - RNA, 22 nt, synthetic construct
  misc_feature 1..22: note = Targeting domain
  source 1..22: mol_type = other RNA
tgggagaagaaaactagctaaa

SEQ ID NO: 822 - RNA, 23 nt, synthetic construct
  misc_feature 1..23: note = Targeting domain
  source 1..23: mol_type = other RNA
gtgggagaagaaaactagctaaa

SEQ ID NO: 823 - RNA, 24 nt, synthetic construct
  misc_feature 1..24: note = Targeting domain
  source 1..24: mol_type = other RNA
ggtgggagaagaaaactagctaaa

SEQ ID NO: 824 - RNA, 18 nt, synthetic construct
  misc_feature 1..18: note = Targeting domain
  source 1..18: mol_type = other RNA
aaaattggaatgactgaa

SEQ ID NO: 825 - RNA, 19 nt, synthetic construct
  misc_feature 1..19: note = Targeting domain
  source 1..19: mol_type = other RNA
aaaaattggaatgactgaa

SEQ ID NO: 826 - RNA, 20 nt, synthetic construct
  misc_feature 1..20: note = Targeting domain
  source 1..20: mol_type = other RNA
gaaaaattggaatgactgaa

SEQ ID NO: 827 - RNA, 21 nt, synthetic construct
  misc_feature 1..21: note = Targeting domain
  source 1..21: mol_type = other RNA
agaaaaattggaatgactgaa

SEQ ID NO: 828 - RNA, 22 nt, synthetic construct
  misc_feature 1..22: note = Targeting domain
  source 1..22: mol_type = other RNA
gagaaaaattggaatgactgaa

SEQ ID NO: 829 - RNA, 23 nt, synthetic construct
  misc_feature 1..23: note = Targeting domain
  source 1..23: mol_type = other RNA
agagaaaaattggaatgactgaa

SEQ ID NO: 830 - RNA, 24 nt, synthetic construct
  misc_feature 1..24: note = Targeting domain
  source 1..24: mol_type = other RNA
tagagaaaaattggaatgactgaa

SEQ ID NO: 831 - RNA, 18 nt, synthetic construct
  misc_feature 1..18: note = Targeting domain
  source 1..18: mol_type = other RNA
gagaagaaaactagctaa

SEQ ID NO: 832 - RNA, 19 nt, synthetic construct
  misc_feature 1..19: note = Targeting domain
  source 1..19: mol_type = other RNA
ggagaagaaaactagctaa

SEQ ID NO: 833 - RNA, 21 nt, synthetic construct
  misc_feature 1..21: note = Targeting domain
  source 1..21: mol_type = other RNA
tgggagaagaaaactagctaa

SEQ ID NO: 834 - RNA, 22 nt, synthetic construct
  misc_feature 1..22: note = Targeting domain
  source 1..22: mol_type = other RNA
gtgggagaagaaaactagctaa

SEQ ID NO: 835 - RNA, 23 nt, synthetic construct
  misc_feature 1..23: note = Targeting domain
  source 1..23: mol_type = other RNA
ggtgggagaagaaaactagctaa

SEQ ID NO: 836 - RNA, 24 nt, synthetic construct
  misc_feature 1..24: note = Targeting domain
  source 1..24: mol_type = other RNA
tggtgggagaagaaaactagctaa

SEQ ID NO: 837 - RNA, 18 nt, synthetic construct
  misc_feature 1..18: note = Targeting domain
  source 1..18: mol_type = other RNA
ggagaagaaaactagcta

SEQ ID NO: 838 - RNA, 19 nt, synthetic construct
  misc_feature 1..19: note = Targeting domain
  source 1..19: mol_type = other RNA
gggagaagaaaactagcta

SEQ ID NO: 839 - RNA, 20 nt, synthetic construct
  misc_feature 1..20: note = Targeting domain
  source 1..20: mol_type = other RNA
tgggagaagaaaactagcta

SEQ ID NO: 840 - RNA, 21 nt, synthetic construct
  misc_feature 1..21: note = Targeting domain
  source 1..21: mol_type = other RNA
gtgggagaagaaaactagcta

SEQ ID NO: 841 - RNA, 22 nt, synthetic construct
  misc_feature 1..22: note = Targeting domain
  source 1..22: mol_type = other RNA
ggtgggagaagaaaactagcta

SEQ ID NO: 842 - RNA, 23 nt, synthetic construct
  misc_feature 1..23: note = Targeting domain
  source 1..23: mol_type = other RNA
tggtgggagaagaaaactagcta

SEQ ID NO: 843 - RNA, 24 nt, synthetic construct
  misc_feature 1..24: note = Targeting domain
  source 1..24: mol_type = other RNA
atggtgggagaagaaaactagcta

SEQ ID NO: 844 - RNA, 18 nt, synthetic construct
  misc_feature 1..18: note = Targeting domain
  source 1..18: mol_type = other RNA
ttaagcagcagtatcctc

SEQ ID NO: 845 - RNA, 19 nt, synthetic construct
  misc_feature 1..19: note = Targeting domain
  source 1..19: mol_type = other RNA
attaagcagcagtatcctc

SEQ ID NO: 846 - RNA, 20 nt, synthetic construct
  misc_feature 1..20: note = Targeting domain
  source 1..20: mol_type = other RNA
aattaagcagcagtatcctc

SEQ ID NO: 847 - RNA, 21 nt, synthetic construct
  misc_feature 1..21: note = Targeting domain
  source 1..21: mol_type = other RNA
aaattaagcagcagtatcctc

SEQ ID NO: 848 - RNA, 22 nt, synthetic construct
  misc_feature 1..22: note = Targeting domain
  source 1..22: mol_type = other RNA
aaaattaagcagcagtatcctc

SEQ ID NO: 849 - RNA, 23 nt, synthetic construct
  misc_feature 1..23: note = Targeting domain
  source 1..23: mol_type = other RNA
aaaaattaagcagcagtatcctc

SEQ ID NO: 850 - RNA, 24 nt, synthetic construct
  misc_feature 1..24: note = Targeting domain
  source 1..24: mol_type = other RNA
aaaaaattaagcagcagtatcctc

SEQ ID NO: 851 - RNA, 18 nt, synthetic construct
  misc_feature 1..18: note = Targeting domain
  source 1..18: mol_type = other RNA
tatcttctatggtgggag

SEQ ID NO: 852 - RNA, 19 nt, synthetic construct
  misc_feature 1..19: note = Targeting domain
  source 1..19: mol_type = other RNA
gtatcttctatggtgggag

SEQ ID NO: 853 - RNA, 20 nt, synthetic construct
  misc_feature 1..20: note = Targeting domain
  source 1..20: mol_type = other RNA
ggtatcttctatggtgggag

SEQ ID NO: 854 - RNA, 21 nt, synthetic construct
  misc_feature 1..21: note = Targeting domain
  source 1..21: mol_type = other RNA
tggtatcttctatggtgggag

SEQ ID NO: 855 - RNA, 22 nt, synthetic construct
  misc_feature 1..22: note = Targeting domain
  source 1..22: mol_type = other RNA
ctggtatcttctatggtgggag

SEQ ID NO: 856 - RNA, 23 nt, synthetic construct
  misc_feature 1..23: note = Targeting domain
  source 1..23: mol_type = other RNA
cctggtatcttctatggtgggag

SEQ ID NO: 857 - RNA, 24 nt, synthetic construct
  misc_feature 1..24: note = Targeting domain
  source 1..24: mol_type = other RNA
tcctggtatcttctatggtgggag

SEQ ID NO: 858 - RNA, 18 nt, synthetic construct
  misc_feature 1..18: note = Targeting domain
  source 1..18: mol_type = other RNA
tcctggtatcttctatgg

SEQ ID NO: 859 - RNA, 19 nt, synthetic construct
  misc_feature 1..19: note = Targeting domain
  source 1..19: mol_type = other RNA
gtcctggtatcttctatgg

SEQ ID NO: 860 - RNA, 21 nt, synthetic construct
  misc_feature 1..21: note = Targeting domain
  source 1..21: mol_type = other RNA
aagtcctggtatcttctatgg

SEQ ID NO: 861 - RNA, 22 nt, synthetic construct
  misc_feature 1..22: note = Targeting domain
  source 1..22: mol_type = other RNA
gaagtcctggtatcttctatgg

SEQ ID NO: 862 - RNA, 23 nt, synthetic construct
  misc_feature 1..23: note = Targeting domain
  source 1..23: mol_type = other RNA
agaagtcctggtatcttctatgg

SEQ ID NO: 863 - RNA, 24 nt, synthetic construct
  misc_feature 1..24: note = Targeting domain
  source 1..24: mol_type = other RNA
aagaagtcctggtatcttctatgg

SEQ ID NO: 864 - RNA, 18 nt, synthetic construct
  misc_feature 1..18: note = Targeting domain
  source 1..18: mol_type = other RNA
tggtatcttctatggtgg

SEQ ID NO: 865 - RNA, 19 nt, synthetic construct
  misc_feature 1..19: note = Targeting domain
  source 1..19: mol_type = other RNA
ctggtatcttctatggtgg

SEQ ID NO: 866 - RNA, 20 nt, synthetic construct
  misc_feature 1..20: note = Targeting domain
  source 1..20: mol_type = other RNA
cctggtatcttctatggtgg

SEQ ID NO: 867 - RNA, 21 nt, synthetic construct
  misc_feature 1..21: note = Targeting domain
  source 1..21: mol_type = other RNA
tcctggtatcttctatggtgg

SEQ ID NO: 868 - RNA, 22 nt, synthetic construct
  misc_feature 1..22: note = Targeting domain
  source 1..22: mol_type = other RNA
gtcctggtatcttctatggtgg

SEQ ID NO: 869 - RNA, 23 nt, synthetic construct
  misc_feature 1..23: note = Targeting domain
  source 1..23: mol_type = other RNA
agtcctggtatcttctatggtgg

SEQ ID NO: 870 - RNA, 24 nt, synthetic construct
  misc_feature 1..24: note = Targeting domain
  source 1..24: mol_type = other RNA
aagtcctggtatcttctatggtgg

SEQ ID NO: 871 - RNA, 18 nt, synthetic construct
  misc_feature 1..18: note = Targeting domain
  source 1..18: mol_type = other RNA
gtcctggtatcttctatg

SEQ ID NO: 872 - RNA, 19 nt, synthetic construct
  misc_feature 1..19: note = Targeting domain
  source 1..19: mol_type = other RNA
agtcctggtatcttctatg

SEQ ID NO: 873 - RNA, 20 nt, synthetic construct
  misc_feature 1..20: note = Targeting domain
  source 1..20: mol_type = other RNA
aagtcctggtatcttctatg

SEQ ID NO: 874 - RNA, 21 nt, synthetic construct
  misc_feature 1..21: note = Targeting domain
  source 1..21: mol_type = other RNA
gaagtcctggtatcttctatg

SEQ ID NO: 875 - RNA, 22 nt, synthetic construct
  misc_feature 1..22: note = Targeting domain
  source 1..22: mol_type = other RNA
agaagtcctggtatcttctatg

SEQ ID NO: 876 - RNA, 23 nt, synthetic construct
  misc_feature 1..23: note = Targeting domain
  source 1..23: mol_type = other RNA
aagaagtcctggtatcttctatg

SEQ ID NO: 877 - RNA, 24 nt, synthetic construct
  misc_feature 1..24: note = Targeting domain
  source 1..24: mol_type = other RNA
aaagaagtcctggtatcttctatg

SEQ ID NO: 878 - RNA, 18 nt, synthetic construct
  misc_feature 1..18: note = Targeting domain
  source 1..18: mol_type = other RNA
aaattggaatgactgaat

SEQ ID NO: 879 - RNA, 19 nt, synthetic construct
  misc_feature 1..19: note = Targeting domain
  source 1..19: mol_type = other RNA
aaaattggaatgactgaat

SEQ ID NO: 880 - RNA, 21 nt, synthetic construct
  misc_feature 1..21: note = Targeting domain
  source 1..21: mol_type = other RNA
gaaaaattggaatgactgaat

SEQ ID NO: 881 - RNA, 22 nt, synthetic construct
  misc_feature 1..22: note = Targeting domain
  source 1..22: mol_type = other RNA
agaaaaattggaatgactgaat

SEQ ID NO: 882 - RNA, 23 nt, synthetic construct
  misc_feature 1..23: note = Targeting domain
  source 1..23: mol_type = other RNA
gagaaaaattggaatgactgaat

SEQ ID NO: 883 - RNA, 24 nt, synthetic construct
  misc_feature 1..24: note = Targeting domain
  source 1..24: mol_type = other RNA
agagaaaaattggaatgactgaat

SEQ ID NO: 884 - RNA, 18 nt, synthetic construct
  misc_feature 1..18: note = Targeting domain
  source 1..18: mol_type = other RNA
taagcagcagtatcctct

SEQ ID NO: 885 - RNA, 19 nt, synthetic construct
  misc_feature 1..19: note = Targeting domain
  source 1..19: mol_type = other RNA
ttaagcagcagtatcctct

SEQ ID NO: 886 - RNA, 21 nt, synthetic construct
  misc_feature 1..21: note = Targeting domain
  source 1..21: mol_type = other RNA
aattaagcagcagtatcctct

SEQ ID NO: 887 - RNA, 22 nt, synthetic construct
  misc_feature 1..22: note = Targeting domain
  source 1..22: mol_type = other RNA
aaattaagcagcagtatcctct

SEQ ID NO: 888 - RNA, 23 nt, synthetic construct
  misc_feature 1..23: note = Targeting domain
  source 1..23: mol_type = other RNA
aaaattaagcagcagtatcctct

SEQ ID NO: 889 - RNA, 24 nt, synthetic construct
  misc_feature 1..24: note = Targeting domain
  source 1..24: mol_type = other RNA
aaaaattaagcagcagtatcctct

SEQ ID NO: 890 - RNA, 18 nt, synthetic construct
  misc_feature 1..18: note = Targeting domain
  source 1..18: mol_type = other RNA
cctggtatcttctatggt

SEQ ID NO: 891 - RNA, 19 nt, synthetic construct
  misc_feature 1..19: note = Targeting domain
  source 1..19: mol_type = other RNA
tcctggtatcttctatggt

SEQ ID NO: 892 - RNA, 21 nt, synthetic construct
  misc_feature 1..21: note = Targeting domain
  source 1..21: mol_type = other RNA
agtcctggtatcttctatggt

SEQ ID NO: 893 - RNA, 22 nt, synthetic construct
  misc_feature 1..22: note = Targeting domain
  source 1..22: mol_type = other RNA
aagtcctggtatcttctatggt

SEQ ID NO: 894 - RNA, 23 nt, synthetic construct
  misc_feature 1..23: note = Targeting domain
  source 1..23: mol_type = other RNA
gaagtcctggtatcttctatggt

SEQ ID NO: 895 - RNA, 24 nt, synthetic construct
  misc_feature 1..24: note = Targeting domain
  source 1..24: mol_type = other RNA
agaagtcctggtatcttctatggt

SEQ ID NO: 896 - RNA, 18 nt, synthetic construct
  misc_feature 1..18: note = Targeting domain
  source 1..18: mol_type = other RNA
aagcagcagtatcctctt

SEQ ID NO: 897 - RNA, 19 nt, synthetic construct
  misc_feature 1..19: note = Targeting domain
  source 1..19: mol_type = other RNA
taagcagcagtatcctctt

SEQ ID NO: 898 - RNA, 21 nt, synthetic construct
  misc_feature 1..21: note = Targeting domain
  source 1..21: mol_type = other RNA
attaagcagcagtatcctctt

SEQ ID NO: 899 - RNA, 22 nt, synthetic construct
  misc_feature 1..22: note = Targeting domain
  source 1..22: mol_type = other RNA
aattaagcagcagtatcctctt

SEQ ID NO: 900 - RNA, 23 nt, synthetic construct
  misc_feature 1..23: note = Targeting domain
  source 1..23: mol_type = other RNA
aaattaagcagcagtatcctctt

SEQ ID NO: 901 - RNA, 24 nt, synthetic construct
  misc_feature 1..24: note = Targeting domain
  source 1..24: mol_type = other RNA
aaaattaagcagcagtatcctctt

SEQ ID NO: 902 - DNA, 4758 bp, Homo sapiens
  misc_feature 2716..2719: note = HPFH deletion site (4 bp del -225 to -222)
  misc_feature 2748..2753: note = GATA1 binding motif
  misc_feature 2762..2767: note = GATA1 binding motif
  misc_feature 2791..2799: note = FKLF transcription factor binding motif
  misc_feature 2823..2830: note = CP1/Coup TFII binding motif
  misc_feature 2824..2836: note = HPFH deletion site (13 bp del -114 to -102)
  misc_feature 2991..2993: note = Start codon
  source 1..4758: mol_type = other DNA
tttaggaagtcaaggtttaggcagggatagccattctattttattaggggcaatactatttccaacggcatctggcttttctcagcccttgtgaggctctacagggaggttgaggtgttagagatcagagcaggaaacaggtttttctttccacggtaactacaatgaagtgatccttactttactaaggaacttttcattttaagtgttgacgcatgcctaaagaggtgaaattaatcccatacccttaagtctacagactggtcacagcatttcaaggaggagacctcattgtaagcttctagggaggtggggacttaggtgaaggaaatgagccagcagaagctcacaagtcagcatcagcgtgtcatgtctcagcagcagaacagcacggtcagatgaaaatatagtgtgaagaatttgtataacattaattgagaaggcagattcactggagttcttatataattgaaagttaatgcacgttaataagcaagagtttagtttaatgtgatggtgttatgaacttaacgcttgtgtctccagaaaattcacatgctgaatccccaactcccaattggctccatttgtgggggaggctttggaaaagtaatcaggtttagaggagctcatgagagcagatccccatcatagaattattttcctcatcagaagcagagagattagccatttctcttccttctggtgaggacacagtgggaagtcagccacctgcaacccaggaagagagccctgaccaggaaccagcagaaaagtgagaaaaaatcctgttgttgaagtcacccagtctatgctattttgttatagcaccttgcactaagtaaggcagatgaagaaagagaaaaaaataagcttcggtgttcagtggattagaaaccatgtttatctcaggtttacaaatctccacttgtcctctgtgtttcagaataaaataccaactctactactctcatctgtaagatgcaaatagtaagcctgagcccttctgtctaactttgaattctattttttcttcaacgtactttaggcttgtaatgtgtttatatacagtgaaatgtcaagttctttctttatatttctttctttcttttttttcctcagcctcagagttttccacatgcccttcctactttcaggaacttctttctccaaacgtcttctgcctggctccatcaaatcataaaggacccacttcaaatgccatcactcactaccatttcacaattcgcactttctttctttgtcctttttttttttagtaaaacaagtttataaaaaattgaaggaataaatgaatggctacttcataggcagagtagacgcaagggctactggttgccgatttttattgttatttttcaatagtatgctaaacaaggggtagattatttatgctgcccatttttagaccataaaagataacttcctgatgttgccatggcatttttttccttttaattttatttcatttcattttaatttcgaaggtacatgtgcaggatgtgcaggcttgttacatgggtaaatgtgtgtctttctggccttttagccatctgtatcaatgagcagatataagctttacacaggatcatgaaggatgaaagaatttcaccaatattataataatttcaatcaacctgatagcttaggggataaactaatttgaagatacagcttgcctccgataagccagaattccagagcttctggcattataatctagcaaggttagagatcatggatcactttcagagaaaaacaaaaacaaactaaccaaaagcaaaacagaaccaaaaaaccaccataaatacttcctaccctgttaatggtccaatatgtcagaaacagcactgtgttagaaataaagctgtctaaagtacactaatattcgagttataatagtgtgtggactattagtcaataaaaacaacccttgcctctttagagttgttttccatgtacacgcacatcttatgtcttagagtaagattccctgagaagtgaacctagcatttatacaagataattaattctaatccacagtacctgccaaagaacattctaccatcatctttactgagcatagaagagctacgccaaaaccctgggtcatcagccagcacacacacttatccagtggtaaatacacatcatctggtgtatacatacatacctgaatatggaatcaaatatttttctaagatgaaacagtcatgatttatttcaaataggtacggataagtagatattgaggtaagcattaggtcttatattatgtaacactaatctattactgcgctgaaactgtggctttatagaaattgttttcactgcactattgagaaattaagagataatggcaaaagtcacaaagagtatattcaaaaagaagtatagcactttttccttagaaaccactgctaactgaaagagactaagatttgtcccgtcaaaaatcctggacctatgcctaaaacacatttcacaatccctgaacttttcaaaaattggtacatgctttagctttaaactacaggcctcactggagctagagacaagaaggtaaaaaacggctgacaaaagaagtcctggtatcctctatgatgggagaaggaaactagctaaagggaagaataaattagagaaaaactggaatgactgaatcggaacaaggcaaaggctataaaaaaaattagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccacaaagcacctggatgatctcaagggcacctttgcccagctgagtgaactgcactgtgacaagctgcatgtggatcctgagaacttcaaggtgagtccaggagatgtttcagccctgttgcctttagtctcgaggcaacttagacaacggagtattgatctgagcacagcagggtgtgagctgtttgaagatactggggttgggggtgaagaaactgcagaggactaactgggctgagacccagtggtaatgttttagggcctaaggagtgcctctaaaaatctagatggacaattttgactttgagaaaagagaggtggaaatgaggaaaatgacttttctttattagattccagtagaaagaactttcatctttccctcatttttgttgttttaaaacatctatctggaggcaggacaagtatggtcgttaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgcgcgcgcgcgtgtgtgtgtgtgtgtcagcgtgtgtttcttttaacgtcttcagcctacaacatacagggttcatggtggcaagaagatagcaagatttaaattatggccagtgactagtgcttgaaggggaacaactacctgcatttaatgggaaggcaaaatctcaggctttgagggaagttaacataggcttgattctgggtggaagcttggtgtgtagttatctggaggccaggctggagctctcagctcactatgggttcatctttattgtctcctttcatctcaacagctcctgggaaatgtgctggtgaccgttttggcaatccatttcggcaaagaattcacccctgaggtgcaggcttcctggcagaagatggtgactgcagtggccagtgccctgtcctccagataccactgagctcactgcccatgattcagagctttcaaggataggctttattctgcaagcaatacaaataataaatctattctgctgagagatcacacatgattttcttcagctcttttttttacatctttttaaatatatgagccacaaagggtttatattgagggaagtgtgtatgtgtatttctgcatgcctgtttgtgtttgtggtgtgtgcatgctcctcatttatttttatatgagatgtgcattttgatgagcaaataaaagcagtaaagacacttgtacacgggagttctgcaagtgggagtaaatggtgtaggagaaatccg

SEQ ID NO: 903 - DNA, 4773 bp, Homo sapiens
  misc_feature 1233..1238: note = GATA1 binding site
  misc_feature 2672..2677: note = GATA1 binding site
  misc_feature 2686..2691: note = GATA1 binding site
  misc_feature 2715..2723: note = FKLF transcription factor binding motif
  misc_feature 2747..2754: note = CP1/Coup TFII binding motif
  misc_feature 2748..2760: note = HPFH deletion site (13 bp del -114 to -102)
  misc_feature 2915..2917: note = Start codon
  source 1..4773: mol_type = other DNA
ttatgtcattaccagagttaaaattctataatggcttctcactccctaccactgaggacaagtttatgtccttaggtttatgcttccctgaaacaataccacctgctattctccactttacatatcaacggcactggttctttatctaactctctggcacagcaggagtttgttttcttctgcttcagagctttgaatttactatttcagcttctaaactttatttggcaatgccttcccatggcagattccttctgtcattttgcctctgttcgaatactttctccttaatttcattcttagttaataatatctgaaattattttgttgtttaacttaattattaattttatgtatgttctacctagattataatcttcagaggaaagttttattctctgacttatttaacttaaatgcccactactttaaaaattatgacatttatttaacagatatttgctgaacaaatgtttgaaaatacatgggaaagaatgcttgaaaacacttgaaattgcttgtgtaaagaaacagttttatcagttaggatttaatcaatgtcagaagcaatgatataggaaaaatcgaggaataagacagttatggataaggagaaatcaacaaactcttaaaagatattgcctcaaaagcataagaggaaataagggtttatacatgacttttagaacactgccttggtttttggataaatggggaagttgtttgaaaacaggagggatcctagatattccttagtctgaggaggagcaattaagattcacttgtttagaggctgggagtggtggctcacgcctgtaatcccagaattttgggaggccaaggcaggcagatcacctgaggtcaagagttcaagaccaacctggccaacatggtgaaatcccatctctacaaaaatacaaaaattagacaggcatgatggcaagtgcctgtaatcccagctacttgggaggctgaggaaggagaattgcttgaacctggaaggcaggagttgcagtgagccgagatcataccactgcactccagcctgggtgacagaacaagactctgtctcaaaaaaaaaaaagagagattcaaaagattcacttgtttaggccttagcgggcttagacaccagtctctgacacattcttaaaggtcaggctctacaaatggaacccaaccagactctcagatatggccaaagatctatacacacccatctcacagatcccctatcttaaagagaccctaatttgggttcacctcagtctctataatctgtaccagcataccaataaaaatctttctcacccatccttagattgagagaagtcacttattattatgtgagtaactggaagatactgataagttgacaaatctttttctttcctttcttattcaacttttattttaacttccaaagaacaagtgcaatatgtgcagctttgttgcgcaggtcaacatgtatctttctggtcttttagccgcctaacactttgagcagatataagccttacacaggattatgaagtctgaaaggattccaccaatattattataattcctatcaacctgataggttaggggaaggtagagctctcctccaataagccagatttccagagtttctgacgtcataatctaccaaggtcatggatcgagttcagagaaaaaacaaaagcaaaaccaaacctaccaaaaaataaaaatcccaaagaaaaaataaagaaaaaaacagcatgaatacttcctgccatgttaagtggccaatatgtcagaaacagcactgagttacagataaagatgtctaaactacagtgacatcccagctgtcacagtgtgtggactattagtcaataaaacagtccctgcctcttaagagttgttttccatgcaaatacatgtcttatgtcttagaataagattccctaagaagtgaacctagcatttatacaagataattaattctaatccatagtatctggtaaagagcattctaccatcatctttaccgagcatagaagagctacaccaaaaccctgggtcatcagccagcacatacacttatccagtgataaatacacatcatcgggtgcctacatacatacctgaatataaaaaaaatacttttgctgagatgaaacaggcgtgatttatttcaaataggtacggataagtagatattgaagtaaggattcagtcttatattatattacataacattaatctattcctgcactgaaactgttgctttataggatttttcactacactaatgagaacttaagagataatggcctaaaaccacagagagtatattcaaagataagtatagcacttcttatttggaaaccaatgcttactaaatgagactaagacgtgtcccatcaaaaatcctggacctatgcctaaaacacatttcacaatccctgaacttttcaaaaattggtacatgctttaactttaaactacaggcctcactggagctacagacaagaaggtgaaaaacggctgacaaaagaagtcctggtatcttctatggtgggagaagaaaactagctaaagggaagaataaattagagaaaaattggaatgactgaatcggaacaaggcaaaggctataaaaaaaattaagcagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaaggtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgcgcgcgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagcttggtgtgtagttatctggaggccaggctggagctctcagctcactatgggttcatctttattgtctcctttcatctcaacagctcctgggaaatgtgctggtgaccgttttggcaatccatttcggcaaagaattcacccctgaggtgcaggcttcctggcagaagatggtgactggagtggccagtgccctgtcctccagataccactgagctcactgcccatgatgcagagctttcaaggataggctttattctgcaagcaatcaaataataaatctattctgctaagagatcacacatggttgtcttcagttctttttttatgtctttttaaatatatgagccacaaagggttttatgttgagggatgtgtttatgtgtatttatacatggctatgtgtgtttgtgtcatgtgcacactccacacttttttgtttacgttagatgtgggttttgatgagcaaataaaagaactaggcaataaagaaacttgtacatgggagttctgcaagtgggagtaaaaggtgcaggagaaatctggttggaagaaagacctctataggacaggactcctcagaaacagatgttttggaagagatggggaaaggttcagtgaa

SEQ ID NO: 904 - DNA, 87 bp, synthetic construct
  misc_feature 1..87: note = ssODN1 5' homology arm
  source 1..87: mol_type = other DNA
gggtgcttccttttattcttcatccctagccagccgccggcccctggcctcactggatactctaagactattggtcaagtttgcctt

SEQ ID NO: 905 - DNA, 89 bp, synthetic construct
  misc_feature 1..89: note = ssODN1 3' homology arm
  source 1..89: mol_type = other DNA
gtcaaggcaaggctggccaacccatgggtggagtttagccagggaccgtttcagacagatatttgcattgagatagtgtggggaagggg

SEQ ID NO: 906 - DNA, 176 bp, synthetic construct
  misc_feature 1..176: note = ssODN1
  source 1..176: mol_type = other DNA
gggtgcttccttttattcttcatccctagccagccgccggcccctggcctcactggatactctaagactattggtcaagtttgccttgtcaaggcaaggctggccaacccatgggtggagtttagccagggaccgtttcagacagatatttgcattgagatagtgtggggaagggg

SEQ ID NO: 907 - DNA, 87 bp, synthetic construct; identical to SEQ ID NO: 904
  misc_feature 1..87: note = PhTx ssODN1 5' homology arm
  misc_feature 1: note = Modified to contain phosphorothioate
  source 1..87: mol_type = other DNA

SEQ ID NO: 908 - DNA, 89 bp, synthetic construct; identical to SEQ ID NO: 905
  misc_feature 1..89: note = PhTx ssODN1 3' homology arm
  misc_feature 89: note = Modified to contain phosphorothioate
  source 1..89: mol_type = other DNA

SEQ ID NO: 909 - DNA, 176 bp, synthetic construct; identical to SEQ ID NO: 906
  misc_feature 1..176: note = PhTx ssODN1
  misc_feature 1: note = Modified to contain phosphorothioate
  misc_feature 176: note = Modified to contain phosphorothioate
  source 1..176: mol_type = other DNA

SEQ ID NO: 910 - DNA, 17 bp, synthetic construct
  misc_feature 1..17: note = targeting domain
  source 1..17: mol_type = other DNA
ggctattggtcaaggca

SEQ ID NO: 911 - DNA, 20 bp, synthetic construct
  misc_feature 1..20: note = targeting domain
  source 1..20: mol_type = other DNA
caaggctattggtcaaggca

SEQ ID NO: 912 - DNA, 17 bp, synthetic construct
  misc_feature 1..17: note = targeting domain
  source 1..17: mol_type = other DNA
tgccttgtcaaggctat

SEQ ID NO: 913 - DNA, 20 bp, synthetic construct
  misc_feature 1..20: note = targeting domain
  source 1..20: mol_type = other DNA
gtttgccttgtcaaggctat

SEQ ID NO: 914 - DNA, 17 bp, synthetic construct
  misc_feature 1..17: note = targeting domain
  source 1..17: mol_type = other DNA
gaccaatagccttgaca

SEQ ID NO: 915 - DNA, 20 bp, synthetic construct
  misc_feature 1..20: note = targeting domain
  source 1..20: mol_type = other DNA
cttgaccaatagccttgaca

SEQ ID NO: 916 - DNA, 17 bp, synthetic construct
  misc_feature 1..17: note = targeting domain
  source 1..17: mol_type = other DNA
gtcaaggctattggtca

SEQ ID NO: 917 - DNA, 20 bp, synthetic construct
  misc_feature 1..20: note = targeting domain
  source 1..20: mol_type = other DNA
cttgtcaaggctattggtca

SEQ ID NO: 918 - DNA, 17 bp, synthetic construct
  misc_feature 1..17: note = targeting domain
  source 1..17: mol_type = other DNA
tcaagtttgccttgtca

SEQ ID NO: 919 - DNA, 20 bp, synthetic construct
  misc_feature 1..20: note = targeting domain
  source 1..20: mol_type = other DNA
tggtcaagtttgccttgtca

SEQ ID NO: 920 - RNA, 20 nt, synthetic construct
  misc_feature 1..20: note = targeting domain plus PAM (NGG)
  source 1..20: mol_type = other RNA
ggctattggtcaaggcaagg

SEQ ID NO: 921 - RNA, 23 nt, synthetic construct
  misc_feature 1..23: note = targeting domain plus PAM (NGG)
  source 1..23: mol_type = other RNA
caaggctattggtcaaggcaagg

SEQ ID NO: 922 - RNA, 20 nt, synthetic construct
  misc_feature 1..20: note = targeting domain plus PAM (NGG)
  source 1..20: mol_type = other RNA
tgccttgtcaaggctattgg

SEQ ID NO: 923 - RNA, 23 nt, synthetic construct
  misc_feature 1..23: note = targeting domain plus PAM (NGG)
  source 1..23: mol_type = other RNA
gtttgccttgtcaaggctattgg

SEQ ID NO: 924 - RNA, 20 nt, synthetic construct
  misc_feature 1..20: note = targeting domain plus PAM (NGG)
  source 1..20: mol_type = other RNA
gaccaatagccttgacaagg

SEQ ID NO: 925 - RNA, 23 nt, synthetic construct
  misc_feature 1..23: note = targeting domain plus PAM (NGG)
  source 1..23: mol_type = other RNA
cttgaccaatagccttgacaagg

SEQ ID NO: 926 - RNA, 20 nt, synthetic construct
  misc_feature 1..20: note = targeting domain plus PAM (NGG)
  source 1..20: mol_type = other RNA
gtcaaggctattggtcaagg

SEQ ID NO: 927 - RNA, 23 nt, synthetic construct
  misc_feature 1..23: note = targeting domain plus PAM (NGG)
  source 1..23: mol_type = other RNA
cttgtcaaggctattggtcaagg

SEQ ID NO: 928 - RNA, 20 nt, synthetic construct
  misc_feature 1..20: note = targeting domain plus PAM (NGG)
  source 1..20: mol_type = other RNA
tcaagtttgccttgtcaagg

SEQ ID NO: 929 - RNA, 23 nt, synthetic construct
  misc_feature 1..23: note = targeting domain plus PAM (NGG)
  source 1..23: mol_type = other RNA
tggtcaagtttgccttgtcaagg

SEQ ID NO: 930 - DNA, 20 bp, synthetic construct
  misc_feature 1..20: note = targeting domain plus PAM (NGG)
  source 1..20: mol_type = other DNA
ggctattggtcaaggcaagg

SEQ ID NO: 931 - DNA, 23 bp, synthetic construct
  misc_feature 1..23: note = targeting domain plus PAM (NGG)
  source 1..23: mol_type = other DNA
caaggctattggtcaaggcaagg

SEQ ID NO: 932 - DNA, 20 bp, synthetic construct
  misc_feature 1..20: note = targeting domain plus PAM (NGG)
  source 1..20: mol_type = other DNA
tgccttgtcaaggctattgg

SEQ ID NO: 933 - DNA, 23 bp, synthetic construct
  misc_feature 1..23: note = targeting domain plus PAM (NGG)
  source 1..23: mol_type = other DNA
gtttgccttgtcaaggctattgg

SEQ ID NO: 934 - DNA, 20 bp, synthetic construct
  misc_feature 1..20: note = targeting domain plus PAM (NGG)
  source 1..20: mol_type = other DNA
gaccaatagccttgacaagg

SEQ ID NO: 935 - DNA, 23 bp, synthetic construct
  misc_feature 1..23: note = targeting domain plus PAM (NGG)
  source 1..23: mol_type = other DNA
cttgaccaatagccttgacaagg

SEQ ID NO: 936 - DNA, 20 bp, synthetic construct
  misc_feature 1..20: note = targeting domain plus PAM (NGG)
  source 1..20: mol_type = other DNA
gtcaaggctattggtcaagg

SEQ ID NO: 937 - DNA, 23 bp, synthetic construct
  misc_feature 1..23: note = targeting domain plus PAM (NGG)
  source 1..23: mol_type = other DNA
cttgtcaaggctattggtcaagg

SEQ ID NO: 938 - DNA, 20 bp, synthetic construct
  misc_feature 1..20: note = targeting domain plus PAM (NGG)
  source 1..20: mol_type = other DNA
tcaagtttgccttgtcaagg

SEQ ID NO: 939 - DNA, 23 bp, synthetic construct
  misc_feature 1..23: note = targeting domain plus PAM (NGG)
  source 1..23: mol_type = other DNA
tggtcaagtttgccttgtcaagg

SEQ ID NO: 940 - RNA, 17 nt, synthetic construct
  misc_feature 1..17: note = targeting domain
  source 1..17: mol_type = other RNA
agtatccagtgaggcca

SEQ ID NO: 941 - RNA, 20 nt, synthetic construct
  misc_feature 1..20: note = targeting domain
  source 1..20: mol_type = other RNA
ggcaaggctggccaacccat

SEQ ID NO: 942 - RNA, 20 nt, synthetic construct
  misc_feature 1..20: note = targeting domain
  source 1..20: mol_type = other RNA
tatttgcattgagatagtgt

SEQ ID NO: 943 - DNA, 17 bp, synthetic construct
  misc_feature 1..17: note = targeting domain
  source 1..17: mol_type = other DNA
agtatccagtgaggcca

SEQ ID NO: 944 - DNA, 20 bp, synthetic construct
  misc_feature 1..20: note = targeting domain
  source 1..20: mol_type = other DNA
ggcaaggctggccaacccat

SEQ ID NO: 945 - DNA, 20 bp, synthetic construct
  misc_feature 1..20: note = targeting domain
  source 1..20: mol_type = other DNA
tatttgcattgagatagtgt

SEQ ID NO: 946 - RNA, 20 nt, synthetic construct
  misc_feature 1..20: note = targeting domain plus PAM (NGG)
  source 1..20: mol_type = other RNA
agtatccagtgaggccaggg

SEQ ID NO: 947 - RNA, 23 nt, synthetic construct
  misc_feature 1..23: note = targeting domain plus PAM (NGG)
  source 1..23: mol_type = other RNA
ggcaaggctggccaacccatggg

SEQ ID NO: 948 - RNA, 23 nt, synthetic construct
  misc_feature 1..23: note = targeting domain plus PAM (NGG)
  source 1..23: mol_type = other RNA
tatttgcattgagatagtgtggg

SEQ ID NO: 949 - DNA, 20 bp, synthetic construct
  misc_feature 1..20: note = targeting domain plus PAM (NGG)
  source 1..20: mol_type = other DNA
agtatccagtgaggccaggg

SEQ ID NO: 950 - DNA, 23 bp, synthetic construct
  misc_feature 1..23: note = targeting domain plus PAM (NGG)
  source 1..23: mol_type = other DNA
ggcaaggctggccaacccatggg

SEQ ID NO: 951 - DNA, 23 bp, synthetic construct
  misc_feature 1..23: note = targeting domain plus PAM (NGG)
  source 1..23: mol_type = other DNA
tatttgcattgagatagtgtggg

SEQ ID NO: 952 - RNA, 20 nt, synthetic construct
  misc_feature 1..20: note = targeting domain
  source 1..20: mol_type = other RNA
ctaacagttgcttttatcac

SEQ ID NO: 953 - RNA, 20 nt, synthetic construct
  misc_feature 1..20: note = targeting domain
  source 1..20: mol_type = other RNA
gggcgtgggtggggtagaag

SEQ ID NO: 954 - RNA, 20 nt, synthetic construct
  misc_feature 1..20: note = targeting domain
  source 1..20: mol_type = other RNA
ctcttagacataacacacca

SEQ ID NO: 955 - RNA, 20 nt, synthetic construct
  misc_feature 1..20: note = targeting domain
  source 1..20: mol_type = other RNA
atcagaggccaaacccttcc

SEQ ID NO: 956 - DNA, 20 bp, synthetic construct
  misc_feature 1..20: note = targeting domain
  source 1..20: mol_type = other DNA
ctaacagttgcttttatcac

SEQ ID NO: 957 - DNA, 20 bp, synthetic construct
  misc_feature 1..20: note = targeting domain
  source 1..20: mol_type = other DNA
gggcgtgggtggggtagaag

SEQ ID NO: 958 - DNA, 20 bp, synthetic construct
  misc_feature 1..20: note = targeting domain
  source 1..20: mol_type = other DNA
ctcttagacataacacacca

SEQ ID NO: 959 - DNA, 20 bp, synthetic construct
  misc_feature 1..20: note = targeting domain
  source 1..20: mol_type = other DNA
atcagaggccaaacccttcc

SEQ ID NO: 960 - RNA, 23 nt, synthetic construct
  misc_feature 1..23: note = targeting domain plus PAM (NGG)
  source 1..23: mol_type = other RNA
ctaacagttgcttttatcacagg

SEQ ID NO: 961 - RNA, 23 nt, synthetic construct
  misc_feature 1..23: note = targeting domain plus PAM (NGG)
  source 1..23: mol_type = other RNA
gggcgtgggtggggtagaagagg

SEQ ID NO: 962 - RNA, 23 nt, synthetic construct
  misc_feature 1..23: note = targeting domain plus PAM (NGG)
  source 1..23: mol_type = other RNA
ctcttagacataacacaccaggg

SEQ ID NO: 963 - RNA, 23 nt, synthetic construct
  misc_feature 1..23: note = targeting domain plus PAM (NGG)
  source 1..23: mol_type = other RNA
atcagaggccaaacccttcctgg

SEQ ID NO: 964 - DNA, 23 bp, synthetic construct
  misc_feature 1..23: note = targeting domain plus PAM (NGG)
  source 1..23: mol_type = other DNA
ctaacagttgcttttatcacagg

SEQ ID NO: 965 - DNA, 23 bp, synthetic construct
  misc_feature 1..23: note = targeting domain plus PAM (NGG)
  source 1..23: mol_type = other DNA
gggcgtgggtggggtagaagagg

SEQ ID NO: 966 - DNA, 23 bp, synthetic construct
  misc_feature 1..23: note = targeting domain plus PAM (NGG)
  source 1..23: mol_type = other DNA
ctcttagacataacacaccaggg

SEQ ID NO: 967 - DNA, 23 bp, synthetic construct
  misc_feature 1..23: note = targeting domain plus PAM (NGG)
  source 1..23: mol_type = other DNA
atcagaggccaaacccttcctgg

SEQ ID NO: 968 - DNA, 115 bp, synthetic construct
  misc_feature 1..115: note = □DHS enhancer
  source 1..115: mol_type = other DNA
caactttgaagctagtctagtgcaagctaacagttgcttttatcacaggctccaggaagggtttggcctctgattagggtgggggcgtgggtggggtagaagaggactggcagac

SEQ ID NO: 969 - DNA, 801 bp, synthetic construct
  misc_feature 1..801: note = □DHS enhancer region including ˜500 bp upstream and ˜200 bp downstream
  source 1..801: mol_type = other DNA
aggtaccttttgtgtgtatgtgctgattgagggcccattgagaatattttgacttttagggaagctccaaactctcaaaccacagggatcacaacacatacgtgtgtctgttatgacgttatatgtaagcatcacaacaggcagagaatgtctgcaccccaccctggaaaacagcctgactgtgccccatgggcaaaccagactagtttatagggggttctactctgaggtactgatggaccttgggtgctattcctgtgataaggaaggcagctagacaggacttgggagttatctgtagtgagatggctgaaaagcgatacagggctggctctatgccccaggtgtgcataagtaagagcagatagctgattccagtgcaaagtccatacaggtaataacataggccagaaaagagatatggcatctactcttagacataacacaccagggtcaatacaactttgaagctagtctagtgcaagctaacagttgcttttatcacaggctccaggaagggtttggcctctgattagggtgggggcgtgggtggggtagaagaggactggcagacctctccatcggtggccgtttgcccaggggggcctctttcggaaggctctcttggtgatggagaattggattttatttctcaatgggaatgaaataatttgtatgccatgccgtgtggactcccaaaattgtaaaggaggtgaagcttcccctgtctgcactctcccctcctcataattgtccatttttcatctgtcgggctgtccacccatccatcacatataggca

SEQ ID NO: 970 - RNA, 100 nt, synthetic construct
  misc_feature 1..100: note = Unmodified Sp35 gRNA (OLI7066)
  source 1..100: mol_type = other RNA
cttgtcaaggctattggtcagttttagagctagaaatagcaagttaaaataaggctagtccgttatcaacttgaaaaagtggcaccgagtcggtgctttt

SEQ ID NO: 971 - RNA, 100 nt, synthetic construct; identical to SEQ ID NO: 970
  misc_feature 1..100: note = Sp35 gRNA w/ 5' PS (OLI8568)
  misc_feature 1: note = Modified to contain phosphorothioate
  misc_feature 2: note = Modified to contain phosphorothioate
  misc_feature 3: note = Modified to contain phosphorothioate
  source 1..100: mol_type = other RNA

SEQ ID NO: 972 - RNA, 100 nt, synthetic construct; identical to SEQ ID NO: 970
  misc_feature 1..100: note = Sp35 gRNA w/ 5' OMe (OLI8569)
  misc_feature 1: note = Modified to contain 2-o-methyl
  misc_feature 2: note = Modified to contain 2-o-methyl
  misc_feature 3: note = Modified to contain 2-o-methyl
  source 1..100: mol_type = other RNA

SEQ ID NO: 973 - RNA, 100 nt, synthetic construct; identical to SEQ ID NO: 970
  misc_feature 1..100: note = Sp35 gRNA w/ 3' PS/OMe (OLI8396)
  misc_feature 97: note = Modified to contain phosphorothioate, 2-o-methyl
  misc_feature 98: note = Modified to contain phosphorothioate, 2-o-methyl
  misc_feature 99: note = Modified to contain phosphorothioate, 2-o-methyl
  source 1..100: mol_type = other RNA

SEQ ID NO: 974 - RNA, 100 nt, synthetic construct; identical to SEQ ID NO: 970
  misc_feature 1..100: note = Sp35 gRNA w/ 3' PS/OMe (OLI8395)
  misc_feature 1: note = Modified to contain phosphorothioate, 2-o-methyl
  misc_feature 2: note = Modified to contain phosphorothioate, 2-o-methyl
  misc_feature 3: note = Modified to contain phosphorothioate, 2-o-methyl
  source 1..100: mol_type = other RNA

SEQ ID NO: 975 - RNA, 100 nt, synthetic construct; identical to SEQ ID NO: 970
  misc_feature 1..100: note = Sp35 gRNA w/ 5' and 3' PS/OMe (OLI8394)
  misc_feature 1: note = Modified to contain phosphorothioate, 2-o-methyl
  misc_feature 2: note = Modified to contain phosphorothioate, 2-o-methyl
  misc_feature 3: note = Modified to contain phosphorothioate, 2-o-methyl
  misc_feature 97: note = Modified to contain phosphorothioate, 2-o-methyl
  misc_feature 98: note = Modified to contain phosphorothioate, 2-o-methyl
  misc_feature 99: note = Modified to contain phosphorothioate, 2-o-methyl
  source 1..100: mol_type = other RNA

SEQ ID NO: 976 - RNA, 97 nt, synthetic construct
  misc_feature 1..97: note = Sp35 gRNA 97mer (OLI8018)
  source 1..97: mol_type = other RNA
gtcaaggctattggtcagttttagagctagaaatagcaagttaaaataaggctagtccgttatcaacttgaaaaagtggcaccgagtcggtgctttt

SEQ ID NO: 977 - RNA, 98 nt, synthetic construct
  misc_feature 1..98: note = Sp35 gRNA 98mer (OLI8019)
  source 1..98: mol_type = other RNA
tgtcaaggctattggtcagttttagagctagaaatagcaagttaaaataaggctagtccgttatcaacttgaaaaagtggcaccgagtcggtgctttt

SEQ ID NO: 978 - RNA, 99 nt, synthetic construct
  misc_feature 1..99: note = Sp35 gRNA 99mer (OLI8020)
  source 1..99: mol_type = other RNA
ttgtcaaggctattggtcagttttagagctagaaatagcaagttaaaataaggctagtccgttatcaacttgaaaaagtggcaccgagtcggtgctttt

SEQ ID NO: 979 - RNA, 100 nt, synthetic construct
  misc_feature 1..100: note = Sp35 gRNA w/ 5' C->A (OLI8397)
  source 1..100: mol_type = other RNA
attgtcaaggctattggtcagttttagagctagaaatagcaagttaaaataaggctagtccgttatcaacttgaaaaagtggcaccgagtcggtgctttt

SEQ ID NO: 980 - DNA, 100 bp, synthetic construct
  misc_feature 1..100: note = Unmodified Sp35 gRNA (OLI7066)
  source 1..100: mol_type = other DNA
cttgtcaaggctattggtcagttttagagctagaaatagcaagttaaaataaggctagtccgttatcaacttgaaaaagtggcaccgagtcggtgctttt

SEQ ID NO: 981 - DNA, 100 bp, synthetic construct; identical to SEQ ID NO: 980
  misc_feature 1..100: note = Sp35 gRNA w/ 5' PS (OLI8568)
  misc_feature 1: note = Modified to contain phosphorothioate
  misc_feature 2: note = Modified to contain phosphorothioate
  misc_feature 3: note = Modified to contain phosphorothioate
  source 1..100: mol_type = other DNA

SEQ ID NO: 982 - DNA, 100 bp, synthetic construct; identical to SEQ ID NO: 980
  misc_feature 1..100: note = Sp35 gRNA w/ 5' OMe (OLI8569)
  misc_feature 1: note = Modified to contain 2-o-methyl
  misc_feature 2: note = Modified to contain 2-o-methyl
  misc_feature 3: note = Modified to contain 2-o-methyl
  source 1..100: mol_type = other DNA

SEQ ID NO: 983 - DNA, 100 bp, synthetic construct; identical to SEQ ID NO: 980
  misc_feature 1..100: note = Sp35 gRNA w/ 3' PS/OMe (OLI8396)
  misc_feature 97: note = Modified to contain phosphorothioate, 2-o-methyl
  misc_feature 98: note = Modified to contain phosphorothioate, 2-o-methyl
  misc_feature 99: note = Modified to contain phosphorothioate, 2-o-methyl
  source 1..100: mol_type = other DNA

SEQ ID NO: 984 - DNA, 100 bp, synthetic construct; identical to SEQ ID NO: 980
  misc_feature 1..100: note = Sp35 gRNA w/ 3' PS/OMe (OLI8395)
  misc_feature 97: note = Modified to contain phosphorothioate, 2-o-methyl
  misc_feature 98: note = Modified to contain phosphorothioate, 2-o-methyl
  misc_feature 99: note = Modified to contain phosphorothioate, 2-o-methyl
  source 1..100: mol_type = other DNA

SEQ ID NO: 985 - DNA, 100 bp, synthetic construct; identical to SEQ ID NO: 980
  misc_feature 1..100: note = Sp35 gRNA w/ 5' and 3' PS/OMe (OLI8394)
  misc_feature 1: note = Modified to contain phosphorothioate, 2-o-methyl
  misc_feature 2: note = Modified to contain phosphorothioate, 2-o-methyl
  misc_feature 3: note = Modified to contain phosphorothioate, 2-o-methyl
  misc_feature 97: note = Modified to contain phosphorothioate, 2-o-methyl
  misc_feature 98: note = Modified to contain phosphorothioate, 2-o-methyl
  misc_feature 99: note = Modified to contain phosphorothioate, 2-o-methyl
  source 1..100: mol_type = other DNA

SEQ ID NO: 986 - DNA, 97 bp, synthetic construct
  misc_feature 1..97: note = Sp35 gRNA 97mer (OLI8018)
  source 1..97: mol_type = other DNA
gtcaaggctattggtcagttttagagctagaaatagcaagttaaaataaggctagtccgttatcaacttgaaaaagtggcaccgagtcggtgctttt

SEQ ID NO: 987 - DNA, 98 bp, synthetic construct
  misc_feature 1..98: note = Sp35 gRNA 98mer (OLI8019)
  source 1..98: mol_type = other DNA
tgtcaaggctattggtcagttttagagctagaaatagcaagttaaaataaggctagtccgttatcaacttgaaaaagtggcaccgagtcggtgctttt

SEQ ID NO: 988 - DNA, 99 bp, synthetic construct
  misc_feature 1..99: note = Sp35 gRNA 99mer (OLI8020)
  source 1..99: mol_type = other DNA
ttgtcaaggctattggtcagttttagagctagaaatagcaagttaaaataaggctagtccgttatcaacttgaaaaagtggcaccgagtcggtgctttt

SEQ ID NO: 989 - DNA, 100 bp, synthetic construct
  misc_feature 1..100: note = Sp35 gRNA w/ 5' C->A (OLI8397))
  source 1..100: mol_type = other DNA
attgtcaaggctattggtcagttttagagctagaaatagcaagttaaaataaggctagtccgttatcaacttgaaaaagtggcaccgagtcggtgctttt

SEQ ID NO: 990 - DNA, 180 bp, synthetic construct
  misc_feature 1..180: note = PhTx ssODN 13nt - positive strand (OLI16414)
  misc_feature 1: note = Modified to contain phosphorothioate
  misc_feature 179: note = Modified to contain phosphorothioate
  source 1..180: mol_type = other DNA
aagggtgcttccttttattcttcatccctagccagccgccggcccctggcctcactggatactctaagactattggtcaagtttgccttgtcaaggcaaggctggccaacccatgggtggagtttagccagggaccgtttcagacagatatttgcattgagatagtgtggggaaggggcc

SEQ ID NO: 991 - DNA, 90 bp, synthetic construct
  misc_feature 1..90: note = 5' homology arm: PhTx ssODN 13nt - positive strand (OLI16414)
  misc_feature 1: note = Modified to contain phosphorothioate
  source 1..90: mol_type = other DNA
aagggtgcttccttttattcttcatccctagccagccgccggcccctggcctcactggatactctaagactattggtcaagtttgccttg

SEQ ID NO: 992 - DNA, 90 bp, synthetic construct
  misc_feature 1..90: note = 3' homology arm: PhTx ssODN 13nt - positive strand (OLI16414)
  misc_feature 89: note = Modified to contain phosphorothioate
  source 1..90: mol_type = other DNA
tcaaggcaaggctggccaacccatgggtggagtttagccagggaccgtttcagacagatatttgcattgagatagtgtggggaaggggcc

SEQ ID NO: 993 - DNA, 180 bp, synthetic construct
  misc_feature 1..180: note = PhTx ssODN 13nt - negative strand (OLI16412)
  misc_feature 1: note = Modified to contain phosphorothioate
  misc_feature 179: note = Modified to contain phosphorothioate
  source 1..180: mol_type = other DNA
ggccccttccccacactatctcaatgcaaatatctgtctgaaacggtccctggctaaactccacccatgggttggccagccttgccttgacaaggcaaacttgaccaatagtcttagagtatccagtgaggccaggggccggcggctggctagggatgaagaataaaaggaagcaccctt

SEQ ID NO: 994 - DNA, 90 bp, synthetic construct
  misc_feature 1..90: note = 5' homology arm: PhTx ssODN 13nt - negative strand (OLI16412)
  misc_feature 1: note = Modified to contain phosphorothioate
  source 1..90: mol_type = other DNA
ggccccttccccacactatctcaatgcaaatatctgtctgaaacggtccctggctaaactccacccatgggttggccagccttgccttga

SEQ ID NO: 995 - DNA, 90 bp, synthetic construct
  misc_feature 1..90: note = 3' homology arm: PhTx ssODN 13nt - negative strand (OLI16412)
  modified_base 89: mod_base = OTHER; note = Modified to contain phosphorothioate
  source 1..90: mol_type = other DNA
caaggcaaacttgaccaatagtcttagagtatccagtgaggccaggggccggcggctggctagggatgaagaataaaaggaagcaccctt

SEQ ID NO: 996 - DNA, 125 bp, synthetic construct
  misc_feature 1..125: note = synthetic
  source 1..125: mol_type = other DNA
gaggtctgccagtcctcttctaccccacccacgcccccaccctaatcagaggccaaacccttcctggagcctgtgataaaagcaactgttagcttgcactagactagcttcaaagttgtattgac